(19) United States
(12) Patent Application Publication
(10) Pub. No.: US 2026/0217791 A1
(43) Pub. Date: Jul. 30, 2026

(54) AGENTS AND METHODS FOR TREATING COMPLEMENT DISEASES

(71) Applicant: Complement Therapeutics Limited, London (GB)

(72) Inventors: Simon J. Clark, White City, London (GB); Mustafa Munye, White City, London (GB); Sonika Rathi, White City, London (GB)

(73) Assignee: Complement Therapeutics Limited, London (GB)

(21) Appl. No.: 19/145,261

(22) PCT Filed: Jan. 5, 2024
(86) PCT No.: PCT/EP2024/050230

§ 371 Date: Jul. 2, 2025

(30) Foreign Application Priority Data
Jan. 5, 2023 (GB) 2300147.2

(51) Int. Cl. C07K 14/705; A61K 48/00; C07K 14/005; C12N 15/86

(52) U.S. Cl. CPC C07K 14/70596; A61K 48/0033; C07K 14/005; C12N 15/86; C12N 2750/14122; C12N 2750/14143; C12N 2830/48; C12N 2830/50

(57) ABSTRACT

Polypeptide and nucleic acid agents for treating diseases and conditions associated with complement are disclosed. Also disclosed are expression vectors, compositions and cells comprising the agents, and methods using the agents, e.g. for treatment.

### RELATED APPLICATIONS

This application is a national stage filing under 35 U.S.C. § 371 of international application number PCT/EP2024/050230, filed Jan. 5, 2024, which claims the benefit of United Kingdom application number GB 2300147.2, filed Jan. 5, 2023, each of which is herein incorporated by reference in its entirety.

### REFERENCE TO AN ELECTRONIC SEQUENCE LISTING

The content of the electronic sequence listing (C176770007US00-SEQ-KZM.xml; Size: 77,186 bytes; and Date of Creation: Jul. 2, 2025) is herein incorporated by reference in its entirety.

### FIELD OF THE INVENTION

The present invention relates to the fields of molecular biology and medicine. More specifically, the present invention relates to agents and methods for treating complement-related diseases.

### BACKGROUND

The complement system contributes to innate host immune defence by assisting in the rapid recognition and elimination of microbial intruders. However, dysregulation of complement can contribute to inflammatory, immune-related, and age-related conditions. As a result inappropriate regulation of the complement system has been implicated in a wide variety of diseases in humans e.g. diseases of the eye and kidney, as well as neurological diseases and cancer (Morgan, B. P., Semin Immunopathol, 2018. 40 (1): p. 113-124; Halbgebauer, R., et al., Semin Immunol, 2018. 37: p. 12-20; Ma, Y., et al., Aging Dis, 2019. 10 (2): p. 429-462; and Kleczko, E. K., et al., Front Immunol, 2019. 10: p. 954).

Complement pathway activation and control is regulated by a complex interplay between pathway activators and inhibitors. These activators and inhibitors are commonly enzymes which cleave and inactivate complement molecules on biological surfaces and/or in solution to maintain steady regulation of complement activating species. The complement pathways are in a constant state of flux and balance, and disturbances to this balance can lead to inappropriate activation and the consequences above.

One activating molecule is complement component 3 (C3), a member of the alternative complement pathway and amplification loop. C3 comprises a β chain and an a′ chain which associate through interchain disulphide bonds. During complement activation, C3 is cleaved to generate two functional fragments, C3a and C3b. C3a is a potent anaphylatoxin. Deposition of C3b on biological surfaces, e.g. extracellular matrix and cell surfaces, is an activating mechanism of the alternative pathway. C3b is a potent opsonin, targeting pathogens, antibody-antigen immune complexes and apoptotic cells for phagocytosis by phagocytes and NK cells. Surface-linked C3b also reacts with other complement proteins to form active convertase enzymes that are able to produce further (surface-attachable) C3b molecules, serving to activate and amplify complement responses (Clark, S. J., et al., J Immunol, 2014. 193 (10): p. 4962-70). C3b associates with Factor B to form the C3bBb-type C3 convertase and with C3bBb to form the C3bBb3b-type C5 convertase. Proteolytic cleavage of C3 also produces C3a and C3b through the classical complement pathway and the lectin pathway.

Insufficient control of C3 convertases results in massive production of C3b and C3a molecules and a shift of the complement cascade to its terminal lytic pathway. This produces the potent anaphylatoxin, C5a, and the cell lytic protein complex termed the membrane attack complex; both providing strong inflammatory signals (Clark, S. J., et al., supra). This ultimately leads to cell/tissue destruction and a local inflammatory response.

C3b activation of complement is regulated by complement protein factor I (FI). FI prevents complement activation by cleaving C3b to a proteolytically-inactive form, designated iC3b, which is unable to participate in convertase assembly, and further to downstream products iC3dg, C3dg and finally C3d. With every cleavage by FI, the C3b breakdown products have reduced affinity for receptors expressed on the surface of engaging innate immune cells. Despite iC3b being unable to contribute to the amplification loop of complement, and therefore often being considered the end of complement activation, it remains a potent opsonin and continues to interact with accumulated immune cells.

FI requires the presence of a cofactor, examples of which include the blood-borne Factor H (FH) protein or factor-H like protein 1 (FHL-1), and the membrane-bound surface co-factor ‘complement factor 1’ (CR1; CD35) or membrane co-factor protein (MCP; CD46).

### SUMMARY

The present invention provides polynucleotides, expression cassettes and vectors (among other articles) which encode for and are capable of expressing polypeptides that comprise a portion of complement receptor 1 (CR1) that is capable of binding to C3b. In some embodiments the encoded/expressed polypeptides comprise or consist of CCP (SCR/Sushi) domains 8-10 or 15-17 of human CR1.

In one aspect the invention provides a polynucleotide comprising, in 5′ to 3′ or 3′ to 5′ order:
(i) a first nucleotide sequence comprising a promoter;
(ii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO: 6 or 5; and
(iii) a polyadenylation signal sequence.

In some embodiments, the polynucleotide comprises a 5′ inverted terminal repeat (ITR) and/or a 3′ ITR. In some embodiments, at least one of the ITRs is an AAV1, AAV2, AAV3, AAV4, AAV5, AAV6, AAB7, AAV8, AAV9, AAV10, AAV11, AAV12, AAV-DJ, AAV-DJ/8, AAV-rh 10, AAVrh.39, AAV-retro, AAV-PHP.B, AAV8-PHP.eB or AAV-PHP.S ITR. In some embodiments, at least one of the ITRs in an AAV2 ITR.

In some embodiments, the 5′ ITR comprises or consists of a sequence having at least 80% sequence identity to SEQ ID NO: 25 or 26. In some embodiments, the 3′ ITR comprises or consists of a sequence having at least 80% sequence identity to SEQ ID NO: 27 or 28.

In some embodiments, the 5′ ITR and/or the 3′ ITR is a mutant ITR. In some embodiments, the 5′ ITR comprises or consists of a sequence having at least 80% sequence identity to SEQ ID NO: 26 and/or the 3′ ITR comprises or consists of a sequence having at least 80% sequence identity to SEQ ID NO: 28.

In some embodiments, the promoter is a mammalian promoter, i.e. is capable of expressing the second nucleotide sequence or transgene in a mammalian cell. In some embodiments, the promoter is a human promoter, i.e. is capable of expressing the second nucleotide sequence or transgene in a human cell. In some embodiments, the promoter is selected from: a CBh promoter, a CAG promoter, a truncated CAG promoter, a CMV promoter, a SV40 promoter, a UBC promoter, a EF1A promoter, and a PGK promoter.

In some embodiments, the promoter comprises or consists of a sequence having at least 80% sequence identity to any one of SEQ ID NO: 14, 15, 16, 17 or 18.

In some embodiments, the polyadenylation signal sequence is derived from bovine growth hormone (bGH), human growth hormone (hGH) or SV40. In some embodiments, the polyadenylation signal sequence comprises or consists of a sequence having at least 80% sequence identity to any one of SEQ ID NO: 19, 20 or 21.

In some embodiments, the polynucleotide comprises a post-transcriptional regulatory element (PRE) between sequences (ii) and (iii). In some embodiments, the polynucleotide comprises a woodchuck hepatitis virus (WHV) post-transcriptional regulatory element (WPRE) between sequences (ii) and (iii). In some embodiments, the WPRE is a mutant WPRE e.g. as described herein. In some embodiments, the WPRE comprises or consists of a sequence having at least 80% sequence identity to SEQ ID NO:22.

In some embodiments, the polynucleotide comprises a stuffer sequence, i.e. an inert sequence to increase the size of the expression cassette to optimise vector stability and transgene expression. In some embodiments, the stuffer sequence is positioned between the second nucleotide sequence (ii) and the polyadenylation signal sequence (iii). In some embodiments, the polynucleotide comprises a third nucleotide sequence between sequences (ii) and (iii), wherein the third nucleotide sequence comprises or consists of a stuffer sequence.

In some embodiments, the stuffer sequence is derived from an intron of a mammalian gene. In some embodiments, the intron lacks the splice acceptor and splice donor sequences. That is, the splice acceptor and donor sequences have been removed. In some embodiments, the stuffer sequence is derived from a VMD2 (BEST1) intron. In some embodiments, the stuffer sequence comprises or consists of a sequence having at least 80% sequence identity to SEQ ID NO:23. In some embodiments, the stuffer sequence is derived from a RLBP1 intron. In some embodiments, the stuffer sequence comprises or consists of a sequence having at least 80% sequence identity to SEQ ID NO:24.

The present invention provides the following polynucleotides:

A polynucleotide comprising or consisting of (e.g. in 5′ to 3′ or 3′ to 5′ order):
(i) an ITR, optionally comprising or consisting of a sequence having at least 80% sequence identity to SEQ ID NO:26 or 28;
(ii) a first nucleotide sequence comprising a promoter, optionally comprising or consisting of a sequence having at least 80% sequence identity to SEQ ID NO:14;
(iii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5;
(iv) a polyadenylation signal sequence, optionally comprising or consisting of a sequence having at least 80% sequence identity to SEQ ID NO:19; and
(v) an ITR, optionally comprising or consisting of a sequence having at least 80% sequence identity to SEQ ID NO:28 or 26.

A polynucleotide comprising or consisting of (e.g. in 5′ to 3′ or 3′ to 5′ order):
(i) an ITR, optionally comprising or consisting of a sequence having at least 80% sequence identity to SEQ ID NO:25 or 27;
(ii) a first nucleotide sequence comprising a promoter, optionally comprising or consisting of a sequence having at least 80% sequence identity to SEQ ID NO:16;
(iii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5;
(iv) a post-transcription regulatory element, preferably a WPRE, optionally comprising or consisting of a sequence having at least 80% sequence identity to SEQ ID NO:22;
(v) a polyadenylation signal sequence, optionally comprising or consisting of a sequence having at least 80% sequence identity to SEQ ID NO:20;
(vi) a stuffer sequence, optionally comprising or consisting of a sequence having at least 80% sequence identity to SEQ ID NO:23; and
(vii) an ITR, optionally comprising or consisting of a sequence having at least 80% sequence identity to SEQ ID NO:27 or 25.

A polynucleotide comprising or consisting of (e.g. in 5′ to 3′ or 3′ to 5′ order):
(i) an ITR, optionally comprising or consisting of a sequence having at least 80% sequence identity to SEQ ID NO:25 or 27;
(ii) a first nucleotide sequence comprising a promoter, optionally comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5;
(iv) a post-transcription regulatory element, preferably a WPRE, optionally comprising or consisting of a sequence having at least 80% sequence identity to SEQ ID NO:22;
(v) a polyadenylation signal sequence, optionally comprising or consisting of a sequence having at least 80% sequence identity to SEQ ID NO:20; and
(vi) an ITR, optionally comprising or consisting of a sequence having at least 80% sequence identity to SEQ ID NO:27 or 25.

A polynucleotide comprising or consisting of (e.g. in 5′ to 3′ or 3′ to 5′ order):
(i) an ITR, optionally comprising or consisting of a sequence having at least 80% sequence identity to SEQ ID NO:25 or 27;
(ii) a first nucleotide sequence comprising a promoter, optionally comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5;
(iv) a post-transcription regulatory element, preferably a WPRE, optionally comprising or consisting of a sequence having at least 80% sequence identity to SEQ ID NO:22;
(v) a polyadenylation signal sequence, optionally comprising or consisting of a sequence having at least 80% sequence identity to SEQ ID NO:20;
(vi) a stuffer sequence, optionally comprising or consisting of a sequence having at least 80% sequence identity to SEQ ID NO:23; and
(vii) an ITR, optionally comprising or consisting of a sequence having at least 80% sequence identity to SEQ ID NO:27 or 25.

A polynucleotide comprising or consisting of (e.g. in 5′ to 3′ or 3′ to 5′ order):
(i) an ITR, optionally comprising or consisting of a sequence having at least 80% sequence identity to SEQ ID NO:25 or 27;
(ii) a first nucleotide sequence comprising a promoter, optionally comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO:6 or 5;
(iv) a polyadenylation signal sequence, optionally comprising or consisting of a sequence having at least 80% sequence identity to SEQ ID NO:20;
(v) a stuffer sequence, optionally comprising or consisting of a sequence having at least 80% sequence identity to SEQ ID NO:23; and
(vi) an ITR, optionally comprising or consisting of a sequence having at least 80% sequence identity to SEQ ID NO:27 or 25.

In some embodiments there is provided a polynucleotide that comprises or consists of a sequence having at least 80% sequence identity to one or more of SEQ ID NO:29, 30, 31, 32 or 33.

The present invention provides a vector or plasmid comprising a polynucleotide described herein.

The present invention provides a recombinant AAV (rAAV) comprising:
(i) A polynucleotide described herein; and
(ii) At least one AAV capsid protein.

In some embodiments, the rAAV is a self-complementary AAV (scAAV).

In some embodiments, the at least one AAV capsid protein has a tropism for eye cells and/or kidney cells. In some embodiments, the at least one AAV capsid protein is selected from AAV1, AAV2, AAV3, AAV4, AAV5, AAV6, AAB7, AAV8, AAV9, AAV10, AAV11, AAV12, AAV-DJ, AAV-DJ/8, AAV-rh10, AAVrh.39, AAV-retro, AAV-PHP.B, AAV8-PHP.eB, AAV-PHP.S, AAV-Anc80, AAV2.5, R100, AAV2.7m8, AAV-LK05 and AAVtYF, or a variant or hybrid thereof. In some embodiments, the at least one AAV capsid protein is selected from AAV2 and AAV8. In some embodiments, the polynucleotide comprises at least one AAV2 ITR and the at least one capsid protein is selected from AAV2 and AAV8.

In some embodiments, the rAAV is AAV2/2, AAV2/8, AAV2/1, AAV2/6, AAV2/5, AAV2/7, AAV2/9, rAAV2/8 Y733F, rAAV2/2 Y444F, or rAAV2/2 (containing mutations Y252F, Y272F, Y444F, Y500F, Y704F, Y730F).

Also provided is a pharmaceutical composition comprising the polynucleotide, vector, plasmid or rAAV described herein. In some embodiments, the pharmaceutical composition comprises a pharmaceutically acceptable excipient.

Also provided is a cell comprising or expressing the polynucleotide, vector, plasmid or rAAV described herein. In some embodiments, the cell is a human cell. In some embodiments, the cell is a cell of the eye, kidney or CNS.

The present invention provides a polynucleotide, vector, plasmid, rAAV, composition or cell according to the present disclosure for use as a medicament. The present invention provides a polynucleotide, vector, plasmid, rAAV, composition or cell according to the present disclosure for use in a method of treating a complement-related disorder in a subject.

Also provided is a method of treating a complement-related disorder in a subject, the method comprising administering a therapeutically effective amount of a polynucleotide, vector, plasmid, rAAV, composition or cell according to the present disclosure.

Also provided is the use of a polynucleotide, vector, plasmid, rAAV, composition or cell according to the present disclosure in the manufacture of a medicament for treating or preventing a complement-related disorder in a subject.

In some embodiments, the treatment comprises expressing the transgene in at least one cell of the subject. In some embodiments, the treatment comprises expressing a polypeptide encoded by the transgene in at least one cell of the subject. In some embodiments, the treatment comprises expressing a polypeptide comprising or consisting of CCPs 8-10 of CR1. In some embodiments, the treatment comprises administering the polynucleotide, vector, plasmid, rAAV, composition or cell according to the present disclosure to a subject such that the transgene and/or polypeptide is expressed in at least one cell of the subject.

In some embodiments, the polynucleotide, vector, plasmid, rAAV, composition or cell is administered by subretinal, intraocular, intravitreal, intraconjunctival, suprachoroidal, choroidal, or intravenous administration.

In some embodiments, the complement-related disorder is selected from: macular degeneration, Age-related Macular Degeneration (AMD), Geographic Atrophy (‘dry’ or non-exudative AMD), early AMD, early onset macular degeneration (EOMD), intermediate AMD, late/advanced AMD, ‘wet’ (neovascular or exudative) AMD, choroidal neovascularisation (CNV), retinal dystrophy, glaucoma (open-angle or closed-angle), neuromyelitis optica (neuromyelitis optica spectrum disorder (NMOSD)), diabetic retinopathy, Stargardt disease, autoimmune uveitis, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), glomerular diseases, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), ANCA vasculitis, autoimmune hemolytic anemia (AIHA), systemic lupus erythematosus (SLE), cutaneous lupus erythematosus (CLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, skin diseases e.g. inflammatory skin diseases, atherosclerosis, inflammatory disease, inflammatory bowel disease (IBD), autoimmune disease, neurodegeneration/neurodegenerative disease, dementia, frontotemporal dementia, multiple sclerosis (MS), stroke, Parkinson's disease, Alzheimer's disease, Lewy body disease, Amyotrophic lateral sclerosis (ALS), Huntington's disease, epilepsy, schizophrenia, acute brain trauma e.g. traumatic brain injury, neonatal hypoxic ischemic encephalopathy (HIE), myasthenia gravis (MG), Guillain-Barré syndrome (GBS), prion diseases, cancer, lung cancer, glioblastoma multiforme (GBM), an infectious disease, insulin resistance, diabetes, SARS-COV-2 infection and/or COVID-19.

In some embodiments, the subject has been determined to have, or be at risk of, a complement-related disorder, e.g. by a method disclosed herein.

In some embodiments, prior to the administration of the polynucleotide, vector, plasmid, rAAV, composition or cell, the method of treatment comprises:
(a) determining the level of one or more of FHR1, FHR2, FHR3, FHR4 and/or FHR5 in a sample obtained from the subject; and
(b) determining that the subject has or is likely to develop a complement-related disorder if the level of the protein determined in (a) is elevated as compared to the level of that protein in a sample from a control subject that does not have a complement-related disorder or a control subject that has a complement-related disorder that is not associated with elevated levels of said FHR protein(s).

The present invention also provides a method for expressing a transgene or polypeptide (as described herein) in a cell, the method comprising introducing a polynucleotide, vector, plasmid, rAAV, or composition according to the present disclosure into the cell. In some embodiments, the cell is an eye cell, a kidney cell or a cell of the CNS. The cell may be in vitro, ex vivo or in vivo.

### DESCRIPTION

The present invention relates to polynucleotides, expression cassettes and vectors comprising nucleic acid sequences that encode for a C3b binding domain of CR1 (complement receptor 1). The inventors have generated viral vectors containing a codon-optimised transgene sequence which express polypeptides comprising the CR1 C3b binding domain in vitro and in vivo, and which demonstrate reduction of the formation and deposition of the membrane attack complex (MAC) in vivo. The MAC is an immune activation complex that, when the usual controls fail, can deposit on nearby cells and tissues and initiate inflammatory responses. Such polynucleotides, expression cassettes and vectors (and the resulting protein products) may be used as therapeutic agents to treat complement-related disorders.

Complement-driven immune responses are naturally prevented through the proteolytic cleavage of C3b by the blood borne serine protease complement factor I (FI) into sequential, smaller breakdown products: inactive C3b (iC3b), C3dg, and finally C3d. It is becoming increasingly evident that the main driver of complement-associated disease pathogenesis is the deposition of iC3b, and not C3b as previously thought. For example, much of complement-mediated inflammation results from the interaction of iC3b with the complement receptors CR3 and CR4 on neutrophils, where removal of iC3b also removes the observed immunopathology.

Of the common FI co-factors, only CR1 is capable of driving complete degradation of deposited C3b all the way through to C3d, and thus prevents complement-mediated inflammation and immune cell recruitment (through conversion of C3b into iC3b), as well as preventing tissue remodelling resulting from opsonisation of surfaces (through cleavage of iC3b into C3dg and ultimately into C3d).

CR1 is the only natural complement regulator able to mediate opsonisation and iC3b deposition in vivo while simultaneously switching off the amplification loop and thus reducing inflammation. Thus, nucleic acids disclosed herein that encode polypeptides with functional CR1 co-factor activity hold promise for treating diseases associated with complement activation, including but not limited to AMD, kidney diseases, and neurodegenerative diseases.

The transgenes described herein encode polypeptides that possess potent FI co-factor activity and drive complete C3b degradation, that can diffuse across human BrM to access all sites of complement activation in the eye, and that remain functionally active on both sides of the membrane barrier. The polypeptides maintain cofactor activity in the presence of high levels of FHR proteins, which are known to be associated with the progression of complement mediated diseases and which inhibit the breakdown of C3b. In vivo efficacy is demonstrated by the reduction of complement activation and membrane attack complex (MAC) deposition in the laser-induced CNV mouse and rat models.

Delivery of these polypeptides as a gene therapy offers a one-time treatment that avoids the burden associated with the long-term, regular administrations required by other forms of therapy. Clinical experience with anti-VEGF agents for treating wet AMD demonstrates that a regimen of monthly or bimonthly intravitreal injections is difficult to maintain over time, impacting long-term efficacy.

### Complement System

Complement is a central part of the innate immunity that serves as a first line of defence against foreign and altered host cells. Complement is activated upon infection with microorganisms to induce inflammation and promote elimination of the pathogens. The complement system is composed of plasma proteins produced mainly by the liver or membrane proteins expressed on cell surface. Complement operates in plasma, in tissues, or within cells. For a review of the complement system, see e.g. Merle N S et al., Front Immunol. 2015 Jun. 2; 6:262, which is hereby incorporated by reference in its entirety.

The complement system can be activated via three distinct pathways: the classical pathway (CP), alternative pathway (AP) and lectin binding pathway (LP). In a healthy individual, the AP is permanently active at low levels to survey for presence of pathogens but host cells are protected against complement attack and are resistant to persistent low-level activation. C3b molecules bound to host cells are inactivated rapidly by a group of membrane-bound or plasma complement regulators.

In response to the recognition of molecular components of microorganisms, complement proteins become sequentially activated in an enzyme cascade: the activation of one protein enzymatically cleaves and activates the next protein in the cascade.

The three pathways converge into the generation of a C3 convertase, which cleaves the central complement component C3 into activation products C3b, a large fragment that acts as an opsonin (binds to foreign microorganisms to increase their susceptibility to phagocytosis), and C3a, an anaphylatoxin that promotes inflammation. Along with factor B (FB), C3b forms the C3 convertase (C3bBb) which cleaves further C3 molecules, generates more C3b and C3a, and amplifies C3b deposition on cell surfaces. This is the complement amplification loop. C3b deposition and activation of complement may occur on acellular structures (i.e. on extracellular matrix), such as Bruch's membrane (BrM) and the intercapillary septa of the choriocapillaris in the eye.

Activated C3 can trigger the lytic pathway, which can damage the plasma membranes of cells and some bacteria. C5a, another anaphylatoxin produced by this process, attracts macrophages and neutrophils and also activates mast cells.

Once activated, the complement system needs tight control, as newly generated complement activation products, e.g. C3b, can induce severe inflammation and cell damage to the host. A number of soluble as well as membrane bound complement regulators ensure regulation of complement activation at the surface of host cells and control different activation phases and sites of action (Skerka et al., Mol Immunol 2013, 56:170-180). Complement regulators are described further herein.

### C3, C3b, C4b and Breakdown Products

C3 is the central complement component. The pathways by which C3 is processed into various downstream products can lead to activation of complement, e.g. including inflammation and immune responses, or to the inactivation and regulation of complement.

Processing of C3 is described, for example, in Foley et al. J Thromb Haemostasis (2015) 13:610-618, which is hereby incorporated by reference in its entirety. Human C3 (UniProt: P01024) comprises a 1,663 amino acid sequence (including an N-terminal, 22 amino acid signal peptide). Amino acids 23 to 667 encode C3 β chain, and amino acids 749 to 1,663 encode C3b α′ chain. C3 β chain and C3 α′ chain associate through interchain disulphide bonds (formed between cysteine 559 of C3 β chain, and cysteine 816 of the C3 α′ chain) to form C3b. C3a is a 77 amino acid fragment corresponding to amino acid positions 672 to 748 of C3, generated by proteolytic cleavage of C3 to form C3b.

Processing of C3b to the inactive form iC3b, which cannot itself promote further complement amplification, involves proteolytic cleavage of the C3b α′ chain at amino acid positions 1303 and 1320 to form an a′ chain fragment 1 (corresponding to amino acid positions 749-1663 of C3), and an a′ chain fragment 2 (corresponding to amino acid positions 1321 to 1,663 of C3). Thus, iC3b comprises the C3 β chain, C3 α′ chain fragment 1 and C3 α′ chain fragment 2 (associated via disulphide bonds). Cleavage of the α′ chain also liberates C3f, which corresponds to amino acid positions 1304 to 1320 of C3.

iC3b is processed further to C3c comprising the C3 β chain, C3 α′ chain fragment 2 and C3c a′ chain fragment 1 (corresponding to amino acid positions 749-954 of C3). This cleavage event produces fragment C3dg (corresponding to amino acid positions 955-1303 of C3), which is itself broken down into fragments C3g (corresponding to amino acid positions 955-1001 of C3) and C3d (corresponding to amino acid positions 1002-1303 of C3). It is advantageous to encourage further processing of iC3b to the later molecules because iC3b deposition on biological surfaces can still trigger inflammatory responses.

Processing of C3b to iC3b and beyond is performed by Complement Factor I (FI; encoded in humans by the gene CFI). Human Complement Factor I (UniProt: P05156; SEQ ID NO:35) has a 583 amino acid sequence (including an N-terminal, 18 amino acid signal peptide). Amino acids 340 to 574 of the light chain encode the proteolytic domain of FI, which is a serine protease containing the catalytic triad responsible for cleaving C3b to produce iC3b (Ekdahl et al., J Immunol (1990) 144 (11): 4269-74). The complete coding sequence for human FI protein is provided in GenBank J02770.1 (GI 182606, version 1).

FI also mediates cleavage of C4b to C4c and C4d. C4b is a component of the C3 and C5 convertases and is essential for the propagation of the classical complement pathway.

Proteolytic cleavage of C3b and C4b by FI is facilitated by co-factors, including FH, CR1, C4 bp, and possibly some of the FHR proteins. Co-factors for FI typically bind to C3b and/or FI, and potentiate processing of C3b to iC3b by FI.

### CR1 Nucleic Acids, Vectors, Polypeptides and Cells

The invention relates to molecules and articles, such as nucleic acids, expression cassettes, vectors, polypeptides, and cells for example that are useful for treating complement-related disorders, as described herein.

Provided herein are nucleic acid sequences that encode polypeptides which comprise or consist of portions of human Complement Receptor 1 (CR1). Any polypeptide, or portion of polypeptide, described herein may be encoded by, or partly encoded by, a nucleic acid as described herein. Thus, reference to ‘a polypeptide’ or ‘polypeptides’ herein may be taken to apply to a polypeptide encoded by a nucleic acid or nucleotide sequence, such as those disclosed herein. Equally, reference to a ‘nucleic acid’ or ‘nucleotide sequence’ herein may be taken to apply to a nucleic acid or nucleotide sequence that encodes a polypeptide(s) such as those disclosed herein.

The nucleotide sequence of the human CR1 gene is located on chromosome 1, at positions 207,496,147-207,641,765 (forward strand, see e.g. Ensembl ID ENSG00000203710, Release: 105). CR1 transcript variant S mRNA is provided at NCBI NM_000651.6 (GI 1731160520, version 6). CR1 transcript variant F mRNA is provided at NCBI NM_000573.4 (GI 1677499597, version 4). CR1 is also called CD35.

Human CR1 protein (UniProt: P17927 (Entry version 205 (23 Feb. 2022), Sequence version 3 (2 Mar. 2010)); SEQ ID NO:1) has a 2,039 amino acid sequence (including an N-terminal, 41 amino acid signal peptide), and comprises 30 complement control protein (CCP) domains (also known as sushi domains or short consensus repeats (SCRs)). The 28 N-terminal CCP domains are organised into four long homologous repeat (LHR) domains each comprising 7 CCPs: LHR-A, LHR-B, LHR-C and LHR-D. The C3b binding regions of CR1 are found in CCPs 8-10 in LHR-B (UniProt: P17927 positions 491 to 684; SEQ ID NO:3), and CCPs 15-17 in LHR-C (UniProt: P17927 positions 941 to 1134; SEQ ID NO:4). CCPs 8-10 and 15-17 differ in sequence by three amino acid residues. CCPs 8-10 and 15-17 are also able to bind to C4b. CCPs1-4 also provide a C4b binding site, see e.g. Krych et al., J Biol Chem. 1994 May 6; 269 (18): 13273-8, which is hereby incorporated by reference in its entirety.

Provided herein are nucleic acid sequences that encode polypeptides that are capable of binding to C3b. In some embodiments the polypeptides can act as cofactors for Factor I, e.g. during Factor I-mediated breakdown/inactivation of C3b. In some embodiments, the nucleic acid sequences of the disclosure encode polypeptides that are capable of binding to C4b (instead of or as well as binding to C3b). In some embodiments, the polypeptides can modulate C3 convertase, and for example attenuate activation of the classical pathway by preventing C3 cleavage into C3a and C3b. In some embodiments the polypeptides comprise or consist of fragments of CR1. Further functional properties of the nucleic acids and polypeptides are described herein below.

The polypeptides may comprise or consist of C3b-binding portions of CR1 (indicated by underline in SEQ ID NO:1). The polypeptides may comprise or consist of one or more CR1 CCP domains that bind to C3b. The polypeptides may comprise or consist of a sequence corresponding to CR1 LHR-B (e.g. positions 491-939 of SEQ ID NO:1), or a portion thereof, and/or CR1 LHR-C (positions 941-1389 of SEQ ID NO:1), or a portion thereof.

Provided herein is a nucleotide sequence encoding a polypeptide that comprises or consists of an amino acid sequence corresponding to CCPs 8-10 of CR1 (i.e. SEQ ID NO:3). Provided herein is a nucleotide sequence encoding a polypeptide that comprises or consists of an amino acid sequence corresponding to CCPs 15-17 of CR1 (i.e. SEQ ID NO:4). Provided herein is a nucleotide sequence encoding a polypeptide that comprises or consists of an amino acid sequence corresponding to SEQ ID NO:2 (consensus sequence for SEQ ID NO:3 and 4), wherein X₁ is A or T, X₂ is P or L, and/or X₃ is G or R. Provided herein is a nucleotide sequence encoding a polypeptide that comprises or consists of, or an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to one or more of SEQ ID NO:2, 3 and/or 4.

In some embodiments the nucleic acid/nucleotide sequences are codon optimised, i.e. comprise synonymous codons based on an organism's or cell's codon bias without altering the amino acid sequence of the translated protein. Codon optimisation can improve translational efficiency and protein expression.

Thus, provided herein is a nucleotide sequence based on SEQ ID NO:7 or 8 (wild-type nucleic acid encoding CR1 CCPs 8-10, the latter including wild-type Factor H signal peptide sequence) in which codons have been optimised for protein expression in mammalian cells, e.g. human cells. The cells may be any cells that are affected by a complement related disorder, e.g. as described herein. The cells may be in a tissue or organ affected by a complement related disorder. The nucleic acid sequence may be codon-optimised for expression in human cells, e.g. of the eye, kidney, vascular system, blood, muscle, skin, oesophagus, small or large intestine, intestinal tract, pharynx, trachea, lungs, bronchi, bronchioles, central nervous system, or brain.

The cells may be RPE, photoreceptor, retinal or ganglion cells. The cells may be epithelial cells. The cells may be endothelial cells. The cells may be kidney cells, such as glomerular endothelial cells, macula densa cells, mesangial cells, parietal epithelial cells, podocytes, or tubule epithelial cells. The cells may be cells of the CNS, e.g. astrocytes, oligodendrocytes, ependymal cells, or microglia.

The present invention provides a nucleotide sequence comprising or consisting of SEQ ID NO:5. Also provided is a nucleotide sequence comprising or consisting of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:5.

The nucleotide sequence may comprise a nucleic acid sequence encoding a secretory pathway sequence (also known as a signal peptide or signal sequence). As used herein, a secretory pathway sequence/signal peptide is an amino acid sequence which directs secretion of a polypeptide. Polypeptides secreted by mammalian cells generally have a signal peptide fused to the N-terminus of the polypeptide, which is cleaved from the translated polypeptide to produce a “mature” form of the polypeptide. Secretory pathway sequences/signal peptides normally consist of a sequence of 5-30 hydrophobic amino acids, which form a single alpha helix. The sequence encoding the signal peptide may be part of or joined to another nucleotide sequence described herein (e.g. with at least 80% sequence identity to SEQ ID NO:5 or 6), or may be arranged separately to said nucleotide sequence within a polynucleotide. The signal peptide may be derived from the same protein that is encoded by the nucleotide sequence, or from a different protein.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

In some embodiments the signal peptide is from Factor H (FH), e.g. SEQ ID NO:9 or 10. In some embodiments the signal peptide is codon optimised, e.g. for optimal expression and secretion in mammalian cells. In some embodiments the signal peptide is a codon optimised nucleic acid sequence encoding the signal peptide from FH, e.g. SEQ ID NO:11. Thus, provided is a nucleotide sequence comprising or consisting of SEQ ID NO:6. Also provided is a nucleotide sequence comprising or consisting of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:6.

In some embodiments, a nucleotide sequence encoding a polypeptide described herein has a sequence having less than 80%, less than 79%, less than 78%, less than 77%, less than 76%, or less than 75% sequence identity with SEQ ID NO:7 (wild type nucleic acid sequence encoding CCPs 8-10 of CR1).

In some embodiments, a nucleotide sequence encoding a polypeptide described herein has a sequence having less than 80%, less than 79%, less than 78% or less than 77% sequence identity with the wild type coding sequence for CR1 CCPs 8-10, e.g. nucleotides 1498 to 2079 of GenBank: Y00816.1, Version 1, GI: 30185.

The present invention provides expression cassettes comprising nucleic acid/nucleotide sequences encoding the polypeptides described herein.

Thus, in one aspect, there is provided a polynucleotide comprising, in 5′ to 3′ or 3′ to 5′ order:
(i) a first nucleotide sequence comprising a promoter;
(ii) a second nucleotide sequence (e.g. encoding a polypeptide as described herein) comprising a transgene, wherein the transgene comprises or consists of a nucleotide sequence having at least 80% sequence identity to SEQ ID NO:6 or 5 (i.e. as described above); and
(iii) a polyadenylation (polyA) signal sequence.

In some embodiments the polynucleotide is isolated and/or substantially purified. In some embodiments, the polynucleotide is a polydeoxyribonucleotide. In some embodiments, the polynucleotide is a polyribonucleotide, such that thymine residues in the SEQ ID NOs can accordingly be uracil.

In some embodiments the second nucleotide sequence, i.e. containing a transgene to be expressed, is operably linked to the first nucleic sequence. The term “operably linked” may include the situation where the first and second nucleotide sequences are covalently linked in such a way as to place the expression of the second nucleotide sequence under the influence or control of the first nucleotide sequence, such that first nucleotide sequence is thus capable of effecting transcription of the second nucleotide sequence. The resulting transcript(s) may then be translated into a desired peptide(s)/polypeptide(s), e.g. as described herein.

In some embodiments the second nucleotide sequence comprises a stop codon or termination codon that can signal the termination of protein synthesis. In some embodiments the stop codon is positioned at the distal end of the second nucleotide sequence compared to the position of the first nucleotide sequence. In some embodiments the polynucleotide comprises a stop codon at the distal end of the polyA signal sequence compared to the position of the second nucleotide sequence. The stop codon may be TAA, TAG or TGA. In some embodiments the stop codon is TAA.

Thus, in some embodiments there is provided a polynucleotide comprising, in 5′ to 3′ or 3′ to 5′ order:
(i) a first nucleotide sequence comprising a promoter;
(ii) a second nucleotide sequence comprising a transgene and a stop codon, wherein the transgene comprises or consists of a nucleotide sequence having at least 80% sequence identity to SEQ ID NO:6 or 5; and
(iii) a polyadenylation (polyA) signal sequence.

In some embodiments the second nucleotide sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:38 or 37.

The first nucleotide sequence may comprise any suitable promoter for driving expression of the second nucleotide sequence, e.g. in mammalian cells. The promoter may be a mammalian promoter, i.e. is able to drive transcription of the second nucleotide sequence or transgene in a mammalian cell. The promoter may be a human promoter i.e. is able to drive transcription of the second nucleotide sequence or transgene in a human cell. Suitable promoters will be known to a skilled person, including constitutive promoters such as the simian virus 40 early promoter (SV40), cytomegalovirus immediate-early promoter (CMV), human Ubiquitin C promoter (UBC), human elongation factor 1a promoter (EF1A), mouse phosphoglycerate kinase 1 promoter (PGK), chicken β-Actin promoter (CBA), and chicken β-Actin promoter (CBA) coupled with CMV early enhancer (CAG or CAGG), see e.g. Qin et al., PLOS One. 2010; 5 (5): e10611, which is hereby incorporated by reference in its entirety.

The chimeric introns used in the CAG and CBA promoters are ~1 kb making the promoters relatively large (~1.6 kb) and so smaller introns may be used, for example to fit within the ~4.8 kb packaging capacity of AAV vectors. Smaller introns may include the SV40 intron (~97 bp) or a chimeric intron that is a chimera between introns from human β-globin and immunoglobulin heavy chain genes (~133 bp).

Sequences for the promoters above are publicly available. For example, the CMV enhancer with the chicken β-actin core promoter (pCAGGS plasmid; GenBank: LT727518.1), the SV40 intron (pTR-CBA-EGFP; GenBank: MK225672.1), chimeric human β-globin and immunoglobulin heavy chain intron (pCI plasmid; GenBank: U47119.2), and the CAG promoter (pCAGGS vector; GenBank: LT727518.1)

In some embodiments the first nucleotide sequence comprises a promoter that drives expression in a specific eye cell type, e.g. in rod, cone, RPE, or ganglion cells. In some embodiments the first nucleotide sequence comprises a fragment of the proximal mouse opsin promoter (mOP), or the human G-protein-coupled receptor protein kinase 1 promoter (hGRK1), as described for example in Beltran W A, et al. Gene Ther. 2010; 17:1162-74 and Boye S E, et al. Hum Gene Ther. 2012; 23:1101-15, which are hereby incorporated by reference in their entirety.

In some embodiments, the first nucleotide sequence comprises a promoter that drives expression, e.g. of the second nucleotide sequence, in retinal pigment epithelial (RPE) cells. In some embodiments, the promoter is a human RPE65 promoter, a shorted RPE65 (NA65) promoter combined with an SV40 intron, or a VMD2 promoter, or modified versions thereof, see e.g. Wang et al., Sci Rep. 2019 Oct. 31; 9 (1): 15732; Georgiadis et al., Gene Ther. 2016 December; 23 (12): 857-862.

In some embodiments the first nucleotide sequence comprises an inducible promoter, i.e. gene expression is activated by the promoter only in the presence or absence of a particular molecule. Suitable inducible promoters will be known to the skilled person, such as the TRE promoter which can be activated by the rtTA transcriptional activator in a doxycycline-inducible manner (Qin et al., PLOS One. 2010; 5 (5): e10611). Further examples of inducible promoters are described in e.g. Le at al. Invest Ophthalmol Vis Sci. 2008, 49 (3): 1248-1253 and McGee Sanftner et al. Mol Ther. 2001. 3 (5): 688-696; which are hereby incorporated by reference in their entirety.

In some embodiments the first nucleotide sequence comprises a shortened or hybrid form of the CBA promoter, for example containing a CMV enhancer, CBA promoter, and hybrid intron of chicken β-Actin and minute virus of mouse (MVM), as described in e.g. Gray et al., Hum Gene Ther. 2011 September; 22 (9): 1143-53, which is hereby incorporated by reference in its entirety. Such a promoter may be called a CBh, mini CBA, hybrid CBA, or short CBA promoter. In some embodiments the first nucleotide sequence comprises or consists of SEQ ID NO:14. In some embodiments the first nucleotide sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:14.

In some embodiments the first nucleotide sequence comprises a CAG synthetic promoter containing a CMV immediate early enhancer, chicken β-actin (CBA) promoter, and a synthetic chimeric intron containing the first exon plus splice donor and intron of chicken β-actin (CBA) promoter with an intron and splice acceptor of rabbit β-globin. In some embodiments the first nucleotide sequence comprises or consists of SEQ ID NO:15. In some embodiments the first nucleotide sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:15.

In some embodiments the first nucleotide sequence comprises a truncated CAG synthetic promoter, for example containing a CMV immediate early enhancer, chicken β-actin (CBA) promoter, and a shortened synthetic chimeric intron with beta actin splice donor and beta globin splice acceptor. In some embodiments the first nucleotide sequence comprises or consists of SEQ ID NO:16. In some embodiments the first nucleotide sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:16.

In some embodiments the first nucleotide sequence comprises a truncated CAG synthetic promoter containing a CMV immediate early enhancer, chicken β-actin (CBA) promoter, and a synthetic chimeric intron derived from human β-globin and immunoglobulin heavy chain genes. In some embodiments the first nucleotide sequence comprises or consists of SEQ ID NO:17. In some embodiments the first nucleotide sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:17.

In some embodiments the first nucleotide sequence comprises a human elongation factor-1 alpha (EF1A) promoter. In some embodiments the first nucleotide sequence comprises or consists of SEQ ID NO:18. In some embodiments the first nucleotide sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:18.

Polyadenylation of mRNA transcripts is required for efficient nuclear export and to provide mRNA stability. The polyadenylation (polyA) signal sequence may be any suitable sequence, such as the bovine growth hormone (bGH), human growth hormone (hGH) and SV40 late polyA sequences, all of which are readily available to the skilled person, see e.g. Azzoni et al., J Gene Med. 2007 May; 9 (5): 392-402, which is hereby incorporated by reference in its entirety. The bGH sequence has been used for ocular indications and in therapeutic agents such as voretigene neparvovec (Spark Therapeutics), timrepigene emparvovec (Biogen) and cotoretigene toliparvovec (Biogen).

In some embodiments the polyadenylation (polyA) signal sequence comprises or consists of SEQ ID NO:19 (SV40). In some embodiments the polyadenylation (polyA) signal sequence comprises or consists of SEQ ID NO:20 (bGH). In some embodiments the polyadenylation (polyA) signal sequence comprises or consists of SEQ ID NO:21 (hGH). In some embodiments the polyadenylation (polyA) signal sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:19, 20 or 21.

In some embodiments the polynucleotide comprises a post-transcriptional regulatory element (PRE). Such elements can be important for viral gene expression and act to support nuclear export of viral RNA. In some embodiments the polynucleotide comprises a Woodchuck Hepatitis Virus (WHV) Posttranscriptional Regulatory Element (WPRE). In some embodiments the polynucleotide comprises a Hepatitis B Posttranscriptional Regulatory Element (HPRE).

Wild-type WPRE sequence is not recommended as it contains a cryptic promoter which drives expression of the viral X antigen that has been linked to increased tumour susceptibility in mice. A mutated WPRE has been generated by introduction of six point-mutations located in the start codon and putative promoter region of the X protein and is widely used as a PRE for gene expression, see e.g. Zanta-Boussif et al., Gene Ther. 16, 605-619 (2009), which is hereby incorporated by reference in its entirety.

In some embodiments the PRE is positioned between the second nucleotide sequence and the polyA signal sequence. Thus, provided is a polynucleotide comprising, e.g. in 5′ to 3′ or 3′ to 5′ order:
(i) a first nucleotide sequence comprising a promoter;
(ii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO:6 or 5;
(iii) a post-transcription regulatory element, preferably a WPRE, more preferably a mutant WPRE; and
(iv) a polyadenylation signal sequence.

In some embodiments the WPRE sequence comprises or consists of SEQ ID NO:22. In some embodiments the WPRE sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:22.

In some embodiments the polynucleotide does not comprise a post-transcription regulatory element, such as a WPRE or HPRE.

In some embodiments the polynucleotide comprises a stuffer or filler sequence. The expression cassettes/polynucleotides provided herein may be used in viral vectors, e.g. AAV vectors, as described herein. In such cases, it is important to ensure that the AAV genome (including ITR sequences) does not fall below ~4.0 kb for single stranded (ss) AAV vectors and ~2 kb for self-complementary (sc) AAV vectors. Ideally, a ssAAV gnome would be kept close to the native size of ~4.7 kb. This is to ensure efficient packaging of the genome into the AAV particles and supports the manufacture and storage stability of high-quality recombinant AAV vectors. Since the nucleotide sequence of the transgene is small, polynucleotides described herein may contain a stuffer sequence to increase the size of the expression cassette. In some embodiments, a stuffer sequence is placed after the polyA sequence in the polynucleotide to avoid unfavourable secondary structures, potential transcription, potential translation, or unexpected splicing events in the 5′UTR.

In some embodiments the polynucleotide comprises a stuffer sequence based on a synthetic VMD2 (BEST1) intron. In some embodiments the stuffer sequence comprises 12 bp of VMD2 untranslated exon-1 and exon-2 sequences. In some embodiments the stuffer sequence does not contain splice donor and acceptor sites or repetitive sequences. In some embodiments stuffer sequence comprises or consists of SEQ ID NO:23. In some embodiments the stuffer sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:23.

In some embodiments the polynucleotide comprises a stuffer sequence based on a synthetic RLBP1 intron. In some embodiments the stuffer sequence does not contain splice donor and acceptor sites or repetitive sequences. In some embodiments the stuffer sequence comprises or consists of SEQ ID NO:24. In some embodiments the stuffer sequence comprises or consists of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:24.

In some embodiments the polynucleotide does not comprise a stuffer/filler sequence.

In some aspects of the present invention, a polynucleotide provided herein is designed for expression in a vector or plasmid. Suitable vectors and plasmids are described herein. In some embodiments a polynucleotide described herein is designed for expression using a viral vector, such as an adeno-associated virus (AAV) or adenovirus vectors. Thus, in some embodiments a polynucleotide or expression cassette provided herein comprises inverted terminal repeat (ITR) sequences for use in an AAV or adenovirus vector.

ITRs are 145 nucleotide, palindromic sequences located at the termini of an adenovirus or AAV genome, see e.g. Earley et al., Hum Gene Ther. February 2020; 31 (3-4): 151-162, which is hereby incorporated by reference in its entirety. In wild type AAV, ITRs are important for the regulation and priming of viral DNA replication and contain secondary structures including the Rep binding element (RBE) and a terminal resolution site (TRS), which together constitute the AAV origin of replication. They also facilitate recombination of the viral genome with the cellular genome of the host and are required for packaging/genome encapsidation and vector persistence, see e.g. Maurer and Weitzman, Hum Gene Ther. 2020 May; 31 (9-10): 499-511, which is hereby incorporated by reference in its entirety.

In recombinant AAV (rAAV) vectors, the internal wildtype AAV genes are removed and replaced by an expression cassette of interest, leaving only the ITR sequences.

Thus, in some embodiments a polynucleotide described herein comprises at least one ITR sequence. In some embodiments the polynucleotide comprises a 5′ ITR and/or a 3′ ITR. In some embodiments the 5′ and/or 3′ ITR is selected from an AAV1, AAV2, AAV3, AAV4, AAV5, AAV6, AAB7, AAV8, AAV9, AAV10, AAV11, AAV12, AAV-DJ, AAV-DJ/8, AAV-rh10, AAVrh.39, AAV-retro, AAV-PHP.B, AAV8-PHP.eB, AAV-PHP.S, AAV-Anc80, AAV2.5, AAV Spark100, R100, AAV2.7m8, AAV-LK05 and AAVtYF ITR. In some embodiments the 5′ and/or 3′ ITR is selected from an AAV1, AAV2, AAV4, AAV5, or AAV8 ITR. The 5′ and 3′ ITRs may be from the same AAV serotype, or from different AAV serotypes.

In some embodiments the 5′ and/or 3′ ITR is an AAV2 ITR. In some embodiments both ITRs are AAV2 ITRs. In some embodiments the 5′ and/or 3′ ITR is an AAV8 ITR. In some embodiments both ITRs are AAV8 ITRs.

A wild type AAV genome is single stranded (between the two dsDNA hairpin ITRs at each end) and needs to be converted into double-stranded DNA prior to expression, which can limit the efficacy and stability of ssAAV vectors, see e.g. McCarty et al., Mol Ther. 2008, 16 (10): 1648-56, which is hereby incorporated by reference in its entirety. The synthesis of a complementary DNA strand can be avoided by using self-complementary vectors, which comprise an inverted repeat genome that can fold into double stranded DNA without the need for DNA synthesis or base paring between multiple vector genomes. scAAV genomes can be generated by deleting the terminal resolution site (TRS) site from one ITR, such that replication is initiated at the other (wild type) ITR, continues through the mutant ITR to form a hairpin and then proceeds back towards the first ITR. This generates a dsDNA molecule with a wild type ITR at each end and the mutated ITR in the middle. This dimeric inverted repeat can then undergo normal rounds of replication from the two wild-type ITR ends, with each displaced daughter strand comprising a ssDNA inverted repeat with a complete ITR at each end and a mutated ITR in the middle. Production of scAAV from constructs with one mutated ITR typically yields >90% dimeric genomes.

In some embodiments, the 5′ ITR or the 3′ ITR is a mutant ITR, see e.g. McCarty et al. Gene Ther. 2003 December; 10 (26): 2112-8, which is hereby incorporated by reference in its entirety. In some embodiments, the 5′ ITR or the 3′ ITR lacks a functional terminal resolution site. That is, in some embodiments, the polynucleotide comprises one wild type ITR and one mutant ITR. In such cases, the polynucleotide is capable of forming a self-complementary AAV vector, e.g. is configured for forming a self-complementary AAV vector. In some embodiments the polynucleotide results in the formation of a self-complementary AAV vector.

In some embodiments, one ITR, e.g. the 5′ ITR, comprises or consists of SEQ ID NO:25 or SEQ ID NO: 26 (or a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:25 or SEQ ID NO:26). In some embodiments, one ITR, e.g. the 3′ ITR, comprises or consists of SEQ ID NO:27 or SEQ ID NO:28 (or a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:27 or SEQ ID NO:28).

In some embodiments a first ITR, e.g. a 5′ ITR, comprises or consists of SEQ ID NO:25 (or a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto) and a second ITR, e.g. a 3′ ITR, comprises or consists of SEQ ID NO:27 (or a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto). Such sequences are based on AAV2 ITRs.

In some embodiments a first ITR, e.g. a 5′ ITR, comprises or consists of SEQ ID NO:26 (or a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto) and a second ITR, e.g. a 3′ ITR, comprises or consists of SEQ ID NO:28 (or a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto). Such sequences are based on AAV2 ITRs, in which one ITR is a mutant ITR, such that the polynucleotide is capable of forming a self-complementary AAV vector.

Thus, the present invention provides a polynucleotide comprising, e.g. in 5′ to 3′ or 3′ to 5′ order:
(i) an ITR
(ii) a first nucleotide sequence comprising a promoter;
(iii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5;
(iv) a polyadenylation signal sequence; and/or
(v) an ITR.

Either one of the ITRs could be a mutant ITR.

The present invention provides a polynucleotide comprising, e.g. in 5′ to 3′ or 3′ to 5′ order:
(i) an ITR, e.g. a 5′ ITR, e.g. as described herein;
(ii) a first nucleotide sequence comprising a promoter, e.g. as described herein;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO: 6 or 5;
(iv) a post-transcription regulatory element, e.g. as described herein, preferably a WPRE, more preferably a mutant WPRE;
(v) a polyadenylation signal sequence, e.g. as described herein; and/or
(vi) an ITR, e.g. a 3′ ITR, optionally a mutant ITR, e.g. as described herein.

The present invention provides a polynucleotide comprising, e.g. in 5′ to 3′ or 3′ to 5′ order:
(i) an ITR, e.g. a 5′ ITR, e.g. as described herein;
(ii) a first nucleotide sequence comprising a promoter, e.g. as described herein;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO: 6 or 5;
(iv) a polyadenylation signal sequence, e.g. as described herein;
(v) a stuffer sequence, e.g. as described herein; and/or
(vi) an ITR, e.g. a 3′ ITR, e.g. as described herein.

The present invention provides a polynucleotide comprising, e.g. in 5′ to 3′ or 3′ to 5′ order:
(i) an ITR, e.g. a 5′ ITR, e.g. as described herein;
(ii) a first nucleotide sequence comprising a promoter, e.g. as described herein;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO: 6 or 5;
(iv) a post-transcription regulatory element, e.g. as described herein, preferably a WPRE, more preferably a mutant WPRE;
(v) a polyadenylation signal sequence, e.g. as described herein;
(vi) a stuffer sequence, e.g. as described herein; and/or
(vii) an ITR, e.g. a 3′ ITR, e.g. as described herein.

A polynucleotide described herein may also comprise further expression control sequences, such as sequences that stabilize cytoplasmic mRNA; sequences that enhance translation efficiency (i.e., Kozak consensus sequence); and sequences that enhance protein stability. The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. The choice and design of appropriate sequences is within the ability and discretion of the skilled person.

The present invention provides a polynucleotide comprising, e.g. in 5′ to 3′ or 3′ to 5′ order:
(i) an ITR, e.g. comprising or consisting of a sequence having at least 80% sequence identity to SEQ ID NO:26 or 28;
(ii) a first nucleotide sequence comprising a promoter, e.g. comprising or consisting of a sequence having at least 80% sequence identity to SEQ ID NO:14;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO:6 or 5;
(iv) a polyadenylation signal sequence, e.g. comprising or consisting of a sequence having at least 80% sequence identity to SEQ ID NO:19; and
(v) an ITR, e.g. comprising or consisting of a sequence having at least 80% sequence identity to SEQ ID NO:28 or 26.

In some embodiments there is provided a polynucleotide comprising or consisting of a nucleic acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:29.

The present invention provides a polynucleotide comprising, e.g. in 5′ to 3′ or 3′ to 5′ order:
(i) an ITR, e.g. comprising or consisting of a sequence having at least 80% sequence identity to SEQ ID NO:25 or 27;
(ii) a first nucleotide sequence comprising a promoter, e.g. comprising or consisting of a sequence having at least 80% sequence identity to SEQ ID NO:16;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO:6 or 5;
(iv) a post-transcription regulatory element, preferably a WPRE, e.g. comprising or consisting of a sequence having at least 80% sequence identity to SEQ ID NO:22;
(v) a polyadenylation signal sequence, e.g. comprising or consisting of a sequence having at least 80% sequence identity to SEQ ID NO:20;
(vi) a stuffer sequence, e.g. comprising or consisting of a sequence having at least 80% sequence identity to SEQ ID NO:23; and
(vii) an ITR, e.g. comprising or consisting of a sequence having at least 80% sequence identity to SEQ ID NO:27 or 25.

In some embodiments there is provided a polynucleotide comprising or consisting of a nucleic acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:30.

The present invention provides a polynucleotide comprising, e.g. in 5′ to 3′ or 3′ to 5′ order:
(i) an ITR, e.g. comprising or consisting of a sequence having at least 80% sequence identity to SEQ ID NO:25 or 27;
(ii) a first nucleotide sequence comprising a promoter, e.g. comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO:6 or 5;
(iv) a post-transcription regulatory element, preferably a WPRE, e.g. comprising or consisting of a sequence having at least 80% sequence identity to SEQ ID NO:22;
(v) a polyadenylation signal sequence, e.g. comprising or consisting of a sequence having at least 80% sequence identity to SEQ ID NO:20; and
(vi) an ITR, e.g. comprising or consisting of a sequence having at least 80% sequence identity to SEQ ID NO:27 or 25.

In some embodiments there is provided a polynucleotide comprising or consisting of a nucleic acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:31.

The present invention provides a polynucleotide comprising, e.g. in 5′ to 3′ or 3′ to 5′ order:
(i) an ITR, e.g. comprising or consisting of a sequence having at least 80% sequence identity to SEQ ID NO:25 or 27;
(ii) a first nucleotide sequence comprising a promoter, e.g. comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO:6 or 5;
(iv) a post-transcription regulatory element, preferably a WPRE, e.g. comprising or consisting of a sequence having at least 80% sequence identity to SEQ ID NO:22;
(v) a polyadenylation signal sequence, e.g. comprising or consisting of a sequence having at least 80% sequence identity to SEQ ID NO:20;
(vi) a stuffer sequence, e.g. comprising or consisting of a sequence having at least 80% sequence identity to SEQ ID NO:23; and
(vii) an ITR, e.g. comprising or consisting of a sequence having at least 80% sequence identity to SEQ ID NO:27 or 25.

In some embodiments there is provided a polynucleotide comprising or consisting of a nucleic acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:32.

The present invention provides a polynucleotide comprising, e.g. in 5′ to 3′ or 3′ to 5′ order:
(i) an ITR, e.g. comprising or consisting of a sequence having at least 80% sequence identity to SEQ ID NO:25 or 27;
(ii) a first nucleotide sequence comprising a promoter, e.g. comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO:6 or 5;
(iv) a polyadenylation signal sequence, e.g. comprising or consisting of a sequence having at least 80% sequence identity to SEQ ID NO:20;
(v) a stuffer sequence, e.g. comprising or consisting of a sequence having at least 80% sequence identity to SEQ ID NO:23; and
(vi) an ITR, e.g. comprising or consisting of a sequence having at least 80% sequence identity to SEQ ID NO:27 or 25.

In some embodiments there is provided a polynucleotide comprising or consisting of a nucleic acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:33.

The present invention provides a polynucleotide comprising, e.g. in 5′ to 3′ or 3′ to 5′ order:
(i) an ITR, e.g. comprising or consisting of a sequence having at least 80% sequence identity to SEQ ID NO:26 or 28;
(ii) a first nucleotide sequence comprising a promoter, e.g. comprising or consisting of a sequence having at least 80% sequence identity to SEQ ID NO:17;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO:6 or 5;
(iv) a post-transcription regulatory element, preferably a WPRE, e.g. comprising or consisting of a sequence having at least 80% sequence identity to SEQ ID NO:22;
(v) a polyadenylation signal sequence, e.g. comprising or consisting of a sequence having at least 80% sequence identity to SEQ ID NO:20; and
(vi) an ITR, e.g. comprising or consisting of a sequence having at least 80% sequence identity to SEQ ID NO:28 or 26.

In some embodiments there is provided a polynucleotide comprising or consisting of a nucleic acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:34.

The present invention provides a polynucleotide comprising, e.g. in 5′ to 3′ or 3′ to 5′ order:
(i) an ITR, e.g. a 5′ ITR, e.g. comprising or consisting of a sequence having at least 80% sequence identity to SEQ ID NO:25 or 27;
(ii) a first nucleotide sequence comprising a promoter, e.g. comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO: 6 or 5;
(iv) a post-transcription regulatory element, preferably a WPRE, e.g. comprising or consisting of a sequence having at least 80% sequence identity to SEQ ID NO:22;
(v) a polyadenylation signal sequence, e.g. comprising or consisting of a sequence having at least 80% sequence identity to SEQ ID NO:20;
(vi) a stuffer sequence, e.g. comprising or consisting of a sequence having at least 80% sequence identity to SEQ ID NO:24; and
(vii) an ITR, e.g. comprising or consisting of a sequence having at least 80% sequence identity to SEQ ID NO:27 or 25.

The present invention provides a polynucleotide comprising, e.g. in 5′ to 3′ or 3′ to 5′ order:
(i) an ITR, e.g. comprising or consisting of a sequence having at least 80% sequence identity to SEQ ID NO:26 or 28;
(ii) a first nucleotide sequence comprising a promoter, e.g. comprising or consisting of a sequence having at least 80% sequence identity to SEQ ID NO:16;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO: 6 or 5;
(iv) a polyadenylation signal sequence, e.g. comprising or consisting of a sequence having at least 80% sequence identity to SEQ ID NO:19; and
(v) an ITR, e.g. comprising or consisting of a sequence having at least 80% sequence identity to SEQ ID NO:28 or 26.

The present invention provides a polynucleotide comprising, e.g. in 5′ to 3′ or 3′ to 5′ order:
(i) an ITR, e.g. comprising or consisting of a sequence having at least 80% sequence identity to SEQ ID NO:26 or 28;
(ii) a first nucleotide sequence comprising a promoter, e.g. comprising or consisting of a sequence having at least 80% sequence identity to SEQ ID NO:16;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO: 6 or 5;
(iv) a polyadenylation signal sequence, e.g. comprising or consisting of a sequence having at least 80% sequence identity to SEQ ID NO:20; and
(v) an ITR, e.g. comprising or consisting of a sequence having at least 80% sequence identity to SEQ ID NO:28 or 26.

The present invention provides a polynucleotide comprising, e.g. in 5′ to 3′ or 3′ to 5′ order:
(i) an ITR, e.g. comprising or consisting of a sequence having at least 80% sequence identity to SEQ ID NO:26 or 28;
(ii) a first nucleotide sequence comprising a promoter, e.g. comprising or consisting of a sequence having at least 80% sequence identity to SEQ ID NO:18;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO: 6 or 5;
(iv) a polyadenylation signal sequence, e.g. comprising or consisting of a sequence having at least 80% sequence identity to SEQ ID NO:19;
(v) an ITR, e.g. comprising or consisting of a sequence having at least 80% sequence identity to SEQ ID NO:28 or 26.

Where nucleotide sequences/polynucleotides are described herein to contain more than one element, e.g. as in the paragraphs above, said elements (ITRs, first nucleotide sequence, second nucleotide sequence, regulatory elements etc as described herein) may be positioned in 5′ to 3′ order or in 3′ to 5′ order. That is, the elements may follow one another in the orders as described herein. For example, in 5′ to 3′ order or in 3′ to 5′ order, an ITR may be followed by a first nucleotide sequence, which is followed by a second nucleotide sequence, which is optionally followed by a post-transcription regulatory element, which is followed by a polyA signal sequence, which is optionally followed by a stuffer sequence, which is followed by an ITR sequence.

Any ITR, first nucleotide sequence, second nucleotide sequence, third nucleotide sequence, stuffer sequence, post-transcriptional regulatory element, and/or polyadenylation signal sequence described herein may be combined with any one or more other such sequences/elements as described herein.

A polynucleotide/expression cassette described herein may be contained in a vector or plasmid, e.g. for introduction into a cell, such as a human cell. Thus the present invention provides a vector or plasmid comprising a polynucleotide/expression cassette as described herein.

A “vector” as used herein is a molecule, e.g. a nucleic acid molecule, used as a vehicle to transfer exogenous nucleic acid into a cell. The vector may be a vector for expression of the nucleic acid in the cell, i.e. an expression vector. The vector may be suitable for gene therapy.

Suitable vectors include plasmids, binary vectors, DNA vectors, mRNA vectors, eukaryotic vectors, viral vectors, transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in its entirety. In some embodiments, the lentiviral vector may be pELNS, or may be derived from pELNS. In some embodiments, the vector may be a vector encoding CRISPR/Cas9. Viral and non-viral delivery systems for introducing genetic material into cells are reviewed, for example, in Nayerossadat et al., Adv Biomed Res. 2012; 1:27; MacLaren et al. Ophthalmology. 2016, 123 (10 Suppl): S98-S106; Petit and Punzo, Discov Med. 2016, 22 (121): 221-229; Aguirre, Invest Ophthalmol Vis Sci. 2017, 58 (12): 5399-5411; Lundstrom, Diseases. 2018, 6 (2): 42; which are hereby incorporated by reference in their entirety. Any suitable vector or plasmid can be used in the context of the present invention.

The vector may be a viral vector, such as a gammaretroviral vector (e.g. murine Leukemia virus (MLV)-derived vector), a lentiviral vector, a retroviral vector, an adenovirus vector, an adeno-associated virus (AAV) vector, a vaccinia virus vector or a herpesvirus vector, e.g. Herpes Simplex Virus vector. In some embodiments, the vector is a lentiviral vector.

In some embodiments, e.g. wherein the polynucleotide comprises ITR sequences, the vector is a adenovirus or adeno-associated virus (AAV) vector. Production of recombinant AAV (rAAV) vectors requires the AAV construct containing the transgene expression cassette to be flanked by two ITRs, e.g. a polynucleotide as described herein, along with a source of the AAV Rep and Cap genes (which express proteins required for replication/packaging and the capsid proteins, respectively). The AAV capsid is composed of three viral proteins (VPs): VP1, VP2, and VP3. Each capsid serotype exhibits a unique tissue tropism and transduction efficiency. AAV ITRs are also serotype-specific.

In some embodiments, the AAV vector is selected from AAV serotype 1, 2, 3, 4, 5, 6, 7, 8, 9, 10, 11, 12, AAV-DJ, AAV-DJ/8, AAV-rh10, AAVrh.39, AAV-retro, AAV-PHP.B, AAV8-PHP.eB, AAV-PHP.S, and AAV-Anc80, or variants, hybrids and/or mutants thereof. Suitable variants/hybrids/mutants are available to the skilled person, such as AAV2.5, AAV Spark100, R100, AAV2.7m8, AAV-LK05 and AAVtYF. Such vectors may be single stranded or self-complementary AAV vectors.

In some embodiments, the AAV vector exhibits tropism to eye tissue/cells. In some embodiments, the AAV vector is selected from serotype AAV1, AAV2, AAV4, AAV5 or AAV8. In some embodiments, the AAV vector exhibits tropism to kidney tissue/cells. In some embodiments, the AAV vector is selected from serotype AAV2, AAV-DJ, AAV-LK05, and AAV-Anc80. In some embodiments, the AAV vector exhibits tropism to the CNS/brain. In some embodiments, the AAV vector is selected from serotype AAV1, AAV2, AAV4, AAV5, AAV6, AAV7, AAV8, AAV9, AAV-rh10, e.g. as described in Castle et al., Methods Mol Biol. 2016; 1382:133-149, which is hereby incorporated by reference in its entirety.

In some embodiments, the AAV vector is an AAV serotype 2 (AAV2) vector, or a hybrid and/or mutant thereof. In some embodiments, the AAV vector is an AAV serotype 8 (AAV8) vector, or a hybrid and/or mutant thereof.

AAV vectors can be pseudotyped such that they contain the genome of one AAV variant packaged in the capsid of another. In some embodiments, the AAV vector is AAV2/2, AAV2/8, AAV2/1, AAV2/6, AAV2/5, AAV2/7, AAV2/9 (in which the ITRs of the first serotype are packaged in the capsid of the second), see e.g. Lebherz et al., J Gene Med. 2008 April; 10 (4): 375-382, which is hereby incorporated by reference in its entirety.

Recently the development of tyrosine mutant serotypes has greatly enhanced transduction efficiencies of rAAV vectors. In some embodiments, the rAAV vector is rAAV2/8 Y733F, rAAV2/2 Y444F or sextuple mutant rAAV2/2 Y252F, Y272F, Y444F, Y500F, Y704F, Y730F.

Thus, the present invention provides an AAV vector, e.g. a rAAV, comprising:
(i) a polynucleotide as described herein, e.g. comprising at least one ITR; and
(ii) at least one capsid protein.

In some embodiments the AAV vector is a self-complementary AAV vector.

In some embodiments the at least one capsid protein is selected from AAV1, AAV2, AAV3, AAV4, AAV5, AAV6, AAB7, AAV8, AAV9, AAV10, AAV11, AAV12, AAV-DJ, AAV-DJ/8, AAV-rh10, AAVrh.39, AAV-retro, AAV-PHP.B, AAV8-PHP.eB, AAV-PHP.S, AAV-Anc80, AAV2.5, AAV Spark100, R100, AAV2.7m8, AAV-LK05 and AAVtYF, or a variant/hybrid/mutant thereof.

In some embodiments the AAV vector is a pseudotyped AAV vector. In some embodiments the AAV vector is AAV2/2, AAV2/8, AAV2/1, AAV2/6, AAV2/5, AAV2/7, AAV2/9.

In some embodiments the AAV vector comprises AAV2 ITRs and at least one AAV2 capsid protein (AAV2/2). In some embodiments the AAV vector comprises AAV2 ITRs and at least one AAV8 capsid protein (AAV2/8).

Any nucleic acid described herein may comprise additional nucleotide sequence(s), in addition to a nucleotide sequence described hereinabove. Sequence identity of a nucleotide sequence to a SEQ ID NO provided herein may be assessed over the whole nucleic acid. Alternatively, sequence identity may be assessed over the specified nucleotide sequence only (e.g. over the sequence of SEQ ID NO:5, 6, 7 or 8 only). In some cases, the nucleic acid may comprise additional nucleotide sequence(s) that are not taken into account when assessing sequence identity, e.g. to SEQ ID NO: 5, 6, 7 and/or 8.

A nucleic acid sequence, e.g. polynucleotide or vector, or nucleotide sequence described herein may be defined by its length. A nucleic acid may be defined by its ‘total length’, e.g. including any nucleotide sequence comprised therein and any additional sequence(s), from 5′ to 3′.

Alternatively, a nucleic acid disclosed herein may be defined by the length of the nucleotide sequence or transgene comprised therein (i.e. the nucleotide sequence comprising or consisting of a sequence having at least 80% sequence identity to SEQ ID NO:6 and/or 5). In such cases, the nucleic acid may comprise additional nucleotide sequence(s) that renders the sequence of the entire nucleic acid longer than the specified length of said nucleotide sequence/transgene having at least 80% sequence identity to SEQ ID NO:6 and/or 5.

In some embodiments the polynucleotide sequence is 5 kb or fewer, 4.8 kb or fewer, 4.6 kb or fewer, 4.4 kb or fewer, 4.2 kb or fewer, 4.0 kb or fewer, 3.8 kb or fewer, 3.6 kb or fewer, 3.4 kb or fewer, 3.2 kb or fewer, 3.0 kb or fewer, 2.8 kb or fewer, 2.6 kb or fewer, 2.4 kb or fewer, 2.2 kb or fewer, or 2.0 kb or fewer in length.

In some embodiments the second nucleotide sequence (within the polynucleotide sequence) comprises a sequence having at least 80% sequence identity to SEQ ID NO:6 or 5, and the second nucleotide sequence is 1000 bp or fewer, 990 bp or fewer, 980 bp or fewer, 970 bp or fewer, 960 bp or fewer, 950 bp or fewer, 940 bp or fewer, 930 bp or fewer, 920 bp or fewer, 910 bp or fewer, 900 bp or fewer, 890 bp or fewer, 880 bp or fewer, 870 bp or fewer, 860 bp or fewer, 850 bp or fewer, 840 bp or fewer, 830 bp or fewer, 820 bp or fewer, 810 bp or fewer, 800 bp or fewer, 790 bp or fewer, 780 bp or fewer, 770 bp or fewer, 760 bp or fewer, 750 bp or fewer, 740 bp or fewer, 730 bp or fewer, 720 bp or fewer, 710 bp or fewer, 700 bp of fewer, 690 bp or fewer, 680 bp or fewer, 670 bp or fewer, 660 bp or fewer, 650 bp or fewer, 640 bp or fewer, 630 bp or fewer, 620 bp or fewer, 610 bp or fewer, or 600 bp or fewer in length. This disclosure also provides for a nucleic acid, or nucleotide sequence as above, that comprises or consists of a nucleotide sequence having ‘up to’ any length of bp described herein, e.g. up to 600 bp, up to 610 bp etc. Any two end points described herein may be used to make a range of lengths for the nucleic acid or nucleotide sequence, for example 600-800 bp in length or any combination of end points above.

The second nucleotide sequence may comprise or consist of a nucleotide sequence that is 500-1000 bp, 500-950 bp, 500-900 bp, 500-850 bp, 500-800 bp, 500-750 bp, 500-700 bp, 500-650 bp, 525-800 bp, 550-700 bp, or 550-650 bp in length. In some cases, the second nucleotide sequence has a length of 550-600 bp or 620-660 bp.

As will be evident from this disclosure, a polynucleotide or second nucleotide sequence described herein may be longer than the transgene sequence comprised therein (said transgene having at least 80% sequence identity to SEQ ID NO: 6 or 5). Thus, a nucleic acid provided herein may have one length above, whilst said transgene may be of a shorter length. Any of the lengths and/or ranges above can be combined. For example, the second nucleotide sequence may be 1000 bp or fewer in length, whilst the nucleotide sequence having at least 80% sequence identity to SEQ ID NO: 6 or 5 may be e.g. 550-700 bp in length.

Nucleic acids (polynucleotides/expression cassettes/nucleotide sequences) described herein are designed to express a polypeptide that binds to C3b. Nucleic acids (polynucleotides/expression cassettes/nucleotide sequences) described herein are designed to encode a polypeptide that binds to C3b. Nucleic acids (polynucleotides/expression cassettes/nucleotide sequences) described herein are designed to encode a polypeptide fragment of CR1.

In some embodiments the second nucleotide sequence described herein encodes a polypeptide comprising or consisting of an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 2, 3, 4, 12 or 13. In some embodiments the second nucleotide sequence described herein encodes a polypeptide comprising or consisting of an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 3 or 12.

The polypeptide may comprise additional sequence at the N terminus and/or C terminus of the amino acid sequence above. The additional sequence(s) may be sequence from CR1 or may be unrelated to CR1. The sequence identity may be assessed over the whole polypeptide sequence, i.e. including the polypeptide with sequence identity with SEQ ID NO: 2, 3, 4, 12 or 13 and any additional amino acid sequence. Alternatively, the polypeptide may comprise additional sequence that is not taken into account when assessing sequence identity with SEQ ID NO: 2, 3, 4, 12 or 13.

A polypeptide described herein, e.g. that is/can be encoded by a nucleotide sequence provided herein, may be defined by its length. In certain embodiments provided herein, the size of the polypeptide is important so that it can access sites of pathological complement activation. For example, the polypeptide may need to traverse Bruch's membrane to reach sites of unwanted complement activation in the eye, or may need to traverse the glomerular basement membrane to reach sites of unwanted complement activation in the kidney.

The polypeptide may be defined by its ‘total length’, e.g. including any amino acid sequence comprised therein and any additional sequence(s), from the N-terminus to the C-terminus. The ‘total length’ may include or exclude any moieties attached or conjugated to said polypeptide, e.g. that are used to target the polypeptide to particular cells, tissues or sites of complement activation. The ‘total length’ may include or exclude other amino acid sequences or protein domains that are fused to said polypeptide.

Alternatively, the polypeptide may be defined by the length of the amino acid sequence comprised therein (i.e. the amino acid sequence comprising or consisting of a sequence having at least 80% sequence identity to SEQ ID NO: 2, 3, 4, 12 or 13). In such cases, the polypeptide may comprise additional sequence(s) that renders the sequence of the entire polypeptide longer than the specified length of said amino acid sequence.

The polypeptide, or the amino acid sequence contained within the polypeptide, may be 1000 amino acids or fewer in length. The polypeptide, or said amino acid sequence, may comprise or consist of an amino acid sequence of 990 or fewer, 980 or fewer, 970 or fewer, 960 or fewer, 950 or fewer, 940 or fewer, 930 or fewer, 920 or fewer, 910 or fewer, 900 or fewer, 890 or fewer, 880 or fewer, 870 or fewer, 860 or fewer, 850 or fewer, 840 or fewer, 830 or fewer, 820 or fewer, 810 or fewer, 800 or fewer, 790 or fewer, 780 or fewer, 770 or fewer, 760 or fewer, 750 or fewer, 740 or fewer, 730 or fewer, 720 or fewer, or 710 or fewer amino acids.

The polypeptide, or the amino acid sequence contained within the polypeptide, may be 700 amino acids or fewer in length. The polypeptide, or said amino acid sequence, may comprise or consist of an amino acid sequence of 690 or fewer, 680 or fewer, 670 or fewer, 660 or fewer, 650 or fewer, 640 or fewer, 630 or fewer, 620 or fewer, 610 or fewer, 600 or fewer, 590 or fewer, 580 or fewer, 570 or fewer, 560 or fewer, 550 or fewer, 540 or fewer, 530 or fewer, 520 or fewer, 510 or fewer, 500 or fewer, 490 or fewer, 480 or fewer, 470 or fewer, 460 or fewer, 450 or fewer, 440 or fewer, 430 or fewer, 420 or fewer, 410 or fewer, 400 or fewer, 390 or fewer, 380 or fewer, 370 or fewer, 360 or fewer, 350 or fewer, 340 or fewer, 330 or fewer, 320 or fewer, 310 or fewer, 300 or fewer, 290 or fewer, 280 or fewer, 270 or fewer, 260 or fewer, 250 or fewer, 240 or fewer, 230 or fewer, 220 or fewer, 210 or fewer, or 200 or fewer amino acids. This disclosure also provides for a polypeptide, or amino acid sequence as above, that comprises or consists of an amino acid sequence having ‘up to’ any length of amino acids described herein, e.g. up to 1000 amino acids or up to 700 amino acids. Any two end points described herein may be used to make a range of lengths for the polypeptide or amino acid sequence, for example 200-700 amino acids or any combination of end points above.

The polypeptide, or the amino acid sequence contained within the polypeptide, may be 100-700 amino acids in length. The polypeptide, or the amino acid sequence contained within the polypeptide, may be 100-690, 100-680, 100-670, 100-660, 100-650, 100-640, 100-630, 100-620, 100-610, 100-600, 100-590, 100-580, 100-570, 100-560, 100-550, 100-540, 100-530, 100-520, 100-510, 100-500, 100-490, 100-480, 100-470, 100-460, 100-450, 100-440, 100-430, 100-420, 100-410, 100-400, 100-390, 100-380, 100-370, 100-360, 100-350, 100-340, 100-330, 100-320, 100-310, 100-300, 100-290, 100-280, 100-270, 100-260, 100-250, 100-240, 100-230, 100-220, 100-210, or 100-200 amino acids.

The polypeptide, or the amino acid sequence contained within the polypeptide, may be 180-700 amino acids in length. The polypeptide, or the amino acid sequence contained within the polypeptide, may be 180-690, 180-680, 180-670, 180-660, 180-650, 180-640, 180-630, 180-620, 180-610, 180-600, 180-590, 180-580, 180-570, 180-560, 180-550, 180-540, 180-530, 180-520, 180-510, 180-500, 180-490, 180-480, 180-470, 180-460, 180-450, 180-440, 180-430, 180-420, 180-410, 180-400, 180-390, 180-380, 180-370, 180-360, 180-350, 180-340, 180-330, 180-320, 180-310, 180-300, 180-290, 180-280, 180-270, 180-260, 180-250, 180-240, 180-230, 180-220, 180-210, or 180-200 amino acids.

The polypeptide, or the amino acid sequence contained within the polypeptide, may be 194-700 amino acids in length. The polypeptide, or the amino acid sequence contained within the polypeptide, may be 194-690, 194-680, 194-670, 194-660, 194-650, 194-640, 194-630, 194-620, 194-610, 194-600, 194-590, 194-580, 194-570, 194-560, 194-550, 194-540, 194-530, 194-520, 194-510, 194-500, 194-490, 194-480, 194-470, 194-460, 194-450, 194-440, 194-430, 194-420, 194-410, 194-400, 194-390, 194-380, 194-370, 194-360, 194-350, 194-340, 194-330, 194-320, 194-310, 194-300, 194-290, 194-280, 194-270, 194-260, 194-250, 194-240, 194-230, 194-220, 194-210, or 194-200 amino acids.

As will be evident from this disclosure, a polypeptide described herein may be longer than the amino acid sequence comprised therein (said amino acid sequence having at least 80% sequence identity to SEQ ID NO: 2, 3, 4, 12 or 13). Thus, a polypeptide provided herein may have one length above, whilst said amino acid sequence may be of a shorter length. Any of the lengths and/or ranges above can be combined. For example, a polypeptide may be 700 or fewer amino acids in length, whilst the amino acid sequence having at least 80% sequence identity to SEQ ID NO: 2, 3, 4, 12 or 13 may be 400 or fewer amino acids in length. As another example, a polypeptide may be 620 or fewer amino acids in length, whilst the amino acid sequence having at least 80% sequence identity to SEQ ID NO: 2, 3, 4, 12 or 13 may be 100-220 amino acids in length.

The polypeptide, or the amino acid sequence contained within the polypeptide, may have a length of 194 or 212 amino acids.

Nucleic acids (polynucleotides/expression cassettes/nucleotide sequences) and polypeptides provided herein may comprise modifications and/or additional nucleotide or amino acid sequences. The additional sequences may or may not contribute to the length/total length of the nucleic acid/polypeptide as described herein.

In some embodiments, the secretory pathway sequence may comprise or consist of a secretory pathway sequence (also known as a signal peptide or signal sequence). The signal peptide may be present in the newly-translated polypeptide (e.g. prior to processing to remove the signal peptide). The signal peptide may be cleaved from the mature polypeptide once export of the polypeptide chain across the rough endoplasmic reticulum is initiated. In some embodiments, the secretory pathway sequence is derived from Complement Factor H (FH). The polypeptide may comprise a secretory pathway sequence comprising or consisting of SEQ ID NO: 9. The polypeptide may comprise or consist of an amino acid sequence having at least 80% sequence identity to SEQ ID NO:12, e.g. in combination with any one of the lengths of sequence provided herein.

In some embodiments, a polypeptide according to the present invention may additionally comprise a cleavage site for removing the secretory pathway sequence from the polypeptide. Thus, in some embodiments a nucleic acid described herein comprises a nucleotide sequence encoding a cleavage site for removing the secretory pathway sequence from the translated polypeptide. In some embodiments, the cleavage site for removing the secretory pathway sequence from the polypeptide is a cleavage site for an endoprotease. In some embodiments, the cleavage site is for an endoprotease expressed by the cell in which the nucleic acid or polypeptide is expressed. In some embodiments, the cleavage site is a signal peptidase cleavage site. In some embodiments, the cleavage site is a protease cleavage site, e.g. a cleavage site for an endoprotease expressed by cells intended to express the nucleic acid or polypeptide.

A polypeptide according to the present disclosure is preferably soluble. Preferably, it can pass through extracellular membranes, such as Bruch's membrane in the eye or the glomerular basement membrane (GBM) in the kidney, i.e. so it can reach sites of complement activation.

In some embodiments, a polypeptide according to the present invention is not glycosylated. In some embodiments, a polypeptide according to the present invention lacks one or more sites for glycosylation. In some embodiments, the polypeptide of the present invention lacks one or more sites for N-linked glycosylation. In some embodiments, a polypeptide according to the present invention lacks N-linked glycans. In some embodiments, a polypeptide according to the present invention is expressed and/or secreted by cells that are unable to glycosylate or fully glycosylate polypeptides. For example, cells may lack functional glycosyl transferase enzymes. In some embodiments, the polypeptide is aglycosyl (i.e. is not glycosylated). In some embodiments, the polypeptide has been deglycosylated, e.g. by treatment with a glycosidase (e.g. Peptide N-Glycosidase). Deglycosylation is preferably non-denaturing. In some embodiments a polypeptide according to the present invention is partially glycosylated, non-glycosylated or de-glycosylated.

In some embodiments, a polypeptide described herein may comprise amino acid sequence(s) to facilitate expression, folding, trafficking, processing, purification or detection of the polypeptide. For example, the polypeptide may comprise a sequence encoding a protein tag, e.g. a His, (e.g. 6Xhis; SEQ ID NO:41), FLAG, Myc, GST, MBP, HA, E, or Biotin tag, optionally: at the N- or C-terminus of the polypeptide; in a linker; or at the N- or C-terminus of a linker. In some embodiments the polypeptide comprises a detectable moiety, e.g. a fluorescent, luminescent, radio, chemical, nucleic acid or enzymatic label. In some embodiments, the detectable moiety facilitates detection of the polypeptide in a sample obtained from a subject, e.g. following administration to the subject of the polypeptide, nucleic acid, vector, cell or pharmaceutical composition according to the present invention. The sample may be any biological sample obtained from a subject. In some embodiments the sample is a liquid biopsy, such as ocular fluid (tear fluid, aqueous humour, or vitreous), blood, plasma, etc. In some embodiments the sample is a cytological sample or a tissue sample such as a surgical sample, e.g. of ocular cells/tissue.

A polypeptide described herein may be conjugated to other moieties, such as other therapeutic agents. A polypeptide described herein may be conjugated to targeting molecules for administration to particular cells or tissues. Targeting molecules may include, for example, nanoparticles, liposomes, micelles, beads, polymers, metal particles, dendrimers, antibodies, aptamers, nanotubes or micro-sized silica rods. Such targeting molecules may be attached to the polypeptide after expression from a nucleic acid provided herein. Targeting molecules may also be expressed as part of the polypeptide, e.g. from one nucleic acid/polypeptide/expression cassette described herein. In such cases, the targeting molecule may not be considered part of the polypeptide for the assessment of its length, as described herein.

In some embodiments, any additional amino acid sequence described herein (i.e. outside the region of amino acid sequence having at least 80% sequence identity with SEQ ID NO: 2, 3, 4, 12, or 13) lacks substantial sequence identity to one or more of amino acid sequences 1-490, 685-940 and/or 1135-2039 of human CR1 (SEQ ID NO:1, numbered according to Uniprot P17927; Entry version 205 (23 Feb. 2022), Sequence version 3 (2 Mar. 2010)). In some embodiments, the polypeptide/additional amino acid sequence lacks substantial sequence identity to CR1 CCP domains 1-7, 11-14 and/or 18-30. In some embodiments, the additional amino acid sequence has less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity to one or more of amino acid sequences at positions 1-490, 685-940 and/or 1135-2039 of human

CR1 (shown in SEQ ID NO:1). In some embodiments the polypeptide/additional amino acid sequence lacks substantial sequence identity to CR1 long homologous repeat (LHR) domains LHR-A (amino acid positions 1-489 of SEQ ID NO:1) and/or LHR-D (amino acid positions 1394-1842 of SEQ ID NO:1). In some embodiments the polypeptide/additional amino acid sequence lacks substantial sequence identity to LHR-A (amino acid positions 1-489 of SEQ ID NO: 1) and/or LHR-D (amino acid positions 1394-1842 of SEQ ID NO:1).

In some embodiments the polypeptide does not comprise or consist of the whole extracellular domain of CR1 (e.g. sCR1), e.g. SEQ ID NO:3 as described in U.S. Pat. No. 8,664,176B2. In some embodiments, the polypeptide, or amino acid sequence, comprises a sequence that has less than 20%, less than 15%, less than 14%, less than 13%, less than 12%, or less than 11% sequence identity with the entire sequence of SEQ ID NO:3 as described in U.S. Pat. No. 8,664,176B2. In some embodiments, the polypeptide, or amino acid sequence, comprises a sequence that has less than 40%, less than 35%, less than 34% or less than 33% sequence identity to amino acids 1 to 449 of SEQ ID NO:3 as described in U.S. Pat. No. 8,664,176B2. In some embodiments, the polypeptide, or amino acid sequence, comprises a sequence that has less than 30%, less than 29%, less than 28% or less than 27% sequence identity to amino acids 644 to 899 of SEQ ID NO:3 as described in U.S. Pat. No. 8,664,176B2. In some embodiments, the polypeptide, or amino acid sequence, comprises a sequence that has less than 20%, less than 16%, less than 15% or less than 14% sequence identity to amino acids 1094 to 1931 of SEQ ID NO:3 as described in U.S. Pat. No. 8,664,176B2.

In some embodiments the polypeptide/additional amino acid sequence does not comprise a sequence corresponding to CCPs 11-15 of CR1, or amino acid positions 685 to 940 of SEQ ID NO: 1 herein.

In some embodiments, a nucleic acid or nucleotide sequence described herein lacks substantial sequence identity to one or more nucleotide sequences of CR1 mRNA or cDNA outside the sequences provided in SEQ ID NO:5, 6, 7 or 8.

In some embodiments the polypeptide encoded by the nucleic acids described herein is a detached/discrete/separate/individual/isolated molecule. In some embodiments, the polypeptide is not a multi-domain polypeptide. In some embodiments, the polypeptide is a single contiguous amino acid sequence that is unconnected, i.e. not joined, fused or attached, to another amino acid sequence. In some embodiments the polypeptide is not attached by an amino acid linker or a non-amino acid linker to another polypeptide or amino acid sequence. In some embodiments the polypeptide is not a section, part or region of a longer amino acid sequence, i.e. it is not part of an amino acid sequence that exceeds the maximum, specified, polypeptide length. In some embodiments the polypeptide is not part of, or does not form a section of, a fusion protein. In some embodiments the polypeptide may comprise a sequence provided herein and one or more additional amino acids, as long as the maximum length of the polypeptide is not exceeded. The short length of the polypeptides described herein enables the polypeptides to pass through the BrM and reach sites of complement activation.

In some embodiments, the polypeptide/additional amino acid sequence does not comprise a domain or amino acid sequence that binds to VEGF. The polypeptide may not comprise a domain or amino acid sequence that inhibits VEGF. The polypeptide may not comprise a half-life prolonging domain, e.g. an Fc domain as described in WO 2013/082563 A1. In some embodiments, the polypeptide does not comprise, or is not conjugated to, an antibody or antigen-binding molecule. In some embodiments, the polypeptide does not comprise, or is not conjugated to, an antibody or antigen-binding molecule that binds to C3d, e.g. as described in U.S. Pat. No. 11,053,305. In some embodiments, the polypeptide does not comprise all or part of a convertase decay accelerating domain (e.g. from DAF or CD55) and/or all or part of a host cell recognition domain, e.g. as described in WO 2018/002131 A1.

In some embodiments, the mature polypeptide or additional amino acid sequence does not comprise an amino acid sequence from Factor H (other than optionally the signal peptide sequence of SEQ ID NO:9). In some embodiments, the polypeptide/additional amino acid sequence has less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity to SEQ ID NO: 39 herein. In some embodiments, the polypeptide/additional amino acid sequence lacks substantial sequence identity to amino acids 19 to 1213 of SEQ ID NO:39 herein. In some embodiments, the polypeptide/additional amino acid sequence has less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity to amino acids 19 to 1213, amino acids 19 to 264, amino acids 324 to 507, amino acids 987 to 1230 and/or amino acids 1107 to 1230 of SEQ ID NO:39 herein.

In some embodiments the polypeptide/additional amino acid sequence has less than 25% sequence identity to CCPs 1-4 of Factor H (positions 19 to 264 of SEQ ID NO:39 herein). In some embodiments the polypeptide/additional amino acid sequence has less than 20% sequence identity to CCPs 6-8 of Factor H (positions 324 to 507 of SEQ ID NO:39 herein). In some embodiments the polypeptide/additional amino acid sequence has less than 10% sequence identity to CCPs 19-20 of Factor H (positions 1107 to 1230 of SEQ ID NO:39 herein).

In some embodiments, the mature polypeptide or additional amino acid sequence lacks substantial sequence identity to FHL-1 (SEQ ID NO:40). In some embodiments, the polypeptide/additional amino acid sequence has less than 80%, less than 75%, less than 70%, less than 65%, less than 60%, less than 55%, less than 50%, less than 45%, less than 40%, less than 35%, or less than 30% sequence identity to SEQ ID NO:40 herein.

Also provided is at least one cell comprising and/or expressing a nucleic acid, polynucleotide, expression cassette, transgene, vector, plasmid, rAAV, polypeptide or composition described herein. Also provided is a method for expressing a polypeptide in a cell, the method comprising introducing a polynucleotide, expression cassette, transgene, vector, plasmid, rAAV, or composition described herein.

The cell may be any suitable cell to express said molecules/articles. The cell may be a mammalian cell, e.g. human, rodent, non-human primate etc, an insect cell, a plant cell or a bacterial cell. The cell may be in vitro, ex vivo, or in vivo.

The at least one cell may be a cell (e.g. human cell) of the eye, kidney, vascular system, blood, muscle, skin, oesophagus, small or large intestine, intestinal tract, pharynx, trachea, lungs, bronchi, bronchioles, or central nervous system. The at least one cell may be a cancer cell or a tumor cell.

The cell may be one or more eye cells. The cell may be one or more RPE cells, retinal cells, photoreceptor cells (e.g. rod and/or cone cells), or ganglion cells.

The cell may be one or more kidney cells. The cell may be one or more glomerular endothelial cells, macula densa cells, mesangial cells, parietal epithelial cells, podocytes, and/or tubule epithelial cells.

The cell may be one or more cells of the central nervous system (CNS). The cell may be one or more neurons or glial cells (e.g. astrocytes, oligodendrocytes, ependymal cells, and microglia).

Any of the nucleic acids, polynucleotides, expression cassettes, vectors, polypeptides, or cells described herein may be isolated and/or substantially purified.

### Properties of Nucleic Acids and Polypeptides

A polypeptide described herein, e.g. encoded by a polynucleotide or nucleotide sequence provided herein, may possess or demonstrate one or more of the following properties (e.g. as determined in an appropriate assay for said property). A nucleic acid described herein may encode a polypeptide that possesses one or more of said properties. A polypeptide or nucleic acid with one or more such properties may be described as being ‘capable of’ demonstrating said property/properties. As an example, a polypeptide (e.g. encoded by a nucleic acid) may bind to C3b and/or C4b and thus may be described as being ‘capable of binding’ to C3b and/or C4b.

Binds to C3b/C4b;
binds to C3b/C4b in the region of C3b/C4b bound by a cofactor for FI;
binds to C3b/C4b in the region of C3b/C4b bound by Complement Receptor 1 (or a fragment thereof);
binds to C3b/C4b in the region of C3b/C4b bound by Complement Receptor 1 CCP domains 8-10 and/or 15-17 (or a fragment thereof);
binds to C3b/C4b with an affinity of binding which is similar to the affinity of binding to C3b/C4b displayed by a co-factor for FI (or a fragment thereof);
binds to C3b/C4b with an affinity of binding which is higher than the affinity of binding to C3b/C4b displayed by a co-factor for FI (or a fragment thereof);
binds to C3b/C4b with an affinity of binding which is higher than the affinity of binding to C3b/C4b displayed by one or more of FHR1, FHR2, FHR3, FHR4 and/or FHR5;
competes for C3b/C4b binding with one or more of FHR1, FHR2, FHR3, FHR4 and/or FHR5;
reduces the ability of one or more of FHR1, FHR2, FHR3, FHR4 and/or FHR5 to bind to C3b/C4b;
binds to C3b/C4b with an affinity of binding which is higher than the affinity of binding to C3b/C4b displayed by FH and/or FHL-1;
competes for C3b/C4b binding with FH and/or FHL-1; acts as a cofactor for FI;
enables FI-mediated inactivation of C3b/C4b;
enables FI-mediated inactivation of iC3b;
reduces the amount of C3b/C4b, e.g. via FI;
produces, or increases the amount of, iC3b, e.g. via FI;
produces, or increases the amount of, iC3b breakdown products e.g. C3dg, C3d, C3f, and/or C3g, e.g. via FI;
decreases the amount of iC3b via FI compared to the amount of iC3b produced by FH and/or FHL-1, e.g. via FI;
increases the ratio of C3dg to iC3b, e.g. via FI;
increases the ratio of C3d to iC3b, e.g. via FI;
increases the ratio of C3g to iC3b, e.g. via FI;
increases the ratio of C3dg to iC3b, e.g. via FI;
produces, or increases the amount of, C4c and/or C4d, e.g. via FI;
is capable of inhibiting complement activation;
is capable of modulating C3 convertase activity;
is capable of modulating and/or inactivating a complement pathway e.g. the alternative complement pathway and/or the classical complement pathway;
reduces formation and/or deposition of the membrane attack complex (MAC), e.g. in a rodent model of laser-induced CNV;
inhibits formation and/or deposition of the membrane attack complex (MAC), e.g. in a rodent model of laser-induced CNV;
reduces retinal leakage area, e.g. in a rodent model of laser-induced CNV;
reduces retinal lesion area, e.g. in a rodent model of laser-induced CNV;
diffuses through extracellular (EC) membranes, such as Bruch's membrane (BrM) and/or the glomerular basement membrane (GBM);
displays superior ability to diffuse through extracellular (EC) membranes compared to a co-factor for FI (or a fragment thereof), e.g. FH, FHL-1, CR1, CD46;
displays similar ability to diffuse through EC membranes compared to a co-factor for Complement Factor I (or a fragment thereof), e.g. FH, FHL-1, CR1, CD46;
displays superior ability to diffuse through EC membranes compared to FH;
displays similar ability to diffuse through EC membranes compared to FHL-1;
displays superior ability to diffuse through EC membranes compared to FHL-1;
displays similar ability to diffuse through EC membranes compared to soluble CR1;
displays superior ability to diffuse through EC membranes compared to soluble CR1;
displays superior ability to diffuse through EC membranes compared to a multi-domain fusion protein;
acts as a co-factor to enable FI-mediated reduction/prevention of the formation of a functional C3bBb-type C3 convertase;
acts as a co-factor to enable FI-mediated reduction/prevention of the formation of a functional C3bBb3b-type C5 convertase;
acts as a co-factor to enable FI-mediated reduction/prevention of the formation of a functional C4b2a3b-type C5 convertase;
acts as a co-factor to enable FI-mediated reduction of C3bBb-type C3 convertase activity;
acts as a co-factor to enable F-mediated reduction of C3bBb3b-type C5 convertase activity;
acts as a co-factor to enable FI-mediated reduction of C4b2a3b-type C5 convertase activity;
acts as a co-factor to enable FI-mediated reduction of the amount of C3bBb-type C3 convertase;
acts as a co-factor to enable FI-mediated reduction of the amount of C3bBb3b-type C5 convertase;
acts as a co-factor to enable FI-mediated reduction of the amount of C4b2a3b-type C5 convertase;
reduces/prevents the formation of a functional C3bBb-type C3 convertase;
reduces/prevents the formation of a functional C4bC2b-type C3 convertase;
reduces/prevents the formation of a functional C3bBb3b-type C5 convertase;
enables reduction/prevention of the formation of a functional C4b2a3b-type C5 convertase;
reduces/prevents C3bBb-type C3 convertase activity;
reduces/prevents C4bC2b-type C3 convertase activity;
reduces/prevents C3bBb3b-type C5 convertase activity;
reduces/prevents C4b2a3b-type C5 convertase activity;
reduces the amount of C3bBb-type C3 convertase;
reduces the amount of C4bC2b-type C3 convertase;
reduces the amount of C3bBb3b-type C5 convertase; and/or
reduces the amount of C4b2a3b-type C5 convertase . . .

Whether a given polypeptide possesses one or more of the functional properties above can be analysed, for example, as described herein.

The ability of a polypeptide to bind to C3b can be analysed using techniques well known to the person skilled in the art, including ELISA, Surface Plasmon Resonance (SPR; see e.g. Hearty et al., Methods Mol Biol (2012) 907:411-442; or Rich et al., Anal Biochem. 2008 Feb. 1; 373 (1): 112-20), Bio-Layer Interferometry (see e.g. Lad et al., (2015) J Biomol Screen 20 (4): 498-507; or Concepcion et al., Comb Chem High Throughput Screen. 2009 September; 12 (8): 791-800), MicroScale Thermophoresis (MST) analysis (see e.g. Jerabek-Willemsen et al., Assay Drug Dev Technol. 2011 August; 9 (4): 342-353), or by a radiolabelled antigen binding assay (RIA). Through such analysis binding to a given target can be determined and quantified. In some embodiments, the binding may be the response detected in a given assay.

In some embodiments, a polypeptide according to the present invention displays binding to C3b in such an assay which is greater than 1 times, e.g. one of >1.01, >1.02, >1.03, >1.04, >1.05, >1.06, >1.07, >1.08, >1.09, >1.1, >1.2, >1.3, >1.4, >1.5, >1.6, >1.7, >1.8, >1.9, >2, >3, >4, >5, >6, >7, >8, >9, >10, >15, >20, >25, >30, >35, >40, >45, >50, >60, >70, >80, >90, or >100 times the level of binding signal detected in such an assay to a negative control molecule to which the polypeptide does not bind.

In some embodiments a polypeptide according to the present invention is capable of binding to C3b with an affinity of binding which is higher than the affinity of binding to C3b displayed by a co-factor for Complement Factor I (or a fragment thereof) in a given assay. In some embodiments a polypeptide according to the present invention is capable of binding to C3b with an affinity of binding which is at least 1.5 times, at least 2 times, at least 2.5 times, at least 3 times, at least 3.5 times, at least 4 times, at least 4.5 times, at least 5 times, at least 5.5 times, at least 6 times, at least 6.5 times, at least 7 times, at least 7.5 times, at least 8 times, at least 8.5 times, at least 9 times, at least 9.5 times, at least 10 times, at least 15 times, at least 20 times, at least 25 times, at least 30 times, at least 35 times, at least 40 times, at least 45 times, at least 50 times, at least 75 times, at least 100 times, at least 150 times, at least 200 times, at least 250 times, at least 300 times, at least 350 times, at least 400 times, at least 450 times, at least 500 times, at least 550 times, at least 600 times, at least 650 times, at least 700 times, at least 750 times, at least 800 times, at least 850 times, at least 900 times, at least 950 times, or at least 1000 times the affinity of binding to C3b displayed by a co-factor for Complement Factor I (or a fragment thereof) in a given assay. In some embodiments a polypeptide according to the present invention is capable of binding to C3b with an affinity of binding which is 2, 3, 4, 5, 6, 7, 8, 9 or 10 order(s) of magnitude greater than the affinity of binding to C3b displayed by a co-factor for Complement Factor I (or a fragment thereof) in a given assay. In some embodiments the co-factor for Complement Factor I is Complement Factor H or truncated FH isoform FHL-1. The co-factor for Complement Factor I may be CR1.

In some embodiments a polypeptide according to the present invention is capable of binding to C3b with an affinity of binding which is higher than the affinity of binding to C3b displayed by one or more FHR proteins, e.g. one or more of FHR1, FHR2, FHR3, FHR4 and/or FHR5. In some embodiments a polypeptide according to the present invention is capable of binding to C3b with an affinity of binding which is at least 1.5 times, at least 2 times, at least 2.5 times, at least 3 times, at least 3.5 times, at least 4 times, at least 4.5 times, at least 5 times, at least 5.5 times, at least 6 times, at least 6.5 times, at least 7 times, at least 7.5 times, at least 8 times, at least 8.5 times, at least 9 times, at least 9.5 times, at least 10 times, at least 15 times, at least 20 times, at least 25 times, at least 30 times, at least 35 times, at least 40 times, at least 45 times, at least 50 times, at least 75 times, at least 100 times, at least 150 times, at least 200 times, at least 250 times, at least 300 times, at least 350 times, at least 400 times, at least 450 times, at least 500 times, at least 550 times, at least 600 times, at least 650 times, at least 700 times, at least 750 times, at least 800 times, at least 850 times, at least 900 times, at least 950 times, or at least 1000 times the affinity of binding to C3b displayed by one or more FHR proteins.

The level of complement activation/over-activation may be determined by the assays described herein, e.g. abnormal levels of complement components, or by tests/assays that are known by one skilled in the art, e.g. as described in Shih and Murali Am. J. Hematol. 2015, 90:1180-1186; Kirschfink and Mollnes, Clin Diagn Lab Immunol. 2003, 10 (6): 982-989; Nilsson and Ekdahl, Clinical and Developmental Immunology, 2012, Article ID 962702; which are hereby incorporated by reference in their entirety.

The ability of a given polypeptide to diffuse through extracellular membranes, e.g. BrM or GBM, can be analysed e.g. in vitro, e.g. as described in Clark et al J. Immunol (2014) 193, 4962-4970, hereby incorporated by reference in its entirety. The diffusion through such membranes may be detected by measuring the rate of diffusion through to the diffusate chamber and/or detecting the proportion of polypeptide present in the diffusate chamber at the end of the experiment. A similar ability to diffuse through EC membranes may be indicated by detecting a rate of diffusion through to the diffusate chamber which is within 30%, e.g. within one of 25%, 20%, 15%, or 10% of the rate of diffusion for a reference polypeptide, and/or by detecting a proportion of the polypeptide of the present invention present in the diffusate chamber at the end of the experiment that is within 30%, e.g. within one of 25%, 20%, 15%, or 10% of the proportion of a reference polypeptide present in the diffusate chamber. A superior ability to diffuse through EC membranes may be indicated by detecting a rate of diffusion through to the diffusate chamber which is higher (e.g. at least 10%, at least 20%, at least 30%, at least 40%, or at least 50% higher) than the rate of diffusion for a reference polypeptide, and/or by detecting a proportion of the polypeptide of the present invention present in the diffusate chamber at the end of the experiment that is higher (e.g. at least 10%, at least 20%, at least 30%, at least 40%, or at least 50% higher) than the proportion of a reference polypeptide present in the diffusate chamber.

The ability of a given polypeptide to act as a cofactor for Factor I can be determined as described herein, e.g. in Example 3. The presence/absence/level(s) of C3, C3b, iC3b and downstream products can be detected by any suitable method available to the skilled person, such as immunoprecipitation, ELISA, Western blotting etc.

The ability of a given polypeptide to reduce/inhibit the formation and/or deposition of the MAC can be determined as described herein, e.g. in Example 2.6.

The ability of a given polypeptide to reduce retinal leakage and/or retinal lesions in a rodent model of laser-induced CNV can be determined as described herein, e.g. in Example 2.6. Fluorescein angiography (FA) and spectral-domain optical coherence tomography (SD-OCT) may be used to identify leaky CNV lesions and lesion area.

### Therapeutic Applications

The molecules and articles disclosed herein, e.g. nucleic acids, polynucleotides, expression cassettes, vectors, plasmids, polypeptides, cells and compositions, find use in therapeutic and/or prophylactic methods.

The present invention provides a nucleic acid/polynucleotide (or plurality thereof), expression cassette (or plurality thereof), vector (or plurality thereof), polypeptide (or plurality thereof), cell (or plurality thereof), or composition(s) described herein for use in a method of medical treatment or prophylaxis. Also provided is a nucleic acid/polynucleotide (or plurality thereof), expression cassette (or plurality thereof), vector (or plurality thereof), polypeptide (or plurality thereof), cell (or plurality thereof), or composition(s) described herein for use as a medicament. Also provided is the use of a nucleic acid (or plurality thereof), expression cassette (or plurality thereof), vector (or plurality thereof), polypeptide (or plurality thereof), cell (or plurality thereof), or composition(s) described herein in the manufacture of a medicament for treating or preventing a disease or condition. Also provided is a method of treating or preventing a disease or condition, comprising administering to a subject a therapeutically or prophylactically effective amount of a nucleic acid (or plurality thereof), expression cassette (or plurality thereof), vector (or plurality thereof), polypeptide, cell (or plurality thereof), or composition(s) described herein.

The methods may be effective to reduce the development or progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the symptoms of the disease/condition or reduction in some other correlate of the severity/activity of the disease/condition. In some embodiments the methods may prevent development of the disease/condition to a later stage (e.g. a late stage, chronic stage or metastasis).

The terms “disorder”, “disease” and “condition” may be used interchangeably herein and refer to a pathological issue of a body part, organ or system which may be characterised by an identifiable group of signs or symptoms. The term “complement-related disorder” refers to disorders, diseases or conditions that comprise or arise from deficiencies or abnormalities in the complement system. In some embodiments, the complement-related disorder is a disorder driven by complement activation or complement over-activation. The terms “develop”, “developing”, and “development”, e.g. of a disorder, as used herein refer both to the onset of a disease as well as the progression, exacerbation or worsening of a disease state. The term “biomarker(s)” as used herein refers to one or more measurable indicators of a biological state or condition.

It will be appreciated that the molecules and articles of the present invention may be used for the treatment/prevention of any disease/condition/disorder in which the complement system, or activation/over-activation/dysregulation of the complement system, is pathologically implicated. The disease/condition/disorder may be any disease/condition/disorder described herein.

In some embodiments, the disease/condition to be treated/prevented in accordance with the present invention is a disorder characterised by activation/over-activation/dysregulation of the complement system. In some embodiments, an overactive complement response is linked to the presence of C3b and/or iC3b. In some embodiments the disease/condition to be treated or prevented is a complement-related disorder. In some embodiments the disease/condition to be treated or prevented is pathologically associated with complement activation. In some embodiments the disease/condition to be treated or prevented is pathologically associated with complement over-activation. In some embodiments the disease/condition to be treated or prevented is driven by complement activation or over-activation. In some embodiments the disease/condition is complement activation or over-activation.

The disease or condition to be treated or prevented may be a disease/condition which would benefit from one or more of: a reduction in the level or activity of C3bBb-type C3 convertase, C3bBb3b-type C5 convertase or C4b2a3b-type C5 convertase; a reduction in the level of C3b, C5b or C5a; an increase in the level of iC3b, C3f, C3dg, C3g and/or C3d; or a reduction in the level or activity of C3b and/or iC3b and an increase in the level of C3f, C3dg, C3g and/or C3d.

In some embodiments, the disease or condition to be treated or prevented may be a disease/condition associated with C3b or a C3b-containing complex, an activity/response associated with C3b or a C3b-containing complex, or a product of an activity/response associated with C3b or a C3b-containing complex. That is, in some embodiments, the disease or condition to be treated or prevented is a disease/condition in which C3b, a C3b-containing complex, an activity/response associated with C3b or a C3b-containing complex, or the product of said activity/response is pathologically implicated. In some embodiments, the disease/condition may be associated with an increased level of C3b or a C3b-containing complex, an increased level of an activity/response associated with C3b or a C3b-containing complex, or increased level of a product of an activity/response associated with C3b or a C3b-containing complex as compared to the control state. In some embodiments, the disease/condition may be associated with a decreased level of C3 or a C3-containing complex, a decreased level of an activity/response associated with C3 or a C3-containing complex, or a decreased level of a product of an activity/response associated with C3 or a C3-containing complex as compared to the control state.

The treatment may be aimed at reducing the level of C3b or a C3b-containing complex, an activity/response associated with C3b or a C3b-containing complex, or a product of an activity/response associated with C3b or a C3b-containing complex. In some embodiments, the treatment is aimed at: reducing the level or activity of C3bBb-type C3 convertase, C3bBb3b-type C5 convertase or C4b2a3b-type C5 convertase; reducing the level of C3b, C5b or C5a; increasing the level of iC3b, C3f, C3dg, C3g and/or C3d, or reducing the level of C3b and/or iC3b and increasing the level of C3f, C3dg, C3g and/or C3d.

Administration of the articles of the present invention may cause a reduction in the level of C3b or a C3b-containing complex, a reduction in the activity/response associated with C3b or a C3b-containing complex, or a reduction in the product of an activity/response associated with C3b or a C3b-containing complex through cleavage of C3b.

In some embodiments, the treatment may be aimed at reducing the level of C3b or a C3b-containing complex, an activity/response associated with C3b or a C3b-containing complex, or a product of an activity/response associated with C3b or a C3b-containing complex in a subject, e.g. at a particular location, in a particular organ, tissue, structure or cell type, such as eye, kidney, CNS, or skin.

In some embodiments, the treatment may be aimed at reducing the level of C3b or a C3b-containing complex, an activity/response associated with C3b or a C3b-containing complex, or a product of an activity/response associated with C3b or a C3b-containing complex in the eye, e.g. in the retina, choroid, RPE, macula and/or at the BrM/RPE interface.

In some embodiments, the treatment may be aimed at reducing the level of C3b or a C3b-containing complex, an activity/response associated with C3b or a C3b-containing complex, or a product of an activity/response associated with C3b or a C3b-containing complex in the kidney, e.g. in the glomerulus, glomerular basement membrane (GBM), tubules, tubulointerstitium, vasculature, microvasculature, glomerular endothelial cells, macula densa, mesangial cells, parietal epithelial cells, podocytes, and/or tubule epithelial cells.

Administration of the articles of the present invention may cause a reduction in the level of iC3b or an iC3b-containing complex, a reduction in the activity/response associated with iC3b or an iC3b-containing complex, or a reduction in the product of an activity/response associated with iC3b or an iC3b-containing complex through cleavage of iC3b.

In some embodiments, the treatment may be aimed at reducing the level of iC3b or an iC3b-containing complex, an activity/response associated with iC3b or an iC3b-containing complex, or a product of an activity/response associated with iC3b or an iC3b-containing complex in a subject, e.g. at a particular location, in a particular organ, tissue, structure or cell type, such as eye, kidney, CNS, or skin.

In some embodiments, the treatment may be aimed at reducing the level of iC3b or an iC3b-containing complex, an activity/response associated with iC3b or an iC3b-containing complex, or a product of an activity/response associated with iC3b or an iC3b-containing complex in the eye, e.g. in the retina, choroid, RPE, RPE cells, macula and/or at the BrM/RPE interface.

In some embodiments, the treatment may be aimed at reducing the level of iC3b or an iC3b-containing complex, an activity/response associated with iC3b or an iC3b-containing complex, or a product of an activity/response associated with iC3b or an iC3b-containing complex in the kidney, e.g. in the glomerulus, glomerular basement membrane (GBM), tubules, tubulointerstitium, vasculature, microvasculature, glomerular endothelial cells, macula densa, mesangial cells, parietal epithelial cells, podocytes, and/or tubule epithelial cells.

In some embodiments, the treatment may be aimed at reducing the level of iC3b or an iC3b-containing complex, an activity/response associated with iC3b or an iC3b-containing complex, or a product of an activity/response associated with iC3b or an iC3b-containing complex in the CNS, e.g. in neurons, glial cells, astrocytes, oligodendrocytes, ependymal cells, and/or microglia.

“Treatment” may refer to treating, preventing, or reducing the likelihood of a complement-related disorder, such as those described herein.

The complement-related disorder may comprise disruption of the classical, alternative and/or lectin complement pathways. In some cases, the disorder may be associated with deficiencies in, abnormalities in, or absence of regulatory components of the complement system. In some embodiments, the disorder may be a disorder associated with the alternative complement pathway, disruption of the alternative complement pathway and/or associated with deficiencies in, abnormalities in, or absence of regulatory components of the alternative complement pathway. In some cases the disorder is associated with the complement amplification loop. In some cases the disorder is associated with inappropriate activation, over-activation, or dysregulation of the complement system, in whole or in part, e.g. C3 convertase assembly, C3b production, C3b deposition, and/or the amplification loop.

In some cases, the disorder is associated with any one or more of C3, C3b, iC3b, FI, FH, FHL-1, or FHR1-FHR5. In some cases, the disorder is associated with deficiencies or abnormalities in the level and/or activity of any one or more of C3, C3b, iC3b, FI, FH, FHL-1, or FHR1-FHR5. In some cases one or more of these proteins are pathologically implicated in the disorder, e.g. have raised or lower levels compared with a reference/control value.

In some embodiments the disorder is associated with increased levels of any one or more of C3, C3b, C3 convertase and/or C3bBb as compared to a control state. In some embodiments the disorder is associated with decreased levels of any one or more of C3, C3b, C3 convertase and/or C3bBb as compared to a control state. In some embodiments, the disorder is associated with increased levels of iC3b as compared to a control state. In some embodiments, the disorder is associated with decreased levels of iC3b as compared to a control state. In some embodiments the disorder is associated with increased levels of any one or more of C3a, C3f, C3c, C3dg, C3d, and/or C3g as compared to a control state. In some embodiments the disorder is associated with decreased levels of any one or more of C3a, C3f, C3c, C3dg, C3d, and/or C3g as compared to a control state.

The disorder may be characterised by elevated levels of any one or more FH family proteins, e.g. any one or more of FHR1, FHR2, FHR3, FHR4 and/or FHR5. The elevated levels may be in a subject, e.g. may be/have been detected in a subject. That is, the subject to be assessed or treated may have (or be/have been determined to have) elevated levels of one or more of FHR1, FHR2, FHR3, FHR4 and/or FHR5, e.g. assessed by a method provided herein. The disorder may be characterised by elevated circulating levels of one or more of FHR1, FHR2, FHR3, FHR4 and/or FHR5, i.e. in a sample as described herein. The sample may be a blood-derived sample. The sample may be plasma or serum. The sample may be taken/obtained from the CNS, eye or kidney. The sample may be CSF or vitreous fluid. The disorder may be characterised by elevated expression of one or more of FHR1, FHR2, FHR3, FHR4 and/or FHR5 by hepatocytes. The disorder may be characterised by elevated levels of one or more of FHR1, FHR2, FHR3, FHR4 and/or FHR5 detected in a tissue of interest, e.g. eye, kidney, brain, CNS, lung, tumor, vascular tissue. Elevated levels can be determined by comparison to a control value(s)/subject(s) as described herein.

Not all subjects with a complement-related disorder may have elevated levels of one or more FHR proteins. Thus, some subjects with a complement-related disorder may have elevated levels of one or more FHR proteins, and some subjects with the same complement-related disorder may not. In some cases, e.g. as described herein, the presence of elevated levels of one or more FHR proteins can indicate a worse prognosis. Determining the levels of one or more FHR proteins therefore may provide a distinct population of patients who will benefit in particular from treatment with the molecule/articles described herein, e.g. as compared to patients with normal levels of FHR proteins.

The complement-related disorder may be characterised by altered levels of FH and/or FHL-1, either up or down, e.g. in addition to the elevated levels of one or more FHR proteins.

In some cases, the disorder is associated with one or more of CR1, CD46, CD55, C4BP, Factor B (FB), Factor D (FD), SPICE, VCP (or VICE) and/or MOPICE. In some cases, the disorder is associated with deficiencies or abnormalities in the activity of one or more of CR1, CD46, CD55, C4BP, Factor B, Factor D, SPICE, VCP (or VICE) and/or MOPICE, or where one or more of these proteins are pathologically implicated.

In some embodiments, the disorder may be a disorder associated with any one or more of FH, FHL-1, FI, FHR1-FHR5, FB, FD, CR1 and/or CD46, an activity/response associated with any one or more of FH, FHL-1, FI, FHR1-FHR5, FB, FD, CR1 and/or CD46 or a product of an activity/response associated with any one or more of FH, FHL-1, FI, FHR1-FHR5, FB, FD, CR1 and/or CD46. In some embodiments, the disorder is a disorder in which any one or more of FH, FHL-1, FI, FHR1-FHR5, FB, FD, CR1 and/or CD46, an activity/response associated with any one or more of FH, FHL-1, FI, FHR1-FHR5, FB, FD, CR1 and/or CD46, or the product of said activity/response is pathologically implicated. In some embodiments, the disorder may be associated with a decreased level of any one or more of FH, FHL-1, FI, FHR1-FHR5, FB, FD, CR1 and/or CD46, a decreased level of an activity/response associated with any one or more of FH, FHL-1, FI, FHR1-FHR5, FB, FD, CR1 and/or CD46, or a decreased level of a product of an activity/response associated with any one or more of FH, FHL-1, FI, FHR1-FHR5, FB, FD, CR1 and/or CD46 as compared to a control state.

In some embodiments, the disorder may be associated with an increased level of any one or more of FHR1, FHR2, FHR3, FHR4 and/or FHR5, an increased level of an activity/response associated with any one or more of FHR1, FHR2, FHR3, FHR4 and/or FHR5, or an increased level of a product of an activity/response associated with any one or more of FHR1, FHR2, FHR3, FHR4 and/or FHR5 as compared to a control state, see e.g. Zhu et al., Kidney Int. 2018 July; 94 (1): 150-158; Pouw et al., Front Immunol. 2018 Apr. 24; 9:848; both hereby incorporated by reference in their entirety. Methods of treatment may comprise determining the systemic level of any combination of FHR1 to FHR5.

In some embodiments, the disorder may be associated with an increased level of any one or more of FHR1, FHR2 and/or FHR3, an increased level of an activity/response associated with any one or more of FHR1, FHR2 and/or FHR3, or an increased level of a product of an activity/response associated with any one or more of FHR1, FHR2 and/or FHR3. In some embodiments the disorder may be associated with an increased level of FHR4, an increased level of an activity/response associated with FHR4, or an increased level of a product of an activity/response associated with FHR4 as compared to a control state, see e.g. WO 2019/215330 and Cipriani et al., Nat Commun 11, 778 (2020), both hereby incorporated by reference in their entirety. In some embodiments the disorder may be associated with an increased level of FHL-1.

In some cases, the methods described herein find use in treating or preventing a disorder which would benefit from a reduction in the level or activity of one or more of FHR1, FHR2, FHR3, FHR4 and/or FHR5, and optionally FHL-1.

In some embodiments the complement-related disorder that may be treated as described herein is selected from: macular degeneration, age related macular degeneration (AMD), geographic atrophy (‘dry’ (i.e. non-exudative) AMD), early AMD, early onset macular degeneration (EOMD), intermediate AMD, late/advanced AMD, ‘wet’ (neovascular or exudative) AMD, choroidal neovascularisation (CNV), retinal dystrophy, glaucoma (open-angle or closed-angle), neuromyelitis optica (neuromyelitis optica spectrum disorder (NMOSD)), diabetic retinopathy, Stargardt disease, autoimmune uveitis, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), kidney injury/damage/dysfunction, glomerular diseases, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), ANCA vasculitis, autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, skin diseases e.g. inflammatory skin diseases, atherosclerosis, inflammatory diseases, neurodegeneration/neurodegenerative disease, dementia, frontotemporal dementia, multiple sclerosis (MS), Lewy body disease, Amyotrophic lateral sclerosis (ALS), Parkinson's disease, Alzheimer's disease, Huntington's disease, epilepsy, schizophrenia, acute brain trauma e.g. traumatic brain injury, neonatal hypoxic ischemic encephalopathy (HIE), myasthenia gravis (MG), Guillain-Barré syndrome (GBS), prion diseases, cancer, lung cancer, cancer, glioblastoma e.g. glioblastoma multiforme (GBM), stroke, insulin resistance, diabetes, and an infectious disease.

All references in the following paragraphs are hereby incorporated by reference in their entirety.

The disorder to be treated may be an ocular disorder. In some embodiments, a disease or condition that is assessed, diagnosed, treated or prevented as described herein is a complement-related ocular disease. In some embodiments, the disorder is selected from: macular degeneration, age related macular degeneration (AMD), geographic atrophy (‘dry’ (i.e. non-exudative) AMD), early AMD, early onset macular degeneration (EOMD), intermediate AMD, late/advanced AMD, ‘wet’ (neovascular or exudative) AMD, choroidal neovascularisation (CNV), retinal dystrophy, glaucoma (open-angle or closed-angle), neuromyelitis optica, diabetic retinopathy, Stargardt disease, and autoimmune uveitis,

In some embodiments, the disorder is macular degeneration. In some embodiments, the disorder may be selected from, i.e. is one or more of: age-related macular degeneration (AMD), choroidal neovascularisation (CNV), macular dystrophy, and diabetic maculopathy. As used herein, the term “AMD” includes early AMD, intermediate AMD, late/advanced AMD, geographic atrophy (‘dry’ (i.e. non-exudative) AMD), and ‘wet’ (i.e. exudative or neovascular) AMD, each of which may be a disorder in its own right that can be detected, treated and/or prevented as described herein. In some embodiments the disease or condition to be treated or prevented is a combination of the diseases/conditions above, e.g. ‘dry’ and ‘wet’ AMD. In some embodiments the disease or condition to be treated or prevented is not ‘wet’ AMD or choroidal neovascularisation. AMD is commonly-defined as causing vision loss in subjects age 50 and older. In some embodiments a subject to be treated is age 50 or older, i.e. is at least 50 years old.

Macular degeneration is believed to be driven in part by complement-mediated attack on ocular tissues. A major driver of AMD risk is genetic variation at the RCA locus resulting in dysregulation of the complement cascade. AMD is the leading cause of blindness in the developed world: currently responsible for 8.7% of all global blind registrations. It is estimated that 196 million people will be affected by 2020, increasing to 288 million by 2040 (Wong et al. Lancet Glob Heal (2014) 2: e106-16). AMD manifests as the progressive destruction of the macula, the central part of the retina at the back of the eye, leading to loss of central visual acuity. Early stages of the disease see morphological changes in the macula such as the loss of blood vessels in the choriocapillaris (Whitmore et al., Prog Retin Eye Res (2015) 45:1-29); a layer of capillaries found in the choroid (a highly vascularized layer that supplies oxygen and nutrition to the outer retina). The choriocapillaris is separated from the metabolically active retinal pigment epithelium (RPE) by Bruch's membrane (BrM); a thin (2-4 μm), acellular, five-layered sheet of extracellular matrix. The BrM serves two major functions: the substratum of the RPE and a blood vessel wall. The structure and function of BrM is reviewed e.g. in Curcio and Johnson, Structure, Function and Pathology of Bruch's Membrane, In: Ryan et al. (2013), Retina, Vol. 1, Part 2: Basic Science and Translation to Therapy. 5th ed. London: Elsevier, pp466-481, which is hereby incorporated by reference in its entirety.

The role of complement in AMD is reviewed, for example, by Zipfel et al. Chapter 2, in Lambris and Adamis (eds.), Inflammation and Retinal Disease: Complement Biology and Pathology, Advances in Experimental Medicine and Biology 703, Springer Science+Business Media, LLC (2010), which is hereby incorporated by reference in its entirety. The key characteristics of AMD are indicative of over-active complement, including cell/tissue destruction and a local inflammatory response. Hallmark lesions of early AMD, termed drusen, develop within BrM adjacent to the RPE layer (Bird et al, Surv Ophthalmol 1995, 39 (5): 367-374). Drusen are formed from the accumulation of lipids, proteins and cellular debris, and include a swathe of complement activation products (Anderson et al., Prog Retin Eye Res 2009, 29:95-112; Whitcup et al., Int J Inflam 2013, 1-10). The presence of drusen within BrM disrupts the flow of nutrients from the choroid across this extracellular matrix to the RPE cells, which leads to cell dysfunction and eventual death, leading to the loss of visual acuity.

As used herein “early AMD” refers to a stage of AMD characterised by the presence of medium-sized drusen, commonly having a diameter of up to ~200 μm, within Bruch's membrane adjacent to the RPE layer. Subjects with early AMD typically do not present with significant vision loss. As used herein “intermediate AMD” refers to a stage of AMD characterised by large drusen and/or pigment changes in the retina. Intermediate AMD may be accompanied by some vision loss. As used herein “late AMD” refers to a stage of AMD characterised by the presence of drusen and vision loss, e.g. severe central vision loss, due to damage to the macula. In all stages of AMD, ‘reticular pseudodrusen’ (RPD) or ‘reticular drusen’ (also referred to as subretinal drusenoid deposits (SDD)) may be present, referring to the accumulation of extracellular material in the subretinal space between the neurosensory retina and RPE. “Late AMD” encompasses ‘dry’ and ‘wet’ AMD. In ‘dry’ AMD (also known as geographic atrophy), there is a gradual breakdown of the light-sensitive cells in the macula that convey visual information to the brain and of the supporting tissue beneath the macula. In ‘wet’ AMD (also known as choroidal neovascularization, neovascular and exudative AMD), abnormal blood vessels grow underneath and into the retina. These vessels can leak fluid and blood which can lead to swelling and damage of the macula and subsequent scar formation. The damage may be rapid and severe.

‘Dry’ AMD, also known as (or involving) geographic atrophy (GA), represents around 50% of late-stage AMD cases. In the remaining percentage of late-stage cases, choroidal neovascularisation (CNV) develops, in which the increased synthesis of vascular endothelial growth factor (VEGF) by RPE cells promotes new blood vessel growth from the choroid/choriocapillaris that breaks through BrM into the retina. These new blood vessels leak and eventually form scar tissue; this is referred to as ‘wet’ (neovascular or exudative) AMD. ‘Wet’ AMD is the most virulent form of late-stage AMD and has different disease characteristics to ‘dry’ AMD. There are treatments for wet AMD, e.g. Macugen, Avastin and Lucentis, where for example the injection of anti-VEGF agents into the vitreous of the eye can slow or reverse the growth of these blood vessels, although it cannot prevent their formation in the first place.

Potential treatments for GA have focused on PEGylated molecules. For example, pegcetacoplan is a synthetic peptide-based inhibitor of C3 containing two cyclic peptides linked by a polyethylene glycol chain. As another example, avacincaptad pegol is an RNA aptamer covalently bound to a branched polyethylene glycol (PEG) molecule that targets C5. Both of these therapies, delivered regularly by monthly intravitreal (IVT) injections, see a moderate slowing of GA lesion progression. However, both still have drawbacks, including the regular administration regime and increased incidence of CNV conversation (~9% in both treatments, thought to be due to their pegylated formulation). Part of the efficacy ceiling problem observed by both therapies may lie in the fact that they only address part of the consequence of complement over-activation: they both prevent the continuing inflammatory amplification loop and anaphylatoxin generation by targeting either C3 or C5. They do not, however, address the opsonisation of surrounding cells and tissues, i.e. the deposition of C3b and iC3b onto surfaces which then labels such surfaces for destruction.

In contrast, the articles of the present disclosure are capable of driving complete degradation of deposited C3b all the way through to C3d, and thus prevent complement-mediated inflammation and immune cell recruitment (through conversion of C3b into iC3b), and are expected to prevent tissue remodelling resulting from opsonisation of surfaces (through cleavage of iC3b into C3dg and ultimately into C3d).

In some embodiments the disorder is early-onset macular degeneration (EOMD). As used herein “EOMD” refers to a phenotypically severe sub-type of macular degeneration that demonstrates a much earlier age of onset than classical AMD and results in many more years of substantial visual loss. Sufferers may show an early-onset drusen phenotype comprising uniform small, slightly raised, yellow subretinal nodules randomly scattered in the macular, also known as ‘basal laminar drusen’ or ‘cuticular drusen’. EOMD may also be referred to as “middle-onset macular degeneration”. The EOMD patient subset is described in e.g. Boon C J et al. Am J Hum Genet 2008; 82 (2): 516-23; van de Ven J P, et al. Arch Ophthalmol 2012; 130 (8): 1038-47; and Taylor, R. L. et al., Ophthalmol. 2019, 126, 1410-1421, all of which are hereby incorporated by reference in their entirety. Similar to other types of macular degeneration, EOMD is related to complement dysregulation and disrupted Factor H activity. In some embodiments a subject to be treated is age 49 or younger. In some embodiments a subject to be treated is between ages 15 and 49, i.e. is between 15 and 49 years old. In some embodiments the disease or condition to be treated is a macular dystrophy. A macular dystrophy can be a genetic condition, usually caused by a mutation in a single gene, that results in degeneration of the macula.

The assessment methods described herein may be used for determining whether a subject is at risk of onset of macular degeneration, e.g. EOMD and/or AMD, and/or is at risk of EOMD and/or AMD progression. In some cases, the disorder is selected from EOMD, AMD, geographic atrophy (‘dry’ (i.e. non-exudative) AMD), early AMD, intermediate AMD, late/advanced AMD, ‘wet’ (neovascular or exudative) AMD, choroidal neovascularisation (CNV) and retinal dystrophy. In some cases, the subject has or is suspected to have a complement-related disorder. In some cases the disorder is AMD. In some cases the disorder is EOMD.

The role of complement in ocular diseases is reviewed in e.g. Jha et al., Mol Immunol. 2007 September; 44 (16): 3901-3908. The role of complement in glaucoma is discussed in e.g. Reinehr et al., Front Cell Neurosci. 2021; 15:718087; and Hoppe et al., Investigative Ophthalmology & Visual Science June 2022, Vol. 63, 1609-A0432. The role of complement in neuromyelitis optica is discussed in e.g. Asavapanumas et al., Expert Opin Biol Ther. 2021 August; 21 (8): 1073-1086. The role of complement in Stargardt disease is reported in e.g. Hu et al., Redox Biol. 2020 October: 37:101787. All references above are hereby incorporated by reference in their entirety.

In some embodiments the disorder is one associated with the kidney, e.g. nephropathy/a nephropathic disorder.

Numerous complement proteins have been implicated in complement-related kidney disorders, see e.g. De Vriese et al., J Am Soc Nephrol. 2015 December; 26 (12): 2917-2929. In some cases a kidney disorder is characterised by the deposition of C3, e.g. the glomerular pathologies (see e.g. Skerka et al 2013, supra). FH, FHL-1, FHR1, FHR2, FHR3 and FHR5 have been implicated in IgA nephropathy (see e.g. Poppelaars et al., J Clin Med. 2021, 10 (20): 4715; Zhu et al., Kidney Int. 2018 July, 94 (1): 150-158). Poppelaars et al suggest that FHR1 and FHR5 compete with the regulatory function of Factor H, such that the FHR proteins amplify alternative pathway activation and thereby stimulate development and progression of IgA nephropathy. FHR5 has been implicated in C3 glomerulopathy and renal impairment (see e.g. Medjeral-Thomas et al., Kidney Int Rep. 2019, 4 (10): 1387-1400), as well as glomerular damage and kidney injury (e.g. Malik et al., PNAS, 2021, 118 (13) e2022722118). Abnormal FHR hybrid proteins have also been reported in C3 glomerulopathy, and are thought to compete with FH for C3b binding and regulation (see e.g. Wong & Kavanagh, Semin Immunopathol. 2018; 40 (1): 49-64). Wong & Kavanagh also discuss the involvement of FH, FHR1 and FHR3 in atypical Haemolytic Uremic Syndrome (aHUS) and paroxysmal nocturnal hemoglobinuria (PNH). FHR1 and FHR5 were detected in the glomeruli of patients with Dense Deposit Disease (DDD)/membranoproliferative glomerulonephritis type II, see e.g. Sethi et al., Kidney Int. 2009, 75 (9): 952-60, and Abrera-Abeleda et al., J Med Genet. 2006, 43 (7): 582-589. Elevated FHR1 levels have been implicated in ANCA vasculitis (see e.g. Skerka et al., Br J Pharmacol. 2021 July; 178 (14): 2823-2831).

In some cases, the disorder is associated with autoimmunity, e.g. an autoimmune disease.

Autoimmune diseases are associated with complement dysregulation. Schafer et al., Front Immunol. 2016, 7:542 describes the role of elevated FHR3 in a selection of autoimmune diseases, including kidney diseases and rheumatoid patients. Goicoechea de Jorge et al., PNAS 2013, 110 (12): 4685-90 discloses that CFH mutations increase susceptibility to aHUS, DDD, and meningococcal sepsis, and that FHR3 is involved in the pathogenesis of systemic lupus erythematosus (SLE). It also reports that dimerization of the FHR proteins, e.g. FHR1, FHR2 and FHR5, enhanced their ability to compete with FH for C3b binding and deregulate complement activation. Legatowicz-Koprowska et al., Reumatologia. 2020, 58 (6): 357-366 reports the absence of complement cascade proteins in patients with primary Sjögren's syndrome. The role of complement in autoimmune diseases is reviewed in e.g. Thurman and Yapa, Front Immunol. 2019; 10:672, which is hereby incorporated by reference in its entirety.

Activation of the complement alternative pathway has been suggested as having a role in the development of atherosclerosis (see e.g. Speidl et al., J Thromb Haemost. 2011, 9 (3): 428-40; Malik et al., Circulation. 2010, 122 (19): 1948-56; and Machalińska et al., Acta Ophthalmol. 2012, 90 (8): 695-703).

In some cases, the disorder is cancer. Any cancer may be treated as described herein. The cancer may be a liquid or blood cancer, such as leukemia, lymphoma or myeloma. In other cases, the cancer is a solid cancer, such as breast cancer, lung cancer, liver cancer, colorectal cancer, nasopharyngeal cancer, kidney cancer or glioma. In some cases, the cancer is located in the liver, bone marrow, lung, spleen, brain, pancreas, stomach or intestine. In some cases the cancer is lung cancer. In some embodiments the complement-related disorder is an indoleamine 2,3-dioxygenase 1 (IDO)-expressing cancer.

Complement activation plays a role in the development and progression of cancer. DeCordova et al., Immunobiology. 2019, 224 (5): 625-631 reports that FHR5 is secreted by primary tumor cells derived from Glioblastoma multiforme (GBM) patients and may be used by the cells to resist complement mediated lysis. Afshar-Kharghan, J Clin Invest. 2017, 127 (3): 780-789 reports that expression of complement factors is increased in malignant tumors, including the FHR proteins which would outcompete FH and lead to complement dysregulation. Alternatively, if tumors become hypoxic, then this can lead to a downregulation in FH expression and thus an increase in complement inflammatory activity, such that inhibition of complement activation is a therapeutic option (e.g. Pio et al., Semin Immunol. 2013, 25 (1): 54-64). FH has been reported as a biomarker for lung cancer, squamous lung cancer, bladder cancer, ovarian cancer, liver cancer and SCC (e.g. Revel et al., Antibodies (Basel). 2020, 9 (4): 57).

In some cases the cancer is glioblastoma e.g. glioblastoma multiforme (GBM). Glioblastoma (GBM) is the most common malignant primary central nervous system (CNS) cancer in adults. GBM is among the malignancies that are uniquely unresponsive to cancer immunotherapy.

As described in WO 2022/058447 A1, indoleamine 2,3-dioxygenase 1 (IDO) activity in tumour cells increased expression of FH and FHL-1, which were found to be associated with expression of immunosuppressive genes, suppression of anti-tumour immune responses, poorer survival outcomes for glioma patients and a faster rate to GBM recurrence. See also Zhai et al., Clin Cancer Res, 2021 DOI: 10.1158/1078-0432.CCR-21-1392. Thus, in some embodiments the complement-related disorder is IDO-expressing GBM. In some embodiments the complement-related disorder is isocitrate dehydrogenase (IDH)-expressing GBM. In some embodiments the GBM expresses both IDO and IDH. In some embodiments the cancer, e.g. GBM, comprises tumour cells with increased expression of FH and/or FHL-1.

In some cases, the disorder is inflammation. In some cases, the disorder is associated with inflammation. In some cases the disorder is an inflammatory disease. The complement system is designed to induce a series of inflammatory responses that help to fight infection. Unwanted or pathological inflammation can be caused by pathological activation of complement. Any disorder described herein may be associated with inflammation, e.g. as a result of complement over-activation. The disorder may be selected from hereditary angioedema (HAE), acquired angioedema (AAE), encephalomyelitis, inflammatory skin diseases, psoriasis, acne vulgaris, hidradenitis suppurativa (acne inversa), tissue injury, ischemia/reperfusion (I/R) in injury, myocardial infarction, stroke, hemorrhagic shock, severe trauma, organ transplantation, systemic lupus erythematosus (SLE), cutaneous lupus erythematosus (CLE), inflammatory bowel disease (IBD), urticaria, urticarial vasculitis, auto-immune bullous dermatoses e.g. bullous pemphigoid, autoimmune disorders e.g. that are associated with inflammation, and neuroinflammation. The role of complement in inflammatory diseases is reviewed in e.g. Markiewski and Lambris, Am J Pathol. 2007 September; 171 (3): 715-727; and Giang et al., Front Immunol. 2018 Apr. 16:9:639; which are hereby incorporated by reference in their entirety.

In some cases the disorder is neurodegeneration or neurodegenerative disease. The disorder may affect the central nervous system (CNS). The disorder may comprise progressive atrophy and loss of function of neurons. The disorder may involve inflammation and/or inflammatory responses. The disorder may involve autoimmunity and/or autoimmune responses. The disorder may be selected from Parkinson's disease, Alzheimer's disease, dementia, frontotemporal dementia, stroke, Lewy body disease, Amyotrophic lateral sclerosis (ALS), multiple sclerosis (MS), Huntington's disease, epilepsy, schizophrenia, acute brain trauma e.g. traumatic brain injury, neonatal hypoxic ischemic encephalopathy (HIE), myasthenia gravis (MG), Guillain-Barré syndrome (GBS) and prion diseases. The role of the complement system in neurodegenerative disease is reviewed in e.g. Schartz and Tenner, J Neuroinflammation. 2020 Nov. 25; 17 (1): 354; Chen et al., Biomolecules. 2022 February; 12 (2): 337; and Dalakas et al., Nat Rev Neurol. 2020 November; 16 (11): 601-617, which are hereby incorporated by reference in their entirety. The disorder may involve synaptic pruning, i.e. refinement of synaptic circuits involving the phagocytosis of “weak” or inactive synapses by microglial cells via engagement of synapse-bound iC3b and the microglial CR3 complement receptor (CD11b/CD18), see e.g. Gomez-Arboledas et al., Immunotargets Ther. 2021; 10:373-386, which is hereby incorporated by reference in its entirety. Excessive complement-mediated synaptic pruning results in an excessive elimination of synapses and is associated with cognitive impairment, e.g. in Alzheimer's disease, MS, stroke, Parkinson's disease, traumatic brain injury, ALS, epilepsy, and brain cancer.

Elevated levels of FHR1 and FHR3 have been found in plasma from patients with Alzheimer's disease, see e.g. Chen & Xia, J Alzheimers Dis. 2020, 76 (1): 349-368; and Ashton et al., Alzheimers Dement (Amst). 2015, 1 (1): 48-60 (see also Clark and Bishop J Clin Med. 2015 January; 4 (1): 18-31). Thus, elevated levels of FHR proteins are associated with dementia-related disorders. Increased levels of FHR proteins (FHRs 1, 2 and 5) are associated with multiple sclerosis, see e.g. Loveless et al., Brain Pathol. 2018 July; 28 (4): 507-520. Pouw and Ricklin, Semin Immunopathol. 2021, 43 (6): 757-771 discusses the role of FHR proteins as FH competitors and reviews the adverse effect of complement activation in the central nervous system, such as in the context of Alzheimer's disease, Parkinson's disease, schizophrenia, myasthenia gravis (MG), amyotrophic lateral sclerosis (ALS), and Guillain-Barré syndrome (GBS).

The role of complement in various diseases is described in e.g. Morgan, B. P., Complement in the pathogenesis of Alzheimer's disease. Semin Immunopathol, 2018. 40 (1): p. 113-124; Halbgebauer, R., et al., Janus face of complement-driven neutrophil activation during sepsis. Semin Immunol, 2018. 37: p. 12-20; Ma, Y., et al., Significance of Complement System in Ischemic Stroke: A Comprehensive Review. Aging Dis, 2019. 10 (2): p. 429-462; Bonifati and Kishore, Role of complement in neurodegeneration and neuroinflammation. Mol Immunol. 2007 February; 44 (5): 999-1010; Kleczko, E. K., et al., Targeting the Complement Pathway as a Therapeutic Strategy in Lung Cancer. Front Immunol, 2019. 10: p. 954; and Schafer N. et al., Complement Regulator FHR-3 Is Elevated either Locally or Systemically in a Selection of Autoimmune Diseases, Front Immunol. 2016; 7:542, which are all hereby incorporated by reference in their entirety. For example, FHL-1 is expressed more in certain tumour cell lines than FH (Junnikkala et al (2000) J. Immunol. 164:6075-81) and glioblastoma tumours have been shown to express FHR proteins (DeCordova et al. (2019) Immunobiology 224:625-631), both references hereby incorporated in their entirety.

Increased circulating FH levels in subjects with altered glucose tolerance has been linked to insulin resistance and metabolic disturbances (Moreno-Navarrete et al., Diabetes. 2010, 59 (1): 200-9).

In some embodiments, the complement-related disorder is an infectious disease. Complement is a major component of the innate immune system involved in defending against foreign pathogens, including bacteria, viruses, fungi and parasites. Activation of complement leads to robust and efficient proteolytic cascades, which result in opsonization and lysis of the pathogen as well as in the generation of the classical inflammatory response through the production of potent proinflammatory molecules. The role of complement in innate and adaptive immune responses is reviewed in e.g. Dunkelberger, J., Song, W C. Cell Res 2010; 20, 34-50, and Rus H et al., Immunol Res. 2005; 33 (2): 103-12, which are hereby incorporated by reference in their entirety.

In some embodiments the complement-related disorder is infection by severe acute respiratory syndrome-related coronavirus (SARSr-CoV). In some embodiments the complement-related disorder is infection with SARS-COV-2. In some embodiments the complement-related disorder is a disease/condition caused or exacerbated by SARS-COV-2 infection, e.g. COVID-19 or another disease/condition for which infection with SARS-COV-2 is a contributing factor.

The virology of SARSr-COV and epidemiology of disease associated with SARSr-CoV infection is reviewed, for example, in Cheng et al., Clin Microbiol Rev (2007) 20 (4): 660-694 and de Wit et al., Nat Rev Microbiol (2016) 14:523-534, both of which are hereby incorporated by reference in their entirety. SARSr-COV is a species of coronavirus of the genus Betacoronavirus and subgenus Sarbecoronavirus that infects humans, bats and certain other mammals. It is an enveloped positive-sense single-stranded RNA virus. Two strains of SARSr-COV have caused serious outbreaks of severe respiratory diseases in humans: SARS-COV, which caused an outbreak of severe acute respiratory syndrome (SARS) between 2002 and 2003, and SARS-COV-2, which has caused the coronavirus disease 2019 (COVID-19) pandemic.

As used herein, “SARS-COV-2” refers to the SARS-COV having the nucleotide sequence of GenBank: MN996527.1 (“Severe acute respiratory syndrome coronavirus 2 isolate WIV02, complete genome”), reported in Zhou et al., Nature (2020) 579:270-273, and encompasses variants thereof having a nucleotide sequence with at least 85% sequence identity (e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or greater sequence identity) to the nucleotide sequence of GenBank: MN996527.1. Variants of SARS-COV-2 of particular interest include: (i) the variant designated VUI-202012/01, which belongs to the B.1.1.7 lineage, having the canonical nucleotide sequence of GISAID accession EPI_ISL_601443; (ii) the variant designated 501Y.V2/B.1.351, having the canonical nucleotide sequence of GISAID accession EPI_ISL_768642; (iii) the variant known as B.1.1.248/P.1, having the canonical nucleotide sequence of GISAID accession EPI_ISL_792680; (iv) the variant known as B.1.617.1; and (v) the variant known as B.1.617.2. In some embodiments, the complement-related disorder is a disease/condition caused by infection of SARS-COV-2 variants B.1.1.7, B.1.1.248/P.1, B.1.617.1 and/or B.1.617.2.

The clinical features of COVID-19 are described in Lechien et al., Journal of Internal Medicine (2020) 288 (3): 335-344, International Severe Acute Respiratory and Emerging Infections Consortium (ISARIC). COVID-19 Report: 19 May 2020: ISARIC; 2020 and Docherty et al., BMJ (2020) 369: m1985, which are hereby incorporated by reference in their entirety. Common symptoms include cough, fever, headache, dyspnoea, anosmia, pharyngitis, nasal obstruction, rhinorrhoea, asthenia, myalgia, joint pain, gustatory dysfunction, abdominal pain, vomiting, and diarrhoea. The majority patients present with mild/moderate disease, however hospitalisation is sometimes required in particularly in elderly patients and/or patients having comorbidities such as diabetes and cardiovascular disease. A major complication in COVID-19 is progression to acute respiratory distress syndrome (ARDS), which presents as dyspnoea and acute respiratory failure, with patients requiring mechanical ventilation. In some embodiments the complement-related disorder is ARDS or acute respiratory failure.

Complement activation has been implicated in the pathogenesis of severe SARS-COV-2 infection. Circulating markers of complement activation are elevated in patients with COVID-19 compared to those with influenza and to patients with non-COVID-19 respiratory failure, Patients hospitalized with COVID-19 reportedly have significantly higher median plasma sC5b-9, C5a, and Factor B levels compared to those with influenza, pneumonia or sepsis, and certain markers of complement activation have been associated with worse outcomes in COVID-19 patients, see e.g. Ma L et al., Sci Immunol. 2021 May 13; 6 (59): eabh2259. Patients requiring ICU treatment, or who died from COVID-19 infection, were found to have significantly higher Factor D levels.

The levels of SARS-COV-2 antigens and SARS-COV-2 RNA in the blood reportedly correlates with the level of IL-6, inflammation, respiratory failure and death, see e.g. Brasen C L et al., Clin Chem Lab Med. 2021 Aug. 27. Doi: 10.1515/cclm-2021-0694.

As described in Example 5 of WO 2022/058447 A1, elevated levels of FHL1, FHR1, FHR2, FHR3, FHR4 and FHR5 were observed to correlate with increasing COVID-19 severity, with the highest levels being observed in subjects with clinically severe COVID-19 requiring assisted ventilation. Thus, detection of one or more of FHL1, FHR1, FHR2, FHR3, FHR4 and/or FHR5 may predict the likelihood of a subject developing severe COVID-19. Appropriate treatment and monitoring can thus be deployed. The methods described herein are also useful for predicting the risk of development of conditions associated with SARS-COV-2 infection, as well as predicting the severity of such infection, e.g. the likelihood of developing severe or critical COVID-19 and associated complications such as ARDS.

In some cases, the subject to be treated has or is suspected to have a complement-related disorder, e.g. a disorder described herein.

In some aspects, the present invention provides a method for treating or preventing a complement-related disorder in a subject, the method comprising administering an effective amount of an article/molecule described herein, wherein the subject to be treated has been determined to have atypical presence or levels of one or more complement proteins, e.g. detected/determined as described herein, as compared to a control subject and/or reference value(s). In some aspects the subject has been determined to be at risk of developing a complement-related disorder, and/or identified as having a complement-related disorder.

In other aspects, the present invention provides an article/molecule described herein for use in a method of treating or preventing a complement-related disorder in a subject, the method comprising administering an effective amount of the article/molecule, wherein the subject has/has been determined to have atypical presence or levels of one or more complement proteins, e.g. determined as described herein, as compared to a reference value(s). In some aspects the subject has been determined to be at risk of developing a complement-related disorder, and/or identified as having a complement-related disorder.

In some aspects, provided is the use of an article/molecule described herein in the manufacture of a medicament for treating or preventing a complement-related disorder in a subject, wherein the subject has/has been determined to have atypical presence or levels of one or more complement proteins, e.g. determined as described herein, as compared to a reference value(s). In some aspects the subject has been determined to be at risk of developing a complement-related disorder, and/or identified as having a complement-related disorder.

Also provided is a method of treating or preventing a complement-related disorder in a subject, or an article/molecule described herein for use in a method of treating or preventing a complement-related disorder in a subject, the method comprising administering an effective amount of an article/molecule described herein wherein the subject is selected for treatment if the subject has/has been determined to have atypical presence or levels of one or more complement proteins, e.g. determined as described herein, as compared to a reference value(s). In some aspects the subject has been determined to be at risk of developing a complement-related disorder, and/or identified as having a complement-related disorder.

In various aspects provided herein, the subject to be treated has atypical presence or levels of at least one complement protein, preferably one or more of FH, FHL-1, FHR1, FHR2, FHR3, FHR4, FHR5, FI, C3, C3b, C3a, iC3b, C3f, C3c, C3dg, and/or C3d. In some embodiments, the subject to be treated has atypical presence of levels of one or more of FHR1, FHR2, and/or FHR3, and optionally FHR4 and/or FHR5, and/or FHL-1. The subject may benefit from treatment to reduce the level of any complement proteins that are increased as compared to a reference value(s) and/or from treatment to increase the level of any complement proteins that are decreased as compared to a reference value(s).

As used herein, ‘treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention/prophylaxis of a disease/condition may refer to prevention of a worsening of the condition or prevention of the development of the disease/condition, e.g. preventing an early stage disease/condition developing to a later, chronic, stage.

In some embodiments, the subject is selected for therapeutic or prophylactic treatment with an article/molecule described herein based on their being determined to possess one or more genetic factors for AMD and/or EOMD, e.g. one or more AMD-associated and/or EOMD-associated genetic variants, or for a macular dystrophy. Suitable genetic factors are described herein. In some embodiments, the subject has been determined to have one or more such genetic factors. In some embodiments, the methods provided herein comprise determining whether a subject possesses one or more such genetic factors. Such methods and genetic factors are described herein. Thus, provided herein is a method of diagnosing, treating or preventing a complement-related disorder in a subject, wherein the subject has/has been determined/is determined to possess one or more genetic factors for AMD and/or EOMD, and wherein the subject has/has been determined/is determined to have atypical presence or levels of one or more complement proteins, e.g. detected/determined as described herein, as compared to a reference value(s); optionally wherein the method comprises administering an article/molecule described herein.

The term “subject” refers to a subject, patient or individual and may be any animal or human. The subject is preferably mammalian, more preferably human. The subject may be a non-human mammal, but is more preferably human. The subject may be male or female. The subject may be a patient. Therapeutic uses may be in human or animals (veterinary use). The subject to be treated with a therapeutic substance described herein may be a subject in need thereof.

The subject may be identified, or may have been identified, as having a complement-related disorder or being at risk of developing a complement-related disorder, e.g. by a method described herein.

A subject described herein may belong to a patient subpopulation i.e. the subject may be part of an identifiable, specific portion or subdivision of a population. The population and/or subpopulation may have or be suspected to have a complement-related disorder. The subpopulation may display atypical presence or levels of one or more complement proteins, e.g.

detected/determined as described herein, as compared to the population as a whole. The population and/or subpopulation may have or be suspected to have AMD, EOMD or a macular dystrophy.

In some aspects provided herein, the subject is characterised as having an atypical presence or level of one or more complement proteins, e.g. detected/determined/measured as described herein.

A subject may have, have been determined to have, or be characterised as having elevated levels of a complement protein selected from one or more of FHR1, FHR2, FHR3, FHR4 and/or FHR5, and optionally FHL-1, e.g. as compared to a reference value.

A subject may have, have been determined to have, or be characterised as having elevated levels of a complement protein selected from one or more of C3, C3b, C3a or iC3b, and/or having reduced levels of a complement protein selected from one or more of iC3b, C3f, C3c, C3dg, and/or C3d, e.g. as compared to a reference value.

Provided is a method of treating or preventing a complement-related disorder in a subject, wherein the subject is characterised as having an atypical presence or levels of one or more complement proteins, e.g. detected/determined as described herein.

Also provided is an article/molecule described herein for use in a method of treating or preventing a complement-related disease in a subject, wherein the subject is characterised as having an atypical presence or levels of one or more complement proteins, e.g. detected/determined as described herein.

Methods according to the present invention may be performed outside the human or animal body. Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism. In some embodiments, the determining, detecting, measuring, quantifying, predicting and/or diagnosing steps of the methods provided herein are performed in vitro.

The nucleic acids, polynucleotides, expression cassettes, vectors, polypeptides, and cells described herein may be formulated as pharmaceutical compositions or medicaments for clinical use and may comprise a pharmaceutically acceptable carrier, diluent, excipient or adjuvant. In accordance with the present invention methods are also provided for the production of pharmaceutically useful compositions, such methods of production may comprise one or more steps selected from: isolating an article/molecule as described herein; and/or mixing an article/molecule as described herein with a pharmaceutically acceptable carrier, adjuvant, excipient or diluent.

The nucleic acids, polynucleotides, expression cassettes, vectors, polypeptides, and cells described herein may be formulated for topical, parenteral, systemic, intracavitary, intravenous, intra-arterial, intramuscular, intrathecal, intraocular, intravitreal, intraconjunctival, subretinal, suprachoroidal, choroidal, subcutaneous, intradermal, intrathecal, oral, nasal or transdermal routes of administration which may include injection or infusion, or administration as an eye drop (i.e. ophthalmic administration). Suitable formulations may comprise the agent in a sterile or isotonic medium. Medicaments and pharmaceutical compositions may be formulated in fluid, including gel, form. Fluid formulations may be formulated for administration by injection or infusion (e.g. via catheter) to a selected organ or region of the human or animal body. Also provided is a method of formulating or producing a medicament or pharmaceutical composition for use in a method of medical treatment, the method comprising formulating a pharmaceutical composition or medicament by mixing a nucleic acid, polynucleotide, expression cassette, vector, polypeptide, and/or cell with a pharmaceutically acceptable carrier, adjuvant, excipient or diluent.

A nucleic acid, polynucleotide, expression cassette, vector, polypeptide, cell and/or composition described herein may be formulated in a sustained release delivery system, in order to release the nucleic acid, polynucleotide, expression cassette, vector, polypeptide, cell and/or composition at a predetermined rate. Sustained release delivery systems may maintain a constant drug/therapeutic concentration for a specified period of time. In some embodiments, an agent described herein is formulated in a liposome, gel, implant, device, or drug-polymer conjugate e.g. hydrogel.

Treatment of a complement-related disorder as described herein may involve modifying at least one cell of a subject, including a population of cells, to express or comprise a nucleic acid, polynucleotide, expression cassette, vector or polypeptide described herein. That is, the present invention provides a method of treating a complement-related disorder, the method comprising administering a nucleic acid, polynucleotide, expression cassette, or vector as described herein, e.g. to a cell or population of cells, for example to express a nucleic acid, polynucleotide, expression cassette, transgene, or polypeptide as described herein.

Also provided is a nucleic acid, polynucleotide, expression cassette, or vector as described herein for use in a method of treatment, wherein the method comprises administering a nucleic acid, polynucleotide, expression cassette, or vector as described herein, e.g. to a cell, for example to express a nucleic acid, polynucleotide, expression cassette, transgene, or polypeptide as described herein.

Also provided is the use of a nucleic acid, polynucleotide, expression cassette, or vector as described herein in the manufacture of a medicament for treating or preventing a complement-related disorder as described herein, wherein the treatment or prevention comprises administering a nucleic acid, polynucleotide, expression cassette, or vector as described herein, e.g. to a cell, for example to express a nucleic acid, polynucleotide, expression cassette, transgene, or polypeptide as described herein.

The at least one cell may be a cell, e.g. human cell, of the eye, kidney, vascular system, blood, muscle, skin, oesophagus, small or large intestine, intestinal tract, pharynx, trachea, lungs, bronchi, bronchioles, or central nervous system. The at least one cell may be a cancer cell or a tumor cell.

In some cases, the nucleic acid, polynucleotide, expression cassette, vector, polypeptide, and/or cell is administered to the eye, e.g. to one or more RPE cells, to the vitreous, or to the retina. In some embodiments, administration of the nucleic acid, polynucleotide, expression cassette, vector, polypeptide, and/or cell is intraocular, intravitreal, conjunctival, subretinal, suprachoroidal, or choroidal administration.

In some cases, the nucleic acid, polynucleotide, expression cassette, vector, polypeptide, and/or cell is administered to the kidney, e.g. to one or more of the glomerular endothelial cells, glomerular basement membrane (GBM), macula densa cells, mesangial cells, parietal epithelial cells, podocytes, or tubule epithelial cells.

In some cases, the nucleic acid, polynucleotide, expression cassette, vector, polypeptide, and/or cell is administered to the CNS, e.g. to one or more neurons, glial cells, astrocytes, oligodendrocytes, ependymal cells, and/or microglia.

In some cases, the nucleic acid, polynucleotide, expression cassette, vector, polypeptide, and/or cell is administered to the liver, e.g. to one or more hepatocytes. In some cases, the nucleic acid, polynucleotide, expression cassette, vector, polypeptide, and/or cell is administered to the blood, e.g. systemic administration (i.e. intravenous/intra-arterial administration). In some cases, the nucleic acid, polynucleotide, expression cassette, vector, polypeptide, and/or cell is administered subcutaneously.

In some cases, methods provided herein comprise targeted delivery of the nucleic acid, polynucleotide, expression cassette, vector, polypeptide, and/or cell i.e, wherein the concentration of the nucleic acid, polynucleotide, expression cassette, vector, polypeptide, and/or cell in the subject is increased in some parts of the body relative to other parts and/or wherein the nucleic acid, polynucleotide, expression cassette, vector, polypeptide, and/or cell is delivered via a controlled-release technique. In some cases, the methods comprise intravenous, intra-arterial, intramuscular or subcutaneous administration and wherein the nucleic acid, polynucleotide, expression cassette, vector, polypeptide, and/or cell is formulated in a targeted agent delivery system. Suitable targeted agent delivery systems include, for example, nanoparticles, liposomes, micelles, beads, polymers, metal particles, dendrimers, antibodies, aptamers, nanotubes or micro-sized silica rods. Such systems may comprise a magnetic element to direct the nucleic acid, polynucleotide, expression cassette, vector, polypeptide, and/or cell to the desired organ or tissue. Suitable nanocarriers and delivery systems will be apparent to one skilled in the art. In some cases, the nucleic acid, polynucleotide, expression cassette, vector, polypeptide, and/or cell is formulated for targeted delivery to a specific organ(s) or tissue(s). In some cases, the nucleic acid, polynucleotide, expression cassette, vector, polypeptide, and/or cell is delivered to the eye or kidney. In some cases, the methods comprise intravenous, intra-arterial, intramuscular or subcutaneous administration and wherein the nucleic acid, polynucleotide, expression cassette, vector, polypeptide, and/or cell is formulated for targeted delivery to the eye or kidney.

In some cases, RNA, e.g. nanoparticle based formulations, may be formulated for pulmonary administration for subsequent delivery to non-lung tissues, see e.g. US 2015/0157565 A1, which is herein incorporated in its entirety.

The particular mode and/or site of administration may be selected in accordance with the location where reduction of complement activation is required. In some cases, the methods comprise intravenous and/or intra-arterial administration. In some cases, the methods comprise administration to the eye or kidney.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20^{th} Edition, 2000, pub. Lippincott, Williams & Wilkins.

Administration may be alone or in combination with other treatments (e.g. other therapeutic or prophylactic intervention), either simultaneously or sequentially depending on the condition to be treated. A nucleic acid, polynucleotide, expression cassette, vector, polypeptide, and/or cell described herein and a further complement-targeted therapeutic agent may be administered separately, simultaneously or sequentially.

Simultaneous administration refers to administration of the nucleic acid, polynucleotide, expression cassette, vector, polypeptide, and/or cell and a further therapeutic agent together, for example as a pharmaceutical composition containing both agents (combined preparation), or immediately after each other and optionally via the same route of administration, e.g. to the same tissue, artery, vein or other blood vessel. Sequential administration refers to administration of one of the nucleic acid, polynucleotide, expression cassette, vector, polypeptide, and/or cell followed after a given time interval by separate administration of a further therapeutic agent. It is not required that the two agents are administered by the same route, although this is the case in some embodiments. The time interval may be any time interval. In some embodiments, the polypeptide, nucleic acid, vector, cell or composition and therapeutic agent are administered separately, simultaneously or sequentially to the eye or kidney.

In some embodiments the nucleic acid, polynucleotide, expression cassette, vector, polypeptide, cell and/or composition described herein is administered in combination with a therapeutically effective amount of a polypeptide having the peptidase activity of Complement Factor I (FI), i.e. able to cleave C3b. The FI polypeptide may comprise or consist of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:35. The FI polypeptide may comprise or consist of the proteolytic domain of FI and, for example, comprise or consist of a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:36. The FI polypeptide may be encoded by a nucleic acid sequence, such as a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to GenBank Y00318.1 or J02770.1.

In some embodiments, Complement Factor I is administered to the subject simultaneously or sequentially with administration of nucleic acid, polynucleotide, expression cassette, vector, polypeptide, cell and/or composition described herein. The nucleic acid, polynucleotide, expression cassette, vector, polypeptide, cell and/or composition described herein may be formulated in a composition with a FI polypeptide, or a nucleic acid encoding a FI polypeptide. That is, the present invention provides a composition comprising a nucleic acid, polynucleotide, expression cassette, vector, polypeptide, cell and/or composition described herein and a FI polypeptide, nucleic acid encoding a FI polypeptide or vector comprising nucleic acid encoding a FI polypeptide, optionally with a pharmaceutically acceptable excipient/carrier. In some embodiments a nucleic acid, polynucleotide, or expression cassette described herein and a nucleic acid encoding a FI polypeptide are provided in the same vector, e.g. an AAV vector as described herein. In some embodiments a nucleic acid or polynucleotide described herein and a nucleic acid encoding a FI polypeptide are provided in the same expression cassette, e.g. in the same vector.

Thus, provided herein is a vector comprising a nucleotide sequence encoding a polypeptide as described herein and a nucleotide sequence encoding a FI polypeptide. The two polypeptides may be under the control of the same promoter or different promoters. A vector or polynucleotide described herein (e.g. a second nucleotide sequence) may comprise a nucleotide sequence having at least 80% sequence identity to SEQ ID NO:6 or 5 and a nucleotide sequence having at least 80% sequence identity to GenBank Y00318.1 or J02770.1.

The nucleic acid, polynucleotide, expression cassette, vector, polypeptide, cell and/or composition described herein and the FI polypeptide (or nucleic acid encoding the polypeptide) may be formulated in separate compositions (and/or vectors). The separate compositions may be administered simultaneously or sequentially, e.g. via one or more administration routes described hereinabove.

In some embodiments, the treatment may comprise modifying a cell or population of cells in vitro, ex vivo or in vivo to express and/or secrete Complement Factor I. The cell or population of cells may be the same cell or population of cells as a cell or population of cells modified to comprise/express a nucleic acid, polynucleotide, expression cassette, vector, or polypeptide according to the present invention, for example the treatment may comprise modifying a cell or population of cells in vitro, ex vivo or in vivo to express and/or secrete a nucleic acid, polynucleotide, expression cassette, vector, or polypeptide according to the present invention, and Complement Factor I. Such expression may be simultaneous or sequential. In some embodiments, Complement Factor I (or a nucleic acid encoding a FI polypeptide) is administered to a subject, wherein the subject comprises a cell or population of cells modified to comprise/express a nucleic acid, polynucleotide, expression cassette, vector, or polypeptide of the present invention. In some embodiments, Complement Factor I is administered to a subject wherein the subject has expressed in situ or is expressing in situ a nucleic acid, polynucleotide, expression cassette, vector, or polypeptide of the present invention.

Other therapeutic agents or techniques suitable for use with the present invention may comprise nutritional therapy, photodynamic therapy (PDT), laser photocoagulation, anti-VEGF (vascular endothelial growth factor) therapy, and/or additional therapies known in the art, see e.g. Al-Zamil W M and Yassin S A, Clin Interv Aging. 2017 Aug. 22; 12:1313-1330). Anti-VEGF therapy may comprise agents such as ranibizumab (Lucentis, made by Genentech/Novartis), Avastin (Genentech), bevacizumab (off label Avastin), and aflibercept (Eylea®/VEGF Trap-Eye from Regeneron/Bayer). Further agents or techniques suitable for use with the present invention include APL-2 (Apellis), AdPEDF (GenVec), encapsulated cell technology (ECT; Neurotech), squalamine lactate (EVIZON™, Genaera), OT-551 (antioxidant eye drops, Othera), anecortave actate (Retaane®, Alcon), bevasiranib (siRNA, Acuity Pharmaceuticals), pegaptanib sodium (Macugen®), and AAVCAGsCD59 (Clinical trial identifier: NCT03144999).

Multiple doses of a nucleic acid, polynucleotide, expression cassette, vector, polypeptide, and/or cell may be provided. One or more, or each, of the doses may be accompanied by simultaneous or sequential administration of a further therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

### Methods for Assessing Complement-Related Disorders

Methods described herein, e.g. therapeutic and/or prophylactic methods, may involve detecting the presence of, and/or determining the level of, one or more complement proteins using suitable analytical techniques, e.g. as described herein. In some aspects the invention provides methods for selecting treatment for and/or treating subjects/patients that have a complement-related disorder or have been identified as having a complement-related disorder, e.g. by determining the level of a complement protein as described herein. Such methods can be used to inform treatment of the subject/patient, e.g. using an molecule or article described herein. Such methods may be used prior to administration of a therapeutic agent, such as a nucleic acid, expression cassette, or vector described herein, or after administration of a therapeutic agent as described herein. That is, any assessment method described herein may comprise a step of treating a subject.

Methods for detecting complement proteins are described in WO2022/058447 A1, which is hereby incorporated by reference in its entirety.

The assessment methods described herein may be diagnostic, prognostic and/or predictive of the risk of onset or progression of a complement-related disorder, e.g. a disorder as described herein. Diagnostic methods can be used to determine the diagnosis or severity of a disease, prognostic methods help to predict the likely course of disease in a defined clinical population under standard treatment conditions, and predictive methods predict the likely response to a treatment in terms of efficacy and/or safety, thus supporting clinical decision-making.

Provided herein are methods for assessing the risk of onset, risk of progression, or risk of development of a complement-related disorder. The complement related disorder may be any disorder in which the complement system, or activation/over-activation/dysregulation thereof, is pathologically implicated. The complement related disorder may be any disorder described herein. The methods described herein may be useful in monitoring the success of treatment, including past or ongoing treatment, for complement-related disorders. Such treatment may involve the nucleic acids, expression cassettes, and/or vectors as described herein.

The following methods may be combined with any method of treating a disease/disorder as described herein, e.g. before the treatment to inform therapy, after the treatment to assess treatment outcomes, and/or during the treatment to assess progress.

“Complement protein” may be used interchangeably herein with “complement regulator”, “a regulator of complement”, or “protein of the complement system” and refers to a protein component of the complement system or complement cascade, e.g. as described in Merle et al., Front. Immunol., 2015, 6:262 and Merle et al., Front. Immunol., 2015, 6:257, which are hereby incorporated by reference in their entirety. A “complement protein” referred to herein may be involved in any of the three complement pathways and/or in the amplification loop.

In some embodiments a “complement protein” referred to herein is involved in the alternative pathway and/or the complement activation loop. In some embodiments, a “complement protein” referred to herein is involved in the breakdown, turnover and/or inactivation of C3 or C3b, or is a product of said breakdown, turnover and/or inactivation.

In some embodiments herein, a “complement protein” as used herein may refer to one or more of FH, FHL-1, FHR1, FHR2, FHR3, FHR4, FHR5, FI, C3, C3b, C3a, iC3b, C3f, C3c, C3dg, and/or C3d.

As used herein, any reference to a complement protein, e.g. C3, C3b, C3a, FH, FI etc, refers to said protein from any species and include isoforms, fragments, variants or homologues of said protein from any species. In some embodiments, the protein is a mammalian protein (e.g. cynomolgus, human and/or rodent (e.g. rat and/or murine) protein). Isoforms, fragments, variants or homologues of the complement proteins described herein may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of the immature or mature protein from a given species, e.g. human protein sequences provided herein. Isoforms, fragments, variants or homologues of complement proteins described herein may optionally be functional isoforms, fragments, variants or homologues, e.g. having a functional property/activity of the reference protein, as determined by analysis by a suitable assay for the functional property/activity.

C3, C3b and further breakdown products are described hereinabove.

Factor H (FH) (Uniprot P08603-1) regulates the alternative complement pathway and the amplification loop. It inhibits C3 convertase formation by competing with FB binding to C3b and also acts as a cofactor for C3b inactivation to iC3b by Factor I (FI), thus preventing inappropriate complement activation and inflammation. FH also exerts decay-accelerating activity, which can assist in the deconstruction of already formed C3 convertases, see e.g. Clark et al., J Immunol 2014, 193 (10) 4962-4970, which is hereby incorporated by reference in its entirety. For a review of FH structure and function see e.g. Merle N S et al., Front Immunol. 2015 Jun. 2; 6:262, which is hereby incorporated by reference in its entirety.

Human FH comprises 20 CCP domains. FH is encoded by the CFH gene on human chromosome 1q32 within the RCA (regulators of complement) gene cluster. The CFH gene also produces a truncated form of FH, called FHL-1 (Uniprot: P08603-2), comprising only the first seven CCP domains before terminating with a unique 4-amino acid C terminus (Clark et al, 2014 supra).

In the eye, full-length FH protein is found on the choroidal side of Bruch's membrane (BrM), with particular accumulation in the choriocapillaris (capillary layer in the choroid). Small amounts have also been found in patches on the RPE side of the BrM, but no FH was observed in the BrM itself. FHL-1 on the other hand has been observed throughout BrM and other ECM structures e.g. drusen (Clark et al, 2014 supra). It is likely that FHL-1 confers greater complement protection to BrM than does FH, whereas FH provides the main protection for the ECM of the choroid. It is thought that FHL-1 is therefore a major regulator of complement in the BrM (a key site in AMD pathogenesis). The methods described herein allow for the individual detection and quantitation of FH and FHL-1.

FHR1, FHR2, FHR3, FHR4 and FHR5, encoded by the CFHR genes, are described in e.g. Skerka et al., Mol Immunol 2013, 56:170-180, which is hereby incorporated by reference in its entirety. The five FHR proteins are thought to counter the inhibitory effects of FH and FHL-1, and thus contribute to the pathology of complement-related disorders.

FH, FHL-1 and FHR1-FHR5, are described in e.g. Clark et al., J Clin Med, 2015. 4 (1): 18-31, which is hereby incorporated by reference in its entirety. These proteins are highly related and share a high degree of sequence identity. The N termini share 36-94% sequence identity, whilst the C-terminal domains are very similar to the FH C-terminus (36-100%). The high amino acid identity among family members is demonstrated by the fact that antibodies raised against FH can detect multiple FHR proteins in plasma and that antibodies generated against FHR proteins cross-react with the other FHRs. This cross-reactivity presents a challenge for purification of FHR proteins from plasma, as well as determining their concentration.

FHR proteins are divided into two groups depending on their conserved domains. FHR1 (Uniprot: Q03591), FHR2 (Uniprot: P36980-1, P36980-2), and FHR5 (Uniprot: Q9BXR6) form Group I and are characterised by their conserved N-termini. They exist in plasma as homo- and heterodimers, mediated by the conserved N-terminal domains. Group II contains FHR3 (Uniprot: Q02985-1, Q02985-2) and FHR4 (Uniprot: Q92496-1, Q92496-3) which lack the N-terminal dimerization domains, but which show a high degree of sequence similarity to portions of FH. All five FHR proteins comprise C-termini sequences that act to recognise and bind C3b, and which are very similar to the C-terminus of FH.

Elevated levels of one or more of the five FHR proteins have been implicated in a variety of complement-related disorders affecting different tissues, such as those in the kidney (see e.g. Medjeral-Thomas et al., Kidney Int Rep. 2019, 4 (10): 1387-1400; Wong & Kavanagh, Semin Immunopathol. 2018; 40 (1): 49-64; Sethi et al., Kidney Int. 2009, 75 (9): 952-60; Abrera-Abeleda et al., J Med Genet. 2006, 43 (7): 582-589), autoimmune diseases (see e.g. Goicoechea de Jorge et al., PNAS 2013, 110 (12): 4685-90; Schafer et al., Front Immunol. 2016, 7:542; Legatowicz-Koprowska et al., Reumatologia. 2020, 58 (6): 357-366), atherosclerosis (see e.g. Speidl et al., J Thromb Haemost. 2011, 9 (3): 428-40; Malik et al., Circulation. 2010, 122 (19): 1948-56; Machalińska et al., Acta Ophthalmol. 2012, 90 (8): 695-703), cancer (see e.g., DeCordova et al., Immunobiology. 2019, 224 (5): 625-631; Revel et al., Antibodies (Basel). 2020, 9 (4): 57; Afshar-Kharghan, J Clin Invest. 2017, 127 (3): 780-789) and neurodegenerative disorders (see e.g., Chen & Xia, J Alzheimers Dis. 2020, 76 (1): 349-368; Ashton et al., Alzheimers Dement (Amst). 2015, 1 (1): 48-60; Loveless et al., Brain Pathol. 2018 July; 28 (4): 507-520; Pouw and Ricklin, Semin Immunopathol. 2021, 43 (6): 757-771).

FHR1 is known to compete with FH and FHL-1 for binding to C3b. It is also reported to bind to C3b components of the C5 convertase and interfere with the assembly of the MAC (see e.g. Heinen S et al., Blood (2009) 114 (12): 2439-2447 and Hannan J P et al., PloS One. 2016; 11 (11): e0166200, which are hereby incorporated by reference in their entirety). As used herein, the term “FHR1” includes at least one of FHRA and a second FHR1 isoform (FHRB) with 3 point mutations, and preferably includes both FHR1 isoforms. “FHR1” refers to FHR1 from any species and includes isoforms, fragments, variants or homologues of FHR1 from any species. In preferred embodiments, “FHR1” refers to human FHR1.

FHR2 may inhibit C3 convertase activity, acting to inhibit the amplification loop, but may also activate the amplification loop. There are two FHR2 isoforms. The protein has two glycosylated forms, a single glycosylated form (24 kDa) and a double glycosylated form (28 kDa). As used herein, the term “FHR2” includes at least one of the two isoforms or at least one of the glycosylated forms, and preferably includes both isoforms and any glycosylated forms. “FHR2” refers to FHR2 from any species and includes isoforms, fragments, variants or homologues of FHR2 from any species. In preferred embodiments, “FHR2” refers to human FHR2.

FHR3 binds to C3b and C3d and may have low cofactor activity for FI-mediated cleavage of C3b. FHR3 may also upregulate complement. There are two FHR3 isoforms. FHR3 is detected in plasma in multiple variants (ranging from 35 to 56 kDa), reflecting the existence of four different glycosylated variants of FHR3. As used herein, the term “FHR3” includes at least one of the two isoforms or at least one of the glycosylated variants of FHR3, and preferably includes both isoforms and any glycosylated forms. “FHR3” refers to FHR3 from any species and includes isoforms, fragments, variants or homologues of FHR3 from any species. In preferred embodiments, “FHR3” refers to human FHR3.

The human CFHR4 gene encodes two proteins: FHR4A and FHR4B, an alternative splice variant. WO 2019/215330 A1, hereby incorporated by reference in its entirety, demonstrates that FHR4 is a positive regulator of complement activation and prevents FH-mediated C3b breakdown. High levels of FHR4 in tissues are likely to promote local inflammatory responses and cell lysis, leading to disorders associated with complement activation, and circulating FHR4 levels can be used as an indicator of risk of developing complement-related disorders, see e.g. Cipriani et al., Nat Commun 11, 778 (2020), hereby incorporated by reference in its entirety. As used herein, the term “FHR4” includes at least one of FHR4A isoform 1, FHR4A isoform 2 (G20 point deletion from isoform 1) or FHR4B, and preferably includes FHR4A isoforms 1 and 2 as well as FHR4B. “FHR4” refers to FHR4 from any species and includes isoforms, fragments, variants or homologues of FHR4 from any species. In preferred embodiments, “FHR4” refers to human FHR4.

FHR5 recognises and binds to C3b on self surfaces. FHR5 appears as a glycosylated protein of 62 kDa. As used herein, the term “FHR5” includes any glycosylated variants of FHR5, and preferably includes all isoforms and any glycosylated forms. As used herein, “FHR5” refers to FHR5 from any species and includes isoforms, fragments, variants or homologues of FHR5 from any species. In preferred embodiments, “FHR5” refers to human FHR5.

CFH family members, particularly FHR1-FHR5, can also be used as biomarkers for diagnosing or predicting disorders in which dysregulation of complement is pathologically implicated.

Any method of treatment provided herein may comprise a method of determining a level of a complement protein, e.g. as described herein, to inform treatment.

Thus any method of treatment provided herein may comprise the additional steps of:
(a) determining the level of a complement protein, e.g. selected from one or more of FHR1, FHR2, FHR3, FHR4 and/or FHR5, and optionally FH and/or FHL-1, in a blood, fluid or tissue sample obtained from the subject;
(b) selecting the subject for treatment with a therapeutic molecule/article described herein if the level of the complement protein determined in (a) is altered, e.g. elevated or reduced, as compared to the level of that complement protein(s) in blood, fluid or tissue in a control subject that does not have a complement-related disorder.

In some aspects, provided is a method of identifying a subject having a complement-related disorder or at risk of developing a complement-related disorder, the method comprising:
(a) determining the level of a complement protein, e.g. selected from one or more of FHR1, FHR2, FHR3, FHR4 and/or FHR5, in a blood, fluid or tissue sample obtained from the subject;
(b) determining that the subject has or is likely to develop a complement-related disorder if the level of the complement protein determined in (a) is altered, e.g. elevated or reduced, as compared to the level of that complement protein in blood, fluid or tissue in a control subject that does not have a complement-related disorder.

In some aspects, there is provided a method of determining whether a subject has, or is at risk of developing, a complement-related disorder, the method comprising:
(a) determining the level of a complement protein, e.g. selected from one or more of FHR1, FHR2, FHR3, FHR4 and/or FHR5, in a blood, fluid or tissue sample obtained from the subject;
(b) determining that the subject has or is likely to develop a complement-related disorder if the level of the complement protein determined in (a) is altered, e.g. elevated or reduced, as compared to the level of that complement protein in blood, fluid or tissue in a control subject that does not have a complement-related disorder.

In some aspects, there is provided a method of determining whether a therapeutic agent described herein is a suitable treatment for a subject, the method comprising:
(a) determining the level of a complement protein, e.g. selected from one or more of FHR1, FHR2, FHR3, FHR4 and/or FHR5, in a blood, fluid or tissue sample obtained from the subject;
(b) determining that the therapeutic agent is a suitable treatment for the subject if the level of the complement protein determined in (a) is altered, e.g. elevated or reduced, as compared to the level of that complement protein in blood, fluid or tissue in a control subject that does not have a complement-related disorder.

Any method of treatment provided herein may comprise a method of determining whether a subject comprises one or more mutations and/or polymorphisms in a gene encoding a complement protein, or in a gene associated with complement dysregulation, as described herein.

Any method above may also comprise a step of treating the subject with a molecule or article as described herein, e.g. a polynucleotide, expression cassette, vector, polypeptide, cell or composition.

In some embodiments, the complement protein is one or more of FHR1, FHR2, FHR3, FHR4 and/or FHR5, and the subject is selected for treatment, has, is likely to develop, or is at risk of developing a complement-related disorder if the level of the complement protein is elevated as compared to the level of that complement protein (e.g. in a sample) in a control subject that does not have a complement-related disorder.

In some embodiments, the complement protein is one or more of C3, C3b, C3a, iC3b, C3f, C3c, C3dg, and/or C3d. The subject has, is likely to develop, or is at risk of developing a complement-related disorder if the level of C3, C3b and/or C3a is elevated as compared to the level of that complement protein in blood/fluid/tissue in a control subject that does not have a complement-related disorder. The subject has, is likely to develop, or is at risk of developing a complement-related disorder if the level of iC3b, C3f, C3c, C3dg, and/or C3d is reduced as compared to the level of that complement protein in blood/fluid/tissue in a control subject that does not have a complement-related disorder.

In some aspects, there is provided a method of determining whether a method of treatment described herein has been successful in a subject, the method comprising:
(a) determining the level of a complement protein, e.g. selected from one or more of FHR1, FHR2, FHR3, FHR4 and/or FHR5, in a blood, fluid or tissue sample obtained from the subject;
(b) determining that the method of treatment has been successful if the level of the complement protein determined in (a) is altered, e.g. elevated or reduced, as compared to the level of that complement protein in blood, fluid or tissue in the same subject before treatment.

In some embodiments, the complement protein is one or more of FHR1, FHR2, FHR3, FHR4 and/or FHR5, and the method of treatment is determined to be successful if the level of the complement protein is reduced as compared to the level of that complement protein (e.g. in a sample) in the same subject before treatment.

In some embodiments, the complement protein is one or more of C3, C3b, C3a, iC3b, C3f, C3c, C3dg, and/or C3d. The method of treatment is determined to be successful if the level of C3, C3b and/or C3a is reduced as compared to the level of that complement protein (e.g. in a sample) in the same subject before treatment. The method of treatment is determined to be successful if the level of iC3b, C3f, C3c, C3dg, and/or C3d is elevated as compared to the level of that complement protein (e.g. in a sample) in the same subject before treatment.

In some embodiments step (a) comprises determining the level of two of the complement proteins selected from FHR1, FHR2 and/or FHR3. In some embodiments step (a) comprises determining the level of FHR1, FHR2 and FHR3.

In some embodiments step (a) comprises, or further comprises, determining the level of FHR4 and/or FHR5. The methods described herein may comprise determining that the subject has or is likely to develop a complement-related disorder if the level of FHR4 and/or FHR5 is elevated as compared to the level of that complement protein (e.g. in a sample) in a control subject that does not have a complement-related disorder.

In some embodiments step (a) comprises, or further comprises, determining the level of FH and/or FHL-1. The method may comprise determining the level of FHL-1, alone or in combination with other complement protein(s), and determining that the subject has or is likely to develop a complement-related disorder if the level of FHL-1 is altered, e.g. elevated, as compared to the level of FHL-1 (e.g. in a sample) in a control subject that does not have a complement-related disorder. The level of FH and/or FHL-1 may be increased or decreased compared to a control subject.

Determining the level of two or more complement proteins may be performed simultaneously, concurrently, or sequentially. The complement proteins may be detected in the same assay, or in one or more separate assays. Determining the level of a second or subsequent complement protein may be performed concurrently with, prior to or after determining the level of a first complement protein. In some embodiments, steps (a) and (b) may be repeated one or more times on the same subject at appropriate time intervals in order to assess the progression of a complement-related disorder.

Any aspect or embodiment described herein may comprise determining the level of any one of the following proteins, e.g. in a subject:
a) FHR1;
b) FHR2;
c) FHR3;
d) FHR4;
e) FHR5;
f) FHR1 and FHR2;
g) FHR1 and FHR3;
h) FHR1 and FHR4;
i) FHR1 and FHR5;
j) FHR2 and FHR3;
k) FHR2 and FHR4;
l) FHR2 and FHR5;
m) FHR3 and FHR4;
n) FHR3 and FHR5;
o) FHR4 and FHR5;
p) FHR1, FHR2 and FHR3;
q) FHR1, FHR2 and FHR4;
r) FHR1, FHR2 and FHR5;
S) FHR1, FHR3 and FHR4;
t) FHR1, FHR3 and FHR5;
u) FHR1, FHR4 and FHR5;
v) FHR2, FHR3 and FHR4;
w) FHR2, FHR3 and FHR5;
x) FHR2, FHR4 and FHR5;
y) FHR3, FHR4 and FHR5;
z) FHR1, FHR2, FHR3 and FHR4;
aa) FHR1, FHR2, FHR3 and FHR5;
bb) FHR1, FHR2, FHR4 and FHR5;
cc) FHR2, FHR3, FHR4 and FHR5;
dd) FHR1, FHR3, FHR4 and FHR5; or
ee) FHR1, FHR2, FHR3, FHR4 and FHR5;
   or any of (a) to (ee) in combination with determining the level of FH and/or FHL-1, e.g. FHR1, FHR2, FHR3, plus FH and/or FHL-1; or FHR1, FHR2, FHR3, FHR4, FHR5, plus FH and/or FHL-1.

In some embodiments, reference to ‘FHR1’ herein may refer to the detection of either one or both of FHR1a and/or FHR1b.

In some embodiments the complement protein(s) to be detected/determined is not FHR3. In some embodiments the complement protein(s) to be detected/determined is not FHR4. In some embodiments the complement protein(s) to be detected/determined is not FH. In some embodiments the complement protein(s) to be detected/determined is not FHL-1.

In some embodiments the complement protein(s) is one or more of C3, C3b, C3a, iC3b, C3f, C3c, C3dg, and/or C3d. Any method described herein may comprise determining the level of one or more of C3, C3b, C3a, iC3b, C3f, C3c, C3dg, and/or C3d, e.g. as described herein.

The selection or combination of complement protein(s) detected may depend on the complement-related disorder of interest and the complement protein(s) that are useful biomarkers for an individual disorder. For example, detecting one or more of FHR1, FHR2, FHR3, FHR4, FHR5 and/or FHL-1 is predictive of AMD risk, whereas other particular complement proteins and combinations thereof are predictive for other complement-related disorders, see e.g. the disorders and references described herein. The present disclosure allows the precise detection and distinction of any one or more of the complement proteins described herein, thus allowing the absolute levels of said proteins to inform the likelihood of disorder onset and/or progression according to the variations in protein levels in each disorder.

The complement protein(s) may be detected in a sample obtained from a subject. For example, the sample may be obtained to inform appropriate treatment and/or progression of the disorder.

In some cases, any aspect described herein may comprise determining the level of any one or more complement proteins selected from FHR1, FHR2, FHR3, FHR4, FHR5, FH and/or FHL-1, e.g. in a blood sample obtained from a subject, and then determining that the subject has or is likely to develop a complement-related disorder if the level of the complement protein(s) is altered as compared to the level of that complement protein(s) (e.g. in a blood sample) in a control subject that does not have a complement-related disorder. The term “altered” as used herein refers to the level of the complement protein(s) increasing or decreasing, e.g. the level of one or more complement proteins may be higher or lower as compared to the level of those complement proteins (e.g. in a blood sample) in a control subject that does not have a complement-related disorder. In some cases, the level of the complement protein may be decreased as compared to the level of that complement protein (e.g. in a blood sample) in a control subject that does not have a complement-related disorder. In some cases, where the level of two or more complement proteins is determined, the level of one or more complement proteins may be elevated whilst the level of one or more different complement proteins may be decreased as compared to the levels of those complement proteins (e.g. in a blood sample) in a control subject that does not have a complement-related disorder.

Methods provided herein may be useful for determining the risk of a subject developing a serious complement-related disorder, e.g. the methods are useful for distinguishing between subjects who may develop a mild complement-related disorder and subjects who are at risk of serious disease, and/or identifying subjects who are likely to develop serious disease.

In some embodiments the methods described herein can be used to identify subjects that are at risk of developing a severe disorder associated with SARS-COV-2 infection, e.g. severe COVID-19 or critical COVID-19. Cases of COVID-19 can generally be categorised into five groups: asymptomatic, mild, moderate, severe and critical. Severe COVID-19 includes pneumonia and patients may require supplemental oxygen. Critical COVID-19 includes severe pneumonia and ARDS, and in some cases sepsis. Patients with critical COVID-19 require assisted ventilation.

In some embodiments the methods comprise detecting/determining the level of a complement protein in a sample. The sample may be in vitro or ex vivo. A sample may have been taken from a subject, e.g. from a subject of interest or from a control subject. A sample may be taken from any tissue or bodily fluid. In preferred arrangements the sample is taken from a bodily fluid, more preferably one that circulates through the body. The sample may be referred to as a circulating sample. Accordingly, the sample may be a blood sample or lymph sample. In a particularly preferred arrangement the sample is a blood sample or blood-derived sample. The blood-derived sample may be a selected fraction of a subject's blood, e.g. a selected cell-containing fraction or a plasma or serum fraction. A selected serum fraction may comprise the fluid portion of the blood obtained after removal of the fibrin clot and blood cells. Alternatively the sample may comprise or may be derived from a tissue sample, biopsy or isolated cells from said individual. The sample may be taken from the eye, kidney, brain or liver, e.g. comprising cells from the eye, kidney, brain or liver. The sample may be taken/obtained from the CNS, eye or kidney. The sample may be CSF or vitreous fluid. The sample may comprise retinal tissue. The sample may comprise RPE cells or tissue from Bruch's membrane or the choroid. The sample may comprise drusen or other deposits of complement-related components.

In some embodiments the methods described herein comprise taking or obtaining a sample from a subject, e.g. blood, tissue etc. In some embodiments the methods described herein are performed on a sample that has been obtained/was obtained from a subject, e.g. that has been obtained previously and stored prior to use. Storage of samples, e.g. tissue and/or blood samples, are well known to a skilled person. In some cases the sample is a blood sample. The blood sample may undergo/have undergone processing to obtain a plasma sample or a serum sample. In some cases, the methods comprise obtaining a blood-derived sample from a subject. In some cases, the methods comprise obtaining a plasma or serum sample from a subject. In some cases, the methods comprise obtaining a CNS or eye-derived sample from a subject. In some cases, the methods comprise obtaining a CSF or vitreous fluid sample from a subject. In some embodiments the methods comprise isolating protein, e.g. total protein, from the sample. Suitable techniques to isolate protein from biological samples are well known in the field. In some embodiments the methods do not comprise isolating protein from the sample, e.g. the methods are performed on the unprocessed sample.

Any method described herein may comprise an initial step of obtaining a sample and/or at least one protein, e.g. complement protein, from the subject. Suitable sources of samples are described herein. The methods described herein may comprise determining the level of circulating FHR1, FHR2, and/or FHR3, circulating FHR4 and/or FHR5, and optionally circulating FH and/or FHL-1. Circulating proteins may be present in e.g. blood or lymph.

In some embodiments, the methods are performed in vitro or ex vivo. For example, the presence, level, amount and/or concentration of the complement protein(s) may be detected/determined in vitro. A sample may be obtained from a subject of interest, and/or a control subject, and the determining steps are performed in vitro or ex vivo. Steps of the methods that involve treating a subject may be performed in vivo.

In methods described herein the level of the complement protein(s) is compared to the level of a reference value or level, sometimes called a control. In some cases the level of the complement protein(s) is compared to the level of the same complement protein in a control subject that does not have a complement-related disorder. A reference value may be obtained from a control sample, which itself may be obtained from a control subject. Data or values obtained from the individual to be tested, e.g. from a sample, can be compared to data or values obtained from the control sample. In some cases, the control is a spouse, partner, or friend of the subject.

The level of the complement protein(s) that are determined may be elevated (i.e. higher, increased, greater) compared to the reference value or level. That is, there may be more of the complement protein(s) in the sample tested compared to the reference value. There may be a higher amount or concentration of the complement protein(s) in the tested sample compared to the reference value or in a control sample.

The level of the complement protein(s) that are determined may be reduced (i.e. lower, decreased) compared to the reference value or level. That is, there may be less of the complement protein(s) in a tested sample tested compared to the reference value. There may be a lower amount or concentration of the complement protein(s) in a tested sample compared to the reference value or in a control sample.

As used herein the term “reference value” refers to a known measurement value used for comparison during analysis. In some cases, the reference value is one or a set of test values obtained from an individual or group in a defined state of health. The reference value may be one or a set of test values obtained from a control. In some cases, the reference value is/has been obtained from determining the level of complement proteins in subjects known not to have a complement-related disorder. In some cases, the reference value is/has been obtained from determining the level of complement proteins in subjects which have a complement-related disorder that is not associated with elevated levels of FHR protein(s), e.g. a subset of subjects in which FHR proteins are not considered to be a pathological factor. In some cases, the reference value is set by determining the level or amount of a complement protein previously from the individual to be tested e.g. at an earlier stage of disease progression, or prior to onset of the disease. The reference value may be taken from a sample obtained from the same subject, or a different subject or subject(s). The sample may be derived from the same tissue/cells/bodily fluid as the sample used by the present invention. The reference value may be a standard value, standard curve or standard data set. Values/levels which deviate significantly from reference values may be described as atypical values/levels.

In some cases the control may be a reference sample or reference dataset, or one or more values from said sample or dataset. The reference value may be derived from a reference sample or reference dataset. The reference value may be derived from one or more samples that have previously been obtained from one or more subjects that are known not to have a complement-related disorder and/or known or expected not to be at risk of developing a complement-related disorder. The reference value may be derived from one or more samples that have previously been obtained from one or more subjects that are known to have a complement-related disorder. The reference value may be derived from one or more samples that have previously been obtained from one or more subjects that are known to be at risk of developing a complement-related disorder. The reference value may be consensus level or an average, or mean, value calculated from a reference dataset, e.g. a mean protein level. The reference dataset/value may be obtained from a large-scale study of subjects known to have a complement-related disorder, such as AMD, e.g. as described herein.

The reference value may be derived from one or more samples that have previously been obtained from one or more subjects that are in the same family as the subject of interest, or from one or more subjects that are not in the same family as the subject of interest.

The reference value may be derived from one or more samples that have previously been obtained and/or analysed from the individual/subject/patient to be tested, e.g. a sample was obtained from the individual when they were at an earlier stage of a complement-related disorder, or a sample was obtained from the individual before the onset of a complement-related disorder.

The reference value may be obtained by performing analysis of the sample taken from a control subject in parallel with a sample from the individual to be tested. Alternatively, the control value may be obtained from a database or other previously obtained value. The reference value may be determined concurrently with the methods disclosed herein, or may have been determined previously.

Control subjects from which samples are/have been obtained may have undergone treatment for a complement-related disorder and/or received a complement-related therapy/therapeutic agent.

Controls may be positive controls in which the target molecule is known to be present, or expressed at high level, or negative controls in which the target molecule is known to be absent or expressed at low level.

Samples from one or more control subjects may comprise any one, two, three, four, five, six of seven of FHR1, FHR2, FHR3, FHR4, FHR5, FH and/or FHL-1. In some cases each complement protein is in a separate control sample. In some cases a control sample contains multiple complement proteins. In some cases the methods described herein comprise comparing the level of one of more complement proteins determined as described herein to different, e.g. one or more, samples, each sample containing one or more complement proteins. In some cases the methods described herein comprise comparing the level of one or more complement proteins determined as described herein to a single sample, wherein the sample contains one or more complement proteins.

In some cases control samples are obtained from the same tissue(s) as the sample obtained from the individual to be tested. In some cases control samples are obtained from different tissue(s) as the sample obtained from the individual to be tested. Control samples may be obtained from control subjects at certain time(s) of day, or on certain days. Sample(s) obtained from the individual to be tested are preferably obtained at the same time(s) of day and/or day(s) as the control samples.

In some cases, an increase/decrease of a complement protein, e.g. as described herein, as compared to a reference value indicates an increased risk of developing a complement-related disorder. In some cases, an increase/decrease of a complement protein, e.g. as described herein, indicates an increased risk of developing the disorder when compared to a reference value taken from the same subject at an earlier stage of the disorder, e.g. in a sample from the same subject.

In some embodiments, a method described herein may comprise determining the level of two or more complement proteins and comparing their values e.g. concentrations. The values may be compared to each other, as well as to reference values, e.g. increased levels of C3 and C3b compared to stationary or decreased levels of iC3b and further C3b breakdown products may be indicative of a higher risk of development of a complement-related disorder and/or the need to treat a subject for a complement-related disorder. Decreased levels of C3 and C3b compared to stationary or increased levels of iC3b and further C3b breakdown products may be indicative of a lower risk of development of a complement-related disorder and/or that treatment for a complement-related disorder is effective.

In some cases, a method described herein may comprise comparing the levels of any one or more of FHR1, FHR2, FHR3, FHR4 and/or FHR5, to the level of FH and/or FHL-1 in the subject tested. For example, elevated levels of FHR1, FHR2, FHR3, FHR4 and/or FHR5, and optionally FHL-1, compared to stationary levels of FH (i.e. a statistically non-significant change) in a subject may be indicative of a higher risk of the subject developing a complement-related disorder and/or the need to treat the subject for a complement-related disorder.

In some embodiments, a method provided herein comprises a step of correlating the presence of an atypical or altered amount/level of a complement protein with an increased risk of the subject developing or having a complement-related disorder.

Examples of reference values for complement proteins in human subjects known not to have a complement-related disorder include:
a) FH: ~150 to 500 μg/ml in human blood (Clark et al., J Immunol 2014. 193 (10):4962-70 and unpublished data);
   Mean: 833 nM in human plasma, SD: 149 (derived from the mass spectrometry methods described herein);
b) FHL-1: ~0.5 to 50 μg/ml in human blood (Clark et al., J Immunol 2014. 193 (10):4962-70 and unpublished data);
   Mean: 10.9 nM in human plasma, SD: 2.4 (derived from the mass spectrometry methods described herein);
c) FHR1: ~70 to 100 μg/ml in human plasma (Heinen, S et al., Blood 114, 2439-2447);
   Mean: 33.9 nM in human plasma, SD: 16.5 (derived from the mass spectrometry methods described herein);
d) FHR2: ~15-50 μg/ml in human plasma, or about 1/10 of FH concentration (Skerka et al., Mol Immunol 2013, 56:170-180);
   Mean: 51.2 nM in human plasma, SD: 17.0 (derived from the mass spectrometry methods described herein);
e) FHR3: ~70 to 100 μg/ml in human plasma (Fritsche, L. G. et al., Hum. Mol. Genet. 2010.19, 4694-4704);
   Mean: 20.6 nM in human plasma, SD: 13.8 (derived from the mass spectrometry methods described herein);
f) FHR4: <5 μg/ml in human blood (WO 2019/215330);
   Mean: 46.1 nM in human plasma, SD: 24.4 (derived from the mass spectrometry methods described herein);
g) FHR5: ~1.5 μg/ml in human plasma (van Beek, A E et al., Front Immunol. 2017 Oct. 18; 8:1328);
   Mean: 29.2 nM in human plasma, SD: 8.7 (derived from the mass spectrometry methods described herein);
h) FI: ~35 μg/ml in human plasma;
i) C3: ~0.5-16 mg/ml in human plasma (Engström, G. et al., J Hum Hypertens. 2007 April; 21 (4): 276-82; Lee S H et al., Am J Respir Crit Care Med. 2006 Feb. 15; 173 (4): 370-8);
j) C3a: 46-157 ng/ml (Lee S H et al., Am J Respir Crit Care Med. 2006 Feb. 15; 173 (4): 370-8);
k) iC3b: ~0.7-5 μg/ml (Kim A H J et al., Arthritis Rheumatol. 2019 March; 71 (3):420-430).

In some cases, mean reference values for circulating FH, FHL-1 and FHR1-5 in human subjects known not to have a complement-related disorder, e.g. AMD, include the following (95% CI in parentheses):
a) FH, nM: 737.3 (718.2-756.5)
b) FHL-1, nM: 10.4 (10.1-10.8)
c) FHR-1, nM: 31.2 (29.4-32.9)
d) FHR-2, nM: 45.3 (43.1-47.6)
e) FHR-3, nM: 24.1 (21.7-26.5)
f) FHR-4, nM: 46.1 (42.7-49.6)
g) FHR-5, nM: 25.5 (24.5-26.5).

An ‘elevated’ level of a complement protein, e.g. in a sample, may be elevated/increased/higher when compared to a reference value for that protein, e.g. as above. A ‘reduced’ level of a complement protein, e.g. in a sample, may be reduced/decreased/lower when compared to a reference value for that protein, e.g. as above.

The relative concentrations of one complement protein to another can be determined using their reference values. For example, the ratio of the level of one complement protein to the level of another, or others, can be inferred from the concentrations provided above, e.g. FH: FHL-1, C3: iC3b, C3: C3b etc. The relative concentrations and/or ratios of the level of one complement protein to another, or others, may be altered in complement-related disorders. In some embodiments the methods provided herein involve detecting two or more complement proteins and determining how the levels of the complement proteins change with respect to one another as compared to a reference value(s). For example, the level of a first complement protein may increase as compared to the level of a second complement protein, or vice versa, e.g. FHL-1 vs FH, FHR1 to FHR5 vs FH and/or FHL-1, C3 vs iC3b, C3 vs C3b.

Provided herein is a method for determining whether a subject is at risk of developing macular degeneration, e.g. EOMD and/or AMD, the method comprising:
(a) digesting one or more complement proteins in a sample obtained from the subject, e.g. with endoproteinase GluC, to obtain one or more peptides, e.g. wherein the one or more complement proteins is selected from one or more of FHR1, FHR2, FHR3, FHR4 and/or FHR5, and optionally FH and/or FHL-1;
(b) determining the level of the one or more peptides by mass spectrometry; and
(c) using the results of (b) to determine the level of the one or more complement proteins; and
(d) determining that the subject has, or is at risk of developing, macular degeneration if the level of the FHR1, FHR2, FHR3, FHR4 and/or FHR5, and optionally FHL-1, is elevated as compared to the level of that complement protein(s) in a control subject that does not have a complement-related disorder.

Also provided herein is a method for assessing the propensity/predisposition/risk of a subject to develop a complement-related disorder, comprising steps (a) to (d) above.

In some embodiments the level of a complement protein is determined using any suitable technique known in the art and available to a skilled person. In some embodiments the level of a complement protein is determined by mass spectrometry and/or digesting the protein with endoproteinase GluC (also known as glutamyl endopeptidase; a serine proteinase which preferentially cleaves peptide bonds C-terminal to glutamic acid residues), e.g. as described in WO2021/224430, which is hereby incorporated by reference in its entirety. Determining the level of a complement protein(s) may involve detecting any combination of peptides produced by digestion with GluC, as described therein. Various suitable mass spectrometry techniques are disclosed herein and are within the routine tasks of a skilled person. The level of a complement protein may be determined using, for example, an enzyme-linked immunosorbent assay (ELISA/EIA) e.g. as described in van Beek et al., Front Immunol. 2017; 8:1328; van Beek et al. Front Immunol. 2018; 9:1727; and Pouw et al., PloS One. 2016 Mar. 23; 11 (3): e0152164; which are hereby incorporated by reference in their entirety. The level of a complement protein may be determined using, for example, Western blotting or dot blotting with appropriate antibodies, HPLC, protein immunoprecipitation or immunoelectrophoresis.

In some embodiments a method described herein comprises contacting the complement protein with endoproteinase GluC to obtain one or more peptides, and detecting the one or more peptides by mass spectrometry.

In some embodiments a method described herein comprises contacting, e.g. digesting, the protein with GluC to obtain one or more peptides, and determining the level of the one or more peptides by mass spectrometry. In some cases, the methods involves both detecting a complement protein and determining the level of a complement protein. The protein may be the same protein, or the methods may involve detection of a first complement protein and determining the level of a second complement protein.

In any and all methods described herein, the step of detecting/determining the level of the one or more peptides may consist of detecting/determining the level of/measuring the peptide(s) by mass spectrometry, e.g. as described in WO2021/224430. That is, the step of detecting/determining the level of/measuring the peptide(s) is performed by mass spectrometry only. Measuring the peptide(s) may include detecting the presence or absence of the one or more peptides, and/or determining the level, amount and/or concentration of each peptide in the sample.

In some embodiments, the step of determining in any method described herein comprises:
(i) digesting at least one complement protein in a sample e.g. blood sample obtained from the subject with endoproteinase GluC to obtain one or more peptides;
(ii) determining the presence and/or level of the one or more peptides by mass spectrometry, or performing mass spectrometry to determine the presence and/or level of the one or more peptides; and
(iii) using the results of (ii) to determine whether or not the level of the complement protein(s) is elevated/altered/reduced, e.g. as compared to the level of that complement protein(s) in a sample, e.g. blood sample, from a control subject that does not have a complement-related disorder.

In some cases, the step of determining in any method described herein comprises:
(i) obtaining a sample from a subject, wherein the sample comprises at least one complement protein that has been digested with endoproteinase GluC to obtain one or more peptides;
(ii) determining the presence and/or level of the one or more peptides by mass spectrometry, or performing mass spectrometry to determine the presence and/or level of the one or more peptides; and
(iii) using the results of (ii) to determine whether or not the level of the complement protein(s) is elevated/altered, e.g. as compared to the level of that complement protein(s) in a sample from a control subject that does not have a complement-related disorder.

In some aspects, there is provided a method of determining whether a subject has, or is at risk of developing, a complement-related disorder, the method comprising:
(a) digesting at least one complement protein, e.g. selected from FHR1, FHR2, FHR3 FHR4 and/or FHR5, in a sample obtained from the subject with endoproteinase GluC to obtain one or more peptides;
(b) determining the level of the one or more peptides by mass spectrometry;
(c) using the results of (b) to determine the level of one or more complement proteins; and
(d) determining that the subject has, or is at risk of developing, a complement-related disorder if the level of one or more complement proteins determined in (c) is elevated/altered/reduced as compared to the level of that complement protein(s) in a sample from a control subject that does not have a complement-related disorder.

Provided herein is a sample from a subject, e.g. a subject that has or is suspected to have a complement-related disorder, that comprises at least one complement protein that has been digested with endoproteinase GluC. That is, the sample comprises peptides from complement proteins that have been digested with GluC.

The term “digesting” as used herein refers to placing the protein in contact with GluC under suitable conditions, e.g. temperature, pH etc, and for a suitable time such that the protein is digested, i.e. cleaved, into two or more fragments. In some cases, the digesting involves incubating the protein with GluC under suitable conditions, e.g. as described herein. The protein, e.g. a complement-related protein according to the present disclosure, may be contacted with GluC. That is, the methods provided herein may comprise a step of contacting the protein to be digested with gluC, e.g. at a concentration suitable for digesting the protein into peptides detectable by mass spectrometry.

It will be appreciated that where “complement protein” is referred to herein in the singular (i.e. “a/the complement protein”), pluralities/groups/populations of different complement proteins are also contemplated. For example, any disclosure herein comprising a complement protein also comprises more than one complement protein, i.e. at least one protein, or one or more proteins. In all aspects and embodiments described herein, “a/the complement protein” may refer to “at least one complement protein”.

“Detecting” a protein as used herein refers to identifying/observing the presence, existence or level of the protein, e.g. in a sample, cell, tissue or subject.

The “level” of a complement protein used herein refers to the level, amount or concentration of said protein, e.g. in a sample, cell, tissue or subject. The term “determining the level”, e.g. of a protein, used herein refers to the measurement and/or quantification of the level, amount or concentration of a protein. In some cases, “determining the level” includes calculating the level, amount or concentration of a protein in a sample. The sample may be from a subject. In some cases, “determining the level” includes calculating the level, amount or concentration of a protein in a subject, e.g. using a sample taken from the subject. “Determining the level” of a protein may include digesting the protein with GluC to obtain one or more peptides, detecting the one or more peptides as described herein and then calculating the level, amount or concentration of the protein/peptide, e.g. in a sample.

In some cases, “determining the level” comprises quantifying, i.e. measuring the quantity of, the level, amount or concentration of a protein e.g. in a sample or in a subject. “Determining the level” may include determining the concentration of a protein. Quantification/measuring may include comparing the level, amount or concentration of a protein with a reference value, and/or comparing the level, amount or concentration of a protein with that in a control sample e.g. taken from the subject at a different time point, or taken from a healthy subject, e.g. one known not to have a complement-related disorder.

In some cases the methods involve determining the presence, level, amount and/or concentration of the complement protein(s) in a subject. This may involve performing the methods described herein in vitro, and using the results to calculate the presence, level, amount and/or concentration of the protein(s) in the subject.

Also provided is a method for detecting at least one complement protein in a sample, the method comprising digesting the protein(s) in the sample with endoproteinase GluC to obtain one or more peptides; and using mass spectrometry to detect the one or more peptides in the sample. Any method described herein may comprise a step of detecting at least one complement protein, e.g. detecting the presence of the complement protein.

Also provided is a method for determining the level of at least one complement protein in a sample, the method comprising digesting the protein(s) in the sample with endoproteinase GluC to obtain one or more peptides and using mass spectrometry to determine the level of the one or more peptides in the sample.

In any aspect provided herein, the methods described herein may comprise both detecting at least one complement protein and determining the level of at least one complement protein. The complement protein may be the same protein, and/or the methods may comprise detecting a least a first complement protein and determining the level of at least a second complement protein.

Methods provided herein for assessing the risk of development, i.e. the onset or risk of progression of, or for identifying subjects having/at risk of, a complement-related disorder may be performed in conjunction with additional diagnostic methods and/or tests for such disorders that will be known to one skilled in the art. In some cases, methods for assessing the risk of development of a complement-related disorder comprise further techniques selected from: CH50 or AH50 measurement via haemolytic assay, measurement of neoantigen formation during MAC complex (C5b, C6, C7, C8, C9) generation, C3 deficiency screening, mannose-binding lectin assays, immunochemical assays to quantify individual complement components, flow cytometry to assess cell-bound regulatory proteins e.g. CD55, CD59 and CD35, and/or renal function tests, see e.g. Shih A R and Murali M R, Am. J. Hematol. 2015, 90 (12): 1180-1186, Ogedegbe H O, Laboratory Medicine, 2007, 38 (5): 295-304, and Gowda S et al., N Am J Med Sci. 2010, 2 (4): 170-173, which are herein incorporated by reference in their entirety.

In some cases, methods provided herein for assessing the risk of development of AMD and/or EOMD comprise further assessment techniques selected from: dark adaptation testing, contrast sensitivity testing e.g. Pelli Robson, visual acuity testing using e.g. a Snellen chart and/or Amsler grid, Farnsworth-Munsell 100 hue test and Maximum Color Contrast Sensitivity test (MCCS) for assessing colour acuity and colour contrast sensitivity, preferential hyperacuity perimetry (PHP), fundus photography of the back of the eye, fundus examination, fundus autofluorescence, optical coherence tomography, angiography e.g. fluorescence angiography, fundus fluorescein angiography, indocyanine green angiography, optical coherence tomography angiography, adaptive optics retinal imaging, deep learning analysis of fundus images, electroretinogram methods, and/or methods to measure histological changes such as atrophy, retinal pigment changes, exudative changes e.g. hemorrhages in the eye, hard exudates, subretinal/sub-RPE/intraretinal fluid, and/or the presence of drusen.

Methods described herein may take into account lifestyle factors known to contribute to risk of developing complement-related disorders. For example, lifestyle factors that may cause or contribute to AMD include smoking, being overweight, high blood pressure and having a family history of AMD.

The methods provided herein may comprise determining in a subject the presence or absence of a genetic profile characterised by polymorphisms in the subject's genome associated with complement dysregulation. The polymorphisms may be found within or near genes such as CCL28, FBN2, ADAM12, PTPRC, IGLC1, HS3ST4, PRELP, PPID, SPOCK, APOB, SLC2A2, COL4A1, MYOC, ADAM19, FGFR2, C8A, FCN1, IFNAR2, CINH, C7 and ITGA4. A genetic profile associated with complement dysregulation may comprise one or more, often multiple, single nucleotide polymorphisms, e.g. as set out in Tables I and II of US 2010/0303832, which is hereby incorporated by reference in its entirety.

Genetic factors are thought to play a role in the development of AMD and EOMD. Thus, any of the assessment or therapeutic methods described herein may be performed in conjunction with methods to assess AMD-associated and/or EOMD-associated and/or macular dystrophy-associated genetic variants. In some cases a complement-related disorder described herein may comprise a genetic element and/or a genetic risk factor.

In some aspects of the present disclosure, there is provided a method of identifying a subject having a complement-related disorder or at risk of developing a complement-related disorder, the method comprising determining in a subject the presence or absence of one or more genetic factors associated with AMD and/or EOMD, e.g. one or more AMD- or EOMD-associated genetic variants.

In some cases, any method provided herein may comprise determining in a subject the presence or absence of one or more genetic factors associated with AMD and/or EOMD, e.g. one or more AMD- or EOMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present invention involve determining whether a subject possesses one or more such risk factors, e.g. by obtaining a sample from the subject, or In a sample obtained from the subject.

In some embodiments, the one or more genetic factors may be located on chromosome 1 at or near the RCA locus, e.g. in the CFH/CFHR genes/the CFH locus. In some embodiments, the presence of one or more CFH locus AMD-risk variants increase disease risk via increase of FHR protein levels.

The one or more genetic factors may be located in one or more of: CFH e.g. selected from Y402H (i.e. rs1061170c), rs14109969, 162V (rs800292), A473A (rs2274700), R53C, D90G, D936E (rs1065489), R1210C, IVS1 (rs529825), IVS2 insTT, IVS6 (rs3766404), A307A (rs1061147), IVS10 (rs203674), rs3753396, R1210C, rs148553336, rs191281603, rs35292876, and rs800292; CFHR4 e.g. selected from rs6685931, and rs1409153; CFI e.g. selected from G119R, and rs141853578; CFB e.g. rs4151667, C2 e.g. rs9332739, C9 e.g. P167S; and/or C3 e.g. K155Q. In some embodiments, a genetic factor is Y402H (i.e. rs1061170° C.). In some embodiments, a genetic factor is rs3753396. In some embodiments, a genetic factor is rs6685931 and/or rs1409153. In some embodiments, a genetic factor is at intronic KCNT2 rs61820755. In some embodiments, a genetic factor is not rs6685931.

In some embodiments a genetic factor is rs61820755, and may be associated with FHL-1.

In any embodiment herein, the genetic risk factors may be present in combination with elevated levels of one or more FHR proteins. The one or more genetic factors at the CFH locus may be selected from intergenic CFHR1/CFHR4 rs149369377 and/or rs61820755 for FHR-1, CFHR2 rs4085749 for FHR-2, intronic CFH rs70620 for FHR-3, rs12047098 for FHR-4, intronic KCNT2 rs72732232 for FHR-5. The presence of any one or more of these genetic factors indicates that the subject has or is likely to develop a complement-related disorder.

The one or more genetic risk factors may be selected from rs10922109, rs570618, rs121913059 (R1210C), rs148553336, rs187328863, rs61818925, rs35292876, and rs191281603.

The one or more genetic factors may be selected from one or more of rs113721756 on chromosome 10, rs111260777 on chromosome 11, rs117468955 on chromosome 12, rs200404865 on chromosome 13, rs4790395 on chromosome 17 and rs117115124 on chromosome 19. These factors may be present separately, or in addition to, genetic factors at the CFH locus. These factors may be present in combination with elevated FHR-3 levels.

Any and all combinations of genetic factors are envisaged, e.g. those described herein or additional factors, including their detection/assessment as below).

The methods described herein may involve detecting combinations of risk factors to assess the risk of a subject developing a complement related disorder, e.g. if one or both of the risk factors are present in a subject. For example:
rs10922109 and intergenic CFHR1/CFHR4 rs149369377
rs10922109 and CFHR2 rs4085749
rs10922109 and intronic CFH rs70620
rs10922109 and intergenic CFHR1-CFHR4 rs12047098
rs570618 and intergenic CFHR1/CFHR4 rs149369377
rs570618 and CFHR2 rs4085749
rs148553336 and intronic KCNT2 rs72732232
rs61818925 and CFHR2 rs4085749; and/or
rs61818925 and intergenic CFHR1-CFHR4 rs12047098.

Assessment of the presence of any genetic risk factor provided herein may be combined with the detection of any one or more of FHR1, FHR2, FHR3, FHR4 and/or FHR5, and optionally FHL-1 as described herein. For example, the presence of genetic factor rs10922109 may be assessed in combination with the detection of any one or more of FHR-1, FHR-2, FHR-3, and/or FHR-4; rs570618 may be assessed in combination with the detection of FHR-1 and/or FHR-2; rs61818925 may be assessed in combination with the detection of FHR-2 and/or FHR-4; and rs148553336 may be assessed in combination with the detection of FHR-5.

In some embodiments, the subject may comprise high risk CFH polymorphism T1277C. In some embodiments, a method according to the present disclosure does not comprise detecting the T1277C polymorphism. In some embodiments, the subject does not comprise high risk CFH polymorphism T1277C.

In any embodiment or method herein, a method may comprise a step of determining that the subject has or is likely to develop a complement-related disorder if one or more genetic factors, e.g. those described herein, are present.

Thus provided herein is a method of identifying a subject having a complement-related disorder or at risk of developing a complement-related disorder, the method comprising assessing the subject for one or more genetic risk factors, e.g. any of those described herein or others, and determining that the subject has or is likely to develop a complement-related disorder if the one or more genetic risk factors are present in the subject.

Provided herein is a method of determining whether a subject has, or is at risk of developing, a complement-related disorder, the method comprising assessing the subject for one or more genetic risk factors, e.g. any of those described herein or others, and determining that the subject has or is likely to develop a complement-related disorder if the one or more genetic risk factors are present in the subject.

Also provided is a method for selecting treatment for and/or treating subjects/patients that have a complement-related disorder or have been identified as having a complement-related disorder, e.g. using the steps above.

Also provided is a method for selecting a subject for treatment with an article/molecule described herein; a method for selecting a therapeutic agent, e.g. an article/molecule described herein, for a subject; methods of treatment; an article/molecule described herein for use in a method of treatment; and the use of an article/molecule described herein in the manufacture of a medicament for the treatment of a complement-related disorder, wherein the method uses the steps above to assess genetic risk factors (either alone or in combination with determining the level of a complement protein e.g. an FHR protein as described herein and/or determining whether the level of one or more of FHR1, FHR2, FHR3, FHR4 and/or FHR5, and optionally FHL-1, is elevated (e.g. in a sample) as compared to the level of that complement protein in a control subject (e.g. in a sample) that does not have a complement-related disorder, as described herein.

Any such method comprising detecting and assessing genetic risk factors may comprise a treatment step as described herein, e.g. treating a subject that has been determined to have or be likely to develop a complement-related disorder.

Other suitable genetic risk factors and genetic variants will be known in the art and may be as described in e.g. Edwards A O et al., Science 2005, 308 (5720): 421-4; Hageman G S et al., Proc Natl Acad Sci USA. 2005, 102 (20): 7227-7232; Haines J L et al., Science 2005, 308 (5720): 419-21, Klein R J et al., Science 2005, 308 (5720): 385-389; Fritsche et al., Nat Genet. 2016, 48 (2): 134-43; US 2010/0303832; Clark S et al., J Clin Med. 2015, 4 (1): 18-31, Cipriani, V. et al., Nat Commun. 2020, 11, 778; or Hageman G S et al, Hum Genomics. 2011, 5, 420 (2011), each hereby incorporated by reference in its entirety.

In some cases, the methods provided herein further comprise determining in a subject the presence or absence of one or more genetic factors associated with EOMD, e.g. one or more EOMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present invention involve determining whether a subject possesses one or more such risk factors. In some embodiments the subject may possess one or more risk factors for early-onset macular degeneration (EOMD).

EOMD is thought to be caused by monogenic inheritance of rare variants of the CFH gene (see e.g. Boon C J et al. Am J Hum Genet 2008; 82 (2): 516-23; van de Ven J P, et al. Arch Ophthalmol 2012; 130 (8): 1038-47; Yu Y et al. Hum Mol Genet 2014; 23 (19): 5283-93; Duvvari M R, et al. Mol Vis 2015; 21:285-92; Hughes A E, et al. Acta Ophthalmol 2016; 94 (3): e247-8; Wagner et al. Sci Rep 2016; 6:31531; Taylor R L et al, Ophthalmology. 2019 Mar. 21. Pii: S0161-6420 (18): 33171-3). In some embodiments, the subject may possess one or more of EOMD-associated genetic variants. EOMD-associated genetic variants are described in e.g. Servais A et al. Kidney Int, 2012; 82 (4): 454-64 and Dragon-Durey M A, et al. J Am Soc Nephrol 2004; 15 (3): 787-95; which are hereby incorporated by reference in their entirety. In some embodiments, the subject may possess one or more of the following EOMD-associated genetic variants: CFH c.1243del, p. (Ala415Profs*39) het; CFH c.350+1G>T het; CFH c.619+1G>A het; CFH c.380G>A, p. (Arg127His); CFH c.694C>T, p. (Arg232Ter); or CFH c.1291T>A, p. (Cys431Ser).

In some cases, the methods provided herein comprise screening for deletions within the RCA locus (a region of DNA sequence located on chromosome one that extends from the CFH gene through to the CD46 (MCP) gene) that are associated with AMD and/or EOMD risk or protection.

Methods for determining the presence or absence of genetic factors include restriction fragment length polymorphism identification (RFLPI) of genomic DNA, random amplified polymorphic detection (RAPD) of genomic DNA, amplified fragment length polymorphism detection (AFLPD), multiple locus variable number tandem repeat (VNTR) analysis (MLVA), SNP genotyping, multilocus sequence typing, PCR, DNA sequencing e.g. Sanger sequencing or Next-Generation sequencing, allele specific oligonucleotide (ASO) probes, and oligonucleotide microarrays or beads. Other suitable methods are described in e.g. Edenberg H J and Liu Y, Cold Spring Harb Protoc; 2009; doi: 10.1101/pdb.top62, and Tsuchihashi Z and Dracopoli N C, Pharmacogenomics J., 2002, 2:103-110.

### Sequence Identity

As used herein, a nucleic acid, nucleotide or amino acid sequence which corresponds to a reference nucleic acid, nucleotide or amino acid sequence may comprise at least 60%, e.g. one of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the reference sequence.

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6 (298)) and MAFFT (Katoh and Standley 2013, Molecular Biology and Evolution, 30 (4) 772-780 software. When using such software, the default parameters, e.g. for gap penalty and extension penalty, are preferably used.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof. The invention includes the combination of the aspects and preferred features described except where such a combination is clearly impermissible or expressly avoided.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

The phase “and/or” as used herein encompasses each member of the list individually, as well as any combination of one or members of the list up to and including every member of the list.

For standard molecular biology techniques, see Michael R. Green, Joseph Sambrook. Molecular Cloning, A Laboratory Manual. 4 ed. 2012, Cold Spring Harbor, New York: Cold Spring Harbor Laboratory Press.

Sequences

| SEQ | | |
|---|---|---|
| ID No: | Description | Sequence |
| 1 | Human | MGASSPRSPEPVGPPAPGLPFCCGGSLLAVVVLLALPVAWG |
| | Complement | QCNAPEWLPFARPTNLTDEFEFPIGTYLNYECRPGYSGRPFSII |
| | Receptor 1 | CLKNSVWTGAKDRCRRKSCRNPPDPVNGMVHVIKGIQFGSQ |
| | (UniProt: P17927; | IKYSCTKGYRLIGSSSATCIISGDTVIWDNETPICDRIPCGLPPT |
| | Entry version 205 | ITNGDFISTNRENFHYGSVVTYRCNPGSGGRKVFELVGEPSIY |
| | (23 Feb. 2022), | CTSNDDQVGIWSGPAPQCIIPNKCTPPNVENGILVSDNRSLFS |
| | Sequence version | LNEVVEFRCQPGFVMKGPRRVKCQALNKWEPELPSCSRVCQ |
| | 3 (2 Mar. 2010)); | PPPDVLHAERTQRDKDNFSPGQEVFYSCEPGYDLRGAASMR |
| | residues 1-2039 | CTPQGDWSPAAPTCEVKSCDDFMGQLLNGRVLFPVNLQLG |
| | Including signal | AKVDFVCDEGFQLKGSSASYCVLAGMESLWNSSVPVCEQIF |
| | peptide sequence | CPSPPVIPNGRHTGKPLEVFPFGKTVNYTCDPHPDRGTSFDLI |
| | [C3b-binding | GESTIRCTSDPQGNGVWSSPAPRCGILGHCQAPDHFLFAKLK |
| | domains ‘CCPs 8- | TQTNASDFPIGTSLKYECRPEYYGRPFSITCLDNLVWSSPKDV |
| | 10’ and ‘CCPs | CKRKSCKTPPDPVNGMVHVITDIQVGSRINYSCTTGHRLIGH |
| | 15-17’ indicated | SSAECILSGNAAHWSTKPPICQRIPCGLPPTIANGDFISTNREN |
| | by underline] | FHYGSVVTYRCNPGSGGRKVFELVGEPSIYCTSNDDQVGIW |
| | | SGPAPQCIIPNKCTPPNVENGILVSDNRSLFSLNEVVEFRCQP |
| | | GFVMKGPRRVKCQALNKWEPELPSCSRVCQPPPDVLHAERT |
| | | QRDKDNFSPGQEVFYSCEPGYDLRGAASMRCTPQGDWSPA |
| | | APTCEVKSCDDFMGQLLNGRVLFPVNLQLGAKVDFVCDEG |
| | | FQLKGSSASYCVLAGMESLWNSSVPVCEQIFCPSPPVIPNGR |
| | | HTGKPLEVFPFGKAVNYTCDPHPDRGTSFDLIGESTIRCTSDP |
| | | QGNGVWSSPAPRCGILGHCQAPDHFLFAKLKTQTNASDFPIG |
| | | TSLKYECRPEYYGRPFSITCLDNLVWSSPKDVCKRKSCKTPP |
| | | DPVNGMVHVITDIQVGSRINYSCTTGHRLIGHSSAECILSGNT |
| | | AHWSTKPPICQRIPCGLPPTIANGDFISTNRENFHYGSVVTYR |
| | | CNLGSRGRKVFELVGEPSIYCTSNDDQVGIWSGPAPQCIIPNK |
| | | CTPPNVENGILVSDNRSLFSLNEVVEFRCQPGFVMKGPRRVK |
| | | CQALNKWEPELPSCSRVCQPPPEILHGEHTPSHQDNFSPGQE |
| | | VFYSCEPGYDLRGAASLHCTPQGDWSPEAPRCAVKSCDDFL |
| | | GQLPHGRVLFPLNLQLGAKVSFVCDEGFRLKGSSVSHCVLV |
| | | GMRSLWNNSVPVCEHIFCPNPPAILNGRHTGTPSGDIPYGKEI |
| | | SYTCDPHPDRGMTFNLIGESTIRCTSDPHGNGVWSSPAPRCE |
| | | LSVRAGHCKTPEQFPFASPTIPINDFEFPVGTSLNYECRPGYF |
| | | GKMFSISCLENLVWSSVEDNCRRKSCGPPPEPFNGMVHINTD |
| | | TQFGSTVNYSCNEGFRLIGSPSTTCLVSGNNVTWDKKAPICEI |
| | | ISCEPPPTISNGDFYSNNRTSFHNGTVVTYQCHTGPDGEQLFE |
| | | LVGERSIYCTSKDDQVGVWSSPPPRCISTNKCTAPEVENAIR |
| | | VPGNRSFFSLTEIIRFRCQPGFVMVGSHTVQCQTNGRWGPKL |
| | | PHCSRVCQPPPEILHGEHTLSHQDNFSPGQEVFYSCEPSYDLR |
| | | GAASLHCTPQGDWSPEAPRCTVKSCDDFLGQLPHGRVLLPL |
| | | NLQLGAKVSFVCDEGFRLKGRSASHCVLAGMKALWNSSVP |
| | | VCEQIFCPNPPAILNGRHTGTPFGDIPYGKEISYACDTHPDRG |
| | | MTFNLIGESSIRCTSDPQGNGVWSSPAPRCELSVPAACPHPPK |
| | | IQNGHYIGGHVSLYLPGMTISYICDPGYLLVGKGFIFCTDQGI |
| | | WSQLDHYCKEVNCSFPLFMNGISKELEMKKVYHYGDYVTL |
| | | KCEDGYTLEGSPWSQCQADDRWDPPLAKCTSRTHDALIVGT |
| | | LSGTIFFILLIIFLSWIILKHRKGNNAHENPKEVAIHLHSQGGSS |
| | | VHPRTLQTNEENSRVLP |
| 2 | Human | GHCQAPDHFLFAKLKTQTNASDFPIGTSLKYECRPEYYGRPF |
| | Complement | SITCLDNLVWSSPKDVCKRKSCKTPPDPVNGMVHVITDIQVG |
| | Receptor 1; | SRINYSCTTGHRLIGHSSAECILSGNX₁AHWSTKPPICQRIPCG |
| | consensus | LPPTIANGDFISTNRENFHYGSVVTYRCNX₂GSX₃GRKVFELV |
| | sequence for | GEPSIYCTSNDDQVGIWSGPAPQCII |
| | CCPs 8-10, 15-17 | |
| | (UniProt: P17927 | |
| | residues 491 to | |
| | 684; residues 941 | |
| | to 1134) | |
| | Without signal | |
| | peptide sequence | |
| 3 | Human | GHCQAPDHFLFAKLKTQTNASDFPIGTSLKYECRPEYYGRPF |
| | Complement | SITCLDNLVWSSPKDVCKRKSCKTPPDPVNGMVHVITDIQVG |
| | Receptor 1 CCPs | SRINYSCTTGHRLIGHSSAECILSGNAAHWSTKPPICQRIPCGL |
| | 8-10 (UniProt: | PPTIANGDFISTNRENFHYGSVVTYRCNPGSGGRKVFELVGE |
| | P17927 residues | PSIYCTSNDDQVGIWSGPAPQCII |
| | 491 to 684) | |
| | Without signal | |
| | peptide sequence | |
| 4 | Human | GHCQAPDHFLFAKLKTQTNASDFPIGTSLKYECRPEYYGRPF |
| | Complement | SITCLDNLVWSSPKDVCKRKSCKTPPDPVNGMVHVITDIQVG |
| | Receptor 1 CCPs | SRINYSCTTGHRLIGHSSAECILSGNTAHWSTKPPICQRIPCGL |
| | 15-17 (UniProt: | PPTIANGDFISTNRENFHYGSVVTYRCNLGSRGRKVFELVGE |
| | P17927 residues | PSIYCTSNDDQVGIWSGPAPQCII |
| | 941 to 1134) | |
| | Without signal | |
| | peptide sequence | |
| 5 | Codon-optimised | GGACATTGTCAGGCCCCTGACCACTTCCTGTTCGCCAAGC |
| | nucleic acid | TGAAAACCCAGACCAACGCCAGCGACTTCCCTATCGGCAC |
| | encoding human | CAGCCTGAAGTACGAGTGCAGACCCGAGTACTACGGCAG |
| | CR1 CCPs 8-10 | ACCCTTCAGCATCACCTGTCTGGACAACCTCGTGTGGTCT |
| | | AGCCCCAAGGACGTGTGCAAGAGAAAGAGCTGCAAGACC |
| | | CCTCCTGATCCTGTGAACGGCATGGTGCACGTGATCACCG |
| | | ACATCCAAGTGGGCAGCAGAATCAACTACAGCTGCACCA |
| | | CCGGCCACAGACTGATCGGACACTCTAGCGCCGAGTGTAT |
| | | CCTGAGCGGCAATGCCGCACACTGGTCCACCAAGCCTCCA |
| | | ATCTGCCAGAGAATCCCTTGCGGCCTGCCTCCTACAATCG |
| | | CCAACGGCGATTTCATCAGCACCAACAGAGAGAACTTCC |
| | | ACTACGGCTCCGTGGTCACCTACAGATGCAATCCTGGCAG |
| | | CGGCGGCAGAAAGGTGTTCGAACTTGTGGGCGAGCCCAG |
| | | CATCTACTGCACCAGCAACGATGACCAAGTCGGCATTTGG |
| | | AGCGGCCCTGCTCCTCAGTGCATCATC |
| 6 | Codon-optimised | ATGAGACTGCTGGCCAAGATCATCTGCCTGATGCTGTGGG |
| | nucleic acid | CCATCTGCGTGGCCGGACATTGTCAGGCCCCTGACCACTT |
| | encoding human | CCTGTTCGCCAAGCTGAAAACCCAGACCAACGCCAGCGA |
| | CR1 CCPs 8-10 | CTTCCCTATCGGCACCAGCCTGAAGTACGAGTGCAGACCC |
| | (with codon | GAGTACTACGGCAGACCCTTCAGCATCACCTGTCTGGACA |
| | optimised FH | ACCTCGTGTGGTCTAGCCCCAAGGACGTGTGCAAGAGAA |
| | signal peptide | AGAGCTGCAAGACCCCTCCTGATCCTGTGAACGGCATGGT |
| | sequence | GCACGTGATCACCGACATCCAAGTGGGCAGCAGAATCAA |
| | underlined) | CTACAGCTGCACCACCGGCCACAGACTGATCGGACACTCT |
| | | AGCGCCGAGTGTATCCTGAGCGGCAATGCCGCACACTGGT |
| | | CCACCAAGCCTCCAATCTGCCAGAGAATCCCTTGCGGCCT |
| | | GCCTCCTACAATCGCCAACGGCGATTTCATCAGCACCAAC |
| | | AGAGAGAACTTCCACTACGGCTCCGTGGTCACCTACAGAT |
| | | GCAATCCTGGCAGCGGCGGCAGAAAGGTGTTCGAACTTG |
| | | TGGGCGAGCCCAGCATCTACTGCACCAGCAACGATGACC |
| | | AAGTCGGCATTTGGAGCGGCCCTGCTCCTCAGTGCATCAT |
| | | C |
| 7 | Wild type nucleic | GGTCACTGTCAAGCCCCAGATCATTTTCTGTTTGCCAAGTT |
| | acid encoding | GAAAACCCAAACCAATGCATCTGACTTTCCCATTGGGACA |
| | human CR1 CCPs | TCTTTAAAGTACGAATGCCGTCCTGAGTACTACGGGAGGC |
| | 8-10 | CATTCTCTATCACATGTCTAGATAACCTGGTCTGGTCAAG |
| | | TCCCAAAGATGTCTGTAAACGTAAATCATGTAAAACTCCT |
| | | CCAGATCCAGTGAATGGCATGGTGCATGTGATCACAGAC |
| | | ATCCAGGTTGGATCCAGAATCAACTATTCTTGTACTACAG |
| | | GGCACCGACTCATTGGTCACTCATCTGCTGAATGTATCCT |
| | | CTCGGGCAATGCTGCCCATTGGAGCACGAAGCCGCCAATT |
| | | TGTCAACGAATTCCTTGTGGGCTACCCCCCACCATCGCCA |
| | | ATGGAGATTTCATTAGCACCAACAGAGAGAATTTTCACTA |
| | | TGGATCAGTGGTGACCTACCGCTGCAATCCTGGAAGCGGA |
| | | GGGAGAAAGGTGTTTGAGCTTGTGGGTGAGCCCTCCATAT |
| | | ACTGCACCAGCAATGACGATCAAGTGGGCATCTGGAGCG |
| | | GCCCGGCCCCTCAGTGCATTATA |
| 8 | Wild type nucleic | ATGAGACTTCTAGCAAAGATTATTTGCCTTATGTTATGGG |
| | acid encoding | CTATTTGTGTAGCAGGTCACTGTCAAGCCCCAGATCATTT |
| | human CR1 CCPs | TCTGTTTGCCAAGTTGAAAACCCAAACCAATGCATCTGAC |
| | 8-10 (with wild | TTTCCCATTGGGACATCTTTAAAGTACGAATGCCGTCCTG |
| | type FH signal | AGTACTACGGGAGGCCATTCTCTATCACATGTCTAGATAA |
| | peptide sequence | CCTGGTCTGGTCAAGTCCCAAAGATGTCTGTAAACGTAAA |
| | underlined) | TCATGTAAAACTCCTCCAGATCCAGTGAATGGCATGGTGC |
| | | ATGTGATCACAGACATCCAGGTTGGATCCAGAATCAACTA |
| | | TTCTTGTACTACAGGGCACCGACTCATTGGTCACTCATCT |
| | | GCTGAATGTATCCTCTCGGGCAATGCTGCCCATTGGAGCA |
| | | CGAAGCCGCCAATTTGTCAACGAATTCCTTGTGGGCTACC |
| | | CCCCACCATCGCCAATGGAGATTTCATTAGCACCAACAGA |
| | | GAGAATTTTCACTATGGATCAGTGGTGACCTACCGCTGCA |
| | | ATCCTGGAAGCGGAGGGAGAAAGGTGTTTGAGCTTGTGG |
| | | GTGAGCCCTCCATATACTGCACCAGCAATGACGATCAAGT |
| | | GGGCATCTGGAGCGGCCCGGCCCCTCAGTGCATTATA |
| 9 | Native signal | MRLLAKIICLMLWAICVA |
| | peptide from | |
| | Factor H (Uniprot | |
| | P08603-1) | |
| 10 | Native nucleic | ATGAGACTTCTAGCAAAGATTATTTGCCTTATGTTATGGG |
| | acid encoding | CTATTTGTGTAGCA |
| | signal peptide | |
| | sequence from | |
| | Factor H | |
| 11 | Codon optimised | ATGAGACTGCTGGCCAAGATCATCTGCCTGATGCTGTGGG |
| | nucleic acid | CCATCTGCGTGGCC |
| | encoding signal | |
| | peptide from | |
| | Factor H | |
| 12 | Human | MRLLAKIICLMLWAICVAGHCQAPDHFLFAKLKTQTNASDF |
| | Complement | PIGTSLKYECRPEYYGRPFSITCLDNLVWSSPKDVCKRKSCK |
| | Receptor 1 CCPs | TPPDPVNGMVHVITDIQVGSRINYSCTTGHRLIGHSSAECILS |
| | 8-10 (including | GNAAHWSTKPPICQRIPCGLPPTIANGDFISTNRENFHYGSVV |
| | FH signal peptide | TYRCNPGSGGRKVFELVGEPSIYCTSNDDQVGIWSGPAPQCII |
| | sequence) | |
| 13 | Human | MRLLAKIICLMLWAICVAGHCQAPDHFLFAKLKTQTNASDF |
| | Complement | PIGTSLKYECRPEYYGRPFSITCLDNLVWSSPKDVCKRKSCK |
| | Receptor 1 CCPs | TPPDPVNGMVHVITDIQVGSRINYSCTTGHRLIGHSSAECILS |
| | 15-17 (including | GNTAHWSTKPPICQRIPCGLPPTIANGDFISTNRENFHYGSVV |
| | FH signal peptide | TYRCNLGSRGRKVFELVGEPSIYCTSNDDQVGIWSGPAPQCII |
| | sequence) | |
| 14 | CBh promoter | CGTTACATAACTTACGGTAAATGGCCCGCCTGGCTGACCG |
| | | CCCAACGACCCCCGCCCATTGACGTCAATAGTAACGCCAA |
| | | TAGGGACTTTCCATTGACGTCAATGGGTGGAGTATTTACG |
| | | GTAAACTGCCCACTTGGCAGTACATCAAGTGTATCATATG |
| | | CCAAGTACGCCCCCTATTGACGTCAATGACGGTAAATGGC |
| | | CCGCCTGGCATTGTGCCCAGTACATGACCTTATGGGACTT |
| | | TCCTACTTGGCAGTACATCTACGTATTAGTCATCGCTATTA |
| | | CCATGGTCGAGGTGAGCCCCACGTTCTGCTTCACTCTCCC |
| | | CATCTCCCCCCCCTCCCCACCCCCAATTTTGTATTTATTTA |
| | | TTTTTTAATTATTTTGTGCAGCGATGGGGGCGGGGGGGGG |
| | | GGGGGGGCGCGCGCCAGGCGGGGCGGGGCGGGGCGAGG |
| | | GGCGGGGCGGGGCGAGGCGGAGAGGTGCGGCGGCAGCC |
| | | AATCAGAGCGGCGCGCTCCGAAAGTTTCCTTTTATGGCGA |
| | | GGCGGCGGCGGCGGCGGCCCTATAAAAAGCGAAGCGCGC |
| | | GGCGGGCGGGAGTCGCTGCGCGCTGCCTTCGCCCCGTGCC |
| | | CCGCTCCGCCGCCGCCTCGCGCCGCCCGCCCCGGCTCTGA |
| | | CTGACCGCGTTACTCCCACAGGTGAGCGGGCGGGACGGC |
| | | CCTTCTCCTCCGGGCTGTAATTAGCTGAGCAAGAGGTAAG |
| | | GGTTTAAGGGATGGTTGGTTGGTGGGGTATTAATGTTTAA |
| | | TTACCTGGAGCACCTGCCTGAAATCACTTTTTTTCAGGTTG |
| | | G |
| 15 | CAG promoter | CCATTGACGTCAATAATGACGTATGTTCCCATAGTAACGC |
| | | CAATAGGGACTTTCCATTGACGTCAATGGGTGGAGTATTT |
| | | ACGGTAAACTGCCCACTTGGCAGTACATCAAGTGTATCAT |
| | | ATGCCAAGTACGCCCCCTATTGACGTCAATGACGGTAAAT |
| | | GGCCCGCCTGGCATTATGCCCAGTACATGACCTTATGGGA |
| | | CTTTCCTACTTGGCAGTACATCTACGTATTAGTCATCGCTA |
| | | TTACCATGGTCGAGGTGAGCCCCACGTTCTGCTTCACTCT |
| | | CCCCATCTCCCCCCCCTCCCCACCCCCAATTTTGTATTTAT |
| | | TTATTTTTTAATTATTTTGTGCAGCGATGGGGGCGGGGGG |
| | | GGGGGGGGCGCGCGCCAGGCGGGGCGGGGCGGGGCGAG |
| | | GGGCGGGGCGGGGCGAGGCGGAGAGGTGCGGCGGCAGC |
| | | CAATCAGAGCGGCGCGCTCCGAAAGTTTCCTTTTATGGCG |
| | | AGGCGGCGGCGGCGGCGGCCCTATAAAAAGCGAAGCGCG |
| | | CGGCGGGCGGGAGTCGCTGCGCGCTGCCTTCGCCCCGTGC |
| | | CCCGCTCCGCCGCCGCCTCGCGCCGCCCGCCCCGGCTCTG |
| | | ACTGACCGCGTTACTCCCACAGGTGAGCGGGCGGGACGG |
| | | CCCTTCTCCTCCGGGCTGTAATTAGCGCTTGGTTTAATGAC |
| | | GGCTTGTTTCTTTTCTGTGGCTGCGTGAAAGCCTTGAGGG |
| | | GCTCCGGGAGGGCCCTTTGTGCGGGGGGAGCGGCTCGGG |
| | | GGGTGCGTGCGTGTGTGTGTGCGTGGGGAGCGCCGCGTGC |
| | | GGCTCCGCGCTGCCCGGCGGCTGTGAGCGCTGCGGGCGC |
| | | GGCGCGGGGCTTTGTGCGCTCCGCAGTGTGCGCGAGGGG |
| | | AGCGCGGCCGGGGGCGGTGCCCCGCGGTGCGGGGGGGGC |
| | | TGCGAGGGGAACAAAGGCTGCGTGCGGGGTGTGTGCGTG |
| | | GGGGGGTGAGCAGGGGGTGTGGGCGCGTCGGTCGGGCTG |
| | | CAACCCCCCCTGCACCCCCCTCCCCGAGTTGCTGAGCACG |
| | | GCCCGGCTTCGGGTGCGGGGCTCCGTACGGGGCGTGGCG |
| | | CGGGGCTCGCCGTGCCGGGCGGGGGGTGGCGGCAGGTGG |
| | | GGGTGCCGGGCGGGGCGGGGCCGCCTCGGGCCGGGGAGG |
| | | GCTCGGGGGAGGGGCGCGGCGGCCCCCGGAGCGCCGGCG |
| | | GCTGTCGAGGCGCGGCGAGCCGCAGCCATTGCCTTTTATG |
| | | GTAATCGTGCGAGAGGGCGCAGGGACTTCCTTTGTCCCAA |
| | | ATCTGTGCGGAGCCGAAATCTGGGAGGCGCCGCCGCACC |
| | | CCCTCTAGCGGGCGCGGGGCGAAGCGGTGCGGCGCCGGC |
| | | AGGAAGGAAATGGGCGGGGAGGGCCTTCGTGCGTCGCCG |
| | | CGCCGCCGTCCCCTTCTCCCTCTCCAGCCTCGGGGCTGTCC |
| | | GCGGGGGGACGGCTGCCTTCGGGGGGGACGGGGCAGGGC |
| | | GGGGTTCGGCTTCTGGCGTGTGACCGGCGGCTCTAGAGCC |
| | | TCTGCTAACCATGTTCATGCCTTCTTCTTTTTCCTACAGCT |
| | | CCTGGGCAACGTGCTGGTTGTTGTGCTGTCTCATCATTTTG |
| | | GCAAAGAATTAGATCTTTGTCGATCCTACCATCCACTC |
| 16 | Shortened CAG | CCATTGACGTCAATAATGACGTATGTTCCCATAGTAACGC |
| | promoter (sCAG) | CAATAGGGACTTTCCATTGACGTCAATGGGTGGAGTATTT |
| | | ACGGTAAACTGCCCACTTGGCAGTACATCAAGTGTATCAT |
| | | ATGCCAAGTACGCCCCCTATTGACGTCAATGACGGTAAAT |
| | | GGCCCGCCTGGCATTATGCCCAGTACATGACCTTATGGGA |
| | | CTTTCCTACTTGGCAGTACATCTACGTATTAGTCATCGCTA |
| | | TTACCATGGTCGAGGTGAGCCCCACGTTCTGCTTCACTCT |
| | | CCCCATCTCCCCCCCCTCCCCACCCCCAATTTTGTATTTAT |
| | | TTATTTTTTAATTATTTTGTGCAGCGATGGGGGCGGGGGG |
| | | GGGGGGGGCGCGCGCCAGGCGGGGCGGGGCGGGGCGAG |
| | | GGGCGGGGCGGGGCGAGGCGGAGAGGTGCGGCGGCAGC |
| | | CAATCAGAGCGGCGCGCTCCGAAAGTTTCCTTTTATGGCG |
| | | AGGCGGCGGCGGCGGCGGCCCTATAAAAAGCGAAGCGCG |
| | | CGGCGGGCGGGAGTCGCTGCGCGCTGCCTTCGCCCCGTGC |
| | | CCCGCTCCGCCGCCGCCTCGCGCCGCCCGCCCCGGCTCTG |
| | | ACTGACCGCGTTACTCCCACAGGTGAGCGGGCGGGACGG |
| | | CCCTTCTCCTCCGGGCTGTAATTAGCGCTTGGTTTAATGAC |
| | | GGCTTGTTTCTTTTCTGTGGCTGCGTGAAAGCCTTGAGGG |
| | | GCTCCGGGAGGGCCCTTTGTGCGGGGGGAGCGGCTCGGG |
| | | GCTGTCCGCGGGGGGACGGCTGCCTTCGGGGGGGACGGG |
| | | GCAGGGCGGGGTTCGGCTTCTGGCGTGTGACCGGCGGCTC |
| | | TAGAGCCTCTGCTAACCATGTTCATGCCTTCTTCTTTTTCC |
| | | TACAGCTCCTGGGCAACGTGCTGGTTGTTGTGCTGTCTCA |
| | | TCATTTTGGCAAAGAATTAGATCTTTGTCGATCCTACCATC |
| | | CACTC |
| 17 | Truncated CAG | CCATTGACGTCAATAATGACGTATGTTCCCATAGTAACGC |
| | promoter | CAATAGGGACTTTCCATTGACGTCAATGGGTGGAGTATTT |
| | (mCAG) | ACGGTAAACTGCCCACTTGGCAGTACATCAAGTGTATCAT |
| | | ATGCCAAGTACGCCCCCTATTGACGTCAATGACGGTAAAT |
| | | GGCCCGCCTGGCATTATGCCCAGTACATGACCTTATGGGA |
| | | CTTTCCTACTTGGCAGTACATCTACGTATTAGTCATCGCTA |
| | | TTACCATGGTCGAGGTGAGCCCCACGTTCTGCTTCACTCT |
| | | CCCCATCTCCCCCCCCTCCCCACCCCCAATTTTGTATTTAT |
| | | TTATTTTTTAATTATTTTGTGCAGCGATGGGGGCGGGGGG |
| | | GGGGGGGGGGCCCCCCCCAGGCGGGGCGGGGCGGGGCG |
| | | AGGGGCGGGGCGGGGCGAGGCGGAAAGGTGCGGCGGCA |
| | | GCCAATCAGAGCGGCGCGCTCCGAAAGTTTCCTTTTATGG |
| | | CGAGGCGGCGGCGGCGGCGGCCCTATAAAAAGCGAAGCG |
| | | CGCGGCGGGCGGTAAGTATCAAGGTTACAAGACAGGTTT |
| | | AAGGAGACCAATAGAAACTGGGCTTGTCGAGACAGAGAA |
| | | GACTCTTGCGTTTCTGATAGGCACCTATTGGTCTTACTGAC |
| | | ATCCACTTTGCCTTTCTCTCCACAG |
| 18 | Human EF1A | GGCTCCGGTGCCCGTCAGTGGGCAGAGCGCACATCGCCC |
| | promoter | ACAGTCCCCGAGAAGTTGGGGGGAGGGGTCGGCAATTGA |
| | | ACCGGTGCCTAGAGAAGGTGGCGCGGGGTAAACTGGGAA |
| | | AGTGATGTCGTGTACTGGCTCCGCCTTTTTCCCGAGGGTG |
| | | GGGGAGAACCGTATATAAGTGCAGTAGTCGCCGTGAACG |
| | | TTCTTTTTCGCAACGGGTTTGCCGCCAGAACACAGGTAAG |
| | | TGCCGTGTGTGGTTCCCGCGGGCCTGGCCTCTTTACGGGT |
| | | TATGGCCCTTGCGTGCCTTGAATTACTTCCACCTGGCTGCA |
| | | GTACGTGATTCTTGATCCCGAGCTTCGGGTTGGAAGTGGG |
| | | TGGGAGAGTTCGAGGCCTTGCGCTTAAGGAGCCCCTTCGC |
| | | CTCGTGCTTGAGTTGAGGCCTGGCCTGGGCGCTGGGGCCG |
| | | CCGCGTGCGAATCTGGTGGCACCTTCGCGCCTGTCTCGCT |
| | | GCTTTCGATAAGTCTCTAGCCATTTAAAATTTTTGATGACC |
| | | TGCTGCGACGCTTTTTTTCTGGCAAGATAGTCTTGTAAATG |
| | | CGGGCCAAGATCTGCACACTGGTATTTCGGTTTTTGGGGC |
| | | CGCGGGCGGCGACGGGGCCCGTGCGTCCCAGCGCACATG |
| | | TTCGGCGAGGCGGGGCCTGCGAGCGCGGCCACCGAGAAT |
| | | CGGACGGGGGTAGTCTCAAGCTGGCCGGCCTGCTCTGGTG |
| | | CCTGGCCTCGCGCCGCCGTGTATCGCCCCGCCCTGGGCGG |
| | | CAAGGCTGGCCCGGTCGGCACCAGTTGCGTGAGCGGAAA |
| | | GATGGCCGCTTCCCGGCCCTGCTGCAGGGAGCTCAAAATG |
| | | GAGGACGCGGCGCTCGGGAGAGCGGGCGGGTGAGTCACC |
| | | CACACAAAGGAAAAGGGCCTTTCCGTCCTCAGCCGTCGCT |
| | | TCATGTGACTCCACGGAGTACCGGGCGCCGTCCAGGCACC |
| | | TCGATTAGTTCTCGAGCTTTTGGAGTACGTCGTCTTTAGGT |
| | | TGGGGGGAGGGGTTTTATGCGATGGAGTTTCCCCACACTG |
| | | AGTGGGTGGAGACTGAAGTTAGGCCAGCTTGGCACTTGAT |
| | | GTAATTCTCCTTGGAATTTGCCCTTTTTGAGTTTGGATCTT |
| | | GGTTCATTCTCAAGCCTCAGACAGTGGTTCAAAGTTTTTTT |
| | | CTTCCATTTCAGGTGTCGTGA |
| 19 | SV40 | TAAGATACATTGATGAGTTTGGACAAACCACAACTAGAAT |
| | polyadenylation | GCAGTGAAAAAAATGCTTTATTTGTGAAATTTGTGATGCT |
| | signal sequence | ATTGCTTTATTTGTAACCATTATAAGCTGCAATAAACAAG |
| | | TT |
| 20 | Bovine growth | CTGTGCCTTCTAGTTGCCAGCCATCTGTTGTTTGCCCCTCC |
| | hormone (bGH) | CCCGTGCCTTCCTTGACCCTGGAAGGTGCCACTCCCACTG |
| | polyadenylation | TCCTTTCCTAATAAAATGAGGAAATTGCATCGCATTGTCT |
| | signal sequence | GAGTAGGTGTCATTCTATTCTGGGGGGTGGGGTGGGGCAG |
| | | GACAGCAAGGGGGAGGATTGGGAAGACAATAGCAGGCAT |
| | | GCTGGGGATGCGGTGGGCTCTATGG |
| 21 | Human growth | GGGTGGCATCCCTGTGACCCCTCCCCAGTGCCTCTCCTGG |
| | hormone (hGH) | CCCTGGAAGTTGCCACTCCAGTGCCCACCAGCCTTGTCCT |
| | polyadenylation | AATAAAATTAAGTTGCATCATTTTGTCTGACTAGGTGTCC |
| | signal sequence | TTCTATAATATTATGGGGTGGAGGGGGGTGGTATGGAGCA |
| | | AGGGGCAAGTTGGGAAGACAACCTGTAGGGCCTGCGGGG |
| | | TCTATTGGGAACCAAGCTGGAGTGCAGTGGCACAATCTTG |
| | | GCTCACTGCAATCTCCGCCTCCTGGGTTCAAGCGATTCTC |
| | | CTGCCTCAGCCTCCCGAGTTGTTGGGATTCCAGGCATGCA |
| | | TGACCAGGCTCAGCTAATTTTTGTTTTTTTGGTAGAGACG |
| | | GGGTTTCACCATATTGGCCAGGCTGGTCTCCAACTCCTAA |
| | | TCTCAGGTGATCTACCCACCTTGGCCTCCCAAATTGCTGG |
| | | GATTACAGGCGTGAACCACTGCTCCCTTCCCTGTCCTT |
| 22 | Woodchuck | AATCAACCTCTGGATTACAAAATTTGTGAAAGATTGACTG |
| | Hepatitis Virus | GTATTCTTAACTATGTTGCTCCTTTTACGCTATGTGGATAC |
| | (WHV) | GCTGCTTTAATGCCTTTGTATCATGCTATTGCTTCCCGTAT |
| | Posttranscriptional | GGCTTTCATTTTCTCCTCCTTGTATAAATCCTGGTTGCTGT |
| | Regulatory | CTCTTTATGAGGAGTTGTGGCCCGTTGTCAGGCAACGTGG |
| | Element (WPRE) | CGTGGTGTGCACTGTGTTTGCTGACGCAACCCCCACTGGT |
| | | TGGGGCATTGCCACCACCTGTCAGCTCCTTTCCGGGACTT |
| | | TCGCTTTCCCCCTCCCTATTGCCACGGCGGAACTCATCGCC |
| | | GCCTGCCTTGCCCGCTGCTGGACAGGGGCTCGGCTGTTGG |
| | | GCACTGACAATTCCGTGGTGTTGTCGGGGAAATCATCGTC |
| | | CTTTCCTTGGCTGCTCGCCTGTGTTGCCACCTGGATTCTGC |
| | | GCGGGACGTCCTTCTGCTACGTCCCTTCGGCCCTCAATCC |
| | | AGCGGACCTTCCTTCCCGCGGCCTGCTGCCGGCTCTGCGG |
| | | CCTCTTCCGCGTCTTCGCCTTCGCCCTCAGACGAGTCGGAT |
| | | CTCCCTTTGGGCCGCCTCCCCGC |
| 23 | Synthetic VMD2 | TCCACAAGGTGCCCCAGGTGGACTGGGCTGGGGCTTTGAG |
| | (BEST1) intron | GCCTTCAGGGTTGGATGGCCATCTTGCGTATTTGTGTGGG |
| | flanked by 12 bp | ATATGCACACACAGGCAGCACATGCGCAGGTGTGTGGGC |
| | of VMD2 | ACCTGTGTGTCTGTGCAAATGCCCTGAGGTGGGAATGAGC |
| | untranslated | TTGGTGTGCATCAGGCACAGCCAGCCAGTGTGGCTGCAGC |
| | exon-1 and exon- | AAAACACACAGGGAAAGAATGGAGGGGGCATCAATCACT |
| | 2 sequences | GCTTCAGTAAATTTTTATTGAGCGCCTTCTACGAGAACAC |
| | | AAGAGGAGCTTCCATTCTGAGGATATCCTGTATAATTTCA |
| | | AGTAGTGATAAGTGCTCTCTAGAAATATCAAGCAAGGTG |
| | | AGGAGACACAGAGCACCGGTGGCAGTGGGGCTCTATTTC |
| | | CAGGTTGGATGGTTGGGAACATCCTTTCTAAAGGGAACCT |
| | | GGAGTGGGAAGGAACCATGCAGGTATCTCAGGAAGAGCT |
| | | TCCTCCAGGCAGGAAGATCAGCAGGTGGAAAGGCCCTGG |
| | | AGCCACCATTCAGTAAACATCATTTGAGCATCTCTACCAG |
| | | CTAGGTTCCATTATGGGAATGGGAATATGGTGGTGGACAG |
| | | GGCTGCCTGGTCCCTTCCATACTTCTCACACTAGGGTGGTT |
| | | GAGAGAGCTTGGGAGGGGCTGAGAACACTGCCTAGCCCA |
| | | GAGGACCTGAGCTTAGTGTGTAGACATTGCTGCTGTTACT |
| | | GCCTTTGTCATTGTATTAGACAGAGTTTTGCTCTTCTTACC |
| | | CAGGCTGGAGTGCAATGGCGTGATCTCAGCTCACTGCAAC |
| | | CTCCACCTCCTGGGTTCAAGCGATTCTCCTGCCTCAGCCTC |
| | | CTGAGTAGCTGGGATTACAGGCACCCGCACCACGCCTGG |
| | | ATAATTTTTTTGTATTTTTAGTAGAGACAGGGTTTCACCAT |
| | | GTTGGCCAGGCTGGTCTCGAACTCCTGACCTTAGGTGATC |
| | | CACCTGCCTCGACTTCCCAAAGTGCTGGGATTATAGGCAT |
| | | GAGCCACTGCGCCCAGTGATTATAGAAAGTTAAAGGCAC |
| | | ATGGCAATGCACACGCCTATCTACGTCTTCCCTGCCAAAG |
| | | CAAAGGGCAGCCTCTGGGCTCACTTTCTTGCGTTTCTACTT |
| | | CCAAAAGGCAGTCAGAACTGGCAGGGCCTTGGAGACCAC |
| | | TTCATCCACCTCCTAGGGTCCCTATGGGAGAGTTGAGGTC |
| | | CAGAGCAGGGAAGGGTCCTGACAGGCTCTGACCAGGGCC |
| | | TCTGATCCCTACAAACCCCCAATCGGTGGCGAT |
| 24 | Synthetic RLBP1 | ACCTGGGGCTTGCCTGGGCCAGGGAGCCCAGGACTGGGG |
| | intron | TGAGGACTCAGGGGAGCAGGGAGACCACGTCCCAAGATG |
| | | CCTGTAAAACTGAAACCACCTGGCCATTCTCCAGGTTGAG |
| | | CCAGACCAATTTGATGGCAGATTTAGCAAATAAAAATAC |
| | | AGGACACCCAGTTAAATGTGAATTTCAGATGAACAGCAA |
| | | ATACTTTTTTAGTATTAAAAAAGTTCACATTTAGGCTCAC |
| | | GCCTGTAATCCCAGCACTTTGGGAGGCCGAGGCAGGCAG |
| | | ATCACCTGAGGTCAGGAGTTCGAGACCAGCCTGGCCAAC |
| | | ATGGTGAAACCCCATCTCCACTAAAAATACCAAAAATTAG |
| | | CCAGGCGTGCTGGTGGGCACCTGTAGTTCCAGCTACTCAG |
| | | GAGGCTAAGGCAGGAGAATTGCTTGAACCTGGGAGGCAG |
| | | AGGTTGCAGTGAGCTGAGATCGCACCATTGCACTCTAGCC |
| | | TGGGCGACAAGAACAAAACTCCATCTCAAAAAAGTTCAC |
| | | ATTTAACTGGGCATTCTGTATTTAATTGGTAATCTGAGAT |
| | | GGCAGGGAACAGCATCAGCATGGTGTGAGGGATAGGCAT |
| | | TTTTTCATTGTGTACAGCTTGTAAATCAGTATTTTTAAAAC |
| | | TCAAAGTTAATGGCTTGGGCATATTTAGAAAAGAGTTGCC |
| | | GCACGGACTTGAACCCTGTATTCCTAAAATCTAGGATCTT |
| | | GTTCTGATGGTCTGCACAACTGGCTGGGGGTGTCCAGCCA |
| | | CTGTCCCTCTTGCCTGGGCTCCCCAGGGCAGTTCTGTCAG |
| | | CCTCTCCATTTCCATTCCTGTTCCAGCAAAACCCAACTGAT |
| | | AGCACAGCAGCATTTCAGCCTGTCTACCTCTGTGCCCACA |
| | | TACCTGGATGTCTACCAGCCAGAAAGGTGGCTTAGATTTG |
| | | GTTCCTGTGGGTGGATTATGGCCCCCAGAACTTCCCTGTG |
| | | CTTGCTGGGGGTGTGGAGTGGAAAGAGCAGGAAATGGGG |
| | | GACCCTCCGATACTCTATGGGGGTCCTCCAAGTCTCTTTGT |
| | | GCAAGTTAGGGTAATAATCAATATGGAGCTAA |
| 25 | 5′ AAV2 ITR 1 | GCGCGCTCGCTCGCTCACTGAGGCCGCCCGGGCAAAGCCC |
| | | GGGCGTCGGGCGACCTTTGGTCGCCCGGCCTCAGTGAGCG |
| | | AGCGAGCGCGCAGAGAGGGAGTGGCCAACTCCATCACTA |
| | | GGGGTTCCTTGTAGTTAATGATTAACCCGCCATGCTACTT |
| | | ATCTAC |
| 26 | 5′ AAV2 ITR 2 | GGGCCACTCCCTCTCTGCGCGCTCGCTCGCTCACTGAGGC |
| | | CGGGCGACCAAAGGTCGCCCGACGCCCGGGCTTTGCCCG |
| | | GGCGGCCTCAGTGAGCGAGCGAGCGCGCAGAGAGGGAGT |
| | | GGCCAACTCCATCACTAGGGGTTCCT |
| 27 | 3′ AAV2 ITR | GTAGATAAGTAGCATGGCGGGTTAATCATTAACTACAAG |
| | | GAACCCCTAGTGATGGAGTTGGCCACTCCCTCTCTGCGCG |
| | | CTCGCTCGCTCACTGAGGCCGGGCGACCAAAGGTCGCCCG |
| | | ACGCCCGGGCTTTGCCCGGGCGGCCTCAGTGAGCGAGCG |
| | | AGCGCGC |
| 28 | 3′ mutant AAV2 | CCACTCCCTCTCTGCGCGCTCGCTCGCTCACTGAGGCCGG |
| | ITR | GCGACCAAAGGTCGCCCGACGCCCGGGCTTTGCCCGGGC |
| | | GGCCTCAGTGAGCGAGCGAGCGCGCAGAGAGGGA |
| 29 | CTx001_b | GGGCCACTCCCTCTCTGCGCGCTCGCTCGCTCACTGAGGC |
| | expression | CGGGCGACCAAAGGTCGCCCGACGCCCGGGCTTTGCCCG |
| | cassette | GGCGGCCTCAGTGAGCGAGCGAGCGCGCAGAGAGGGAGT |
| | | GGCCAACTCCATCACTAGGGGTTCCTGGAGGGGTGGAGTC |
| | | GTGACCGTTACATAACTTACGGTAAATGGCCCGCCTGGCT |
| | | GACCGCCCAACGACCCCCGCCCATTGACGTCAATAGTAAC |
| | | GCCAATAGGGACTTTCCATTGACGTCAATGGGTGGAGTAT |
| | | TTACGGTAAACTGCCCACTTGGCAGTACATCAAGTGTATC |
| | | ATATGCCAAGTACGCCCCCTATTGACGTCAATGACGGTAA |
| | | ATGGCCCGCCTGGCATTGTGCCCAGTACATGACCTTATGG |
| | | GACTTTCCTACTTGGCAGTACATCTACGTATTAGTCATCGC |
| | | TATTACCATGGTCGAGGTGAGCCCCACGTTCTGCTTCACT |
| | | CTCCCCATCTCCCCCCCCTCCCCACCCCCAATTTTGTATTT |
| | | ATTTATTTTTTAATTATTTTGTGCAGCGATGGGGGCGGGG |
| | | GGGGGGGGGGGGCGCGCGCCAGGCGGGGCGGGGCGGGG |
| | | CGAGGGGCGGGGCGGGGCGAGGCGGAGAGGTGCGGCGG |
| | | CAGCCAATCAGAGCGGCGCGCTCCGAAAGTTTCCTTTTAT |
| | | GGCGAGGCGGCGGCGGCGGCGGCCCTATAAAAAGCGAAG |
| | | CGCGCGGCGGGCGGGAGTCGCTGCGCGCTGCCTTCGCCCC |
| | | GTGCCCCGCTCCGCCGCCGCCTCGCGCCGCCCGCCCCGGC |
| | | TCTGACTGACCGCGTTACTCCCACAGGTGAGCGGGCGGGA |
| | | CGGCCCTTCTCCTCCGGGCTGTAATTAGCTGAGCAAGAGG |
| | | TAAGGGTTTAAGGGATGGTTGGTTGGTGGGGTATTAATGT |
| | | TTAATTACCTGGAGCACCTGCCTGAAATCACTTTTTTTCAG |
| | | GTTGGAGATCTTTGTCGATCCTACCATCCACTCGACACAC |
| | | CCGCCAGCGGCCGCCGCCACCATGAGACTGCTGGCCAAG |
| | | ATCATCTGCCTGATGCTGTGGGCCATCTGCGTGGCCGGAC |
| | | ATTGTCAGGCCCCTGACCACTTCCTGTTCGCCAAGCTGAA |
| | | AACCCAGACCAACGCCAGCGACTTCCCTATCGGCACCAGC |
| | | CTGAAGTACGAGTGCAGACCCGAGTACTACGGCAGACCC |
| | | TTCAGCATCACCTGTCTGGACAACCTCGTGTGGTCTAGCC |
| | | CCAAGGACGTGTGCAAGAGAAAGAGCTGCAAGACCCCTC |
| | | CTGATCCTGTGAACGGCATGGTGCACGTGATCACCGACAT |
| | | CCAAGTGGGCAGCAGAATCAACTACAGCTGCACCACCGG |
| | | CCACAGACTGATCGGACACTCTAGCGCCGAGTGTATCCTG |
| | | AGCGGCAATGCCGCACACTGGTCCACCAAGCCTCCAATCT |
| | | GCCAGAGAATCCCTTGCGGCCTGCCTCCTACAATCGCCAA |
| | | CGGCGATTTCATCAGCACCAACAGAGAGAACTTCCACTAC |
| | | GGCTCCGTGGTCACCTACAGATGCAATCCTGGCAGCGGCG |
| | | GCAGAAAGGTGTTCGAACTTGTGGGCGAGCCCAGCATCT |
| | | ACTGCACCAGCAACGATGACCAAGTCGGCATTTGGAGCG |
| | | GCCCTGCTCCTCAGTGCATCATCTAACTGACTGATAAGAT |
| | | ACATTGATGAGTTTGGACAAACCACAACTAGAATGCAGT |
| | | GAAAAAAATGCTTTATTTGTGAAATTTGTGATGCTATTGC |
| | | TTTATTTGTAACCATTATAAGCTGCAATAAACAAGTTCTA |
| | | TCCACTAGTCCACTCCCTCTCTGCGCGCTCGCTCGCTCACT |
| | | GAGGCCGGGCGACCAAAGGTCGCCCGACGCCCGGGCTTT |
| | | GCCCGGGCGGCCTCAGTGAGCGAGCGAGCGCGCAGAGAG |
| | | GGA |
| 30 | CTx001_a | GCGCGCTCGCTCGCTCACTGAGGCCGCCCGGGCAAAGCCC |
| | expression | GGGCGTCGGGCGACCTTTGGTCGCCCGGCCTCAGTGAGCG |
| | cassette | AGCGAGCGCGCAGAGAGGGAGTGGCCAACTCCATCACTA |
| | | GGGGTTCCTTGTAGTTAATGATTAACCCGCCATGCTACTT |
| | | ATCTACGGAGGGGTGGAGTCGTGACCTAGGCCATTGACGT |
| | | CAATAATGACGTATGTTCCCATAGTAACGCCAATAGGGAC |
| | | TTTCCATTGACGTCAATGGGTGGAGTATTTACGGTAAACT |
| | | GCCCACTTGGCAGTACATCAAGTGTATCATATGCCAAGTA |
| | | CGCCCCCTATTGACGTCAATGACGGTAAATGGCCCGCCTG |
| | | GCATTATGCCCAGTACATGACCTTATGGGACTTTCCTACTT |
| | | GGCAGTACATCTACGTATTAGTCATCGCTATTACCATGGT |
| | | CGAGGTGAGCCCCACGTTCTGCTTCACTCTCCCCATCTCCC |
| | | CCCCCTCCCCACCCCCAATTTTGTATTTATTTATTTTTTAAT |
| | | TATTTTGTGCAGCGATGGGGGCGGGGGGGGGGGGGGCGC |
| | | GCGCCAGGCGGGGCGGGGCGGGGCGAGGGGCGGGGCGG |
| | | GGCGAGGCGGAGAGGTGCGGCGGCAGCCAATCAGAGCGG |
| | | CGCGCTCCGAAAGTTTCCTTTTATGGCGAGGCGGCGGCGG |
| | | CGGCGGCCCTATAAAAAGCGAAGCGCGCGGCGGGCGGGA |
| | | GTCGCTGCGCGCTGCCTTCGCCCCGTGCCCCGCTCCGCCG |
| | | CCGCCTCGCGCCGCCCGCCCCGGCTCTGACTGACCGCGTT |
| | | ACTCCCACAGGTGAGCGGGCGGGACGGCCCTTCTCCTCCG |
| | | GGCTGTAATTAGCGCTTGGTTTAATGACGGCTTGTTTCTTT |
| | | TCTGTGGCTGCGTGAAAGCCTTGAGGGGCTCCGGGAGGG |
| | | CCCTTTGTGCGGGGGGAGCGGCTCGGGGCTGTCCGCGGG |
| | | GGGACGGCTGCCTTCGGGGGGGACGGGGCAGGGCGGGGT |
| | | TCGGCTTCTGGCGTGTGACCGGCGGCTCTAGAGCCTCTGC |
| | | TAACCATGTTCATGCCTTCTTCTTTTTCCTACAGCTCCTGG |
| | | GCAACGTGCTGGTTGTTGTGCTGTCTCATCATTTTGGCAA |
| | | AGAATTAGATCTTTGTCGATCCTACCATCCACTCGACACA |
| | | CCCGCCAGCGGCCGCCGCCACCATGAGACTGCTGGCCAA |
| | | GATCATCTGCCTGATGCTGTGGGCCATCTGCGTGGCCGGA |
| | | CATTGTCAGGCCCCTGACCACTTCCTGTTCGCCAAGCTGA |
| | | AAACCCAGACCAACGCCAGCGACTTCCCTATCGGCACCA |
| | | GCCTGAAGTACGAGTGCAGACCCGAGTACTACGGCAGAC |
| | | CCTTCAGCATCACCTGTCTGGACAACCTCGTGTGGTCTAG |
| | | CCCCAAGGACGTGTGCAAGAGAAAGAGCTGCAAGACCCC |
| | | TCCTGATCCTGTGAACGGCATGGTGCACGTGATCACCGAC |
| | | ATCCAAGTGGGCAGCAGAATCAACTACAGCTGCACCACC |
| | | GGCCACAGACTGATCGGACACTCTAGCGCCGAGTGTATCC |
| | | TGAGCGGCAATGCCGCACACTGGTCCACCAAGCCTCCAAT |
| | | CTGCCAGAGAATCCCTTGCGGCCTGCCTCCTACAATCGCC |
| | | AACGGCGATTTCATCAGCACCAACAGAGAGAACTTCCACT |
| | | ACGGCTCCGTGGTCACCTACAGATGCAATCCTGGCAGCGG |
| | | CGGCAGAAAGGTGTTCGAACTTGTGGGCGAGCCCAGCAT |
| | | CTACTGCACCAGCAACGATGACCAAGTCGGCATTTGGAGC |
| | | GGCCCTGCTCCTCAGTGCATCATCTAAACTGCTAGCTTGA |
| | | CTGACTGAAATCAACCTCTGGATTACAAAATTTGTGAAAG |
| | | ATTGACTGGTATTCTTAACTATGTTGCTCCTTTTACGCTAT |
| | | GTGGATACGCTGCTTTAATGCCTTTGTATCATGCTATTGCT |
| | | TCCCGTATGGCTTTCATTTTCTCCTCCTTGTATAAATCCTG |
| | | GTTGCTGTCTCTTTATGAGGAGTTGTGGCCCGTTGTCAGG |
| | | CAACGTGGCGTGGTGTGCACTGTGTTTGCTGACGCAACCC |
| | | CCACTGGTTGGGGCATTGCCACCACCTGTCAGCTCCTTTC |
| | | CGGGACTTTCGCTTTCCCCCTCCCTATTGCCACGGCGGAA |
| | | CTCATCGCCGCCTGCCTTGCCCGCTGCTGGACAGGGGCTC |
| | | GGCTGTTGGGCACTGACAATTCCGTGGTGTTGTCGGGGAA |
| | | ATCATCGTCCTTTCCTTGGCTGCTCGCCTGTGTTGCCACCT |
| | | GGATTCTGCGCGGGACGTCCTTCTGCTACGTCCCTTCGGC |
| | | CCTCAATCCAGCGGACCTTCCTTCCCGCGGCCTGCTGCCG |
| | | GCTCTGCGGCCTCTTCCGCGTCTTCGCCTTCGCCCTCAGAC |
| | | GAGTCGGATCTCCCTTTGGGCCGCCTCCCCGCGATATCCT |
| | | GTGCCTTCTAGTTGCCAGCCATCTGTTGTTTGCCCCTCCCC |
| | | CGTGCCTTCCTTGACCCTGGAAGGTGCCACTCCCACTGTC |
| | | CTTTCCTAATAAAATGAGGAAATTGCATCGCATTGTCTGA |
| | | GTAGGTGTCATTCTATTCTGGGGGGTGGGGTGGGGCAGGA |
| | | CAGCAAGGGGGAGGATTGGGAAGACAATAGCAGGCATGC |
| | | TGGGGATGCGGTGGGCTCTATGGCTATCCACATCCACAAG |
| | | GTGCCCCAGGTGGACTGGGCTGGGGCTTTGAGGCCTTCAG |
| | | GGTTGGATGGCCATCTTGCGTATTTGTGTGGGATATGCAC |
| | | ACACAGGCAGCACATGCGCAGGTGTGTGGGCACCTGTGT |
| | | GTCTGTGCAAATGCCCTGAGGTGGGAATGAGCTTGGTGTG |
| | | CATCAGGCACAGCCAGCCAGTGTGGCTGCAGCAAAACAC |
| | | ACAGGGAAAGAATGGAGGGGGCATCAATCACTGCTTCAG |
| | | TAAATTTTTATTGAGCGCCTTCTACGAGAACACAAGAGGA |
| | | GCTTCCATTCTGAGGATATCCTGTATAATTTCAAGTAGTG |
| | | ATAAGTGCTCTCTAGAAATATCAAGCAAGGTGAGGAGAC |
| | | ACAGAGCACCGGTGGCAGTGGGGCTCTATTTCCAGGTTGG |
| | | ATGGTTGGGAACATCCTTTCTAAAGGGAACCTGGAGTGGG |
| | | AAGGAACCATGCAGGTATCTCAGGAAGAGCTTCCTCCAG |
| | | GCAGGAAGATCAGCAGGTGGAAAGGCCCTGGAGCCACCA |
| | | TTCAGTAAACATCATTTGAGCATCTCTACCAGCTAGGTTC |
| | | CATTATGGGAATGGGAATATGGTGGTGGACAGGGCTGCC |
| | | TGGTCCCTTCCATACTTCTCACACTAGGGTGGTTGAGAGA |
| | | GCTTGGGAGGGGCTGAGAACACTGCCTAGCCCAGAGGAC |
| | | CTGAGCTTAGTGTGTAGACATTGCTGCTGTTACTGCCTTTG |
| | | TCATTGTATTAGACAGAGTTTTGCTCTTCTTACCCAGGCTG |
| | | GAGTGCAATGGCGTGATCTCAGCTCACTGCAACCTCCACC |
| | | TCCTGGGTTCAAGCGATTCTCCTGCCTCAGCCTCCTGAGT |
| | | AGCTGGGATTACAGGCACCCGCACCACGCCTGGATAATTT |
| | | TTTTGTATTTTTAGTAGAGACAGGGTTTCACCATGTTGGCC |
| | | AGGCTGGTCTCGAACTCCTGACCTTAGGTGATCCACCTGC |
| | | CTCGACTTCCCAAAGTGCTGGGATTATAGGCATGAGCCAC |
| | | TGCGCCCAGTGATTATAGAAAGTTAAAGGCACATGGCAA |
| | | TGCACACGCCTATCTACGTCTTCCCTGCCAAAGCAAAGGG |
| | | CAGCCTCTGGGCTCACTTTCTTGCGTTTCTACTTCCAAAAG |
| | | GCAGTCAGAACTGGCAGGGCCTTGGAGACCACTTCATCCA |
| | | CCTCCTAGGGTCCCTATGGGAGAGTTGAGGTCCAGAGCAG |
| | | GGAAGGGTCCTGACAGGCTCTGACCAGGGCCTCTGATCCC |
| | | TACAAACCCCCAATCGGTGGCGATGTAGATAAGTAGCAT |
| | | GGCGGGTTAATCATTAACTACAAGGAACCCCTAGTGATGG |
| | | AGTTGGCCACTCCCTCTCTGCGCGCTCGCTCGCTCACTGA |
| | | GGCCGGGCGACCAAAGGTCGCCCGACGCCCGGGCTTTGC |
| | | CCGGGCGGCCTCAGTGAGCGAGCGAGCGCGC |
| 31 | CTx001_d | GCGCGCTCGCTCGCTCACTGAGGCCGCCCGGGCAAAGCCC |
| | expression | GGGCGTCGGGCGACCTTTGGTCGCCCGGCCTCAGTGAGCG |
| | cassette | AGCGAGCGCGCAGAGAGGGAGTGGCCAACTCCATCACTA |
| | | GGGGTTCCTTGTAGTTAATGATTAACCCGCCATGCTACTT |
| | | ATCTACGGAGGGGTGGAGTCGTGACCTAGGCCATTGACGT |
| | | CAATAATGACGTATGTTCCCATAGTAACGCCAATAGGGAC |
| | | TTTCCATTGACGTCAATGGGTGGAGTATTTACGGTAAACT |
| | | GCCCACTTGGCAGTACATCAAGTGTATCATATGCCAAGTA |
| | | CGCCCCCTATTGACGTCAATGACGGTAAATGGCCCGCCTG |
| | | GCATTATGCCCAGTACATGACCTTATGGGACTTTCCTACTT |
| | | GGCAGTACATCTACGTATTAGTCATCGCTATTACCATGGT |
| | | CGAGGTGAGCCCCACGTTCTGCTTCACTCTCCCCATCTCCC |
| | | CCCCCTCCCCACCCCCAATTTTGTATTTATTTATTTTTTAAT |
| | | TATTTTGTGCAGCGATGGGGGCGGGGGGGGGGGGGGCGC |
| | | GCGCCAGGCGGGGCGGGGCGGGGCGAGGGGCGGGGCGG |
| | | GGCGAGGCGGAGAGGTGCGGCGGCAGCCAATCAGAGCGG |
| | | CGCGCTCCGAAAGTTTCCTTTTATGGCGAGGCGGCGGCGG |
| | | CGGCGGCCCTATAAAAAGCGAAGCGCGCGGCGGGCGGGA |
| | | GTCGCTGCGCGCTGCCTTCGCCCCGTGCCCCGCTCCGCCG |
| | | CCGCCTCGCGCCGCCCGCCCCGGCTCTGACTGACCGCGTT |
| | | ACTCCCACAGGTGAGCGGGCGGGACGGCCCTTCTCCTCCG |
| | | GGCTGTAATTAGCGCTTGGTTTAATGACGGCTTGTTTCTTT |
| | | TCTGTGGCTGCGTGAAAGCCTTGAGGGGCTCCGGGAGGG |
| | | CCCTTTGTGCGGGGGGAGCGGCTCGGGGGGTGCGTGCGT |
| | | GTGTGTGTGCGTGGGGAGCGCCGCGTGCGGCTCCGCGCTG |
| | | CCCGGCGGCTGTGAGCGCTGCGGGCGCGGCGCGGGGCTT |
| | | TGTGCGCTCCGCAGTGTGCGCGAGGGGAGCGCGGCCGGG |
| | | GGCGGTGCCCCGCGGTGCGGGGGGGGCTGCGAGGGGAAC |
| | | AAAGGCTGCGTGCGGGGTGTGTGCGTGGGGGGGTGAGCA |
| | | GGGGGTGTGGGCGCGTCGGTCGGGCTGCAACCCCCCCTGC |
| | | ACCCCCCTCCCCGAGTTGCTGAGCACGGCCCGGCTTCGGG |
| | | TGCGGGGCTCCGTACGGGGCGTGGCGCGGGGCTCGCCGT |
| | | GCCGGGCGGGGGGTGGCGGCAGGTGGGGGTGCCGGGCGG |
| | | GGCGGGGCCGCCTCGGGCCGGGGAGGGCTCGGGGGAGGG |
| | | GCGCGGCGGCCCCCGGAGCGCCGGCGGCTGTCGAGGCGC |
| | | GGCGAGCCGCAGCCATTGCCTTTTATGGTAATCGTGCGAG |
| | | AGGGCGCAGGGACTTCCTTTGTCCCAAATCTGTGCGGAGC |
| | | CGAAATCTGGGAGGCGCCGCCGCACCCCCTCTAGCGGGC |
| | | GCGGGGCGAAGCGGTGCGGCGCCGGCAGGAAGGAAATG |
| | | GGCGGGGAGGGCCTTCGTGCGTCGCCGCGCCGCCGTCCCC |
| | | TTCTCCCTCTCCAGCCTCGGGGCTGTCCGCGGGGGGACGG |
| | | CTGCCTTCGGGGGGGACGGGGCAGGGCGGGGTTCGGCTT |
| | | CTGGCGTGTGACCGGCGGCTCTAGAGCCTCTGCTAACCAT |
| | | GTTCATGCCTTCTTCTTTTTCCTACAGCTCCTGGGCAACGT |
| | | GCTGGTTGTTGTGCTGTCTCATCATTTTGGCAAAGAATTA |
| | | GATCTTTGTCGATCCTACCATCCACTCGACACACCCGCCA |
| | | GCGGCCGCCGCCACCATGAGACTGCTGGCCAAGATCATCT |
| | | GCCTGATGCTGTGGGCCATCTGCGTGGCCGGACATTGTCA |
| | | GGCCCCTGACCACTTCCTGTTCGCCAAGCTGAAAACCCAG |
| | | ACCAACGCCAGCGACTTCCCTATCGGCACCAGCCTGAAGT |
| | | ACGAGTGCAGACCCGAGTACTACGGCAGACCCTTCAGCA |
| | | TCACCTGTCTGGACAACCTCGTGTGGTCTAGCCCCAAGGA |
| | | CGTGTGCAAGAGAAAGAGCTGCAAGACCCCTCCTGATCCT |
| | | GTGAACGGCATGGTGCACGTGATCACCGACATCCAAGTG |
| | | GGCAGCAGAATCAACTACAGCTGCACCACCGGCCACAGA |
| | | CTGATCGGACACTCTAGCGCCGAGTGTATCCTGAGCGGCA |
| | | ATGCCGCACACTGGTCCACCAAGCCTCCAATCTGCCAGAG |
| | | AATCCCTTGCGGCCTGCCTCCTACAATCGCCAACGGCGAT |
| | | TTCATCAGCACCAACAGAGAGAACTTCCACTACGGCTCCG |
| | | TGGTCACCTACAGATGCAATCCTGGCAGCGGCGGCAGAA |
| | | AGGTGTTCGAACTTGTGGGCGAGCCCAGCATCTACTGCAC |
| | | CAGCAACGATGACCAAGTCGGCATTTGGAGCGGCCCTGCT |
| | | CCTCAGTGCATCATCTAAACTGCTAGCTTGACTGACTGAA |
| | | ATCAACCTCTGGATTACAAAATTTGTGAAAGATTGACTGG |
| | | TATTCTTAACTATGTTGCTCCTTTTACGCTATGTGGATACG |
| | | CTGCTTTAATGCCTTTGTATCATGCTATTGCTTCCCGTATG |
| | | GCTTTCATTTTCTCCTCCTTGTATAAATCCTGGTTGCTGTC |
| | | TCTTTATGAGGAGTTGTGGCCCGTTGTCAGGCAACGTGGC |
| | | GTGGTGTGCACTGTGTTTGCTGACGCAACCCCCACTGGTT |
| | | GGGGCATTGCCACCACCTGTCAGCTCCTTTCCGGGACTTT |
| | | CGCTTTCCCCCTCCCTATTGCCACGGCGGAACTCATCGCC |
| | | GCCTGCCTTGCCCGCTGCTGGACAGGGGCTCGGCTGTTGG |
| | | GCACTGACAATTCCGTGGTGTTGTCGGGGAAATCATCGTC |
| | | CTTTCCTTGGCTGCTCGCCTGTGTTGCCACCTGGATTCTGC |
| | | GCGGGACGTCCTTCTGCTACGTCCCTTCGGCCCTCAATCC |
| | | AGCGGACCTTCCTTCCCGCGGCCTGCTGCCGGCTCTGCGG |
| | | CCTCTTCCGCGTCTTCGCCTTCGCCCTCAGACGAGTCGGAT |
| | | CTCCCTTTGGGCCGCCTCCCCGCGATATCCTGTGCCTTCTA |
| | | GTTGCCAGCCATCTGTTGTTTGCCCCTCCCCCGTGCCTTCC |
| | | TTGACCCTGGAAGGTGCCACTCCCACTGTCCTTTCCTAAT |
| | | AAAATGAGGAAATTGCATCGCATTGTCTGAGTAGGTGTCA |
| | | TTCTATTCTGGGGGGTGGGGTGGGGCAGGACAGCAAGGG |
| | | GGAGGATTGGGAAGACAATAGCAGGCATGCTGGGGATGC |
| | | GGTGGGCTCTATGGCTATCCACATCCACTGTAGATAAGTA |
| | | GCATGGCGGGTTAATCATTAACTACAAGGAACCCCTAGTG |
| | | ATGGAGTTGGCCACTCCCTCTCTGCGCGCTCGCTCGCTCA |
| | | CTGAGGCCGGGCGACCAAAGGTCGCCCGACGCCCGGGCT |
| | | TTGCCCGGGCGGCCTCAGTGAGCGAGCGAGCGCGC |
| 32 | CTx001_e | GCGCGCTCGCTCGCTCACTGAGGCCGCCCGGGCAAAGCCC |
| | expression | GGGCGTCGGGCGACCTTTGGTCGCCCGGCCTCAGTGAGCG |
| | cassette | AGCGAGCGCGCAGAGAGGGAGTGGCCAACTCCATCACTA |
| | | GGGGTTCCTTGTAGTTAATGATTAACCCGCCATGCTACTT |
| | | ATCTACGGAGGGGTGGAGTCGTGACCTAGGCCATTGACGT |
| | | CAATAATGACGTATGTTCCCATAGTAACGCCAATAGGGAC |
| | | TTTCCATTGACGTCAATGGGTGGAGTATTTACGGTAAACT |
| | | GCCCACTTGGCAGTACATCAAGTGTATCATATGCCAAGTA |
| | | CGCCCCCTATTGACGTCAATGACGGTAAATGGCCCGCCTG |
| | | GCATTATGCCCAGTACATGACCTTATGGGACTTTCCTACTT |
| | | GGCAGTACATCTACGTATTAGTCATCGCTATTACCATGGT |
| | | CGAGGTGAGCCCCACGTTCTGCTTCACTCTCCCCATCTCCC |
| | | CCCCCTCCCCACCCCCAATTTTGTATTTATTTATTTTTTAAT |
| | | TATTTTGTGCAGCGATGGGGGCGGGGGGGGGGGGGGCGC |
| | | GCGCCAGGCGGGGCGGGGCGGGGCGAGGGGCGGGGCGG |
| | | GGCGAGGCGGAGAGGTGCGGCGGCAGCCAATCAGAGCGG |
| | | CGCGCTCCGAAAGTTTCCTTTTATGGCGAGGCGGCGGCGG |
| | | CGGCGGCCCTATAAAAAGCGAAGCGCGCGGCGGGCGGGA |
| | | GTCGCTGCGCGCTGCCTTCGCCCCGTGCCCCGCTCCGCCG |
| | | CCGCCTCGCGCCGCCCGCCCCGGCTCTGACTGACCGCGTT |
| | | ACTCCCACAGGTGAGCGGGCGGGACGGCCCTTCTCCTCCG |
| | | GGCTGTAATTAGCGCTTGGTTTAATGACGGCTTGTTTCTTT |
| | | TCTGTGGCTGCGTGAAAGCCTTGAGGGGCTCCGGGAGGG |
| | | CCCTTTGTGCGGGGGGAGCGGCTCGGGGGGTGCGTGCGT |
| | | GTGTGTGTGCGTGGGGAGCGCCGCGTGCGGCTCCGCGCTG |
| | | CCCGGCGGCTGTGAGCGCTGCGGGCGCGGCGCGGGGCTT |
| | | TGTGCGCTCCGCAGTGTGCGCGAGGGGAGCGCGGCCGGG |
| | | GGCGGTGCCCCGCGGTGCGGGGGGGGCTGCGAGGGGAAC |
| | | AAAGGCTGCGTGCGGGGTGTGTGCGTGGGGGGGTGAGCA |
| | | GGGGGTGTGGGCGCGTCGGTCGGGCTGCAACCCCCCCTGC |
| | | ACCCCCCTCCCCGAGTTGCTGAGCACGGCCCGGCTTCGGG |
| | | TGCGGGGCTCCGTACGGGGCGTGGCGCGGGGCTCGCCGT |
| | | GCCGGGCGGGGGGTGGCGGCAGGTGGGGGTGCCGGGCGG |
| | | GGCGGGGCCGCCTCGGGCCGGGGAGGGCTCGGGGGAGGG |
| | | GCGCGGCGGCCCCCGGAGCGCCGGCGGCTGTCGAGGCGC |
| | | GGCGAGCCGCAGCCATTGCCTTTTATGGTAATCGTGCGAG |
| | | AGGGCGCAGGGACTTCCTTTGTCCCAAATCTGTGCGGAGC |
| | | CGAAATCTGGGAGGCGCCGCCGCACCCCCTCTAGCGGGC |
| | | GCGGGGCGAAGCGGTGCGGCGCCGGCAGGAAGGAAATG |
| | | GGCGGGGAGGGCCTTCGTGCGTCGCCGCGCCGCCGTCCCC |
| | | TTCTCCCTCTCCAGCCTCGGGGCTGTCCGCGGGGGGACGG |
| | | CTGCCTTCGGGGGGGACGGGGCAGGGCGGGGTTCGGCTT |
| | | CTGGCGTGTGACCGGCGGCTCTAGAGCCTCTGCTAACCAT |
| | | GTTCATGCCTTCTTCTTTTTCCTACAGCTCCTGGGCAACGT |
| | | GCTGGTTGTTGTGCTGTCTCATCATTTTGGCAAAGAATTA |
| | | GATCTTTGTCGATCCTACCATCCACTCGACACACCCGCCA |
| | | GCGGCCGCCGCCACCATGAGACTGCTGGCCAAGATCATCT |
| | | GCCTGATGCTGTGGGCCATCTGCGTGGCCGGACATTGTCA |
| | | GGCCCCTGACCACTTCCTGTTCGCCAAGCTGAAAACCCAG |
| | | ACCAACGCCAGCGACTTCCCTATCGGCACCAGCCTGAAGT |
| | | ACGAGTGCAGACCCGAGTACTACGGCAGACCCTTCAGCA |
| | | TCACCTGTCTGGACAACCTCGTGTGGTCTAGCCCCAAGGA |
| | | CGTGTGCAAGAGAAAGAGCTGCAAGACCCCTCCTGATCCT |
| | | GTGAACGGCATGGTGCACGTGATCACCGACATCCAAGTG |
| | | GGCAGCAGAATCAACTACAGCTGCACCACCGGCCACAGA |
| | | CTGATCGGACACTCTAGCGCCGAGTGTATCCTGAGCGGCA |
| | | ATGCCGCACACTGGTCCACCAAGCCTCCAATCTGCCAGAG |
| | | AATCCCTTGCGGCCTGCCTCCTACAATCGCCAACGGCGAT |
| | | TTCATCAGCACCAACAGAGAGAACTTCCACTACGGCTCCG |
| | | TGGTCACCTACAGATGCAATCCTGGCAGCGGCGGCAGAA |
| | | AGGTGTTCGAACTTGTGGGCGAGCCCAGCATCTACTGCAC |
| | | CAGCAACGATGACCAAGTCGGCATTTGGAGCGGCCCTGCT |
| | | CCTCAGTGCATCATCTAAACTGCTAGCTTGACTGACTGAA |
| | | ATCAACCTCTGGATTACAAAATTTGTGAAAGATTGACTGG |
| | | TATTCTTAACTATGTTGCTCCTTTTACGCTATGTGGATACG |
| | | CTGCTTTAATGCCTTTGTATCATGCTATTGCTTCCCGTATG |
| | | GCTTTCATTTTCTCCTCCTTGTATAAATCCTGGTTGCTGTC |
| | | TCTTTATGAGGAGTTGTGGCCCGTTGTCAGGCAACGTGGC |
| | | GTGGTGTGCACTGTGTTTGCTGACGCAACCCCCACTGGTT |
| | | GGGGCATTGCCACCACCTGTCAGCTCCTTTCCGGGACTTT |
| | | CGCTTTCCCCCTCCCTATTGCCACGGCGGAACTCATCGCC |
| | | GCCTGCCTTGCCCGCTGCTGGACAGGGGCTCGGCTGTTGG |
| | | GCACTGACAATTCCGTGGTGTTGTCGGGGAAATCATCGTC |
| | | CTTTCCTTGGCTGCTCGCCTGTGTTGCCACCTGGATTCTGC |
| | | GCGGGACGTCCTTCTGCTACGTCCCTTCGGCCCTCAATCC |
| | | AGCGGACCTTCCTTCCCGCGGCCTGCTGCCGGCTCTGCGG |
| | | CCTCTTCCGCGTCTTCGCCTTCGCCCTCAGACGAGTCGGAT |
| | | CTCCCTTTGGGCCGCCTCCCCGCGATATCCTGTGCCTTCTA |
| | | GTTGCCAGCCATCTGTTGTTTGCCCCTCCCCCGTGCCTTCC |
| | | TTGACCCTGGAAGGTGCCACTCCCACTGTCCTTTCCTAAT |
| | | AAAATGAGGAAATTGCATCGCATTGTCTGAGTAGGTGTCA |
| | | TTCTATTCTGGGGGGTGGGGTGGGGCAGGACAGCAAGGG |
| | | GGAGGATTGGGAAGACAATAGCAGGCATGCTGGGGATGC |
| | | GGTGGGCTCTATGGCTATCCACATCCACAAGGTGCCCCAG |
| | | GTGGACTGGGCTGGGGCTTTGAGGCCTTCAGGGTTGGATG |
| | | GCCATCTTGCGTATTTGTGTGGGATATGCACACACAGGCA |
| | | GCACATGCGCAGGTGTGTGGGCACCTGTGTGTCTGTGCAA |
| | | ATGCCCTGAGGTGGGAATGAGCTTGGTGTGCATCAGGCAC |
| | | AGCCAGCCAGTGTGGCTGCAGCAAAACACACAGGGAAAG |
| | | AATGGAGGGGGCATCAATCACTGCTTCAGTAAATTTTTAT |
| | | TGAGCGCCTTCTACGAGAACACAAGAGGAGCTTCCATTCT |
| | | GAGGATATCCTGTATAATTTCAAGTAGTGATAAGTGCTCT |
| | | CTAGAAATATCAAGCAAGGTGAGGAGACACAGAGCACCG |
| | | GTGGCAGTGGGGCTCTATTTCCAGGTTGGATGGTTGGGAA |
| | | CATCCTTTCTAAAGGGAACCTGGAGTGGGAAGGAACCAT |
| | | GCAGGTATCTCAGGAAGAGCTTCCTCCAGGCAGGAAGAT |
| | | CAGCAGGTGGAAAGGCCCTGGAGCCACCATTCAGTAAAC |
| | | ATCATTTGAGCATCTCTACCAGCTAGGTTCCATTATGGGA |
| | | ATGGGAATATGGTGGTGGACAGGGCTGCCTGGTCCCTTCC |
| | | ATACTTCTCACACTAGGGTGGTTGAGAGAGCTTGGGAGGG |
| | | GCTGAGAACACTGCCTAGCCCAGAGGACCTGAGCTTAGT |
| | | GTGTAGACATTGCTGCTGTTACTGCCTTTGTCATTGTATTA |
| | | GACAGAGTTTTGCTCTTCTTACCCAGGCTGGAGTGCAATG |
| | | GCGTGATCTCAGCTCACTGCAACCTCCACCTCCTGGGTTC |
| | | AAGCGATTCTCCTGCCTCAGCCTCCTGAGTAGCTGGGATT |
| | | ACAGGCACCCGCACCACGCCTGGATAATTTTTTTGTATTTT |
| | | TAGTAGAGACAGGGTTTCACCATGTTGGCCAGGCTGGTCT |
| | | CGAACTCCTGACCTTAGGTGATCCACCTGCCTCGACTTCC |
| | | CAAAGTGCTGGGATTATAGGCATGAGCCACTGCGCCCAGT |
| | | GATTATAGAAAGTTAAAGGCACATGGCAATGCACACGCC |
| | | TATCTACGTCTTCCCTGCCAAAGCAAAGGGCAGCCTCTGG |
| | | GCTCACTTTCTTGCGTTTCTACTTCCAAAAGGCAGTCAGA |
| | | ACTGGCAGGGCCTTGGAGACCACTTCATCCACCTCCTAGG |
| | | GTCCCTATGGGAGAGTTGAGGTCCAGAGCAGGGAAGGGT |
| | | CCTGACAGGCTCTGACCAGGGCCTCTGATCCCTACAAACC |
| | | CCCAATCGGTGGCGATGTAGATAAGTAGCATGGCGGGTT |
| | | AATCATTAACTACAAGGAACCCCTAGTGATGGAGTTGGCC |
| | | ACTCCCTCTCTGCGCGCTCGCTCGCTCACTGAGGCCGGGC |
| | | GACCAAAGGTCGCCCGACGCCCGGGCTTTGCCCGGGCGG |
| | | CCTCAGTGAGCGAGCGAGCGCGC |
| 33 | CTx001_f | GCGCGCTCGCTCGCTCACTGAGGCCGCCCGGGCAAAGCCC |
| | expression | GGGCGTCGGGCGACCTTTGGTCGCCCGGCCTCAGTGAGCG |
| | cassette | AGCGAGCGCGCAGAGAGGGAGTGGCCAACTCCATCACTA |
| | | GGGGTTCCTTGTAGTTAATGATTAACCCGCCATGCTACTT |
| | | ATCTACGGAGGGGTGGAGTCGTGACCTAGGCCATTGACGT |
| | | CAATAATGACGTATGTTCCCATAGTAACGCCAATAGGGAC |
| | | TTTCCATTGACGTCAATGGGTGGAGTATTTACGGTAAACT |
| | | GCCCACTTGGCAGTACATCAAGTGTATCATATGCCAAGTA |
| | | CGCCCCCTATTGACGTCAATGACGGTAAATGGCCCGCCTG |
| | | GCATTATGCCCAGTACATGACCTTATGGGACTTTCCTACTT |
| | | GGCAGTACATCTACGTATTAGTCATCGCTATTACCATGGT |
| | | CGAGGTGAGCCCCACGTTCTGCTTCACTCTCCCCATCTCCC |
| | | CCCCCTCCCCACCCCCAATTTTGTATTTATTTATTTTTTAAT |
| | | TATTTTGTGCAGCGATGGGGGCGGGGGGGGGGGGGGCGC |
| | | GCGCCAGGCGGGGCGGGGCGGGGCGAGGGGCGGGGCGG |
| | | GGCGAGGCGGAGAGGTGCGGCGGCAGCCAATCAGAGCGG |
| | | CGCGCTCCGAAAGTTTCCTTTTATGGCGAGGCGGCGGCGG |
| | | CGGCGGCCCTATAAAAAGCGAAGCGCGCGGCGGGCGGGA |
| | | GTCGCTGCGCGCTGCCTTCGCCCCGTGCCCCGCTCCGCCG |
| | | CCGCCTCGCGCCGCCCGCCCCGGCTCTGACTGACCGCGTT |
| | | ACTCCCACAGGTGAGCGGGCGGGACGGCCCTTCTCCTCCG |
| | | GGCTGTAATTAGCGCTTGGTTTAATGACGGCTTGTTTCTTT |
| | | TCTGTGGCTGCGTGAAAGCCTTGAGGGGCTCCGGGAGGG |
| | | CCCTTTGTGCGGGGGGAGCGGCTCGGGGGGTGCGTGCGT |
| | | GTGTGTGTGCGTGGGGAGCGCCGCGTGCGGCTCCGCGCTG |
| | | CCCGGCGGCTGTGAGCGCTGCGGGCGCGGCGCGGGGCTT |
| | | TGTGCGCTCCGCAGTGTGCGCGAGGGGAGCGCGGCCGGG |
| | | GGCGGTGCCCCGCGGTGCGGGGGGGGCTGCGAGGGGAAC |
| | | AAAGGCTGCGTGCGGGGTGTGTGCGTGGGGGGGTGAGCA |
| | | GGGGGTGTGGGCGCGTCGGTCGGGCTGCAACCCCCCCTGC |
| | | ACCCCCCTCCCCGAGTTGCTGAGCACGGCCCGGCTTCGGG |
| | | TGCGGGGCTCCGTACGGGGCGTGGCGCGGGGCTCGCCGT |
| | | GCCGGGCGGGGGGTGGCGGCAGGTGGGGGTGCCGGGCGG |
| | | GGCGGGGCCGCCTCGGGCCGGGGAGGGCTCGGGGGAGGG |
| | | GCGCGGCGGCCCCCGGAGCGCCGGCGGCTGTCGAGGCGC |
| | | GGCGAGCCGCAGCCATTGCCTTTTATGGTAATCGTGCGAG |
| | | AGGGCGCAGGGACTTCCTTTGTCCCAAATCTGTGCGGAGC |
| | | CGAAATCTGGGAGGCGCCGCCGCACCCCCTCTAGCGGGC |
| | | GCGGGGCGAAGCGGTGCGGCGCCGGCAGGAAGGAAATG |
| | | GGCGGGGAGGGCCTTCGTGCGTCGCCGCGCCGCCGTCCCC |
| | | TTCTCCCTCTCCAGCCTCGGGGCTGTCCGCGGGGGGACGG |
| | | CTGCCTTCGGGGGGGACGGGGCAGGGCGGGGTTCGGCTT |
| | | CTGGCGTGTGACCGGCGGCTCTAGAGCCTCTGCTAACCAT |
| | | GTTCATGCCTTCTTCTTTTTCCTACAGCTCCTGGGCAACGT |
| | | GCTGGTTGTTGTGCTGTCTCATCATTTTGGCAAAGAATTA |
| | | GATCTTTGTCGATCCTACCATCCACTCGACACACCCGCCA |
| | | GCGGCCGCCGCCACCATGAGACTGCTGGCCAAGATCATCT |
| | | GCCTGATGCTGTGGGCCATCTGCGTGGCCGGACATTGTCA |
| | | GGCCCCTGACCACTTCCTGTTCGCCAAGCTGAAAACCCAG |
| | | ACCAACGCCAGCGACTTCCCTATCGGCACCAGCCTGAAGT |
| | | ACGAGTGCAGACCCGAGTACTACGGCAGACCCTTCAGCA |
| | | TCACCTGTCTGGACAACCTCGTGTGGTCTAGCCCCAAGGA |
| | | CGTGTGCAAGAGAAAGAGCTGCAAGACCCCTCCTGATCCT |
| | | GTGAACGGCATGGTGCACGTGATCACCGACATCCAAGTG |
| | | GGCAGCAGAATCAACTACAGCTGCACCACCGGCCACAGA |
| | | CTGATCGGACACTCTAGCGCCGAGTGTATCCTGAGCGGCA |
| | | ATGCCGCACACTGGTCCACCAAGCCTCCAATCTGCCAGAG |
| | | AATCCCTTGCGGCCTGCCTCCTACAATCGCCAACGGCGAT |
| | | TTCATCAGCACCAACAGAGAGAACTTCCACTACGGCTCCG |
| | | TGGTCACCTACAGATGCAATCCTGGCAGCGGCGGCAGAA |
| | | AGGTGTTCGAACTTGTGGGCGAGCCCAGCATCTACTGCAC |
| | | CAGCAACGATGACCAAGTCGGCATTTGGAGCGGCCCTGCT |
| | | CCTCAGTGCATCATCTAAACTGCTAGCTTGACTATCCTGT |
| | | GCCTTCTAGTTGCCAGCCATCTGTTGTTTGCCCCTCCCCCG |
| | | TGCCTTCCTTGACCCTGGAAGGTGCCACTCCCACTGTCCTT |
| | | TCCTAATAAAATGAGGAAATTGCATCGCATTGTCTGAGTA |
| | | GGTGTCATTCTATTCTGGGGGGTGGGGTGGGGCAGGACA |
| | | GCAAGGGGGAGGATTGGGAAGACAATAGCAGGCATGCTG |
| | | GGGATGCGGTGGGCTCTATGGCTATCCACATCCACAAGGT |
| | | GCCCCAGGTGGACTGGGCTGGGGCTTTGAGGCCTTCAGGG |
| | | TTGGATGGCCATCTTGCGTATTTGTGTGGGATATGCACAC |
| | | ACAGGCAGCACATGCGCAGGTGTGTGGGCACCTGTGTGTC |
| | | TGTGCAAATGCCCTGAGGTGGGAATGAGCTTGGTGTGCAT |
| | | CAGGCACAGCCAGCCAGTGTGGCTGCAGCAAAACACACA |
| | | GGGAAAGAATGGAGGGGGCATCAATCACTGCTTCAGTAA |
| | | ATTTTTATTGAGCGCCTTCTACGAGAACACAAGAGGAGCT |
| | | TCCATTCTGAGGATATCCTGTATAATTTCAAGTAGTGATA |
| | | AGTGCTCTCTAGAAATATCAAGCAAGGTGAGGAGACACA |
| | | GAGCACCGGTGGCAGTGGGGCTCTATTTCCAGGTTGGATG |
| | | GTTGGGAACATCCTTTCTAAAGGGAACCTGGAGTGGGAA |
| | | GGAACCATGCAGGTATCTCAGGAAGAGCTTCCTCCAGGC |
| | | AGGAAGATCAGCAGGTGGAAAGGCCCTGGAGCCACCATT |
| | | CAGTAAACATCATTTGAGCATCTCTACCAGCTAGGTTCCA |
| | | TTATGGGAATGGGAATATGGTGGTGGACAGGGCTGCCTG |
| | | GTCCCTTCCATACTTCTCACACTAGGGTGGTTGAGAGAGC |
| | | TTGGGAGGGGCTGAGAACACTGCCTAGCCCAGAGGACCT |
| | | GAGCTTAGTGTGTAGACATTGCTGCTGTTACTGCCTTTGTC |
| | | ATTGTATTAGACAGAGTTTTGCTCTTCTTACCCAGGCTGG |
| | | AGTGCAATGGCGTGATCTCAGCTCACTGCAACCTCCACCT |
| | | CCTGGGTTCAAGCGATTCTCCTGCCTCAGCCTCCTGAGTA |
| | | GCTGGGATTACAGGCACCCGCACCACGCCTGGATAATTTT |
| | | TTTGTATTTTTAGTAGAGACAGGGTTTCACCATGTTGGCC |
| | | AGGCTGGTCTCGAACTCCTGACCTTAGGTGATCCACCTGC |
| | | CTCGACTTCCCAAAGTGCTGGGATTATAGGCATGAGCCAC |
| | | TGCGCCCAGTGATTATAGAAAGTTAAAGGCACATGGCAA |
| | | TGCACACGCCTATCTACGTCTTCCCTGCCAAAGCAAAGGG |
| | | CAGCCTCTGGGCTCACTTTCTTGCGTTTCTACTTCCAAAAG |
| | | GCAGTCAGAACTGGCAGGGCCTTGGAGACCACTTCATCCA |
| | | CCTCCTAGGGTCCCTATGGGAGAGTTGAGGTCCAGAGCAG |
| | | GGAAGGGTCCTGACAGGCTCTGACCAGGGCCTCTGATCCC |
| | | TACAAACCCCCAATCGGTGGCGATGTAGATAAGTAGCAT |
| | | GGCGGGTTAATCATTAACTACAAGGAACCCCTAGTGATGG |
| | | AGTTGGCCACTCCCTCTCTGCGCGCTCGCTCGCTCACTGA |
| | | GGCCGGGCGACCAAAGGTCGCCCGACGCCCGGGCTTTGC |
| | | CCGGGCGGCCTCAGTGAGCGAGCGAGCGCGC |
| 34 | CTx001_c | GGGCCACTCCCTCTCTGCGCGCTCGCTCGCTCACTGAGGC |
| | expression | CGGGCGACCAAAGGTCGCCCGACGCCCGGGCTTTGCCCG |
| | cassette | GGCGGCCTCAGTGAGCGAGCGAGCGCGCAGAGAGGGAGT |
| | | GGCCAACTCCATCACTAGGGGTTCCTGGAGGGGTGGAGTC |
| | | GTGACCTAGGCCATTGACGTCAATAATGACGTATGTTCCC |
| | | ATAGTAACGCCAATAGGGACTTTCCATTGACGTCAATGGG |
| | | TGGAGTATTTACGGTAAACTGCCCACTTGGCAGTACATCA |
| | | AGTGTATCATATGCCAAGTACGCCCCCTATTGACGTCAAT |
| | | GACGGTAAATGGCCCGCCTGGCATTATGCCCAGTACATGA |
| | | CCTTATGGGACTTTCCTACTTGGCAGTACATCTACGTATTA |
| | | GTCATCGCTATTACCATGGTCGAGGTGAGCCCCACGTTCT |
| | | GCTTCACTCTCCCCATCTCCCCCCCCTCCCCACCCCCAATT |
| | | TTGTATTTATTTATTTTTTAATTATTTTGTGCAGCGATGGG |
| | | GGCGGGGGGGGGGGGGGGGCCCCCCCCAGGCGGGGCGG |
| | | GGCGGGGCGAGGGGCGGGGCGGGGCGAGGCGGAAAGGT |
| | | GCGGCGGCAGCCAATCAGAGCGGCGCGCTCCGAAAGTTT |
| | | CCTTTTATGGCGAGGCGGCGGCGGCGGCGGCCCTATAAA |
| | | AAGCGAAGCGCGCGGCGGGCGGTAAGTATCAAGGTTACA |
| | | AGACAGGTTTAAGGAGACCAATAGAAACTGGGCTTGTCG |
| | | AGACAGAGAAGACTCTTGCGTTTCTGATAGGCACCTATTG |
| | | GTCTTACTGACATCCACTTTGCCTTTCTCTCCACAGGCGGC |
| | | CGCCGCCACCATGAGACTGCTGGCCAAGATCATCTGCCTG |
| | | ATGCTGTGGGCCATCTGCGTGGCCGGACATTGTCAGGCCC |
| | | CTGACCACTTCCTGTTCGCCAAGCTGAAAACCCAGACCAA |
| | | CGCCAGCGACTTCCCTATCGGCACCAGCCTGAAGTACGAG |
| | | TGCAGACCCGAGTACTACGGCAGACCCTTCAGCATCACCT |
| | | GTCTGGACAACCTCGTGTGGTCTAGCCCCAAGGACGTGTG |
| | | CAAGAGAAAGAGCTGCAAGACCCCTCCTGATCCTGTGAA |
| | | CGGCATGGTGCACGTGATCACCGACATCCAAGTGGGCAG |
| | | CAGAATCAACTACAGCTGCACCACCGGCCACAGACTGAT |
| | | CGGACACTCTAGCGCCGAGTGTATCCTGAGCGGCAATGCC |
| | | GCACACTGGTCCACCAAGCCTCCAATCTGCCAGAGAATCC |
| | | CTTGCGGCCTGCCTCCTACAATCGCCAACGGCGATTTCAT |
| | | CAGCACCAACAGAGAGAACTTCCACTACGGCTCCGTGGTC |
| | | ACCTACAGATGCAATCCTGGCAGCGGCGGCAGAAAGGTG |
| | | TTCGAACTTGTGGGCGAGCCCAGCATCTACTGCACCAGCA |
| | | ACGATGACCAAGTCGGCATTTGGAGCGGCCCTGCTCCTCA |
| | | GTGCATCATCTAAACTGCTAGCTTGACTGACTGAAATCAA |
| | | CCTCTGGATTACAAAATTTGTGAAAGATTGACTGGTATTC |
| | | TTAACTATGTTGCTCCTTTTACGCTATGTGGATACGCTGCT |
| | | TTAATGCCTTTGTATCATGCTATTGCTTCCCGTATGGCTTT |
| | | CATTTTCTCCTCCTTGTATAAATCCTGGTTGCTGTCTCTTT |
| | | ATGAGGAGTTGTGGCCCGTTGTCAGGCAACGTGGCGTGGT |
| | | GTGCACTGTGTTTGCTGACGCAACCCCCACTGGTTGGGGC |
| | | ATTGCCACCACCTGTCAGCTCCTTTCCGGGACTTTCGCTTT |
| | | CCCCCTCCCTATTGCCACGGCGGAACTCATCGCCGCCTGC |
| | | CTTGCCCGCTGCTGGACAGGGGCTCGGCTGTTGGGCACTG |
| | | ACAATTCCGTGGTGTTGTCGGGGAAATCATCGTCCTTTCC |
| | | TTGGCTGCTCGCCTGTGTTGCCACCTGGATTCTGCGCGGG |
| | | ACGTCCTTCTGCTACGTCCCTTCGGCCCTCAATCCAGCGG |
| | | ACCTTCCTTCCCGCGGCCTGCTGCCGGCTCTGCGGCCTCTT |
| | | CCGCGTCTTCGCCTTCGCCCTCAGACGAGTCGGATCTCCC |
| | | TTTGGGCCGCCTCCCCGCGATATCCTGTGCCTTCTAGTTGC |
| | | CAGCCATCTGTTGTTTGCCCCTCCCCCGTGCCTTCCTTGAC |
| | | CCTGGAAGGTGCCACTCCCACTGTCCTTTCCTAATAAAAT |
| | | GAGGAAATTGCATCGCATTGTCTGAGTAGGTGTCATTCTA |
| | | TTCTGGGGGGTGGGGTGGGGCAGGACAGCAAGGGGGAGG |
| | | ATTGGGAAGACAATAGCAGGCATGCTGGGGATGCGGTGG |
| | | GCTCTATGGCTATCCACAACTAGTCCACTCCCTCTCTGCGC |
| | | GCTCGCTCGCTCACTGAGGCCGGGCGACCAAAGGTCGCCC |
| | | GACGCCCGGGCTTTGCCCGGGCGGCCTCAGTGAGCGAGC |
| | | GAGCGCGCAGAGAGGGA |
| 35 | Complement | MKLLHVFLLFLCFHLRFCKVTYTSQEDLVEKKCLAKKYTHL |
| | Factor I (FI) | SCDKVFCQPWQRCIEGTCVCKLPYQCPKNGTAVCATNRRSF |
| | Uniprot: P05156 | PTYCQQKSLECLHPGTKFLNNGTCTAEGKFSVSLKHGNTDS |
| | Including 18-nuc | EGIVEVKLVDQDKTMFICKSSWSMREANVACLDLGFQQGA |
| | signal peptide | DTQRRFKLSDLSINSTECLHVHCRGLETSLAECTFTKRRTMG |
| | sequence | YQDFADVVCYTQKADSPMDDFFQCVNGKYISQMKACDGIN |
| | Entry version 222 | DCGDQSDELCCKACQGKGFHCKSGVCIPSQYQCNGEVDCIT |
| | (14 Dec. 2022) | GEDEVGCAGFASVTQEETEILTADMDAERRRIKSLLPKLSCG |
| | Sequence version | VKNRMHIRRKRIVGGKRAQLGDLPWQVAIKDASGITCGGIYI |
| | 2 | GGCWILTAAHCLRASKTHRYQIWTTVVDWIHPDLKRIVIEY |
| | | VDRIIFHENYNAGTYQNDIALIEMKKDGNKKDCELPRSIPAC |
| | | VPWSPYLFQPNDTCIVSGWGREKDNERVFSLQWGEVKLISN |
| | | CSKFYGNRFYEKEMECAGTYDGSIDACKGDSGGPLVCMDA |
| | | NNVTYVWGVVSWGENCGKPEFPGVYTKVANYFDWISYHV |
| | | GRPFISQYNV |
| 36 | Human | IVGGKRAQLGDLPWQVAIKDASGITCGGIYIGGCWILTAAHC |
| | Complement | LRASKTHRYQIWTTVVDWIHPDLKRIVIEYVDRIIFHENYNA |
| | Factor I (FI) | GTYQNDIALIEMKKDGNKKDCELPRSIPACVPWSPYLFQPND |
| | proteolytic | TCIVSGWGREKDNERVFSLQWGEVKLISNCSKFYGNRFYEK |
| | domain (UniProt: | EMECAGTYDGSIDACKGDSGGPLVCMDANNVTYVWGVVS |
| | P05156 residues | WGENCGKPEFPGVYTKVANYFDWISYHVG |
| | 340-574) | |
| 37 | Codon-optimised | GGACATTGTCAGGCCCCTGACCACTTCCTGTTCGCCAAGC |
| | nucleic acid | TGAAAACCCAGACCAACGCCAGCGACTTCCCTATCGGCAC |
| | encoding human | CAGCCTGAAGTACGAGTGCAGACCCGAGTACTACGGCAG |
| | CR1 CCPs 8-10 | ACCCTTCAGCATCACCTGTCTGGACAACCTCGTGTGGTCT |
| | (with stop codon) | AGCCCCAAGGACGTGTGCAAGAGAAAGAGCTGCAAGACC |
| | | CCTCCTGATCCTGTGAACGGCATGGTGCACGTGATCACCG |
| | | ACATCCAAGTGGGCAGCAGAATCAACTACAGCTGCACCA |
| | | CCGGCCACAGACTGATCGGACACTCTAGCGCCGAGTGTAT |
| | | CCTGAGCGGCAATGCCGCACACTGGTCCACCAAGCCTCCA |
| | | ATCTGCCAGAGAATCCCTTGCGGCCTGCCTCCTACAATCG |
| | | CCAACGGCGATTTCATCAGCACCAACAGAGAGAACTTCC |
| | | ACTACGGCTCCGTGGTCACCTACAGATGCAATCCTGGCAG |
| | | CGGCGGCAGAAAGGTGTTCGAACTTGTGGGCGAGCCCAG |
| | | CATCTACTGCACCAGCAACGATGACCAAGTCGGCATTTGG |
| | | AGCGGCCCTGCTCCTCAGTGCATCATCTAA |
| 38 | Codon-optimised | ATGAGACTGCTGGCCAAGATCATCTGCCTGATGCTGTGGG |
| | nucleic acid | CCATCTGCGTGGCCGGACATTGTCAGGCCCCTGACCACTT |
| | encoding human | CCTGTTCGCCAAGCTGAAAACCCAGACCAACGCCAGCGA |
| | CR1 CCPs 8-10 | CTTCCCTATCGGCACCAGCCTGAAGTACGAGTGCAGACCC |
| | (with codon | GAGTACTACGGCAGACCCTTCAGCATCACCTGTCTGGACA |
| | optimised FH | ACCTCGTGTGGTCTAGCCCCAAGGACGTGTGCAAGAGAA |
| | signal peptide | AGAGCTGCAAGACCCCTCCTGATCCTGTGAACGGCATGGT |
| | sequence | GCACGTGATCACCGACATCCAAGTGGGCAGCAGAATCAA |
| | underlined and | CTACAGCTGCACCACCGGCCACAGACTGATCGGACACTCT |
| | stop codon) | AGCGCCGAGTGTATCCTGAGCGGCAATGCCGCACACTGGT |
| | | CCACCAAGCCTCCAATCTGCCAGAGAATCCCTTGCGGCCT |
| | | GCCTCCTACAATCGCCAACGGCGATTTCATCAGCACCAAC |
| | | AGAGAGAACTTCCACTACGGCTCCGTGGTCACCTACAGAT |
| | | GCAATCCTGGCAGCGGCGGCAGAAAGGTGTTCGAACTTG |
| | | TGGGCGAGCCCAGCATCTACTGCACCAGCAACGATGACC |
| | | AAGTCGGCATTTGGAGCGGCCCTGCTCCTCAGTGCATCAT |
| | | CTAA |
| 39 | Human | MRLLAKIICLMLWAICVAEDCNELPPRRNTEILTGSWSDQTY |
| | Complement | PEGTQAIYKCRPGYRSLGNVIMVCRKGEWVALNPLRKCQKR |
| | Factor H | PCGHPGDTPFGTFTLTGGNVFEYGVKAVYTCNEGYQLLGEI |
| | (UniProt: | NYRECDTDGWTNDIPICEVVKCLPVTAPENGKIVSSAMEPDR |
| | P08603-1; Entry | EYHFGQAVRFVCNSGYKIEGDEEMHCSDDGFWSKEKPKCV |
| | version 244 | EISCKSPDVINGSPISQKIIYKENERFQYKCNMGYEYSERGDA |
| | (14 Dec. 2022) | VCTESGWRPLPSCEEKSCDNPYIPNGDYSPLRIKHRTGDEITY |
| | Sequence version 4 | QCRNGFYPATRGNTAKCTSTGWIPAPRCTLKPCDYPDIKHG |
| | Including signal | GLYHENMRRPYFPVAVGKYYSYYCDEHFETPSGSYWDHIH |
| | peptide sequence | CTQDGWSPAVPCLRKCYFPYLENGYNQNYGRKFVQGKSID |
| | (underlined) | VACHPGYALPKAQTTVTCMENGWSPTPRCIRVKTCSKSSIDI |
| | | ENGFISESQYTYALKEKAKYQCKLGYVTADGETSGSITCGK |
| | | DGWSAQPTCIKSCDIPVFMNARTKNDFTWFKLNDTLDYECH |
| | | DGYESNTGSTTGSIVCGYNGWSDLPICYERECELPKIDVHLV |
| | | PDRKKDQYKVGEVLKFSCKPGFTIVGPNSVQCYHFGLSPDLP |
| | | ICKEQVQSCGPPPELLNGNVKEKTKEEYGHSEVVEYYCNPRF |
| | | LMKGPNKIQCVDGEWTTLPVCIVEESTCGDIPELEHGWAQLS |
| | | SPPYYYGDSVEFNCSESFTMIGHRSITCIHGVWTQLPQCVAID |
| | | KLKKCKSSNLIILEEHLKNKKEFDHNSNIRYRCRGKEGWIHT |
| | | VCINGRWDPEVNCSMAQIQLCPPPPQIPNSHNMTTTLNYRDG |
| | | EKVSVLCQENYLIQEGEEITCKDGRWQSIPLCVEKIPCSQPPQ |
| | | IEHGTINSSRSSQESYAHGTKLSYTCEGGFRISEENETTCYMG |
| | | KWSSPPQCEGLPCKSPPEISHGVVAHMSDSYQYGEEVTYKC |
| | | FEGFGIDGPAIAKCLGEKWSHPPSCIKTDCLSLPSFENAIPMG |
| | | EKKDVYKAGEQVTYTCATYYKMDGASNVTCINSRWTGRPT |
| | | CRDTSCVNPPTVQNAYIVSRQMSKYPSGERVRYQCRSPYEM |
| | | FGDEEVMCLNGNWTEPPQCKDSTGKCGPPPPIDNGDITSFPL |
| | | SVYAPASSVEYQCQNLYQLEGNKRITCRNGQWSEPPKCLHP |
| | | CVISREIMENYNIALRWTAKQKLYSRTGESVEFVCKRGYRLS |
| | | SRSHTLRTTCWDGKLEYPTCAKR |
| 40 | Human FHL-1 | MRLLAKIICLMLWAICVAEDCNELPPRRNTEILTGSWSDQTY |
| | (Uniprot P08603- | PEGTQAIYKCRPGYRSLGNVIMVCRKGEWVALNPLRKCQKR |
| | 2; Entry version | PCGHPGDTPFGTFTLTGGNVFEYGVKAVYTCNEGYQLLGEI |
| | 244 (14 Dec. 2022) | NYRECDTDGWTNDIPICEVVKCLPVTAPENGKIVSSAMEPDR |
| | Sequence version 4 | EYHFGQAVRFVCNSGYKIEGDEEMHCSDDGFWSKEKPKCV |
| | Including signal | EISCKSPDVINGSPISQKIIYKENERFQYKCNMGYEYSERGDA |
| | peptide sequence | VCTESGWRPLPSCEEKSCDNPYIPNGDYSPLRIKHRTGDEITY |
| | (underlined) | QCRNGFYPATRGNTAKCTSTGWIPAPRCTLKPCDYPDIKHG |
| | | GLYHENMRRPYFPVAVGKYYSYYCDEHFETPSGSYWDHIH |
| | | CTQDGWSPAVPCLRKCYFPYLENGYNQNYGRKFVQGKSID |
| | | VACHPGYALPKAQTTVTCMENGWSPTPRCIRVSFTL |
| 41 | His tag | HHHHHH |

Aspects and embodiments of the present invention will now be discussed with reference to the accompanying figures. Further aspects and embodiments will be apparent to those skilled in the art. All documents mentioned in this text are incorporated herein by reference.

### FIGURES

Embodiments and experiments illustrating the principles of the invention will now be discussed with reference to the accompanying figures in which:

FIGS. 1A and 1B. Graphs showing nucleic acid (1A) and protein expression (1B) of CR1 CCPs 8-10 in ARPE19 cells from plasmids containing codon-optimized nucleotide sequence (CTx001_a, CTx001_d, CTx001_e, CTx001_f) compared to plasmid containing wild-type nucleotide sequence (CTx001_g).

FIG. 2. Graph showing nucleic acid expression of CR1 CCPs 8-10 in HEK293 cells from plasmids containing codon-optimized nucleotide sequence (CTx001_a, CTx001_d, CTx001_e, CTx001_f) compared to plasmid containing wild-type nucleotide sequence (CTx001_g).

FIGS. 3A to 3C. Graphs showing expression of transgenes encoding CR1 CCPs 8-10 by ARPE19 cells transfected with rAAV vectors containing constructs CTx001_a, CTx001_b, CTx001_c, CTx001_d, CTx001_e and CTx001_f at (3A) 10,000, (3B) 50,000 or (3C) 100,000 MOI.

FIG. 4. Western blots performed on supernatant from ARPE19 cells transfected with rAAV vectors containing the constructs listed for FIG. 3 at 100,000 MOI.

FIG. 5. Graph showing expression of miniCR1 in hTERT-RPE cells transfected with rAAV vectors containing constructs CTx001_a, CTx001_b, CTx001_d, CTx001_e and CTx001_f at 50,000 and 100,000 MOI.

FIG. 6. Graph showing expression of miniCR1 in primary RPE cells transfected with rAAV vectors containing constructs CTx001_a, CTx001_b, CTx001_d, CTx001_e and CTx001_f at 50,000 and 100,000 MOI.

FIG. 7. Graph showing miniCR1 expression from rAAV vectors containing constructs CTx001_a, CTx001_b and CTx001_c after subretinal administration into mice, compared to null control.

FIG. 8. Western blot showing that miniCR1 expressed in HEK293 cells acts as a cofactor for Factor I and breaks down C3b into iC3b, and subsequently into C3dg and C3d (Lane R2).

FIG. 9. Graph showing ability of miniCR1 to inhibit MAC formation in human serum.

FIGS. 10A to 10C. Graphs showing (10A) amount of MAC deposition by C5b9 staining on retinal flat mounts following subretinal administration of rAAV2 vectors expressing constructs CTx001_b, CTx001_e and null (negative control) into laser-induced choroidal neovascularization (CNV) mouse model; (10B) miniCR1 expression from constructs CTx001_b, CTx001_e and null after subretinal administration in the laser-induced CNV mouse model; (10C) representative fluorescent images for AAV2 null vector and CTx001_b treated mouse eyes stained for C5b9. Region of laser lesion is outlined in white dotted line.

FIG. 11. Breakdown of C3b in the presence of Factor I and CR1 polypeptide.

FIGS. 12A and 12B. Breakdown of C3b in the presence of Factor I, FHR-4 and the CR1 polypeptide. (12A) Increasing concentrations of FHR-4 were added to monitor the effect on C3b breakdown. (12B) Increasing concentrations of CR1 polypeptide were added to pre-incubated C3b, FI and FHR-4 to monitor the effect on C3b breakdown.

FIGS. 13A and 13B. (13A) Graph showing complement activation levels in activated human serum at fixed miniCR1 concentration and increasing levels of supplemented FHR proteins. Mean values+/−SEM are presented, where: PC=Positive Control (human serum without CTx001 and without addition of FHRs). (13B) Graph showing Wieslab assay of complement alternative pathway activation in the presence of increasing levels of miniCR1 and the presence (closed triangles) or absence (closed circles) of physiological levels of FHR proteins as identified in a large cohort of AMD patients: FHR1 38 nM; FHR2 55 nM; FHR4 54 nM; FHR5 28 nM.

FIG. 14. Graph showing amount of MAC deposition by C5b9 staining on retinal flat mounts following subretinal administration of rAAV2 vector CTx001_b at different doses and null vector into laser-induced choroidal neovascularization (CNV) rat model. Data are mean±/−SEM. Total spots analyzed per group as follows: Null Vector n=22; CTx001 1E7 n=30; CTx001 1E8 n=28; CTx001 5E9 n=27. Statistical analysis was performed by One Way ANOVA with Dunnett's multiple comparisons. * p<0.05.

FIGS. 15A and 15B. Graphs showing (15A) mean body weight or (15B) mean body weight gain for non-human primate (NHP) dosing groups during the pre-study and active study time period.

FIG. 16. Graph showing intraocular pressure (IOP) measurements for NHP dosing groups. IOP was measured on study days 12-13 (Exam 1); days 25-27 (Exam 2); and days 50-54 (Exam 3). Data are presented as mean±/−SD, n=6 eyes total per group.

### EXAMPLES

### Example 1: Small Fragments of CR1 Comprising CCPs 8-10 have Therapeutic Utility

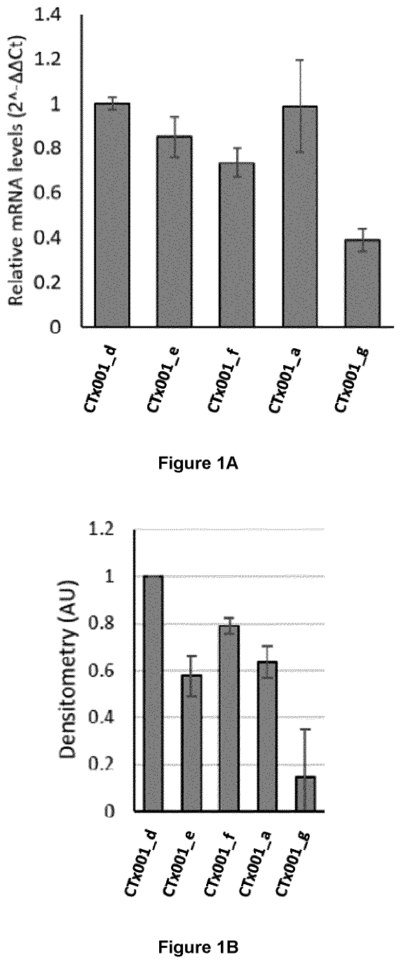

WO 2019/138137 A1, hereby incorporated by reference in its entirety, describes small polypeptides that comprise CCPs (SCRs) 8-10 of human CR1 (see SEQ ID NO:146 herein). These polypeptides can access sites of unwanted pathological complement activation, for example via diffusion between tissue compartments, and can facilitate Factor I-mediated breakdown of C3b into iC3b and further breakdown products, see e.g. FIG. 1 of WO 2022/058447 A1. This helps to reduce undesirable accumulation of both C3b and iC3b. iC3b is a pro-inflammatory molecule which acts to recruit immune cells to the site of complement activation, which in turn cause negative inflammatory effects.

Example 1 of WO 2019/138137 A1 characterises a polypeptide comprising CCPs 8-10 of CR1 attached to an FH signal peptide sequence (designated CR1a/nCR1a in WO 2019/138137 A1 and referred to herein as SEQ ID NO:12).

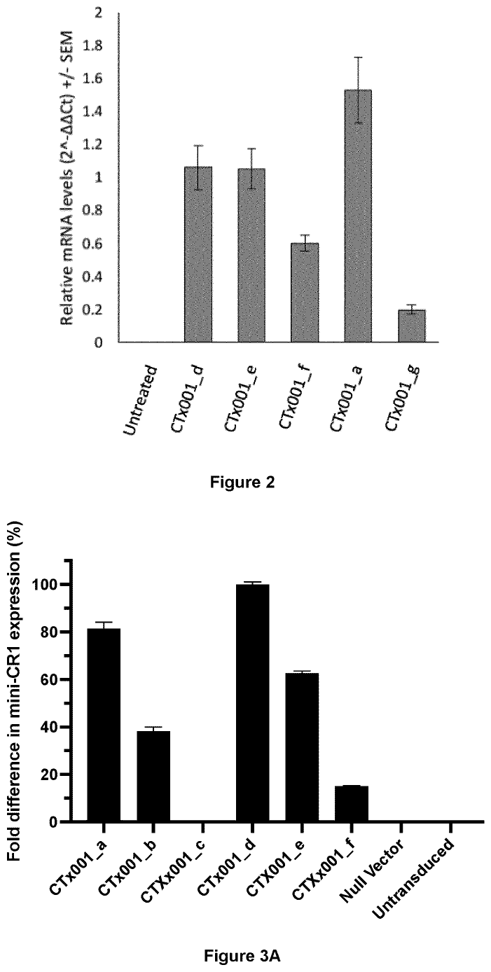

FIG. 2 of WO 2019/138137 A1 shows that CR1a/nCR1a can act as cofactors to assist Factor I (FI) in the cleavage of C3b to iC3b and then further to C3dg. The C3b breakdown using CR1a/nCR1a continues further than the normal native breakdown of C3b observed using FI+FHL-1, which can only produce inflammatory iC3b.

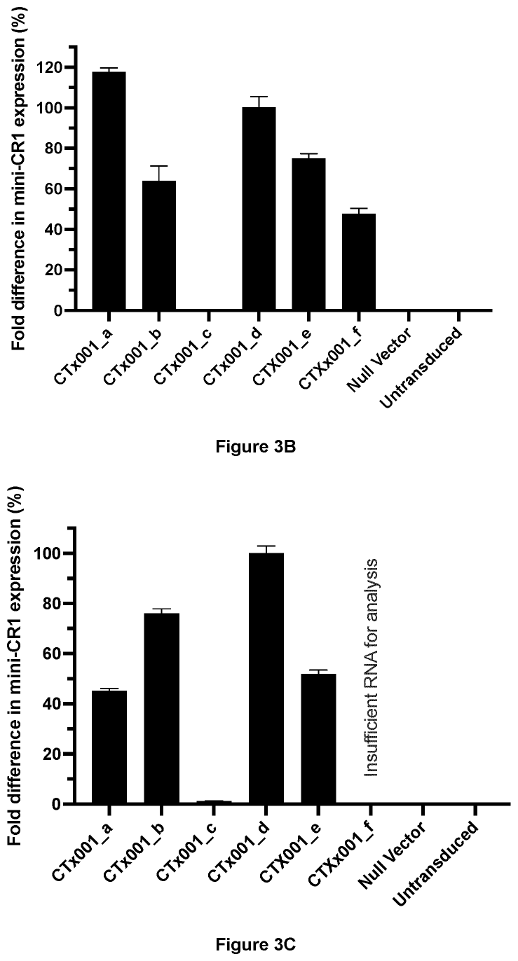

Complement turnover and MAC deposition associated with AMD occurs primarily on and around the ECM of the choriocapillaris underlying the outer blood/retinal barrier. As such, a complement-targeting therapeutic delivered into the eye will be most effective if it can cross and be functionally active on both sides of Bruch's membrane. FIG. 3 of WO 2019/138137 A1 shows that CR1a and nCR1a were able to cross Bruch's membrane from the sample chamber while remaining functionally active cofactors for FI-mediated breakdown of C3b to iC3b and beyond. Example 6 of WO 2019/138137 A1 also demonstrates this property.

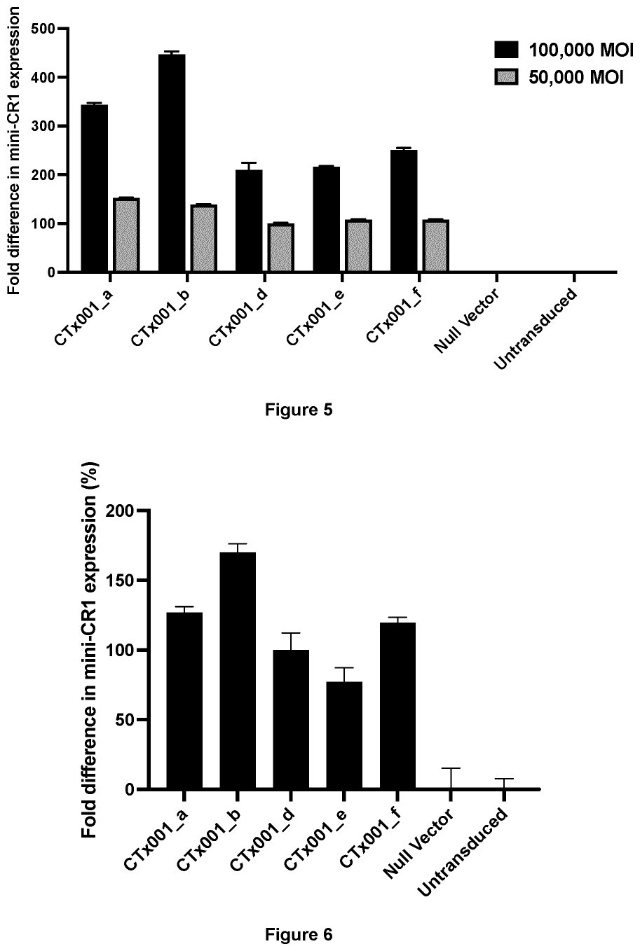

Example 3 and FIG. 5 of WO 2019/138137 A1 demonstrates that the binding affinity of CR1a for C3b was 21 nM. For comparison, the binding affinity for C3b of FH was 580 nM and of FHL-1 was 1.2 μM. CR1a therefore binds significantly more strongly to C3b than either of these native soluble complement regulators. The strong binding affinity of CR1a for C3b means that CR1a is a more effective agent to promote C3b break down than agents based on FH or FHL-1.

A strong binding affinity enables CR1a to promote degradation of iC3b into desirable further downstream products e.g. C3dg. In contrast, FH and FHL-1 cannot cause degradation of C3b beyond iC3b. Further breakdown of iC3b to C3dg by CR1a is thus advantageous and avoids further damage caused by the subject's immune system.

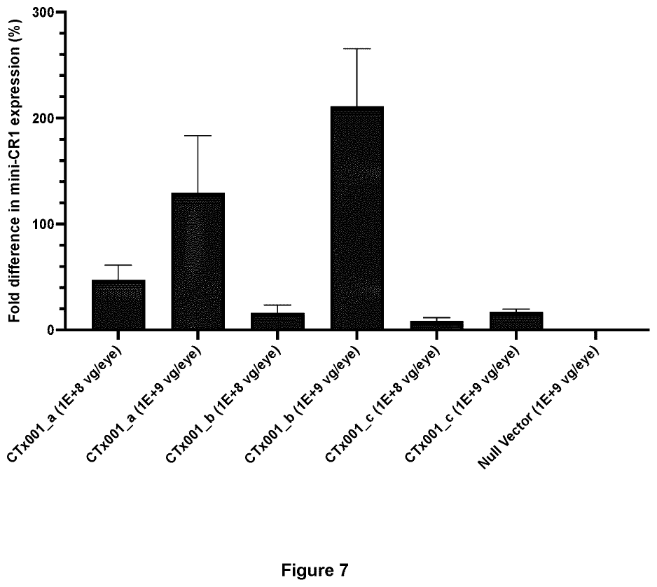

Example 5 and FIG. 7A of WO 2019/138137 A1 show that functional CR1a polypeptide secreted from human APRE-19 cells and HEK cells was able to act as a cofactor for Factor I leading to breakdown of C3b into iC3b and further into C3dg. FIG. 7B demonstrates that media containing CR1a expressed from cells via AAV was found to have increased C3b breakdown capacity for iC3b (product b; lane 3) compared to the native C3b turnover rate in control cell media.

This indicates that CR1a is secreted successfully from cells, e.g. retinal pigment epithelium cells, is functionally active as a FI cofactor, enhances C3b break down to downstream products (iC3b and beyond), is capable of complement regulation, and would provide therapeutic benefit for conditions involving over-activation of complement e.g. an excess of C3b.

### Example 2: Structure and Function of Constructs Encoding CR1 CCPs 8-10

### 2.1 Construct Design

Nucleotide sequences encoding CR1 CCPs 8-10 were introduced into expression cassettes as shown in Table 1.

TABLE 1
Construct designs

| | 5′ | | | | | | | Size |
|---|---|---|---|---|---|---|---|---|
| Name | ITR | Promoter | Transgene | WPRE | PolyA | Stuffer | 3′ ITR | (bp) |
| CTx001_a | ITR | sCAG | CTx | WPRE | bGHpA | intronVMD2^{mod} | ITR | 4316 |
| CTx001_b | ITR | CBh | CTx | — | SV40 | — | sc_ITR | 1943 |
| CTx001_c | ITR | mCAG | CTx | WPRE | bGHpA | — | sc_ITR | 2452 |
| CTx001_d | ITR | CAG | CTx | WPRE | bGHpA | — | ITR | 3624 |
| CTx001_e | ITR | CAG | CTx | WPRE | bGHpA | intronVMD2^{mod} | ITR | 4679 |
| CTx001_f | ITR | CAG | CTx | — | bGHpA | intronVMD2^{mod} | ITR | 4388 |
| CTx001_g | ITR | CAG | CTx (WT) | WPRE | bGHpA | intronVMD2^{mod} | ITR | 4679 |
| CTx001_h | ITR | CAG | CTx | WPRE | bGHpA | intronRLBP1^{mod} | ITR | 5285 |
| CTx001_i | ITR | sCAG | CTx | — | SV40 | — | sc_ITR | 2249 |
| CTx001_j | ITR | sCAG | CTx | — | bGHpA | — | sc_ITR | 2352 |
| CTx001_k | ITR | EF1A | CTx | — | SV40 | — | sc_ITR | 2324 |
| Null | ITR | CAG | — | WPRE | bGHpA | intronVMD2^{mod} | ITR | |

i. 5′ ITR=AAV2 5′ inverted terminal repeat (SEQ ID NO:25 for ssAAV or SEQ ID NO: 26 for scAAV);
ii. CAG: CAG synthetic promoter containing CMV immediate early enhancer, chicken β-actin (CBA) promoter, and a synthetic chimeric intron containing the first exon plus splice donor and intron of chicken β-actin (CBA) promoter with an intron and splice acceptor of rabbit β-globin (SEQ ID NO:15);
iii. sCAG: short CAG synthetic promoter containing CMV immediate early enhancer, chicken β-actin (CBA) promoter, and a shortened synthetic chimeric intron with chicken β-actin splice donor and rabbit β-globin splice acceptor (SEQ ID NO:16);
iv. mCAG: truncated CAG promoter containing CMV enhancer, CBA promoter, and a synthetic chimeric intron from human β-globin and immunoglobulin heavy chain genes (SEQ ID NO:17);
V. CBh: truncated promoter containing CMV enhancer, CBA promoter, and a synthetic hybrid intron from chicken β-Actin and minute virus of mouse (MVM) (SEQ ID NO:14); EF1A: human elongation factor-1 alpha promoter (SEQ ID NO:18); vi.
vii. CTx: codon-optimised nucleic acid encoding CR1 CCPs 8-10 and a Factor H secretory pathway sequence (SEQ ID NO:6), also called ‘miniCR1’ herein;
viii. CTx (WT): wild type nucleic acid encoding CR1 CCPs 8-10 and a wild type Factor H secretory pathway sequence (SEQ ID NO:8);
ix. WPRE: mut6 WPRE (Woodchuck Hepatitis Virus (WHV) Posttranscriptional Regulatory Element) (SEQ ID NO:22);
x. SV40: Simian virus 40 polyadenylation signal sequence (SEQ ID NO:19);
xi. bGHpA: bovine growth hormone polyadenylation signal sequence (SEQ ID NO:20);
xii. intron VMD2mod: inert stuffer sequence derived from intron 1 of the human VMD2 (BEST1) gene with splice acceptor, splice donor, repetitive and TATA like sequences removed (SEQ ID NO:23);
xiii. intronRLBP1mod: inert stuffer sequence derived from human RLBP1 intron with splice acceptor, splice donor, repetitive and TATA like sequences removed (SEQ ID NO:24);
xiv. 3′ ITR: AAV2 3′ inverted terminal repeat (SEQ ID NO:27);
xv. sc_ITR: AAV2 self-complementary ITR (SEQ ID NO:28).

It is important to ensure that the AAV genome (including ITR sequences) does not fall below ~4.0 kb for ssAAV vectors. Ideally, a ssAAV gnome would be kept close to the native size of ~4.7 kb. This is to ensure efficient packaging of the genome into the AAV particles and supports the manufacture and storage stability of high-quality recombinant AAV vectors. A 1259 bp stuffer DNA sequence was generated from the 1343 bp human VMD2 (BEST1) intron-1 or from an intron of human RLBP1. Splice donor and acceptor sites and repetitive sequences were removed to avoid unexpected splicing events. The stuffer sequences were placed after the polyA sequences to avoid unfavourable secondary structures and potential transcription and/or translation in the 5′UTR.

### 2.2 Transient Transfection of Plasmids Comprising Codon-Optimised Transgene

Plasmids were constructed comprising codon-optimised nucleic acid encoding human CR1 CCPs 8-10 (SEQ ID NO:5) attached to a codon-optimised sequence encoding a secretory pathway sequence from Factor H (SEQ ID NO:11). The entire codon-optimised nucleic acid sequence is shown in SEQ ID NO:6. The plasmids express the amino acid sequence of CR1 CCPs 8-10 (shown in SEQ ID NO:12 with the FH secretory pathway sequence, and in SEQ ID NO:3 without said sequence).

As a control, wild type nucleic acid encoding human CR1 CCPs 8-10 (nucleotide sequence as in SEQ ID NO:7) was attached to wild type nucleic acid encoding the FH signal peptide sequence (SEQ ID NO:10), to form the nucleotide sequence shown in SEQ ID NO:8).

### RPE Cells

ARPE19 cells were cultured for 24 hours to 90% confluency before performing transient transfection of plasmids by lipofection. After 24 hours, media was removed and replaced with 500 ul of Optimem+2% FBS. After a further 24 hours, the supernatant was aliquoted and stored at −80° C.

For qPCR analysis, cell pellets were harvested and total RNA extracted using a RNeasy mini kit (Qiagen). Samples were treated with TUrBO™ DNase and cDNA was produced using SuperScript™ IV Reverse Transcriptase. qRT-PCR was performed using Fast SYBR™ Green in triplicate. Primer sequences were designed to target the transgene comprising CR1 CCPs 8-10 and snRNP (Housekeeping gene). ΔCt was calculated relative to snRNP, and ΔΔCt was calculated relative to the CTx001_d plasmid result.

FIG. 1A shows that a fragment of CR1 containing CCPs 8-10 was expressed 48 hours after transient transfection. Plasmids CTx001_d, CTx001_e, CTx001_f and CTx001_a contained codon-optimised nucleic acid sequence, which was expressed at higher levels in each case compared to the wild-type nucleotide sequence (CTx001_g). Error bars represent standard deviation from triplicate repeats.

For protein level analysis, supernatant samples were collected and analysed using a WES system (protein simple). Protein was detected using a rabbit primary antibody targeting CCPs 8-10 of CR1, used at 1:1000 concentration. Total protein levels for each sample were detected using the total protein detection module (Protein simple, DM-TP01). Densitometry analysis was determined using the WES software.

FIG. 1B shows higher expression of CR1 CCPs 8-10 protein from the plasmids containing codon-optimised nucleic acid (CTx001_a, CTx001_d, CTx001_e, CTx001_f), compared to plasmid containing wild type nucleic acid (CTx001_g).

### HEK293 Cells

HEK293 cells were cultured for 24 hours to 80% confluency before performing transient transfection of plasmids by lipofection. After 24 hours, media was removed and replaced with 1 ml of Optimem+2% FBS. After a further 24 hours, the supernatant was aliquoted and stored at −80° C. The cells were harvested using trypsin and pellets were stored at −80° C. for RNA extraction and expression analysis by qPCR, as above.

FIG. 2 shows higher expression of CR1 CCPs 8-10 nucleic acid from plasmids containing codon-optimised nucleic acid (CTx001_a, CTx001_d, CTx001_e, CTx001_f), compared to plasmid CTx001_g containing wild type nucleotide sequence.

2.3 In Vitro Transduction of rAAV Vectors

rAAV vectors comprising constructs in Table 1 were assessed for their transduction efficiency of RPE cells.

ARPE19 cells were cultured in DMEM: F12 Medium supplemented with 10% fetal Bovine serum (FBS superior, Sigma, catalogue number S0615, lot no. 0001652821) and 1% penicillin streptomycin (Pen/Strep, Sigma, catalogue number T3924, lot no. SLCH7198) in 5% CO₂ incubator at 37° C.

Cells were plated in 24-well plates at a sub-confluent density (60-70%) and transduced with rAAV vectors containing the constructs at a multiplicity of infection (MOI) of 10,000, 50,000 or 100,000. rAAV stock was diluted in culture media to achieve the desired MOI and incubated at 37° C. for 24 hours after which rAAV-containing media was replaced with fresh culture media. Transgene expression levels were then determined after incubation at 37° C. for a further 48 hours.

Cells were collected for RNA isolation 72 hours post-transduction. RNA was extracted using the Isolate RNA Mini Kit (Bioline, catalogue number BIO-52072) following manufacturer's instructions. cDNA was synthesised from 1500 ng of RNA using the Superscript IV Vilo Master Mix cat no. 11756050 (Invitrogen) following the manufacturer's instructions.

Quantitative PCR reactions were performed using transgene targeting FAM-labelled TaqMan probes. In brief, 10-100 ng of cDNA was suspended in a reaction mix including 1 ul of FAM labelled TaqMan probe targeting the miniCR1 transgene (custom designed) and 1 ul of VIC labelled TaqMan GAPDH probe (Hs02758991_g1), 10 μl of 2× reaction TaqMan™ Universal Mastermix II, no UNG (Applied Biosystems™, Thermofischer Scientific, cat no. 4440040), in a final reaction volume of 20 μl in 96 well plate (Biozym, catno. 712220). Samples were run in triplicate in 96 well plate using an ABI 7500 Real Time PCR system (Applied Biosystems) using standard cycling conditions. Gene expression was normalised to GAPDH expression and relative expression determined by the ΔΔCt method.

The results are shown in FIGS. 3A to 3C. MiniCR1 transgene expression is shown after transduction with (3A) 10,000, (3B) 50,000 or (3C) 100,000 MOI of AAV2 constructs. CTx001_a, CTx001_b, CTx001_d and CTx001_e provide high levels of CR1 transgene expression across the three MOIs tested.

Western blots were performed on supernatant from cells transduced at 100,000 MOI. Cell culture media was collected 72 hours post transduction and precipitated using an acetone precipitation method to concentrate the protein. For 20 μL of concentrated supernatant sample, 5 μL of loading buffer was added and then samples were denatured at 90°-100° C. for 5 mins. After denaturation, 25 μL samples and 8 μL of protein standard were loaded in their respective well in 4-12% gel and run on 200V for 45-60 mins.

Protein transfer from the gel onto a membrane was performed by electrophoretic transfer using a standard tank-based transfer system (wet transfer). Following the transfer, the membrane was blocked by incubating with 10% milk solution in phosphate-buffered saline (PBS) supplemented with 0.2% bovine serum albumin (BSA). The membrane was subsequently washed with PBST (PBS supplemented with 0.2% Tween 20) and incubated for 1 hour at room temperature with an anti-CR1 CCPs 8-10 polyclonal antibody (diluted 1:500). The membrane was subsequently washed with PBST and incubated for 1 hour at room temperature with an HRP-labelled anti-rabbit polyclonal antibody (diluted 1:10,000). Detection was performed using Pierce ECL Plus Western Blotting substrate.

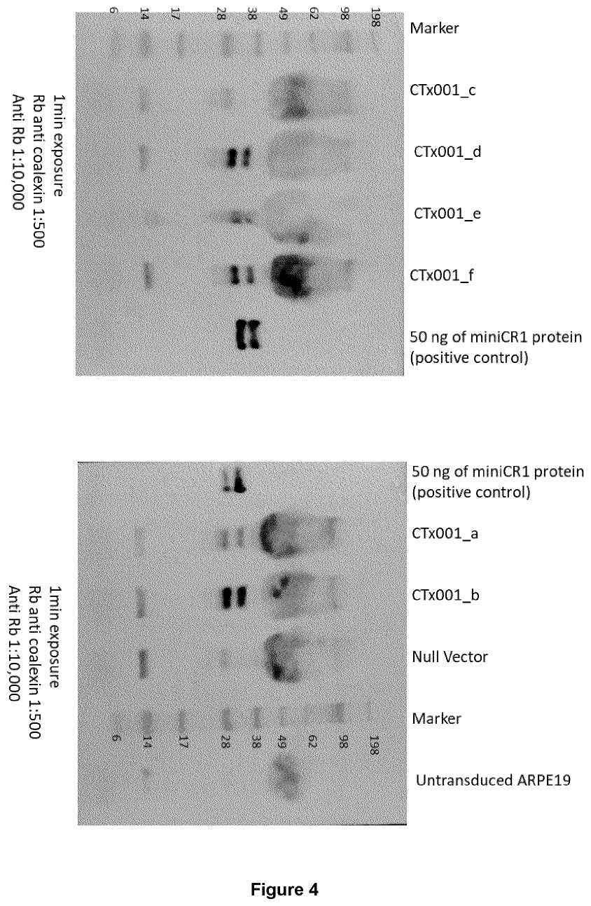

The results are shown in FIG. 4 and confirm that protein containing CCPs 8-10 of CR1 was translated and secreted (as two glycosylated forms at ~28 kDa and 32 kDa) from the AAV constructs tested.

The experiments were repeated in hTERT-RPE cells (FIG. 5) and primary RPE cells (FIG. 6) using CTx001_a, CTx001_b, CTx001_d, CTx001_e and CTx001_f. CTx001_b showed the highest expression in both RPE cell types.

### 2.4 In Vivo Expression Studies

rAAV vectors containing constructs CTx001_a, CTx001_b and CTx001_c were evaluated for expression of miniCR1 transgene after subretinal injection in C57BL/6J mice.

AAV2 vectors were administered as bilateral subretinal injections at 1×10⁸ or 1×10⁹ vg/eye. qPCR was performed to assess the degree of miniCR1 expression.

The results are shown in FIG. 7. CTx001_a and CTx001_b showed excellent, dose-proportionate expression of miniCR1 in the eyes of the C57BL/6J mice. The null vector was included as a negative control.

rAAV vectors containing constructs in Table 1 are assessed for expression of CCPs 8-10 of CR1 following subretinal injection into rat eyes. Protein secretion is evaluated by ELISA measurements of protein in vitreous of rat eyes.

### 2.5 In Vitro Functional Studies

MiniCR1 expressed in HEK293 cells was assessed for its ability to act as a cofactor for human Factor I (FI) and break down human C3b to iC3b and C3d.

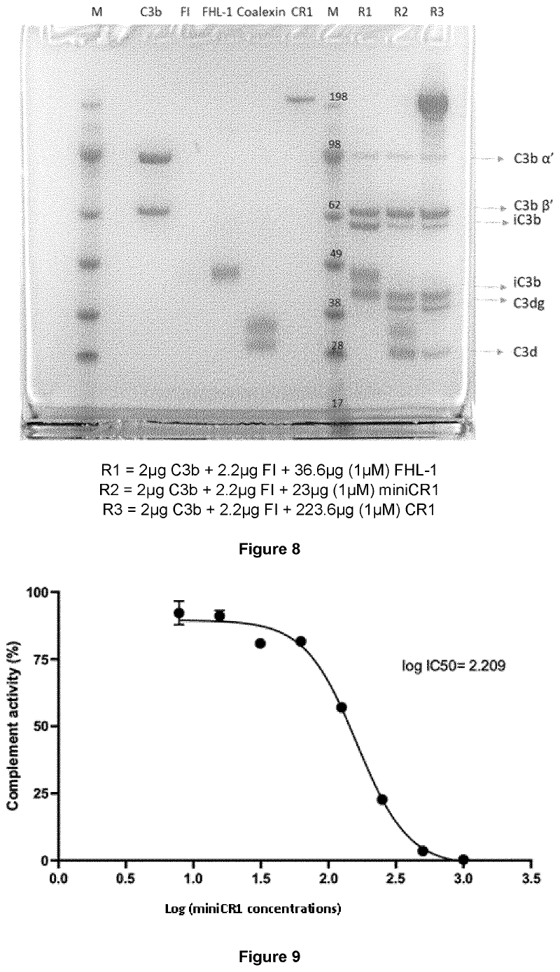

FIG. 8 shows that miniCR1 incubated with human C3b and human FI is able to break down C3b into iC3b, and further into downstream products C3dg and C3d (Lane R2), comparable with FI cofactor FHL-1 (Lane R1) and full-length CR1 cofactor (Lane R3).

Functional complement activity of miniCR1 in human serum was assessed by ELISA experiments performed using the Wieslab® Complement system Screenkit (Svar Life Science) on zymosan activated human serum in the presence of increasing concentrations of mini-CR1. FIG. 9 shows that miniCR1 can inhibit MAC formation in activated human serum, with an IC50 of 125 nM.

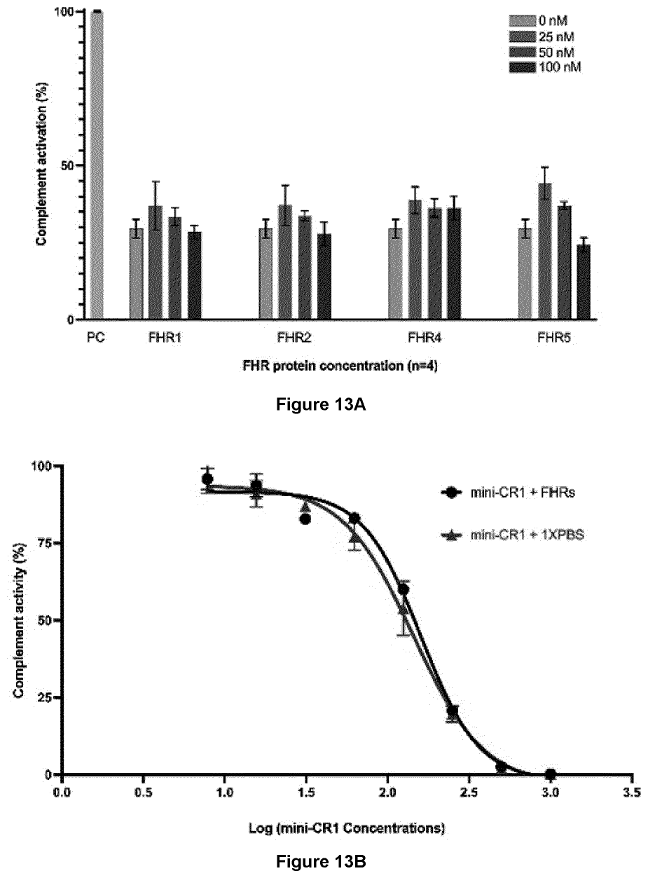

Prevention of MAC formation by miniCR1 was not attenuated in the presence of supraphysiological levels of Factor H related (FHR) proteins, which are elevated in some GA patients and are known antagonists of cofactor driven C3b cleavage (see Example 3, below, and FIGS. 13A and 13B).

### 2.6 In Vivo Functional Studies

rAAV vectors containing constructs CTx001_b, CTx001_e and null (negative control) were assessed for their efficacy via subretinal administration in the laser-induced choroidal neovascularization (CNV) mouse model. Their ability to inhibit membrane attack complex (MAC) formation was assessed. Formation and deposition of the MAC is associated with inflammation and disease pathogenesis in a number of complement-related diseases, including autoimmune diseases (e.g. rheumatoid arthritis and SLE), cancer, nephropathies, AMD (dry and wet forms), Alzheimer's disease, osteoarthritis and atherosclerosis (see e.g. Xie et al., Am J Pathol. 2020 June; 190 (6): 1138-1150, which is hereby incorporated by reference in its entirety). Levels of MAC deposition can be measured as a direct biomarker of complement turnover.

Mice received single unilateral or single bilateral subretinal injections of 5×10⁸ or 5×10⁹ vg/eye AAV2 vectors on Day-28. Spectral-domain optical coherence tomography (SD-OCT) was performed on Day-10 to assess inflammation in the vitreous and topical dexamethasone treatment was applied for 3-5 days if inflammatory cells were present. Choroidal neovascularization (CNV) was induced on Day 0 by perforating Bruch's membrane using a diode laser to promote the growth of subretinal blood vessels from the choroid. Control groups received single unilateral intravitreal injection of aflibercept (Regeneron Pharmaceuticals/Bayer Pharma AG) or aflibercept vehicle on Day 0.

Fluorescein angiography (FA) and spectral-domain optical coherence tomography (SD-OCT) were performed immediately following the rupture of Bruch's membrane at Day 0, and subsequently on Day 3 and Day 5 after CNV induction (Day 4 for control groups) to identify leaky CNV lesions and lesion area. Animals were included in the study if they demonstrated normal morphology at baseline SD-OCT scan, “bubble” formation during lasering, perforation of Bruch's membrane, and presence of leakage on FA just after induction. Animals treated with miniCR1 vectors were euthanised on Day 4 or Day 7 and tissue samples were taken for evaluation. MAC deposition was assessed by C5b9 staining on retinal flat mounts.

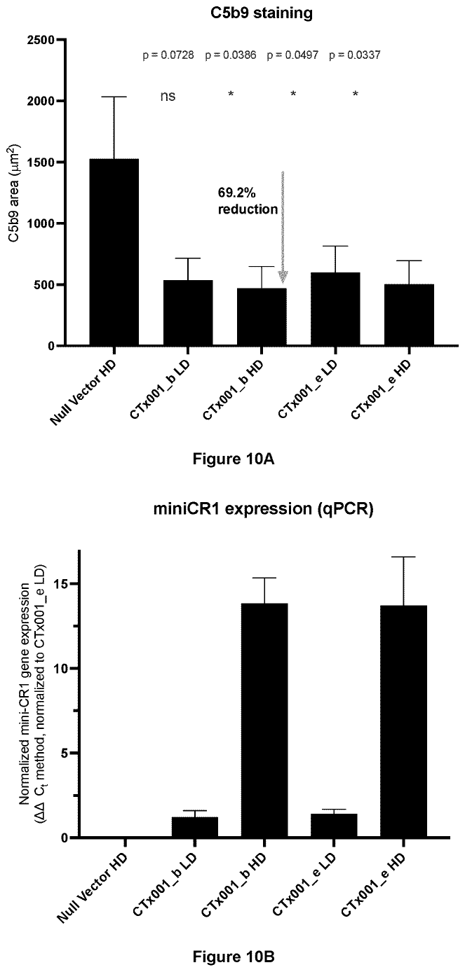

The results are shown in FIG. 10. CTx001_b and CTx001_e AAV2 constructs demonstrated significant reductions in MAC deposition at Day 7 post laser injury compared to the null vector (FIG. 10A). MAC formation was reduced by 65% and 69% relative to null vector by low and high dose CTx001_b, respectively. LD-Low dose (5E+8 vg/eye); HD-High dose (5E+9 vg/eye). Data are mean±/−SEM; n=40-42; *=p<0.05; One-way ANOVA with Dunnett post-hoc analysis comparing all groups to Null Vector.

For comparison, therapeutic agent HMR59 (soluble CD59 gene therapy for AMD; Hemera Biosciences) targets the MAC directly and achieved only 52.5% reduction compared to Null vector when expressed from adenovirus.

The impact of the laser injury on miniCR1 expression was assessed by qPCR. MiniCR1 was found to be well-expressed in the CNV model (FIG. 10B). Analysis of mice indicated that delivery of CTx001 at either dose was well tolerated with no presence of inflammatory cells detected.

A post-hoc Dunnett's multiple comparison analysis was performed, which revealed that the CTx001 groups had significantly lower C5b9-positive area in comparison to the null vector group. FIG. 10C shows representative fluorescent images for AAV2 null vector compared to CTx001 treated mouse eyes stained for C5b9. Region of laser lesion is outlined in white dotted line.

rAAV vectors were also tested in a rat model of laser-induced CNV. Brown Norway rats were utilized for the study. The study outline is presented in Table 2.

TABLE 2
Experimental Design of the Rat Laser CNV in vivo Pharmacology
Study

| | | N/ | Dose, | | | |
|---|---|---|---|---|---|---|
| Group | Treatment | Group | vg/eye | Administration | Laser | Sacrifice |
| 1 | Null vector | 8 | 5 × 10⁹ | Day-28 | Day 0 | Day 3 |
| 2 | CTx001 | 8 | 1 × 10⁷ | Day-28 | Day 0 | Day 3 |
| 3 | CTx001 | 8 | 1 × 10⁸ | Day-28 | Day 0 | Day 3 |
| 4 | CTx001 | 8 | 5 × 10⁹ | Day-28 | Day 0 | Day 3 |

All virus used in the study was produced via triple transfection method in HEK293 cells. Animals received a single, bilateral, subretinal injection of empty vector or CTx001_b on day-28. All study animals received laser injury (4 spots/eye; both eyes per animal) on day 0. Animals were sacrificed on day 3 post laser injury, which was determined to be the optimal timepoint to visualize complement deposition in this model according to natural history data. At the time of sacrifice, vitreous fluid was taken from each study eye of every animal and frozen for bioanalytical analysis. Retinal flatmounts were prepared from all study eyes and MAC staining was performed with a polyclonal antibody specific to a neoepitope found on the C5b9 complex. Phalloidin staining was used to visualize laser lesions, and semi-automated image analysis was performed to quantify MAC staining.

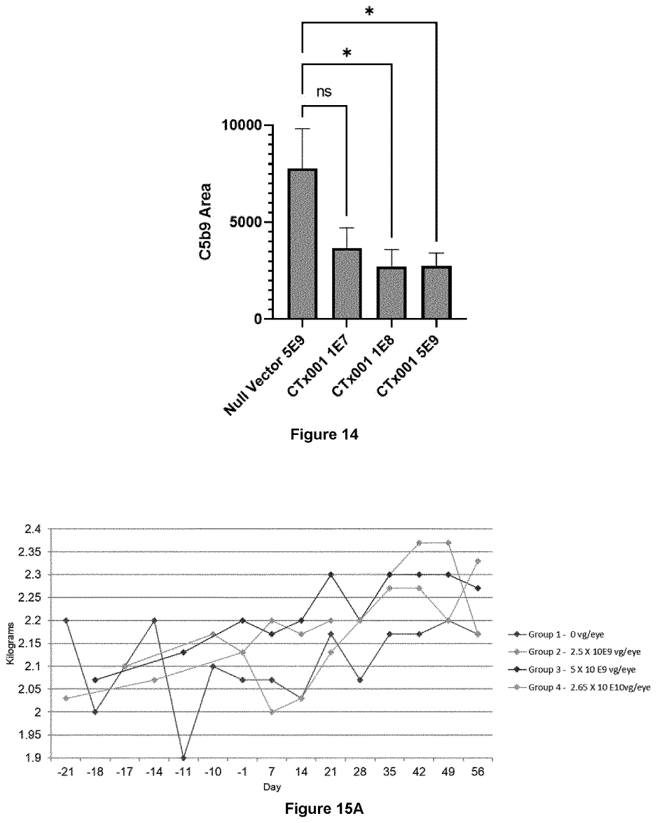

MiniCR1 expression induced a dose responsive decrease in MAC staining (FIG. 14). MiniCR1 (1×10⁸ vg/eye and 5×10⁹ vg/eye) demonstrated significant reductions in MAC deposition at Day 3 post laser injury compared to the null vector (FIG. 14).

### Example 3: Further Characterisation of Polypeptides Comprising CCPs 8-10 of CR1

The fluid-phase activity of a small polypeptide comprising CCPs 8-10 of CR1 (SEQ ID NO:3) was tested in a C3b breakdown assay. 2 μg of C3b, 0.04 μg FI and 1 μg of polypeptide were incubated together in a total volume of 20 μl at 37° C. for 30 min. Reactions were stopped by the addition of 5 μl 5× SDS reducing sample buffer and boiling at 100° C. for 10 minutes. Samples were run on a 4-12% NuPAGE Bis Tris gel at 200V for 60 minutes in order to maximise the separation of the C3b breakdown product bands.

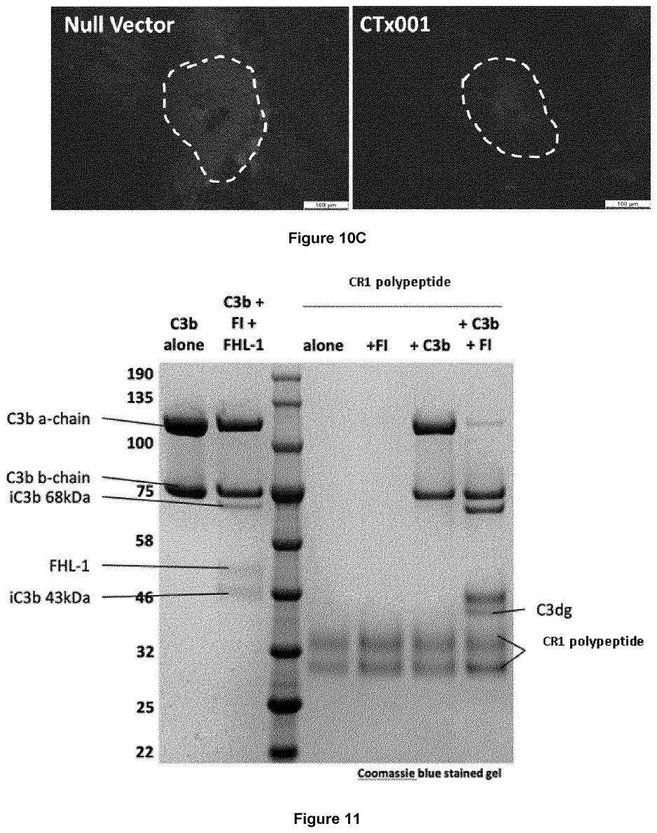

FIG. 11 shows C3b breakdown in the presence of Factor I and the CR1 polypeptide. The C3b alpha-chain is cleaved by FI to form iC3b, then C3dg and finally C3d. The polypeptide acts as a ‘super FI co-factor’, allowing FI to cleave C3b down to the C3dg and C3d level, unlike native FI cofactors which can only mediate cleavage down to the iC3b level.

Increasing concentrations of FHR proteins have been shown to out-compete the binding of native soluble regulators to C3b, thus interfering with FI-mediated C3b breakdown and causing excessive complement activation (Cipriani et al. Nat Commun 11, 778 (2020), hereby incorporated by reference in its entirety). Given that the FHR proteins accumulate in the ECM surrounding the choriocapillaris in human eyes, the CR1 polypeptide was evaluated for its ability to act as a cofactor for FI in the presence of such proteins.

For FHR4 protein competition experiments, the concentration of C3b, FI and the CR1 polypeptide were kept constant and increasing concentrations of FHR4 were included in the reaction to monitor the effect of FHR4 on C3b breakdown. Conversely, C3b, FI and FHR4 were pre-incubated before the addition of increasing concentrations of the CR1 polypeptide to explore at what point the polypeptide could counter-act the prohibitive effects of FHR-4 on C3b breakdown.

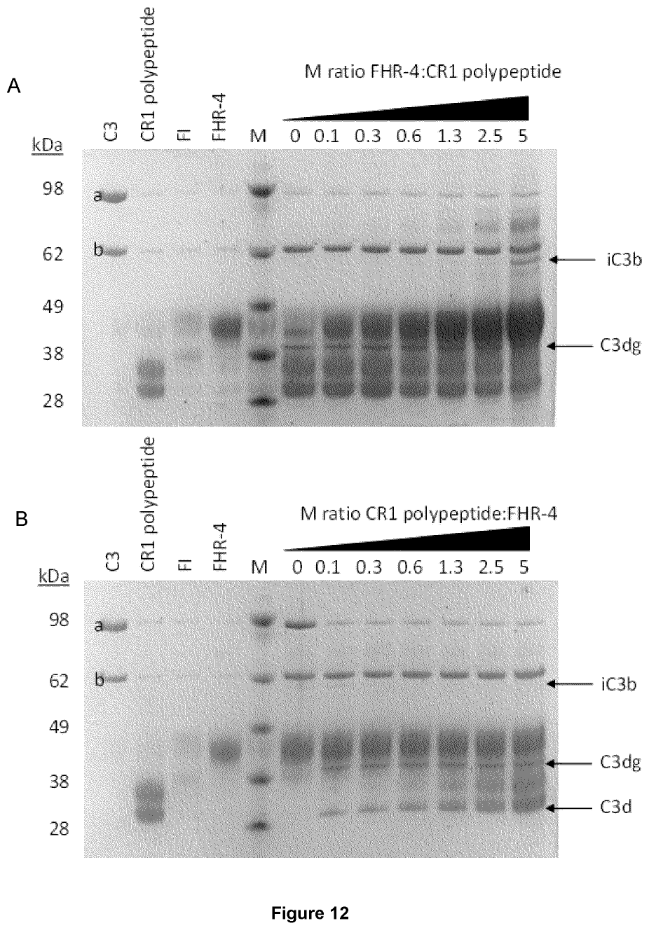

FIG. 12A shows that C3b breakdown by the CR1 polypeptide is not perturbed by excessive FHR4, even in the presence of a 5-fold molar excess of FHR4. FIG. 12B shows that this remains true even when FHR-4 is pre-incubated with the C3b and FI prior to CR1 polypeptide addition. Here, even the lowest concentration of CR1 polypeptide used is enough to overcome the inhibitory function of FHR4 and mediate C3b breakdown into iC3b, C3dg and C3d.

Further FHR protein competition experiments were performed using C3b, FI, the CR1 polypeptide and each of FHR1, FHR2, FHR4 and FHR5 in turn. IC50 values were obtained for the CR1 polypeptide in the presence of FHR proteins. Inhibition of complement activation (e.g. C3b breakdown by the CR1 polypeptide) was found to occur even in supra-physiological concentrations of FHR proteins (FIG. 13A). Similarly, when FHR1, FHR2, FHR4 and FHR5 are pooled and supplemented into human sera at levels matching the elevated concentrations found in AMD patients from previous studies there was no effect on the IC50 value of the CR1 polypeptide (FIG. 13B).

The CR1 polypeptide binds to C3b with a greater affinity than the FHR proteins, such that even in the presence of a large molar excess of FHR protein, it can still bind to C3b and facilitate the FI-mediated cleavage of C3b. This means that the CR1 polypeptide can drive complement inhibition even in the presence of elevated FHR protein concentrations, unlike other native soluble FI co-factors (e.g. FH, FHL-1).

Thus, the CR1 polypeptide finds utility in treating disorders involving undesirable complement activation associated with elevated levels of FHR proteins, which would otherwise out-compete FH and FHL-1 as cofactors for FI-mediated C3b breakdown.

### Example 4: Detection of Peptides Generated from Complement Proteins and their Use in Predicting Disease Risk

Example 1 of WO 2022/058447 A1, hereby incorporated by reference in its entirety, shows that GluC digestion of FH, FHL-1, FHR1-5, FI, C3, C3b and C3b breakdown products achieves distinct peptides for mass spectrometry. Table 1 of WO 2022/058447 A1 provides distinct peptides for Factor H family proteins (FH, FHL-1, FHR1-5) after GluC digestion. FIG. 1 and Tables 2-4 of WO 2022/058447 A1 show the proteoform-specific peptides produced by GluC digestion of C3 and its breakdown products. Table 5 of WO 2022/058447 A1 shows peptides from Factor I after GluC digestion; peptides 8-20 contain 8-21 amino acids and are a good length for MS analysis.

Example 2 of WO 2022/058447 A1 describes mass spectrometry of the peptides generated. FIG. 2 of WO 2022/058447 A1 demonstrates that the method is feasible, specific and has the required sensitivity to distinguish between peptides from these seven proteins, in particular between splice variants FH and FHL-1. FIG. 3 of WO 2022/058447 A1 demonstrates that the GluC digestion produces peptides that can be detected individually and specifically in native serum at endogenous levels. It also shows that the assay is capable of quantifying the level of each protein in the sample. Increasing amounts of protein increase the signal in a predictable manner, allowing determination of the levels, as well as the presence, of each of the proteins. Also demonstrated is that the assay is free from interference.

Example 2.4 and FIG. 4A of WO 2022/058447 A1 show that all peptides in Table 2 can be detected individually in a plasma sample by SRM-MS using at least three transitions. The specificity of the assay for the peptides of interest is confirmed by the relative intensities of the transitions matching the relative intensity of the relevant product ions in an MS/MS scan. FIG. 4B of WO 2022/058447 A1 confirms the specificity of the peptides, showing experiments in which the plasma sample was spiked with crude synthetic peptide which demonstrated the appropriate increase in signal. FIG. 4C of WO 2022/058447 A1 shows the time course of the C3b breakdown via gel electrophoresis. Analysis using MS and the peptides of Table 2 of WO 2022/058447 A1 demonstrates that the formation of C3b fragments iC3b, C3f and C3c, and loss of intact C3b can be clearly detected over time (FIG. 4D).

The data in Example 2 of WO 2022/058447 A1 demonstrate that C3/C3b breakdown can be measured in a quantitative manner using GluC-derived peptides and MS. This enables the presence and levels of complement proteins to be detected in complement-related diseases such as AMD, as well as providing information as to successful treatment outcomes. A single assay which can measure all FH family, C3 fragments and FI proteins allows for the simultaneous analysis of all key proteins in the complement amplification loop from just one sample and with efficient throughput.

Example 3 of WO 2022/058447 A1 describes the detection of the complementome in AMD patients. The data were examined to determine to what extent the clinical outcome (AMD or no AMD) could be predicted based on the different protein level values. Multiple logistic regression analysis was used for this purpose and the findings for different models are shown in FIG. 7 of WO 2022/058447 A1. It was found that a model including all studied proteins (FHR1, FHR2, FHR3, FHR4, FHR5, FHL-1 & CFH protein levels) had the highest discrimination ability (AUROC (area under ROC curve) of 0.7498; FIG. 8 of WO 2022/058447 A1), although all of the models tested were capable of discriminating between subjects with AMD and control subjects.

Genome-wide association analyses were performed of the protein levels that were found to be elevated in advanced AMD cases (i.e., FHL-1 and FHR-1 to FHR-5). FIG. 9 of WO 2022/058447 A1 shows that all GWASs of the five FHR protein levels in 252 controls showed a genome-wide significant (P≤5×10⁻⁸) peak at the CFH locus. For FHR-1, FHR-2, FHR4 and FHR-5, the CFH locus was the only genome-wide significant peak observed.

FIG. 11 of WO 2022/058447 A1 shows the Mendelian randomization estimates of the FHR protein levels obtained using the (one-sample) Wald ratio (if a single instrument was available; FHR1, FHR2, FHR4, FHR5) or the IVW method (if multiple instruments were available; FHR3) together with the traditional epidemiologic estimates of the association of the levels with AMD obtained from logistic regression models and ORs. The Mendelian randomization estimates were statistically significant and of concordant direction with the observational OR estimates for FHR-1, FHR-2, FHR-4 and FHR-5, providing evidence in support of a causal effect. Applying a cut off for ‘high’ expression of FHR proteins according to the 90th percentile of the healthy controls group, it was calculated that ~30% of AMD cases in this cohort displayed elevated levels of at least one FHR protein. This is consistent with the observation that around 34% of AMD patients carry an AMD risk variant at this locus.

Example 4 of WO 2022/058447 A1 describes that increased intratumoral CFH and FHL-1 levels were associated with poorer survival among glioma patients. Human tumor cells utilize IDO non-enzyme activity to enhance the expression level of immunosuppressive CFH and its truncated isoform, FHL-1. It is demonstrated that tumor cell FHL-1: (i) enhanced macrophage maturity, (ii) enhanced macrophage expression for ARG1, CCL2, and IL-6, and (iii) decreased the survival of mice with brain tumors in-part by suppressing the anti-GBM T and NK cell immune response.

Example 5 of WO 2022/058447 A1 describes the analysis of the levels of complement proteins CFH, FHL1, FHR1, FHR2, FHR3, FHR4 and FHR5 in samples of blood obtained from 200 COVID-19 patients having varying severity of disease, and in samples of blood obtained from healthy control subjects (not having COVID-19). FIGS. 24 and 25 of WO 2022/058447 A1 show that statistically-significant elevations (one-way ANOVA) in the levels of FHL1, FHR1, FHR2, FHR3, FHR4 and FHR5 were detected in the blood of COVID-19 patients having severe disease requiring assisted ventilation (group E), relative to levels in the blood of healthy control subjects. There was a general trend towards elevated levels of FHL1, FHR1, FHR2, FHR3, FHR4 and FHR5 with increasing COVID-19 severity. FIG. 25 of WO 2022/058447 A1 shows the comparison of group E patients with uninfected controls. Predictive ability was most pronounced for FHR5, where statistically-significant differences were observed between COVID-19 patients having differing disease severity, although all proteins tested were capable of discriminating between subjects with severe COVID-19 infection and control subjects.

### Example 5: Non-human primate (NHP) study

CTx001 was Administered to Cynomolgus Macaques. AAV-CTx001 Vectors were Delivered to NHP eyes at 2.5×10⁹, 5×10⁹ or 2.65×10¹⁰ vg/eye via a single bilateral, subretinal injection. Vehicle was used as a control. NHPs were sacrificed after 8 weeks and the tolerability and biodistribution of the vector was assessed.

Tolerability to CTx001 was determined based upon in-life observations and terminal procedures. All animals successfully reached scheduled euthanasia. Necropsy was performed and no abnormal macroscopic findings were observed. Preliminary data for the following outcome measures are shown herein: clinical observations, body weight, clinical pathology, intraocular pressure, and ophthalmic exams.

### Clinical Observations

Detailed clinical observations were recorded during the pre-study period and at least weekly once animals had been dosed. In general, all clinical observations were consistent with normal findings for NHP in grouped housing in a laboratory setting. There were no clinical observations (e.g. in fur, skin, or eye findings) specifically linked to CTx001 dosing, either in number or severity.

### Body Weight

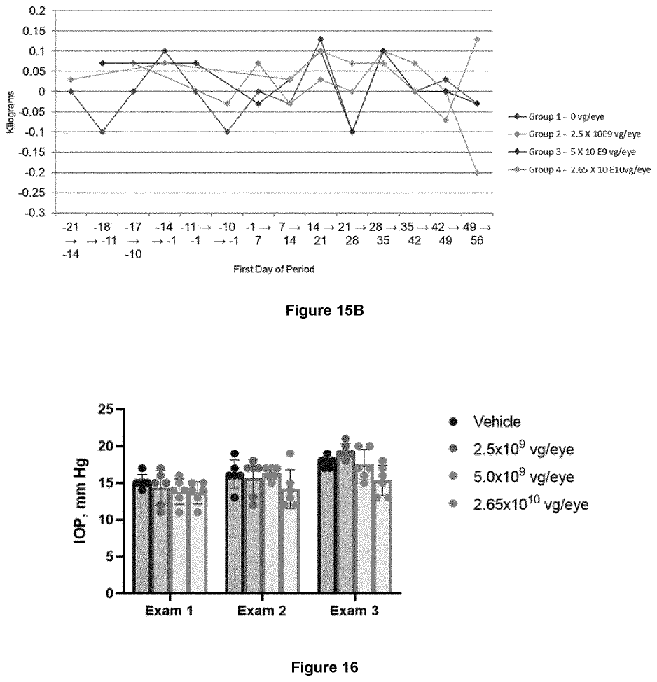

Animal body weights were recorded beginning in the pre-study period and weekly thereafter following dosing. In general, body weights increased slightly following test article administration, comparable to the vehicle treated group, for all CTx001 dosed animals (FIG. 15A). The rate of weight gain over time fluctuated as well, but within 10% of total body weight for all dosing groups at all timepoints (FIG. 15B). Overall, there is no evidence that CTx001 impacts the rate of animal growth or induces weight loss at any dose investigated.

### Clinical Pathology

Clinical pathology results show no observed findings in clinical chemistry, haematology or coagulation parameters. Liver enzymes did not show any evidence of hepatic toxicity. Specific pathways that could putatively be linked to perturbations in complement biology, such as immune regulation and coagulation, did not display any abnormal findings. Overall, there was no evidence of systemic toxicities linked to CTx001 exposure in the clinical pathology data.

### Intraocular Pressure

IOP was assessed in each eye for all animals at three timepoints: Exam 1 (day 12-13), Exam 2 (day 25-27) and Exam 3 (day 50-54). There were no drug related changes of IOP relative to vehicle control at any CTx001 dose investigated at any timepoint (FIG. 16).

### Ophthalmic Exams

All animals were subjected to funduscopic (indirect ophthalmoscopy) and biomicroscopic (slit lamp) examinations. Examinations were performed once during pre-study and again during Day 3 and Weeks 2, 4 and 8. With very few exceptions, pre-study exams presented no findings, and the few findings observed during the pre-study/baseline period were graded as very slight. There were no signs of retinitis by week 8. Ocular findings such as anterior chamber cells, anterior chamber flare, vitreal haemorrhage, and retinal haemorrhage were seen during the first two exams but improved or resolved by Week 8. Retinal haze became apparent at week 2 only in eyes exposed to CTx001. This finding was deemed very slight/slight in severity and persisted until study completion. None of the eyes became overtly inflamed during the eight weeks of the observation period.

In general, subretinal injection of CTx001 at dose levels of 2.5×10⁹, 5×10⁹ and 2.65×10¹⁰ vg/eye was well tolerated in NHPs.

### Summary of Findings

CTx001_b was well tolerated in cynomolgus macaques following a single subretinal injection followed by an 8-week observation period.

No findings seen in the preliminary data indicated systemic toxicity in any form. Clinical observations and clinical pathology were judged normal and comparable to values from the vehicle control. Local toxicity, measured via fundoscopic and biomicroscopic exams, was also mild. The vast majority of findings were deemed very slight to slight. All ophthalmic findings resolved in severity by Week 8 (end of study), at which time there were no group mean scores above slight for any parameter.

1. A polynucleotide comprising, in 5′ to 3′ or 3′ to 5′ order:
(i) a first nucleotide sequence comprising a promoter;
(ii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5; and
(iii) a polyadenylation signal sequence.

2. A polynucleotide according to claim 1, wherein the polynucleotide comprises a 5′ inverted terminal repeat (ITR) and/or a 3′ ITR.

3. A polynucleotide according to claim 2, wherein at least one of the ITRs is an AAV1, AAV2, AAV3, AAV4, AAV5, AAV6, AAB7, AAV8, AAV9, AAV10, AAV11, AAV12, AAV-DJ, AAV-DJ/8, AAV-rh10, AAVrh.39, AAV-retro, AAV-PHP.B, AAV8-PHP.eB or AAV-PHP.S ITR, preferably wherein at least one of the ITRs in an AAV2 ITR.

4. A polynucleotide according to claim 2 or claim 3, wherein the 5′ ITR comprises or consists of a sequence having at least 80% sequence identity to SEQ ID NO: 25 or 26 and/or wherein the 3′ ITR comprises or consists of a sequence having at least 80% sequence identity to SEQ ID NO: 27 or 28.

5. A polynucleotide according to claim 2 or claim 3, wherein the 5′ ITR or the 3′ ITR is a mutant ITR.

6. A polynucleotide according to claim 5, wherein the 5′ ITR comprises or consists of a sequence having at least 80% sequence identity to SEQ ID NO: 26 and/or wherein the 3′ ITR comprises or consists of a sequence having at least 80% sequence identity to SEQ ID NO: 28.

7. A polynucleotide according to any one of claims 1 to 6, wherein the promoter is a mammalian promoter, optionally wherein the promoter is selected from: a CBh promoter, a CAG promoter, a truncated CAG promoter, a CMV promoter, a SV40 promoter, a UBC promoter, a EF1A promoter, and a PGK promoter.

8. A polynucleotide according to any one of claims 1 to 7, wherein the promoter comprises or consists of a sequence having at least 80% sequence identity to any one of SEQ ID NO: 14, 15, 16, 17 or 18.

9. A polynucleotide according to any one of claims 1 to 8, wherein the polyadenylation signal sequence is derived from bovine growth hormone (bGH), human growth hormone (hGH) or SV40.

10. A polynucleotide according to any one of claims 1 to 9, wherein the polyadenylation signal sequence comprises or consists of a sequence having at least 80% sequence identity to any one of SEQ ID NO: 19, 20 or 21.

11. A polynucleotide according to any one of claims 1 to 10, wherein the polynucleotide comprises a post-transcriptional regulatory element (PRE) between sequences (ii) and (iii).

12. A polynucleotide according to any one of claims 1 to 10, wherein the polynucleotide comprises a woodchuck hepatitis virus (WHV) post-transcriptional regulatory element (WPRE) between sequences (ii) and (iii).

13. A polynucleotide according to claim 12, wherein the WPRE comprises or consists of a sequence having at least 80% sequence identity to SEQ ID NO:22.

14. A polynucleotide according to any one of claims 1 to 13, wherein the polynucleotide comprises a third nucleotide sequence between sequences (ii) and (iii), wherein the third nucleotide sequence comprises or consists of a stuffer sequence.

15. A polynucleotide according to claim 14, wherein the stuffer sequence is derived from an intron of a mammalian gene, optionally wherein the intron lacks splice acceptor and splice donor sequences, and optionally where the stuffer sequence is derived from a VMD2 intron.

16. A polynucleotide according to claim 14 or 15, wherein the stuffer sequence comprises or consists of a sequence having at least 80% sequence identity to SEQ ID NO:23.

17. A polynucleotide according to any one of claims 1 to 16, wherein the polynucleotide comprises or consists of:
(i) an ITR, optionally comprising or consisting of a sequence having at least 80% sequence identity to SEQ ID NO:26 or 28;
(ii) a first nucleotide sequence comprising a promoter, optionally comprising or consisting of a sequence having at least 80% sequence identity to SEQ ID NO:14;
(iii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5;
(iv) a polyadenylation signal sequence, optionally comprising or consisting of a sequence having at least 80% sequence identity to SEQ ID NO: 19; and
(v) an ITR, optionally comprising or consisting of a sequence having at least 80% sequence identity to SEQ ID NO:28 or 26.

18. A polynucleotide according to any one of claims 1 to 16, wherein the polynucleotide comprises or consists of:
(i) an ITR, optionally comprising or consisting of a sequence having at least 80% sequence identity to SEQ ID NO:25 or 27;
(ii) a first nucleotide sequence comprising a promoter, optionally comprising or consisting of a sequence having at least 80% sequence identity to SEQ ID NO:16;
(iii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5;
(iv) a post-transcription regulatory element, preferably a WPRE, optionally comprising or consisting of a sequence having at least 80% sequence identity to SEQ ID NO:22;
(v) a polyadenylation signal sequence, optionally comprising or consisting of a sequence having at least 80% sequence identity to SEQ ID NO:20;
(vi) a stuffer sequence, optionally comprising or consisting of a sequence having at least 80% sequence identity to SEQ ID NO:23; and
(vii) an ITR, optionally comprising or consisting of a sequence having at least 80% sequence identity to SEQ ID NO:27 or 25.

19. A polynucleotide according to any one of claims 1 to 16, wherein the polynucleotide comprises or consists of:
(i) an ITR, optionally comprising or consisting of a sequence having at least 80% sequence identity to SEQ ID NO:25 or 27;
(ii) a first nucleotide sequence comprising a promoter, optionally comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5;
(iv) a post-transcription regulatory element, preferably a WPRE, optionally comprising or consisting of a sequence having at least 80% sequence identity to SEQ ID NO:22;
(v) a polyadenylation signal sequence, optionally comprising or consisting of a sequence having at least 80% sequence identity to SEQ ID NO:20; and
(vi) an ITR, optionally comprising or consisting of a sequence having at least 80% sequence identity to SEQ ID NO:27 or 25.

20. A polynucleotide according to any one of claims 1 to 16, wherein the polynucleotide comprises or consists of:
(i) an ITR, optionally comprising or consisting of a sequence having at least 80% sequence identity to SEQ ID NO:25 or 27;
(ii) a first nucleotide sequence comprising a promoter, optionally comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 80% sequence identity to SEQ ID NO:6 or 5;
(iv) a post-transcription regulatory element, preferably a WPRE, optionally comprising or consisting of a sequence having at least 80% sequence identity to SEQ ID NO:22;
(v) a polyadenylation signal sequence, optionally comprising or consisting of a sequence having at least 80% sequence identity to SEQ ID NO:20;
(vi) a stuffer sequence, optionally comprising or consisting of a sequence having at least 80% sequence identity to SEQ ID NO:23; and
(vii) an ITR, optionally comprising or consisting of a sequence having at least 80% sequence identity to SEQ ID NO:27 or 25.

21. A polynucleotide according to any one of claims 1 to 16, wherein the polynucleotide comprises or consists of:
(i) an ITR, optionally comprising or consisting of a sequence having at least 80% sequence identity to SEQ ID NO:25 or 27;
(ii) a first nucleotide sequence comprising a promoter, optionally comprising or consisting of a sequence having at least 80% sequence identity to SEQ ID NO:15;
(iii) a second nucleotide sequence operably linked to the first nucleotide sequence, the second nucleotide sequence comprising a transgene, and wherein the transgene comprises or consists of a nucleotide sequence having at least 90% sequence identity to SEQ ID NO:6 or 5;
(iv) a polyadenylation signal sequence, optionally comprising or consisting of a sequence having at least 80% sequence identity to SEQ ID NO:20;
(v) a stuffer sequence, optionally comprising or consisting of a sequence having at least 80% sequence identity to SEQ ID NO:23; and
(vi) an ITR, optionally comprising or consisting of a sequence having at least 80% sequence identity to SEQ ID NO:27 or 25.

22. A polynucleotide according to any one of claims 1 to 16, wherein the polynucleotide comprises or consists of a sequence having at least 80% sequence identity to one or more of SEQ ID NO:29, 30, 31, 32 or 33.

23. A vector or plasmid comprising a polynucleotide according to any one of claims 1-22.

24. A recombinant AAV (rAAV) comprising:
(i) A polynucleotide according to any one of claims 1-22; and
(ii) At least one AAV capsid protein.

25. A rAAV according to claim 24, wherein the rAAV is a self-complementary AAV (scAAV).

26. A rAAV according to claim 24 or claim 25, wherein
(i) the at least one AAV capsid protein has a tropism for eye cells and/or kidney cells; and/or
(ii) wherein the at least one AAV capsid protein is selected from AAV1, AAV2, AAV3, AAV4, AAV5, AAV6, AAB7, AAV8, AAV9, AAV10, AAV11, AAV12, AAV-DJ, AAV-DJ/8, AAV-rh10, AAVrh.39, AAV-retro, AAV-PHP.B, AAV8-PHP.eB, AAV-PHP.S, AAV-Anc80, AAV2.5, R100, AAV2.7m8, AAV-LK05 and AAVtYF, or a variant or hybrid thereof.

27. A rAAV according to any one of claims 24 to 26, wherein the at least one capsid protein is selected from AAV2 and AAV8.

28. A rAAV according to any one of claims 24 to 27, wherein the polynucleotide comprises at least one AAV2 ITR and the at least one capsid protein is selected from AAV2 and AAV8.

29. A rAAV according to any one of claims 24 to 28, wherein the rAAV is AAV2/2, AAV2/8, AAV2/1, AAV2/6, AAV2/5, AAV2/7, AAV2/9, rAAV2/8 Y733F, rAAV2/2 Y444F, or rAAV2/2 (Y252, 272, 444, 500, 704, 730F).

30. A pharmaceutical composition comprising the polynucleotide, vector, plasmid or rAAV according to any one of claims 1 to 29, and a pharmaceutically acceptable excipient.

31. A cell comprising or expressing the polynucleotide, vector, plasmid or rAAV according to any one of claims 1 to 29.

32. A cell according to claim 31, wherein the cell is a human cell, optionally a cell of the eye or kidney.

33. A polynucleotide, vector, plasmid, rAAV, composition or cell according to any one of claims 1 to 32 for use as a medicament.

34. A polynucleotide, vector, plasmid, rAAV, composition or cell according to any one of claims 1 to 32 for use in a method of treating a complement-related disorder in a subject.

35. A method of treating a complement-related disorder in a subject, the method comprising administering a therapeutically effective amount of a polynucleotide, vector, plasmid, rAAV, composition or cell according to any one of claims 1 to 32.

36. Use of a polynucleotide, vector, plasmid, rAAV, composition or cell according to any one of claims 1 to 32 in the manufacture of a medicament for treating or preventing a complement-related disorder in a subject.

37. A polynucleotide, vector, plasmid, rAAV, composition or cell for use, a method, or use according to any one of claims 34 to 36, wherein the treatment comprises expressing the transgene in at least one cell of the subject.

38. A polynucleotide, vector, plasmid, rAAV, composition or cell for use, a method, or use according to claim 37, wherein the at least one cell is a cell of the eye, kidney or CNS.

39. A polynucleotide, vector, plasmid, rAAV, composition or cell for use, a method, or use according to any one of claims 34 to 38, wherein the polynucleotide, vector, plasmid, rAAV, composition or cell is administered by subretinal, intraocular, intravitreal, intraconjunctival, suprachoroidal, choroidal, or intravenous administration.

40. A polynucleotide, vector, plasmid, rAAV, composition or cell for use, a method, or use according to any one of claims 34 to 39, wherein the complement-related disorder is selected from macular degeneration, Age-related Macular Degeneration (AMD), Geographic Atrophy (‘dry’ or non-exudative AMD), early AMD, early onset macular degeneration (EOMD), intermediate AMD, late/advanced AMD, ‘wet’ (neovascular or exudative) AMD, choroidal neovascularisation (CNV), retinal dystrophy, glaucoma (open-angle or closed-angle), neuromyelitis optica (neuromyelitis optica spectrum disorder (NMOSD)), diabetic retinopathy, Stargardt disease, autoimmune uveitis, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), glomerular diseases, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), ANCA vasculitis, autoimmune hemolytic anemia (AIHA), systemic lupus erythematosus (SLE), cutaneous lupus erythematosus (CLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, skin diseases e.g. inflammatory skin diseases, atherosclerosis, inflammatory disease, inflammatory bowel disease (IBD), autoimmune disease, neurodegeneration/neurodegenerative disease, dementia, frontotemporal dementia, multiple sclerosis (MS), stroke, Parkinson's disease, Alzheimer's disease, Lewy body disease, Amyotrophic lateral sclerosis (ALS), Huntington's disease, epilepsy, schizophrenia, acute brain trauma e.g. traumatic brain injury, neonatal hypoxic ischemic encephalopathy (HIE), myasthenia gravis (MG), Guillain-Barré syndrome (GBS), prion diseases, cancer, lung cancer, glioblastoma multiforme (GBM), an infectious disease, insulin resistance, diabetes, SARS-COV-2 infection and/or COVID-19.

41. A polynucleotide, vector, plasmid, rAAV, composition or cell for use, a method, or use according to any one of claims 34 to 40, wherein the subject has been determined to have, or be at risk of, a complement-related disorder.

42. A polynucleotide, vector, plasmid, rAAV, composition or cell for use, a method, or use according to any one of claims 34 to 40, wherein, prior to the administration of the polynucleotide, vector, plasmid, rAAV, composition or cell, the method comprises:
(a) determining the level of one or more of FHR1, FHR2, FHR3, FHR4 and/or FHR5 in a sample obtained from the subject; and
(b) determining that the subject has or is likely to develop a complement-related disorder if the level of the protein determined in (a) is elevated as compared to the level of that protein in a sample from a control subject that does not have a complement-related disorder or a control subject that has a complement-related disorder that is not associated with elevated levels of said FHR protein(s).

43. A method for expressing a transgene in a cell, the method comprising introducing a polynucleotide, vector, plasmid, rAAV, or composition according to any one of claims 1 to 30 into the cell.

44. A method according to claim 43, wherein the cell is an eye cell, a kidney cell, or a cell of the CNS.


SEQUENCE LISTING (41 sequences)

SEQ ID NO: 1 - protein, 2039 aa, Homo sapiens
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDALIVGTLSGTIFFILLIIFLSWIILKHRKGNNAHENPKEVAIHLHSQGGSSVHPRTLQTNEENSRVLP

SEQ ID NO: 2 - protein, 194 aa, synthetic construct
  VARIANT 111: note = X can be A or T
  VARIANT 157: note = X can be P or L
  VARIANT 160: note = X can be G or R
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNXAHWSTKPPICQRIPCGLPPTIANGDFISTNRENFHYGSVVTYRCNXGSXGRKVFELVGEPSIYCTSNDDQVGIWSGPAPQCII

SEQ ID NO: 3 - protein, 194 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCII

SEQ ID NO: 4 - protein, 194 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 5 - DNA, 582 bp, synthetic construct
  source 1..582: mol_type = other DNA
ggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatc

SEQ ID NO: 6 - DNA, 636 bp, synthetic construct
  source 1..636: mol_type = other DNA
atgagactgctggccaagatcatctgcctgatgctgtgggccatctgcgtggccggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatc

SEQ ID NO: 7 - DNA, 582 bp, synthetic construct
  source 1..582: mol_type = other DNA
ggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattata

SEQ ID NO: 8 - DNA, 636 bp, synthetic construct
  source 1..636: mol_type = other DNA
atgagacttctagcaaagattatttgccttatgttatgggctatttgtgtagcaggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattata

SEQ ID NO: 9 - protein, 18 aa, Homo sapiens
MRLLAKIICLMLWAICVA

SEQ ID NO: 10 - DNA, 54 bp, synthetic construct
  source 1..54: mol_type = other DNA
atgagacttctagcaaagattatttgccttatgttatgggctatttgtgtagca

SEQ ID NO: 11 - DNA, 54 bp, synthetic construct
  source 1..54: mol_type = other DNA
atgagactgctggccaagatcatctgcctgatgctgtgggccatctgcgtggcc

SEQ ID NO: 12 - protein, 212 aa, Homo sapiens
MRLLAKIICLMLWAICVAGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCII

SEQ ID NO: 13 - protein, 212 aa, Homo sapiens
MRLLAKIICLMLWAICVAGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 14 - DNA, 798 bp, synthetic construct
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 15 - DNA, 1624 bp, synthetic construct
  source 1..1624: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattagatctttgtcgatcctaccatccactc

SEQ ID NO: 16 - DNA, 962 bp, synthetic construct
  source 1..962: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattagatctttgtcgatcctaccatccactc

SEQ ID NO: 17 - DNA, 661 bp, synthetic construct
  source 1..661: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggccccccccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcggtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 18 - DNA, 1179 bp, Homo sapiens
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 19 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 20 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 477 bp, Homo sapiens
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 22 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 23 - DNA, 1267 bp, synthetic construct
  source 1..1267: mol_type = other DNA
tccacaaggtgccccaggtggactgggctggggctttgaggccttcagggttggatggccatcttgcgtatttgtgtgggatatgcacacacaggcagcacatgcgcaggtgtgtgggcacctgtgtgtctgtgcaaatgccctgaggtgggaatgagcttggtgtgcatcaggcacagccagccagtgtggctgcagcaaaacacacagggaaagaatggagggggcatcaatcactgcttcagtaaatttttattgagcgccttctacgagaacacaagaggagcttccattctgaggatatcctgtataatttcaagtagtgataagtgctctctagaaatatcaagcaaggtgaggagacacagagcaccggtggcagtggggctctatttccaggttggatggttgggaacatcctttctaaagggaacctggagtgggaaggaaccatgcaggtatctcaggaagagcttcctccaggcaggaagatcagcaggtggaaaggccctggagccaccattcagtaaacatcatttgagcatctctaccagctaggttccattatgggaatgggaatatggtggtggacagggctgcctggtcccttccatacttctcacactagggtggttgagagagcttgggaggggctgagaacactgcctagcccagaggacctgagcttagtgtgtagacattgctgctgttactgcctttgtcattgtattagacagagttttgctcttcttacccaggctggagtgcaatggcgtgatctcagctcactgcaacctccacctcctgggttcaagcgattctcctgcctcagcctcctgagtagctgggattacaggcacccgcaccacgcctggataatttttttgtatttttagtagagacagggtttcaccatgttggccaggctggtctcgaactcctgaccttaggtgatccacctgcctcgacttcccaaagtgctgggattataggcatgagccactgcgcccagtgattatagaaagttaaaggcacatggcaatgcacacgcctatctacgtcttccctgccaaagcaaagggcagcctctgggctcactttcttgcgtttctacttccaaaaggcagtcagaactggcagggccttggagaccacttcatccacctcctagggtccctatgggagagttgaggtccagagcagggaagggtcctgacaggctctgaccagggcctctgatccctacaaacccccaatcggtggcgat

SEQ ID NO: 24 - DNA, 1065 bp, synthetic construct
  source 1..1065: mol_type = other DNA
acctggggcttgcctgggccagggagcccaggactggggtgaggactcaggggagcagggagaccacgtcccaagatgcctgtaaaactgaaaccacctggccattctccaggttgagccagaccaatttgatggcagatttagcaaataaaaatacaggacacccagttaaatgtgaatttcagatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaa

SEQ ID NO: 25 - DNA, 165 bp, synthetic construct
  source 1..165: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctac

SEQ ID NO: 26 - DNA, 144 bp, synthetic construct
  source 1..144: mol_type = other DNA
gggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 27 - DNA, 165 bp, synthetic construct
  source 1..165: mol_type = other DNA
gtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 28 - DNA, 113 bp, synthetic construct
  source 1..113: mol_type = other DNA
ccactccctctctgcgcgctcgctcgctcactgaggccgggcgaccaaaggtcgcccgacgcccgggctttgcccgggcggcctcagtgagcgagcgagcgcgcagagaggga

SEQ ID NO: 29 - DNA, 1911 bp, synthetic construct
  source 1..1911: mol_type = other DNA
gggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgaccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagatctttgtcgatcctaccatccactcgacacacccgccagcggccgccgccaccatgagactgctggccaagatcatctgcctgatgctgtgggccatctgcgtggccggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatctaactgactgataagatacattgatgagtttggacaaaccacaactagaatgcagtgaaaaaaatgctttatttgtgaaatttgtgatgctattgctttatttgtaaccattataagctgcaataaacaagttctatccactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 30 - DNA, 4100 bp, synthetic construct
  source 1..4100: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattagatctttgtcgatcctaccatccactcgacacacccgccagcggccgccgccaccatgagactgctggccaagatcatctgcctgatgctgtgggccatctgcgtggccggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatctaaactgctagcttgactgactgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgatatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctatccacatccacaaggtgccccaggtggactgggctggggctttgaggccttcagggttggatggccatcttgcgtatttgtgtgggatatgcacacacaggcagcacatgcgcaggtgtgtgggcacctgtgtgtctgtgcaaatgccctgaggtgggaatgagcttggtgtgcatcaggcacagccagccagtgtggctgcagcaaaacacacagggaaagaatggagggggcatcaatcactgcttcagtaaatttttattgagcgccttctacgagaacacaagaggagcttccattctgaggatatcctgtataatttcaagtagtgataagtgctctctagaaatatcaagcaaggtgaggagacacagagcaccggtggcagtggggctctatttccaggttggatggttgggaacatcctttctaaagggaacctggagtgggaaggaaccatgcaggtatctcaggaagagcttcctccaggcaggaagatcagcaggtggaaaggccctggagccaccattcagtaaacatcatttgagcatctctaccagctaggttccattatgggaatgggaatatggtggtggacagggctgcctggtcccttccatacttctcacactagggtggttgagagagcttgggaggggctgagaacactgcctagcccagaggacctgagcttagtgtgtagacattgctgctgttactgcctttgtcattgtattagacagagttttgctcttcttacccaggctggagtgcaatggcgtgatctcagctcactgcaacctccacctcctgggttcaagcgattctcctgcctcagcctcctgagtagctgggattacaggcacccgcaccacgcctggataatttttttgtatttttagtagagacagggtttcaccatgttggccaggctggtctcgaactcctgaccttaggtgatccacctgcctcgacttcccaaagtgctgggattataggcatgagccactgcgcccagtgattatagaaagttaaaggcacatggcaatgcacacgcctatctacgtcttccctgccaaagcaaagggcagcctctgggctcactttcttgcgtttctacttccaaaaggcagtcagaactggcagggccttggagaccacttcatccacctcctagggtccctatgggagagttgaggtccagagcagggaagggtcctgacaggctctgaccagggcctctgatccctacaaacccccaatcggtggcgatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 31 - DNA, 3501 bp, synthetic construct
  source 1..3501: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattagatctttgtcgatcctaccatccactcgacacacccgccagcggccgccgccaccatgagactgctggccaagatcatctgcctgatgctgtgggccatctgcgtggccggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatctaaactgctagcttgactgactgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgatatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctatccacatccactgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 32 - DNA, 4762 bp, synthetic construct
  source 1..4762: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattagatctttgtcgatcctaccatccactcgacacacccgccagcggccgccgccaccatgagactgctggccaagatcatctgcctgatgctgtgggccatctgcgtggccggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatctaaactgctagcttgactgactgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgatatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctatccacatccacaaggtgccccaggtggactgggctggggctttgaggccttcagggttggatggccatcttgcgtatttgtgtgggatatgcacacacaggcagcacatgcgcaggtgtgtgggcacctgtgtgtctgtgcaaatgccctgaggtgggaatgagcttggtgtgcatcaggcacagccagccagtgtggctgcagcaaaacacacagggaaagaatggagggggcatcaatcactgcttcagtaaatttttattgagcgccttctacgagaacacaagaggagcttccattctgaggatatcctgtataatttcaagtagtgataagtgctctctagaaatatcaagcaaggtgaggagacacagagcaccggtggcagtggggctctatttccaggttggatggttgggaacatcctttctaaagggaacctggagtgggaaggaaccatgcaggtatctcaggaagagcttcctccaggcaggaagatcagcaggtggaaaggccctggagccaccattcagtaaacatcatttgagcatctctaccagctaggttccattatgggaatgggaatatggtggtggacagggctgcctggtcccttccatacttctcacactagggtggttgagagagcttgggaggggctgagaacactgcctagcccagaggacctgagcttagtgtgtagacattgctgctgttactgcctttgtcattgtattagacagagttttgctcttcttacccaggctggagtgcaatggcgtgatctcagctcactgcaacctccacctcctgggttcaagcgattctcctgcctcagcctcctgagtagctgggattacaggcacccgcaccacgcctggataatttttttgtatttttagtagagacagggtttcaccatgttggccaggctggtctcgaactcctgaccttaggtgatccacctgcctcgacttcccaaagtgctgggattataggcatgagccactgcgcccagtgattatagaaagttaaaggcacatggcaatgcacacgcctatctacgtcttccctgccaaagcaaagggcagcctctgggctcactttcttgcgtttctacttccaaaaggcagtcagaactggcagggccttggagaccacttcatccacctcctagggtccctatgggagagttgaggtccagagcagggaagggtcctgacaggctctgaccagggcctctgatccctacaaacccccaatcggtggcgatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 33 - DNA, 4164 bp, synthetic construct
  source 1..4164: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattagatctttgtcgatcctaccatccactcgacacacccgccagcggccgccgccaccatgagactgctggccaagatcatctgcctgatgctgtgggccatctgcgtggccggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatctaaactgctagcttgactatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctatccacatccacaaggtgccccaggtggactgggctggggctttgaggccttcagggttggatggccatcttgcgtatttgtgtgggatatgcacacacaggcagcacatgcgcaggtgtgtgggcacctgtgtgtctgtgcaaatgccctgaggtgggaatgagcttggtgtgcatcaggcacagccagccagtgtggctgcagcaaaacacacagggaaagaatggagggggcatcaatcactgcttcagtaaatttttattgagcgccttctacgagaacacaagaggagcttccattctgaggatatcctgtataatttcaagtagtgataagtgctctctagaaatatcaagcaaggtgaggagacacagagcaccggtggcagtggggctctatttccaggttggatggttgggaacatcctttctaaagggaacctggagtgggaaggaaccatgcaggtatctcaggaagagcttcctccaggcaggaagatcagcaggtggaaaggccctggagccaccattcagtaaacatcatttgagcatctctaccagctaggttccattatgggaatgggaatatggtggtggacagggctgcctggtcccttccatacttctcacactagggtggttgagagagcttgggaggggctgagaacactgcctagcccagaggacctgagcttagtgtgtagacattgctgctgttactgcctttgtcattgtattagacagagttttgctcttcttacccaggctggagtgcaatggcgtgatctcagctcactgcaacctccacctcctgggttcaagcgattctcctgcctcagcctcctgagtagctgggattacaggcacccgcaccacgcctggataatttttttgtatttttagtagagacagggtttcaccatgttggccaggctggtctcgaactcctgaccttaggtgatccacctgcctcgacttcccaaagtgctgggattataggcatgagccactgcgcccagtgattatagaaagttaaaggcacatggcaatgcacacgcctatctacgtcttccctgccaaagcaaagggcagcctctgggctcactttcttgcgtttctacttccaaaaggcagtcagaactggcagggccttggagaccacttcatccacctcctagggtccctatgggagagttgaggtccagagcagggaagggtcctgacaggctctgaccagggcctctgatccctacaaacccccaatcggtggcgatgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 34 - DNA, 2452 bp, synthetic construct
  source 1..2452: mol_type = other DNA
gggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggccccccccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcggtaagtatcaaggttacaagacaggtttaaggagaccaatagaaactgggcttgtcgagacagagaagactcttgcgtttctgataggcacctattggtcttactgacatccactttgcctttctctccacaggcggccgccgccaccatgagactgctggccaagatcatctgcctgatgctgtgggccatctgcgtggccggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatctaaactgctagcttgactgactgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgatatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctatccacaactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 35 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 36 - protein, 235 aa, Homo sapiens
IVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVG

SEQ ID NO: 37 - DNA, 585 bp, synthetic construct
  source 1..585: mol_type = other DNA
ggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatctaa

SEQ ID NO: 38 - DNA, 639 bp, synthetic construct
  source 1..639: mol_type = other DNA
atgagactgctggccaagatcatctgcctgatgctgtgggccatctgcgtggccggacattgtcaggcccctgaccacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatctaa

SEQ ID NO: 39 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 40 - protein, 449 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 41 - protein, 6 aa, synthetic construct
HHHHHH